(19) United States
(12) Patent Application Publication
(10) Pub. No.: US 2025/0195639 A1
(43) Pub. Date: Jun. 19, 2025

(54) MULTI-EPITOPE MRNA SARS-COV-2 VACCINE FOR BOOSTING IMMUNITY THROUGH THE ACTIVATION OF CD4 AND CD8 T CELLS AS WELL AS B LYMPHOCYTES

(71) Applicant: THE REGENTS OF THE UNIVERSITY OF CALIFORNIA, Oakland, CA (US)

(72) Inventors: Andre E. NEL, Oakland, CA (US); Tian XIA, Oakland, CA (US); Jeffery F. MILLER, Oakland, CA (US); Xiang WANG, Oakland, CA (US)

(73) Assignee: THE REGENTS OF THE UNIVERSITY OF CALIFORNIA, Oakland, CA (US)

(21) Appl. No.: 18/702,692

(22) PCT Filed: Oct. 19, 2022
(86) PCT No.: PCT/US2022/047175

§ 371 Date: Apr. 18, 2024

Related U.S. Application Data
(60) Provisional application No. 63/257,964, filed on Oct. 20, 2021.

(51) Int. Cl. A61K 39/215; A61K 9/51; A61K 39/00

(52) U.S. Cl. CPC A61K 39/215; A61K 9/5123; A61K 9/513; A61K 2039/53

(57) ABSTRACT

In various embodiments immunogenic nanoparticles are provided that are capable of raising an immune response directed against SARS-CoV-2. In certain embodiments the immunogenic nanoparticles comprise mRNA multi-epitope vaccines that can be used in combination with or independent of other covid-19 vaccines (e.g., the spike protein mRNA vaccine(s)) to invoke a strong CD8⁺ or CD4⁺ T-cell as well as neutralizing antibody producing B-cell responses. In certain embodiments this vaccine is based on the rational combination of well-conserved T- and B-cell epitopes identified COVID-19 and viral variants.

### CROSS-REFERENCE TO RELATED APPLICATIONS

This application claims benefit of and priority to U.S. Ser. No. 63/257,964, filed on Oct. 20, 2021, which is incorporated herein by reference in its entirety for all purposes.

### STATEMENT OF GOVERNMENTAL SUPPORT

This invention was made with government support under Grant Numbers CA198846 and ES027237, awarded by the National Institutes of Health. The government has certain rights in the invention.

### INCORPORATION BY REFERENCE OF SEQUENCE LISTING PROVIDED AS A TEXT FILE

[Not Applicable]

### BACKGROUND

An effective vaccine for severe acute respiratory syndrome coronavirus 2 (SARS-CoV-2) constitutes a major global health priority (see, e.g., Nathan et al. (2021) Cell 184: 4401-4413). While multiple vaccine candidates have been developed, using diverse platforms to induce neutralizing antibodies against the viral spike protein (see, e.g., Baden et al. (2021) N. Engl. J. Med. 384: 403-427; Folegatti et al. (2020) Lancet, 396: 467-478; Keech et al. (2020) N. Engl. J. Med. 383: 2320-2332; Polack et al. (2020) N. Engl. J. Med., 383: 2603-2615; Sadoff et al. (2021) N. Engl. J. Med. 384: 2187-2201), the emergence of new SARS-Cov-2 strains such as the B.1.1.7 alpha (Tang et al. (2020) J. Infect. 82: e27-e28), B.1.351 beta (Tegally et al. (2021) Nature, 592: 438-443), P.1 gamma (Voloch et al. (2020) medRxiv, 10.1101/2020.12.23.20248598), B.1.617.2 delta variants (Cherian et al. (2021) bioRxiv (2021) 10.1101/2021.04.22.440932) and Omicron variants (Bowen et al. (2022) Science, 0.1126/science.abq0203; //covdb.stanford.edu/variants/omicron/) have raised substantial new concerns as a result of increased transmissibility (see, e.g., Davies et al. (2021) Science, 372, p. eabg3055; Kumar et al. (2021) bioRxiv, 10.1101/2021.04.29.441933), as well as immune escape from convalescent and vaccine-induced antibodies (Garcia-Beltran et al. (2021) Cell, 184: 2523; Hoffmann et al. (2021) bioRxiv, 10.1101/2021.05.04.442663; Madhi et al. (2021) N. Engl. J. Med., 384: 1885-1898; Wang et al. (2021) Nature, 592: 616-622; Wibmer et al. (2021) Nat. Med., 27: 622-625; and the like). The spike protein has different hotspots of mutation or deletion, among which mutations in the receptor binding domain (RDB) in variants of concern (VOC) such as K417N/T, N439K, L452R, Y453F, S477N, E484K, and N501Y have been shown to be responsible for immune escape in the so-called UK (B.1.1.7), South African (B.1.351) and Brazilian (P.1) CoV2 viral variants strains (see, e.g., Focosi & Magg (2021) Rev. Med. Virol. 31(6): e2231). Moreover, the number of mutations has been further increased in the Omicron lineage, leading to additional decline in the efficacy of neutralizing antibodies (see, e.g., Bowen et al. (3033) Science, 0.1126/science.abq0203; Guo et al. (2022) Mol. Biomed. 3(1): 12). Importantly, the mechanism of antibody escape often involves mutation of antigenic B-cell antigenic epitopes in the spike protein, leading to failure to cross-link or bridge adjacent RDBs, an important neutralizing function for all vaccine-induced immunoglobulins (see, e.g., Planas et al. (2021) Nature 596: 276-280; Barnes et al. (2020) Nature Vol 588; Cohen et al. (2022) Science, 377: 618).

While boosting of immunity with the current mRNA vaccines can overcome the decline in neutralizing antibody efficacy against the variant strains, the efficacy decline over time appears to have been much more rapid than the rate of decline in neutralizing antibody effects to the original viral strain. This is compatible with the most recent demonstration that Omicron escapes a majority of the vast range of SARS-CoV-2 neutralizing antibodies that are generated by vaccination or natural infection. This raises an urgent need to develop broadly neutralizing antibodies recognizing epitopes conserved among SARS-CoV-2 variants as well as other sarbecovirus, particularly heavily mutated strains such as Omicron (Barnes et al. (2020) Nature, Vol 588; Cohen et al. (2020) Science, 377: 618). In addition, given that SARS-CoV-2 represents the third coronavirus outbreak in the past 20 years (in addition to SARS-CoV-1 and Middle East respiratory syndrome coronavirus), significant additional concerns have emerged about the possibility of a future pandemic due to the numerous SARS-like coronaviruses identified in viral reservoirs in the bat (see, e.g., Menachery et al. (2015) Nat. Med., 21: 1508-1513, Menachery et al. (2016) Proc. Natl. Acad. Sci. USA, 113: 3048-3053). Thus, while existing vaccines are critical to curtail the ongoing pandemic, new vaccine candidates are urgently needed for enhancing the protection required against viral variants of concern VOC, including emerging coronaviruses. While the vaccine strategy has shifted to vaccination with hybrid spike proteins from the Wuhan virus plus an Omicron variant, efficacy of these vaccines needs to be proven, as compared to using conserved spike and non-spike SARS CoV2 sequences/epitopes, as well as considering conserved sequences from other beta-coronaviruses.

While a large number of T-cell and B-cell epitopes have been delineated in the literature that could be used to serve as potential vaccine candidates for incorporating into multi-epitope vaccines, there has been no well-designed paradigm to date for epitope selection, epitope combinations and routing towards Type I and type II major histocompatibility complexes (MHC) or extracellular expression for recognition by the B-cell antigen receptor.

The search for epitopes is facilitated by consideration of sequences beyond antibody binding sites from the perspective that CD4 positive T cells (e.g., follicular helper T cells) play a key role in immunoglobulin class switching, hypermutation and memory B-cell development in lymph node germinal centers. Moreover, it is important to consider the contribution of activating CD8 positive T-cells, which are critical for cytotoxic killing and viral clearance, in addition to giving rise to development of tissue-resident T-cells and memory T cells. Apart from accomplishing appropriate epitope selection, and other key design feature involves multi-epitope integration into nucleic acid factors that contain the appropriate instruction for routing the epitopes to the correct major histocompatibility complexes (MHC) or for exterior display to mount B-cell responses, following epitope recognition by the B-cell antigen receptor.

### SUMMARY

As described herein, we illustrate the design of mRNA multi-epitope vaccines that can be used in combination with or independent of other covid-19 vaccines (e.g., the spike protein mRNA vaccine(s)) to invoke a strong CD8⁺ T-cell response. In certain embodiments this vaccine is based on the rational combination of well-conserved T-cell epitopes identified COVID-19 and viral variants. These epitopes have been initiated by Nathan et al. (2021) Cell, 184: 4401-4413), using structure-based network analysis to identify highly conserved CD8⁺ epitopes that could prevent viral escape from the cytotoxic T-cells, thereby improving the ability of the vaccine to control current and future coronavirus infections. We have also identified additional epitopes for incorporation into multi-epitope vaccines. In this regard we noted that the search for epitopes should also consider sequences beyond antibody binding sites from the perspective that CD4 positive T cells (e.g., follicular helper T cells) play a key role in immunoglobulin class switching, hypermutation and memory B-cell development in lymph node germinal centers. Moreover, it was important to consider the contribution of activating CD8 positive T-cells, which are critical for cytotoxic killing and viral clearance, in addition to giving rise to development of tissue-resident T-cells and memory T cells. Apart from accomplishing appropriate epitope selection, and other key vaccine design features, we also demonstrate folding together the multi-epitope nucleic acid constructs to allow epitope processing and separation, in addition to incorporating appropriate instructions for routing the epitopes to the correct major histocompatibility complexes (MHC) or for exterior display to mount B-cell responses

Accordingly, various embodiments provided herein may include, but need not be limited to, one or more of the following:

Embodiment 1: An immunogenic nanoparticle comprising:
a nanoparticle comprising one or more lipid(s) and/or one or more biocompatible polymer(s); and
a nucleic acid encapsulated within or attached to said nanoparticle where said nucleic acid encodes a plurality of covid-19 peptide antigens, wherein said covid-19 peptide antigens comprises one or more CD8+ T cell epitopes and/or one or more CD4+T-follicular helper epitopes and/or one or more CD4 T cell epitopes.

Embodiment 2: The immunogenic nanoparticle of embodiment 1, wherein said plurality of covid-19 peptide antigens comprises one or more CD8+ T cell epitopes.

Embodiment 3: The immunogenic nanoparticle of embodiment 2, wherein said plurality of covid-19 peptide antigens comprises one or more CD8+ T cell epitopes independent selected from the epitopes shown in Table 5 and/or Table 2 and/or Table 33.

Embodiment 4: The immunogenic nanoparticle of embodiment 3, wherein said plurality of covid-19 CD8+ peptide antigens comprises at least 1, or at least 2, or at least 3, or at least 4, or at least 5, or at least 6, or at least 7, or at least 8, or at least 9, or at least 10, or at least 11, or at least 12, or at least 13, or at least 14 amino acid sequences selected from the group consisting of RGVYYPDKVFRSSV (SEQ ID NO:34), KGIYQTSNFRVQPTESIVRF (SEQ ID NO:35), KLNDLCFTNVY (SEQ ID NO:36), FELLHAPATV (SEQ ID NO:37), TSNEVAVLYQDVNCTEV (SEQ ID NO:38), TEILPVSMTKTSVDCTMY (SEQ ID NO:39), PLLTDEMIAQYTSAL (SEQ ID NO:40), YRFNGIGV (SEQ ID NO:41), ALNTLVKQLSSNFGAISSVLNDILSRL (SEQ ID NO:42), KRVDFCGKGYHLMSFPQSAPHGVVF (SEQ ID NO:43), GVFVSNGTHW (SEQ ID NO:44), NPLLYDANYFLCWHTNCYDYCIPYNSVTSSI (SEQ ID NO:45), RLFARTRSMWSFNPETNILLNVPLHGTILTR PLLESELVIGAVILRGHLRIAGHHL (SEQ ID NO:46), NSSPDDQIGYY (SEQ ID NO:47), and RRGPEQTQGNFGDQELIRQGTDYKHWPQI AQFAPSASAFFGM (SEQ ID NO:48).

Embodiment 5: The immunogenic nanoparticle according to any one of embodiments 3-4, wherein said plurality of peptide antigens comprises a peptide antigen comprising the amino acid sequence RGVYYPDKVFRSSV (SEQ ID NO:34).

Embodiment 6: The immunogenic nanoparticle according to any one of embodiments 3-5, wherein said plurality of peptide antigens comprises a peptide antigen comprising the amino acid sequence KGIYQTSNFRVQPTESIVRF (SEQ ID NO:35).

Embodiment 7: The immunogenic nanoparticle according to any one of embodiments 3-6, wherein said plurality of peptide antigens comprises a peptide antigen comprising the amino acid sequence KLNDLCFTNVY (SEQ ID NO:36).

Embodiment 8: The immunogenic nanoparticle according to any one of embodiments 3-7, wherein said plurality of peptide antigens comprises a peptide antigen comprising the amino acid sequence FELLHAPATV (SEQ ID NO:37).

Embodiment 9: The immunogenic nanoparticle according to any one of embodiments 3-8, wherein said plurality of peptide antigens comprises a peptide antigen comprising the amino acid sequence TSNEVAVLYQDVNCTEV (SEQ ID NO:38).

Embodiment 10: The immunogenic nanoparticle according to any one of embodiments 3-9, wherein said plurality of peptide antigens comprises a peptide antigen comprising the amino acid sequence TEILPVSMTKTSVDCTMY (SEQ ID NO:39).

Embodiment 11: The immunogenic nanoparticle according to any one of embodiments 3-10, wherein said plurality of peptide antigens comprises a peptide antigen comprising the amino acid sequence PLLTDEMIAQYTSAL (SEQ ID NO:40).

Embodiment 12: The immunogenic nanoparticle according to any one of embodiments 3-11, wherein said plurality of peptide antigens comprises a peptide antigen comprising the amino acid sequence YRFNGIGV (SEQ ID NO:41).

Embodiment 13: The immunogenic nanoparticle according to any one of embodiments 3-12, wherein said plurality of peptide antigens comprises a peptide antigen comprising the amino acid sequence ALNTLVKQLSSNFGAISSVLNDILSRL (SEQ ID NO:42).

Embodiment 14: The immunogenic nanoparticle according to any one of embodiments 3-13, wherein said plurality of peptide antigens comprises a peptide antigen comprising the amino acid sequence KRVDFCGKGYHLMSFPQSAPHGVVF (SEQ ID NO:43).

Embodiment 15: The immunogenic nanoparticle according to any one of embodiments 3-14, wherein said plurality of peptide antigens comprises a peptide antigen comprising the amino acid sequence GVFVSNGTHW (SEQ ID NO:44).

Embodiment 16: The immunogenic nanoparticle according to any one of embodiments 3-15, wherein said plurality of peptide antigens comprises a peptide antigen comprising the amino acid sequence NPLLYDANYFLCWHTNCYDYCIPYNSVTSSI (SEQ ID NO:45).

Embodiment 17: The immunogenic nanoparticle according to any one of embodiments 3-16, wherein said plurality of peptide antigens comprises a peptide antigen comprising the amino acid sequence RLFARTRSMWSFNPETNILLNVPLHGTILTR PLLESELVIGAVILRGHLRIAGHHL (SEQ ID NO:46).

Embodiment 18: The immunogenic nanoparticle according to any one of embodiments 3-17, wherein said plurality of peptide antigens comprises a peptide antigen comprising the amino acid sequence NSSPDDQIGYY (SEQ ID NO:47).

Embodiment 19: The immunogenic nanoparticle according to any one of embodiments 3-18, wherein said plurality of peptide antigens comprises a peptide antigen comprising the amino acid sequence RRGPEQTQGNFGDQELIRQGTDY KHWPQI AQFAPSASAFFGM (SEQ ID NO:48).

Embodiment 20: The immunogenic nanoparticle of embodiment 3, wherein said plurality of peptide antigens comprises the following amino acid sequences:

(SEQ ID NO: 34)
RGVYYPDKVFRSSV,
(SEQ ID NO: 35)
KGIYQTSNFRVQPTESIVRF,
(SEQ ID NO: 36)
KLNDLCFTNVY,
(SEQ ID NO: 37)
FELLHAPATV,
(SEQ ID NO: 38)
TSNEVAVLYQDVNCTEV,
(SEQ ID NO: 39)
TEILPVSMTKTSVDCTMY,
(SEQ ID NO: 40)
PLLTDEMIAQYTSAL,
(SEQ ID NO: 41)
YRENGIGV,
(SEQ ID NO: 42)
ALNTLVKQLSSNFGAISSVLNDILSRL,
(SEQ ID NO: 43)
KRVDFCGKGYHLMSFPQSAPHGVVF,
(SEQ ID NO: 44)
GVFVSNGTHW,
(SEQ ID NO: 45)
NPLLYDANYFLCWHTNCYDYCIPYNSVTSSI,
(SEQ ID NO: 46)
RLFARTRSMWSFNPETNILLNVPLHGTILTR
PLLESELVIGAVILRGHLRIAGHHL,
(SEQ ID NO: 47)
NSSPDDQIGYY,
and
(SEQ ID NO: 48)
RRGPEQTQGNFGDQELIRQGTDYKHWPQI AQFAPSASAFFGM.

Embodiment 21: The immunogenic nanoparticle according to any one of embodiments 3-20, wherein said plurality of covid-19 CD8+ peptide antigens comprises at least 1, or at least 2, or at least 3, or at least 4, or at least 5, or at least 6, or at least 7, or at least 8, or at least 9, or at least 10, or at least 11, or 12, amino acid sequences independently selected from the group consisting of LSFELLHAPATVCGP (SEQ ID NO:9), VNFNFNGLKGTGVLT (SEQ ID NO:10), PLLTDEMIAQYTSAL (SEQ ID NO:11), KQLSSNFGAISSVLN (SEQ ID NO:12), FAMQMAYRFNGIGVT (SEQ ID NO:13), PTNFTISVTTEILPV (SEQ ID NO:14), FCGKGYHLMSFPQSA (SEQ ID NO:15), KLNDLCFTNVY (SEQ ID NO:16), TSNFRVQPTESIVRF (SEQ ID NO:17), NEVAVLYQDVNCTEV (SEQ ID NO:18), RGVYYPDKVFRSSV (SEQ ID NO:19), and GVFVSNGTHW (SEQ ID NO:20).

Embodiment 22: The immunogenic nanoparticle of embodiment 21, wherein said plurality of covid-19 peptide antigens comprises the amino acid sequence LSFELLHAPATVCGP (SEQ ID NO:9).

Embodiment 23: The immunogenic nanoparticle according to any one of embodiments 21-22, wherein said plurality of covid-19 peptide antigens comprises the amino acid sequence.

Embodiment 24: The immunogenic nanoparticle according to any one of embodiments 21-23, wherein said plurality of covid-19 peptide antigens comprises the amino acid sequence VNFNFNGLKGTGVLT (SEQ ID NO:10).

Embodiment 25: The immunogenic nanoparticle according to any one of embodiments 21-24, wherein said plurality of covid-19 peptide antigens comprises the amino acid sequence PLLTDEMIAQYTSAL (SEQ ID NO:11).

Embodiment 26: The immunogenic nanoparticle according to any one of embodiments 21-25, wherein said plurality of covid-19 peptide antigens comprises the amino acid sequence KQLSSNFGAISSVLN (SEQ ID NO:12).

Embodiment 27: The immunogenic nanoparticle according to any one of embodiments 21-26, wherein said plurality of covid-19 peptide antigens comprises the amino acid sequence FAMQMAYRFNGIGVT (SEQ ID NO:13).

Embodiment 28: The immunogenic nanoparticle according to any one of embodiments 21-27, wherein said plurality of covid-19 peptide antigens comprises the amino acid sequence PTNFTISVTTEILPV (SEQ ID NO:14).

Embodiment 29: The immunogenic nanoparticle according to any one of embodiments 21-28, wherein said plurality of covid-19 peptide antigens comprises the amino acid sequence FCGKGYHLMSFPQSA (SEQ ID NO:15).

Embodiment 30: The immunogenic nanoparticle according to any one of embodiments 21-29, wherein said plurality of covid-19 peptide antigens comprises the amino acid sequence KLNDLCFTNVY (SEQ ID NO:16).

Embodiment 31: The immunogenic nanoparticle according to any one of embodiments 21-30, wherein said plurality of covid-19 peptide antigens comprises the amino acid sequence TSNFRVQPTESIVRF (SEQ ID NO:17).

Embodiment 32: The immunogenic nanoparticle according to any one of embodiments 21-31, wherein said plurality of covid-19 peptide antigens comprises the amino acid sequence NEVAVLYQDVNCTEV (SEQ ID NO:18).

Embodiment 33: The immunogenic nanoparticle according to any one of embodiments 21-32, wherein said plurality of covid-19 peptide antigens comprises the amino acid sequence RGVYYPDKVFRSSV (SEQ ID NO:19).

Embodiment 34: The immunogenic nanoparticle according to any one of embodiments 21-33, wherein said plurality of covid-19 peptide antigens comprises the amino acid sequence GVFVSNGTHW (SEQ ID NO:20).

Embodiment 35: The immunogenic nanoparticle of embodiment 21, wherein said plurality of covid-19 peptide antigens comprises the amino acid sequences

(SEQ ID NO: 9)
LSFELLHAPATVCGP,
(SEQ ID NO: 10)
VNFNFNGLKGTGVLT,
(SEQ ID NO: 11)
PLLTDEMIAQYTSAL,
(SEQ ID NO: 12)
KQLSSNFGAISSVLN,
(SEQ ID NO: 13)
FAMQMAYRENGIGVT,
(SEQ ID NO: 14)
PTNFTISVTTEILPV,
(SEQ ID NO: 15)
FCGKGYHLMSFPQSA,
(SEQ ID NO: 16)
KLNDLCFTNVY,
(SEQ ID NO: 17)
TSNFRVQPTESIVRF,
(SEQ ID NO: 18)
NEVAVLYQDVNCTEV,
(SEQ ID NO: 19)
RGVYYPDKVFRSSV,
and
(SEQ ID NO: 20)
GVFVSNGTHW.

Embodiment 36: The immunogenic nanoparticle according to any one of embodiments 3-35, wherein said plurality of covid-19 CD8+ peptide antigens comprises at least 1, or at least 2, or at least 3, or at least 4, or at least 5, or at least 6, or at least 7, or at least 8, or at least 9, or at least 10, or at least 11, or at least 12, or at least 13, or at least 14, or 15 amino acid sequences selected from the group consisting of YLATALLTL (SEQ ID NO:243), WLMWLIINL (SEQ ID NO:245), SLPGVFCGV (SEQ ID NO:247), KLSYGIATV (SEQ ID NO:249), LLLDDFVEI (SEQ ID NO:251), FVFLVLLPL (SEQ ID NO:253), ALNTLVKQL (SEQ ID NO:255), RLQSLQTYV (SEQ ID NO:257), FIAGLIAIV (SEQ ID NO:259), FLAFVVFLL (SEQ ID NO:261), FLLNKEMYL (SEQ ID NO:263), IIFWFSLEL (SEQ ID NO:265), YIDIGNYTV (SEQ ID NO:267), YINVFAFPF (SEQ ID NO:269), and NVFAFPFTI (SEQ ID NO:271).

Embodiment 37: The immunogenic nanoparticle of embodiment 36, wherein said plurality of covid-19 CD8+ peptide antigens comprise the amino acid sequence YLATALLTL (SEQ ID NO:243).

Embodiment 38: The immunogenic nanoparticle according to any one of embodiments 36-37, wherein said plurality of covid-19 CD8+ peptide antigens comprise the amino acid sequence WLMWLIINL (SEQ ID NO:245).

Embodiment 39: The immunogenic nanoparticle according to any one of embodiments 36-38, wherein said plurality of covid-19 CD8+ peptide antigens comprise the amino acid sequence SLPGVFCGV (SEQ ID NO:247).

Embodiment 40: The immunogenic nanoparticle according to any one of embodiments 36-39, wherein said plurality of covid-19 CD8+ peptide antigens comprise the amino acid sequence KLSYGIATV (SEQ ID NO:249).

Embodiment 41: The immunogenic nanoparticle according to any one of embodiments 36-40, wherein said plurality of covid-19 CD8+ peptide antigens comprise the amino acid sequence LLLDDFVEI (SEQ ID NO:251).

Embodiment 42: The immunogenic nanoparticle according to any one of embodiments 36-41, wherein said plurality of covid-19 CD8+ peptide antigens comprise the amino acid sequence FVFLVLLPL (SEQ ID NO:253).

Embodiment 43: The immunogenic nanoparticle according to any one of embodiments 36-42, wherein said plurality of covid-19 CD8+ peptide antigens comprise the amino acid sequence ALNTLVKQL (SEQ ID NO:255).

Embodiment 44: The immunogenic nanoparticle according to any one of embodiments 36-43, wherein said plurality of covid-19 CD8+ peptide antigens comprise the amino acid sequence RLQSLQTYV (SEQ ID NO:257).

Embodiment 45: The immunogenic nanoparticle according to any one of embodiments 36-44, wherein said plurality of covid-19 CD8+ peptide antigens comprise the amino acid sequence FIAGLIAIV (SEQ ID NO:259).

Embodiment 46: The immunogenic nanoparticle according to any one of embodiments 36-45, wherein said plurality of covid-19 CD8+ peptide antigens comprise the amino acid sequence FLAFVVFLL (SEQ ID NO:261).

Embodiment 47: The immunogenic nanoparticle according to any one of embodiments 36-46, wherein said plurality of covid-19 CD8+ peptide antigens comprise the amino acid sequence FLLNKEMYL (SEQ ID NO:263).

Embodiment 48: The immunogenic nanoparticle according to any one of embodiments 36-47, wherein said plurality of covid-19 CD8+ peptide antigens comprise the amino acid sequence IIFWFSLEL (SEQ ID NO:265).

Embodiment 49: The immunogenic nanoparticle according to any one of embodiments 36-48, wherein said plurality of covid-19 CD8+ peptide antigens comprise the amino acid sequence YIDIGNYTV (SEQ ID NO:267).

Embodiment 50: The immunogenic nanoparticle according to any one of embodiments 36-49, wherein said plurality of covid-19 CD8+ peptide antigens comprise the amino acid sequence YINVFAFPF (SEQ ID NO:269).

Embodiment 51: The immunogenic nanoparticle according to any one of embodiments 36-50, wherein said plurality of covid-19 CD8+ peptide antigens comprise the amino acid sequence NVFAFPFTI (SEQ ID NO:271).

Embodiment 52: The immunogenic nanoparticle according to any one of embodiments 3-51, wherein said epitopes are each encoded by the corresponding nucleic acid sequence(s) shown in Table 5 and/or Table 2.

Embodiment 53: The immunogenic nanoparticle of embodiment 52, wherein said nucleic acid sequence is codon optimized.

Embodiment 54: The immunogenic nanoparticle of embodiment 53, wherein said nucleic acid sequence is optimized using GenSmart Codon Optimization.

Embodiment 55: The immunogenic nanoparticle of embodiment 54, wherein each epitope is encoded by the corresponding nucleic acid sequence shown in Table 10.

Embodiment 56: The immunogenic nanoparticle of embodiment 55, wherein said nucleic acid comprises the sequence shown in in Table 10.

Embodiment 57: The immunogenic nanoparticle according to any one of embodiments 3-56, wherein said nucleic acid encodes one or more CD8 epitopes shown in Table 30 (SEQ ID Nos: 194-211).

Embodiment 58: The immunogenic nanoparticle according to any one of embodiments 3-57, wherein said nucleic acid encodes a peptide sequence that is an MHC-I targeting sequence upstream from said CD8+ T cell epitope(s).

Embodiment 59: The immunogenic nanoparticle of embodiment 58, wherein said MHC-I targeting sequence is a ppCT signal peptide.

Embodiment 60: The immunogenic nanoparticle of embodiment 59, wherein said ppCT signal peptide comprises the amino acid sequence VLLQAGSLHA (SEQ ID NO:2).

Embodiment 61: The immunogenic nanoparticle of embodiment 60, wherein said ppCT signal peptide further comprises a spacer comprising an amino acid or a peptide.

Embodiment 62: The immunogenic nanoparticle of embodiment 61, wherein said spacer comprises the sequence KK and said ppCT signal peptide comprises the amino acid sequence VLLQAGSLHAKK (SEQ ID NO:90).

Embodiment 63: The immunogenic nanoparticle according to any one of embodiments 3-62, wherein said nucleic acid encodes one or more CD4+ epitopes and/or one or more CD4+T-follicular epitopes.

Embodiment 64: The immunogenic nanoparticle of embodiment 63, wherein said nucleic acid encodes one or more CD4+T-follicular epitopes.

Embodiment 65: The immunogenic nanoparticle of embodiment 64, wherein said nucleic acid encodes one or more CD4+T-follicular epitopes shown in Table 23 and/or Table 24.

Embodiment 66: The immunogenic nanoparticle of embodiment 65, wherein said nucleic acid encodes at least one, or at least 2, or at least 3, or at least 4, or at least 5, or at least 6, or at least 7, or at least 8, or at least 9, or at least 10 or at least 11, or 12 CD4+T-follicular epitopes selected from the group consisting of

(SEQ ID NO: 165)
ATRFASVYAWNRKRI,
(SEQ ID NO: 166)
ESNKKFLPFQQFGRD,
(SEQ ID NO: 167)
CSNLLLQYGSFCTQL,
(SEQ ID NO: 168)
LFNKVTLADAGFIKQ,
(SEQ ID NO: 169)
QKGLTVLPPLLTDEM,
(SEQ ID NO: 170)
AQALNTLVKQLSSKF,
(SEQ ID NO: 172)
WNRKRISNCVADYSV,
(SEQ ID NO: 173)
KKFLPFQQFGR,
(SEQ ID NO: 174)
LLQYGSF,
(SEQ ID NO: 175)
FIKQYGDCLGD,
(SEQ ID NO: 176)
LLTDEMIAQYTSALLAGTI,
and
(SEQ ID NO: 177)
QALNTLVKQLS.

Embodiment 67: The immunogenic nanoparticle of embodiment 66, wherein said nucleic acid encodes the CD4+T-follicular epitope ATRFASVYAWNRKRI (SEQ ID NO:165).

Embodiment 68: The immunogenic nanoparticle according to any one of embodiments of embodiments 66-67, wherein said nucleic acid encodes the CD4+T-follicular epitope ESNKKFLPFQQFGRD (SEQ ID NO:166).

Embodiment 69: The immunogenic nanoparticle according to any one of embodiments of embodiments 66-68, wherein said nucleic acid encodes the CD4+T-follicular epitope CSNLLLQYGSFCTQL (SEQ ID NO:167).

Embodiment 70: The immunogenic nanoparticle according to any one of embodiments of embodiments 66-69, wherein said nucleic acid encodes the CD4+T-follicular epitope LFNKVTLADAGFIKQ (SEQ ID NO:168).

Embodiment 71: The immunogenic nanoparticle according to any one of embodiments of embodiments 66-70, wherein said nucleic acid encodes the CD4+T-follicular epitope QKGLTVLPPLLTDEM (SEQ ID NO:169).

Embodiment 72: The immunogenic nanoparticle according to any one of embodiments of embodiments 66-71, wherein said nucleic acid encodes the CD4+T-follicular epitope AQALNTLVKQLSSKF (SEQ ID NO:170).

Embodiment 73: The immunogenic nanoparticle according to any one of embodiments of embodiments 66-72, wherein said nucleic acid encodes the CD4+T-follicular epitope WNRKRISNCVADYSV (SEQ ID NO:172).

Embodiment 74: The immunogenic nanoparticle according to any one of embodiments of embodiments 66-73, wherein said nucleic acid encodes the CD4+T-follicular epitope KKFLPFQQFGR (SEQ ID NO:173).

Embodiment 75: The immunogenic nanoparticle according to any one of embodiments of embodiments 66-74, wherein said nucleic acid encodes the CD4+T-follicular epitope LLQYGSF (SEQ ID NO:174).

Embodiment 76: The immunogenic nanoparticle according to any one of embodiments of embodiments 66-75, wherein said nucleic acid encodes the CD4+T-follicular epitope FIKQYGDCLGD (SEQ ID NO:175).

Embodiment 77: The immunogenic nanoparticle according to any one of embodiments of embodiments 66-76, wherein said nucleic acid encodes the CD4+T-follicular epitope LLTDEMIAQYTSALLAGTI (SEQ ID NO:176).

Embodiment 78: The immunogenic nanoparticle according to any one of embodiments of embodiments 66-77, wherein said nucleic acid encodes the CD4+T-follicular epitope QALNTLVKQLS (SEQ ID NO:177).

Embodiment 79: The immunogenic nanoparticle of embodiment 65, wherein said nucleic acid encodes the CD4+T-follicular epitopes:

(SEQ ID NO: 165)
ATRFASVYAWNRKRI,
(SEQ ID NO: 166)
ESNKKFLPFQQFGRD,
(SEQ ID NO: 167)
CSNLLLQYGSFCTQL,
(SEQ ID NO: 168)
LFNKVTLADAGFIKQ,
(SEQ ID NO: 169)
QKGLTVLPPLLTDEM,
and
(SEQ ID NO: 170)
AQALNTLVKQLSSKF.

Embodiment 80: The immunogenic nanoparticle according to any one of embodiments 3-79, wherein said nucleic acid encodes one or more CD4+ epitopes.

Embodiment 81: The immunogenic nanoparticle of embodiment 80, wherein said nucleic acid encodes one or more CD4 epitopes shown in Table 33 (SEQ ID NOS:273, 275, 277, 279, 281, and 283) and/or Table 31 (SEQ ID Nos:212-230).

Embodiment 82: The immunogenic nanoparticle of embodiment 81, wherein said nucleic acid encodes the CD4 epitope KSAFYILPSIISNEK (SEQ ID NO:273).

Embodiment 83: The immunogenic nanoparticle according to any one of embodiments 81-82, wherein said nucleic acid encodes the CD4 epitope

(SEQ ID NO: 275)
PNMLRIMASLVLARK.

Embodiment 84: The immunogenic nanoparticle according to any one of embodiments 81-83, wherein said nucleic acid encodes the CD4 epitope

(SEQ ID NO: 277)
LSYYKLGASQRVAGD.

Embodiment 85: The immunogenic nanoparticle according to any one of embodiments 81-84, wherein said nucleic acid encodes the CD4 epitope AEILLIIMRTFKVSI (SEQ ID NO:279).

Embodiment 86: The immunogenic nanoparticle according to any one of embodiments 81-85, wherein said nucleic acid encodes the CD4 epitope

(SEQ ID NO: 281)
DFYLCFLAFLLFLVL.

Embodiment 87: The immunogenic nanoparticle according to any one of embodiments 81-86, wherein said nucleic acid encodes the CD4 epitope

(SEQ ID NO: 283)
MKFLVFLGIITTVAA.

Embodiment 88: The immunogenic nanoparticle according to any one of embodiments 81-87, wherein said nucleic acid encodes the CD4 epitope

(SEQ ID NO: 212)
ACFVLAAVYRINWIT.

Embodiment 89: The immunogenic nanoparticle according to any one of embodiments 81-88, wherein said nucleic acid encodes the CD4 epitope

(SEQ ID NO: 213)
AFLPFAMGI1AMSAF.

Embodiment 90: The immunogenic nanoparticle according to any one of embodiments 81-89, wherein said nucleic acid encodes the CD4 epitope FFLYENAFLPFAMGI (SEQ ID NO:214).

Embodiment 91: The immunogenic nanoparticle according to any one of embodiments 81-90, wherein said nucleic acid encodes the CD4 epitope

(SEQ ID NO: 215)
FLPFAMGIIAMSAFA.

Embodiment 92: The immunogenic nanoparticle according to any one of embodiments 81-91, wherein said nucleic acid encodes the CD4 epitope

(SEQ ID NO: 216)
GIAIAMACLVGLMWL.

Embodiment 93: The immunogenic nanoparticle according to any one of embodiments 81-92, wherein said nucleic acid encodes the CD4 epitope

(SEQ ID NO: 217)
IGYYRRATRRIRGGD.

Embodiment 94: The immunogenic nanoparticle according to any one of embodiments 81-93, wherein said nucleic acid encodes the CD4 epitope

(SEQ ID NO: 218)
IKLIFLWLLWPVTLA.

Embodiment 95: The immunogenic nanoparticle according to any one of embodiments 81-94, wherein said nucleic acid encodes the CD4 epitope

(SEQ ID NO: 219)
KLIFLWLLWPVTLAC.

Embodiment 96: The immunogenic nanoparticle according to any one of embodiments 81-95, wherein said nucleic acid encodes the CD4 epitope

(SEQ ID NO: 220)
LPFAMGIIAMSAFAM.

Embodiment 97: The immunogenic nanoparticle according to any one of embodiments 81-96, wherein said nucleic acid encodes the CD4 epitope

(SEQ ID NO: 221)
NAFLPFAMGHAMSA.

Embodiment 98: The immunogenic nanoparticle according to any one of embodiments 81-97, wherein said nucleic acid encodes the CD4 epitope

(SEQ ID NO: 222)
PSDFVRATATIPIQA.

Embodiment 99: The immunogenic nanoparticle according to any one of embodiments 81-98, wherein said nucleic acid encodes the CD4 epitope

(SEQ ID NO: 223)
PVTLACFVLAAVYRI.

Embodiment 100: The immunogenic nanoparticle according to any one of embodiments 81-99, wherein said nucleic acid encodes the CD4 epitope

(SEQ ID NO: 224)
RNRFLYI1KLIFLWL.

Embodiment 101: The immunogenic nanoparticle according to any one of embodiments 81-100, wherein said nucleic acid encodes the CD4 epitope

(SEQ ID NO: 225)
RWVLSFELLHAPAT.

Embodiment 102: The immunogenic nanoparticle according to any one of embodiments 81-101, wherein said nucleic acid encodes the CD4 epitope

(SEQ ID NO: 226)
SDFVRATATIPIQAS.

Embodiment 103: The immunogenic nanoparticle according to any one of embodiments 81-102, wherein said nucleic acid encodes the CD4 epitope T LAC FV LA AVY RI NW (SEQ ID NO:227).

Embodiment 104: The immunogenic nanoparticle according to any one of embodiments 81-103, wherein said nucleic acid encodes the CD4 epitope

(SEQ ID NO: 228)
VHFVCNLLLLFVTVY.

Embodiment 105: The immunogenic nanoparticle according to any one of embodiments 81-104, wherein said nucleic acid encodes the CD4 epitope

(SEQ ID NO: 229)
WLSFELLHAPATVC.

Embodiment 106: The immunogenic nanoparticle according to any one of embodiments 81-105, wherein said nucleic acid encodes the CD4 epitope

(SEQ ID NO: 230)
YRWVLSFELLHAPA.

Embodiment 107: The immunogenic nanoparticle according to any one of embodiments 63-106, wherein said nucleic acid encodes an MHC-II targeting peptide upstream from said CD4+ T cell epitopes or CD4+T-follicular epitopes.

Embodiment 108: The immunogenic nanoparticle of embodiment 107, wherein said MHC-II targeting peptide comprises TFR1-118 or Lil-80.

Embodiment 109: The immunogenic nanoparticle according to any one of embodiments 3-108, wherein said nucleic acid encodes one or more B cell epitopes shown in Table 20 and/or in Table 32 (SEQ ID Nos:231-238).

Embodiment 110: The immunogenic nanoparticle of embodiment 109, wherein said nucleic acid encodes at least 1, or at least 2, or at least 3, or at least 4, or at least 5, or 6 B cell epitopes shown in Table 22, SEQ ID NOS:130, 146, 148, 150, 154, 156.

Embodiment 111: The immunogenic nanoparticle according to any one of embodiments 109-110, wherein said nucleic acid encodes the B cell epitope

(SEQ ID NO: 128)
TRTQSPPAPPAYTNS.

Embodiment 112: The immunogenic nanoparticle according to any one of embodiments 109-111, wherein said nucleic acid encodes the B cell epitope

(SEQ ID NO: 129)
SQCVNLTTRTQLPPAYTNSFTRGVY.

Embodiment 113: The immunogenic nanoparticle according to any one of embodiments 109-112, wherein said nucleic acid encodes the B cell epitope GTNGTKRFD (SEQ ID NO:130).

Embodiment 114: The immunogenic nanoparticle according to any one of embodiments 109-113, wherein said nucleic acid encodes the B cell epitope

(SEQ ID NO: 131)
FSNVTWFHAIHVSGTNGTKRFDN.

Embodiment 115: The immunogenic nanoparticle according to any one of embodiments 109-114, wherein said nucleic acid encodes the B cell epitope

(SEQ ID NO: 132)
DPFLDVYYHKNNKSWME.

Embodiment 116: The immunogenic nanoparticle according to any one of embodiments 109-115, wherein said nucleic acid encodes the B cell epitope

(SEQ ID NO: 133)
HKNNKSWMESEFRVYSSANN.

Embodiment 117: The immunogenic nanoparticle according to any one of embodiments 109-116, wherein said nucleic acid encodes the B cell epitope

(SEQ ID NO: 134)
LTPGDSSSGWTAG.

Embodiment 118: The immunogenic nanoparticle according to any one of embodiments 109-117, wherein said nucleic acid encodes the B cell epitope

(SEQ ID NO: 135)
LTPGDSSSGWTAG.

Embodiment 119: The immunogenic nanoparticle according to any one of embodiments 109-118, wherein said nucleic acid encodes the B cell epitope

(SEQ ID NO: 136)
FPNITNLCPFDEVFNATRFASVYAWNRKRISNCVADYSVLYNFAPFFAFK
CYGVSPTKLNDL.

Embodiment 120: The immunogenic nanoparticle according to any one of embodiments 109-119, wherein said nucleic acid encodes the B cell epitope

(SEQ ID NO: 137)
RFASVYAWNRKRISNCVADY.

Embodiment 121: The immunogenic nanoparticle according to any one of embodiments 109-120, wherein said nucleic acid encodes the B cell epitope

(SEQ ID NO: 138)
RGNEVSQIAPGQTGNIAD.

Embodiment 122: The immunogenic nanoparticle according to any one of embodiments 109-121, wherein said nucleic acid encodes the B cell epitope

(SEQ ID NO: 139)
GQTGNIADYNYKLPDDFTG.

Embodiment 123: The immunogenic nanoparticle according to any one of embodiments 109-122, wherein said nucleic acid encodes the B cell epitope

(SEQ ID NO: 140)
KLDSKVGGNYNY.

Embodiment 124: The immunogenic nanoparticle according to any one of embodiments 109-123, wherein said nucleic acid encodes the B cell epitope

(SEQ ID NO: 141)
DSKVGGNYNYLYRLFRKSNL.

Embodiment 125: The immunogenic nanoparticle according to any one of embodiments 109-124, wherein said nucleic acid encodes the B cell epitope

(SEQ ID NO: 142)
YQAGNKPCN.

Embodiment 126: The immunogenic nanoparticle according to any one of embodiments 109-125, wherein said nucleic acid encodes the B cell epitope YQAGS (SEQ ID NO:143).

Embodiment 127: The immunogenic nanoparticle according to any one of embodiments 109-126, wherein said nucleic acid encodes the B cell epitope

(SEQ ID NO: 144)
YGFRPTYGV.

Embodiment 128: The immunogenic nanoparticle according to any one of embodiments 109-127, wherein said nucleic acid encodes the B cell epitope

(SEQ ID NO: 145)
PLRSYGFRPTYGVGHQPYRV.

Embodiment 129: The immunogenic nanoparticle according to any one of embodiments 109-128, wherein said nucleic acid encodes the B cell epitope FELLHAPATVCGPKKSTNLV (SEQ ID NO:146).

Embodiment 130: The immunogenic nanoparticle according to any one of embodiments 109-129, wherein said nucleic acid encodes the B cell epitope

(SEQ ID NO: 147)
LHAPATVCGPKKSTNLVKNK.

Embodiment 131: The immunogenic nanoparticle according to any one of embodiments 109-130, wherein said nucleic acid encodes the B cell epitope RDIADTTDAVRDP (SEQ ID NO:148).

Embodiment 132: The immunogenic nanoparticle according to any one of embodiments 109-131, wherein said nucleic acid encodes the B cell epitope

(SEQ ID NO: 149)
VCGPKKSTNLVKNKCVNFNFNGLTGTGVLTESNKKFLPFQQFGRDIADTT
DAVRDPQTLEILDITPCSFGGVSVI.

Embodiment 133: The immunogenic nanoparticle according to any one of embodiments 109-132, wherein said nucleic acid encodes the B cell epitope

(SEQ ID NO: 150)
VITPGTNTSN.

Embodiment 134: The immunogenic nanoparticle according to any one of embodiments 109-133, wherein said nucleic acid encodes the B cell epitope

(SEQ ID NO: 151)
GTNTSNQVAVLYQDVNCTEVPVA IHADQLTPTWRVYSTGS.

Embodiment 135: The immunogenic nanoparticle according to any one of embodiments 109-134, wherein said nucleic acid encodes the B cell epitope

(SEQ ID NO: 152)
ASYQTQTKSHRRARSVASQ.

Embodiment 136: The immunogenic nanoparticle according to any one of embodiments 109-135, wherein said nucleic acid encodes the B cell epitope

(SEQ ID NO: 153)
CASYQTQTKSHRRARSVASQ.

Embodiment 137: The immunogenic nanoparticle according to any one of embodiments 109-136, wherein said nucleic acid encodes the B cell epitope

(SEQ ID NO: 154)
LPDPSKPSKR.

Embodiment 138: The immunogenic nanoparticle according to any one of embodiments 109-137, wherein said nucleic acid encodes the B cell epitope

(SEQ ID NO: 155)
ILPDPSKPSKRS.

Embodiment 139: The immunogenic nanoparticle according to any one of embodiments 109-138, wherein said nucleic acid encodes the B cell epitope

(SEQ ID NO: 156)
VNNTVYDPLQPELDSFKEELDKYFKN HTSPDVDLGDISGI.

Embodiment 140: The immunogenic nanoparticle according to any one of embodiments 109-139, wherein said nucleic acid encodes the B cell epitope

(SEQ ID NO: 157)
DSFKEELDKYFKNHTSPDVD.

Embodiment 141: The immunogenic nanoparticle according to any one of embodiments 109-140, wherein said nucleic acid encodes the B cell epitope

(SEQ ID NO: 231)
GIAIAMACLVGLMWLS.

Embodiment 142: The immunogenic nanoparticle according to any one of embodiments 109-141, wherein said nucleic acid encodes the B cell epitope

(SEQ ID NO: 232)
GKNWITGG1A1AMACL.

Embodiment 143: The immunogenic nanoparticle according to any one of embodiments 109-142, wherein said nucleic acid encodes the B cell epitope

(SEQ ID NO: 233)
LWPVTLACFVLAAVYN.

Embodiment 144: The immunogenic nanoparticle according to any one of embodiments 109-143, wherein said nucleic acid encodes the B cell epitope

(SEQ ID NO: 234)
LWPVTLACFVLAAVYR.

Embodiment 145: The immunogenic nanoparticle according to any one of embodiments 109-144, wherein said nucleic acid encodes the B cell epitope

(SEQ ID NO: 235)
RINWITGGIAIAMACL.

Embodiment 146: The immunogenic nanoparticle according to any one of embodiments 109-145, wherein said nucleic acid encodes the B cell epitope

(SEQ ID NO: 236)
WLSFELLHAPATVCG.

Embodiment 147: The immunogenic nanoparticle according to any one of embodiments 109-146, wherein said nucleic acid encodes the B cell epitope

(SEQ ID NO: 237)
YENAFLPFAMGIIAMS.

Embodiment 148: The immunogenic nanoparticle according to any one of embodiments 109-147, wherein said nucleic acid encodes the B cell epitope

(SEQ ID NO: 238)
YFLCWHTNCYDYCIPY.

Embodiment 149: The immunogenic nanoparticle according to any one of embodiments 109-148, wherein said nucleic acid encodes a membrane targeting signal/secretory signal upstream from said B cell epitopes.

Embodiment 150: The immunogenic nanoparticle of embodiment 149, wherein said membrane targeting signal/secretory signal comprises TFR1-118 or MLLLLLLLLLLALALA (SEQ ID NO:1) or the prolactin peptide

(SEQ ID NO: 6)
KGSPWKGSLLLLLMNIVSNLLCQSVAPLP.

Embodiment 151: The immunogenic nanoparticle according to any one of embodiments 1-150, where when said CD4+T-follicular helper epitopes and/or said CD4+ epitopes are present and said B cell epitopes are present, a first cleavage site is disposed between said CD4+T-follicular helper epitopes and/or said CD4 epitopes and said B cell epitopes.

Embodiment 152: The immunogenic nanoparticle of embodiment 151, wherein said first cleavage site comprises a T2A or a P2A cleavage site.

Embodiment 153: The immunogenic nanoparticle according to any one of embodiments 1-150, where when said CD4+T-follicular helper epitopes and/or said CD4+ epitopes are present, and said CD8 epitopes are present, and said B cell epitopes are present, a first cleavage site is disposed between said CD4+T-follicular helper epitopes and/or said CD4 epitopes and said CD8+ epitopes and a second cleavage site is disposed between said CD8 epitopes and said B cell epitopes.

Embodiment 154: The immunogenic nanoparticle of embodiment 153, wherein said first cleavage site comprises a T2A or a P2A cleavage site and said second cleavage site comprises a T2A or a P2A cleavage site.

Embodiment 155: The immunogenic nanoparticle of embodiment 154, wherein said first cleavage site comprises a T2A cleavage site and said second cleavage site comprises a P2A cleavage site, or said first cleavage site comprises a P2A cleavage site and said second cleavage site comprises a T2A cleavage site.

Embodiment 156: The immunogenic nanoparticle according to any one of embodiments 1-155 wherein said nucleic acid encodes a spacer disposed between each of said epitopes wherein said spacer comprises an amino acid or a peptide.

Embodiment 157: The immunogenic nanoparticle of embodiment 156, wherein the spacer(s) disposed between said epitopes comprise amino acid sequence independently selected from the group consisting of AD, AQ, CG, DK, FG, FT, FV, GR, VI, LA, LH, LH, LP, LS, MA, NT, PN, PT, PV, QS, RE, RR, SF, SK, SR, TN, TQ, VE, VT, and IC.

Embodiment 158: The immunogenic nanoparticle of embodiment 157, wherein said spacer sequences independently comprises or consist of an amino acid sequence selected from the group consisting of LHVE, PNPT, ADLS, CGTN, PVVT, ICLP, LAMA, TQAQ, DKQS, LHRE, FVSK, VISF, GRNT, and RRFG.

Embodiment 159: The immunogenic nanoparticle of embodiment 156, wherein the spacer(s) comprise one or more lysines.

Embodiment 160: The immunogenic nanoparticle of embodiment 159, wherein the spacer comprises or consists of lysine.

Embodiment 161: The immunogenic nanoparticle of embodiment 160, wherein the spacer sequences comprise an amino acid or an amino acid sequence selected from the group consisting of L, LL, LLL, and LLLL (SEQ ID NO:305).

Embodiment 162: The immunogenic nanoparticle of embodiment 1, wherein said nucleic acid sequence comprises the sequence shown in Table 11.

Embodiment 163: The immunogenic nanoparticle of embodiment 1, wherein said nucleic acid sequence comprises a codon optimized sequence.

Embodiment 164: The immunogenic nanoparticle of embodiment 163, wherein said nucleic acid comprises a codon optimized sequence shown in Table 12.

Embodiment 165: The immunogenic nanoparticle according to any one of embodiments 1-164, wherein said nucleic acid encodes a furin cleavage site disposed between each epitope expressed by said nucleic acid sequence.

Embodiment 166: The immunogenic nanoparticle of embodiment 165, wherein said furin cleavage site comprise the amino acid sequence Arg-X-Lys/Arg-Arg (SEQ ID NO:94) where X is any amino acid.

Embodiment 167: The immunogenic nanoparticle of embodiment 166, wherein said furin cleavage site comprise the amino acid sequence RVRR (SEQ ID NO:95).

Embodiment 168: The immunogenic nanoparticle of embodiment 167, wherein said furin cleavage site is encoded by a nucleic acid sequence cgcgtgcgccgc (SEQ ID NO: 96).

Embodiment 169: The immunogenic nanoparticle according to any one of embodiments 1-164, wherein said nucleic acid encodes a cathepsin cleavage site disposed between each epitope encoded by said nucleic acid.

Embodiment 170: The immunogenic nanoparticle of embodiment 169, wherein said cathepsin cleavage site comprises the amino acid sequence PMGLP (SEQ ID NO:99).

Embodiment 171: The immunogenic nanoparticle of embodiment 170, wherein said cathepsin cleavage sequence is encoded by the nucleic acid sequence ccgatgggcctgccg (SEQ ID NO:100).

Embodiment 172: The immunogenic nanoparticle according to any one of embodiments 3-171, wherein said nucleic acid sequence comprise a start codon.

Embodiment 173: The immunogenic nanoparticle of embodiment 172, wherein said nucleic acid sequence comprise a start codon having the sequence ATG.

Embodiment 174: The immunogenic nanoparticle according to any one of embodiments 1-173, wherein said nucleic acid sequence comprise a stop codon.

Embodiment 175: The immunogenic nanoparticle of embodiment 174, wherein said nucleic acid sequence comprise a stop codon having the sequence TGA.

Embodiment 176: The immunogenic particle according to any one of embodiments 1-175, wherein said nucleic acid comprises a DNA (e.g., a cDNA).

Embodiment 177: The immunogenic nanoparticle of embodiment 176, wherein said nucleic acid encodes an mRNA comprising a cap, 5′ and 3′ untranslated regions (UTRs), an open-reading frame (ORF) that encodes said plurality of peptide antigens, and a 3′ poly(A) tail.

Embodiment 178: The immunogenic particle according to any one of embodiments 1-175, wherein said nucleic acid comprises an mRNA.

Embodiment 179: The immunogenic particle of embodiment 178, wherein said mRNA comprises a cap, 5′ and 3′ untranslated regions (UTRs), an open-reading frame (ORF) that encodes said plurality of peptide antigens, and a 3′ poly(A) tail.

Embodiment 180: The immunogenic nanoparticle of embodiment 178, wherein said nucleic acid further comprises replication machinery derived from a positive-stranded mRNA virus.

Embodiment 181: The immunogenic nanoparticle of embodiment 176, wherein said nucleic acid encodes replication machinery derived from a positive-stranded mRNA virus.

Embodiment 182: The immunogenic nanoparticle according to any one of embodiment 180-181, wherein said nucleic acid further comprises replication machinery derived from an alphavirus such as Sindbis or Semliki-Forest viruses.

Embodiment 183: An immunogenic nanoparticle comprising: nanoparticle comprising one or more lipid(s) and/or a one or more biocompatible polymer(s); a nucleic encapsulated within or attached to said nanoparticle where said nucleic acid encodes a plurality of peptide antigens independently selected from the peptide antigens shown in Table 30 (SEQ ID Nos: 194-211), Table 31 (SEQ ID Nos: 212-230), and/or Table 32 (SEQ ID Nos:231-238).

Embodiment 184: The immunogenic nanoparticle of embodiment 183, wherein said nucleic acid encodes at least one CD8 epitope from Table 30, and at least one CD4 epitope from Table 31.

Embodiment 185: The immunogenic nanoparticle according to any one of embodiments 183-184, wherein said nucleic acid encodes one or more CD8 epitopes shown in Table 30 (SEQ ID Nos: 194-211).

Embodiment 186: The immunogenic nanoparticle according to any one of embodiments 183-185, wherein said nucleic acid encodes one or more CD4 epitopes shown in Table 31 (SEQ ID Nos: 212-230).

Embodiment 187: The immunogenic nanoparticle according to any one of embodiments 183-186, wherein said nucleic acid encodes one or more B cell epitopes shown in Table 32 (SEQ ID Nos:231-238).

Embodiment 188: The immunogenic nanoparticle according to any one of embodiments 183-187, wherein said nucleic acid sequence is codon optimized.

Embodiment 189: The immunogenic nanoparticle of embodiment 188, wherein said nucleic acid sequence is optimized using GenSmart Codon Optimization.

Embodiment 190: The immunogenic nanoparticle according to any one of embodiments 183-189, wherein said nucleic acid encodes a spacer disposed between each of said epitopes wherein said spacer comprises an amino acid or a peptide.

Embodiment 191: The immunogenic nanoparticle of embodiment 190, wherein the spacer(s) disposed between said epitopes comprise amino acid sequence independently selected from the group consisting of AD, AQ, CG, DK, FG, FT, FV, GR, VI, LA, LH, LH, LP, LS, MA, NT, PN, PT, PV, QS, RE, RR, SF, SK, SR, TN, TQ, VE, VT, and IC.

Embodiment 192: The immunogenic nanoparticle of embodiment 191, wherein said spacer sequences independently comprises or consist of an amino acid sequence selected from the group consisting of LHVE, PNPT, ADLS, CGTN, PVVT, ICLP, LAMA, TQAQ, DKQS, LHRE, FVSK, VISF, GRNT, and RRFG.

Embodiment 193: The immunogenic nanoparticle of embodiment 190, wherein the spacer(s) comprise one or more lysines.

Embodiment 194: The immunogenic nanoparticle of embodiment 193, wherein the spacer comprises or consists of lysine.

Embodiment 195: The immunogenic nanoparticle of embodiment 194, wherein the spacer sequences comprise an amino acid or an amino acid sequence selected from the group consisting of L, LL, LLL, and LLLL (SEQ ID NO:306).

Embodiment 196: The immunogenic nanoparticle according to any one of embodiments 183-195, wherein said nucleic acid encodes CD4 epitope sequences separated from CD8 epitope sequences by a self-cleaving linker peptides (T2A) sequence.

Embodiment 197: The immunogenic nanoparticle of embodiment 196, wherein said self-cleaving linker peptide comprises a T2A sequence

(SEQ ID NO: 239)
GSGEGRGSLLTCGDVEENPG.

Embodiment 198: The immunogenic nanoparticle according to any one of embodiments 183-197, wherein said nucleic acid encodes a secretory signal upstream of one or more B cell epitopes.

Embodiment 199: The immunogenic nanoparticle of embodiment 198, wherein said secretory signal comprises the amino acid sequence

(SEQ ID NO: 1)
MLLLLLLLLLLALALA.

Embodiment 200: The immunogenic nanoparticle according to any one of embodiments 183-199, wherein said nucleic acid further encodes a leader sequence comprising or consisting of the amino acid sequence VLLQAGSLHA (SEQ ID NO:49).

Embodiment 201: The immunogenic nanoparticle of embodiment 200, wherein said leader sequence is joined to the peptide encoded by said nucleic acid by a spacer comprising an amino acid or a peptide.

Embodiment 202: The immunogenic nanoparticle of embodiment 201, wherein said spacer comprises the sequence KK.

Embodiment 203: The immunogenic nanoparticle according to any one of embodiments 183-202, wherein said nucleic acid encodes a furin cleavage site disposed between each epitope expressed by said nucleic acid sequence.

Embodiment 204: The immunogenic nanoparticle of embodiment 203, wherein said furin cleavage site comprise the amino acid sequence Arg-X-Lys/Arg-Arg (SEQ ID NO:94) where X is any amino acid.

Embodiment 205: The immunogenic nanoparticle of embodiment 204, wherein said furin cleavage site comprise the amino acid sequence RVRR (SEQ ID NO:95).

Embodiment 206: The immunogenic nanoparticle of embodiment 205, wherein said furin cleavage site is encoded by a nucleic acid sequence cgcgtgcgccgc (SEQ ID NO: 96).

Embodiment 207: The immunogenic nanoparticle according to any one of embodiments 183-202, wherein said nucleic acid encodes a cathepsin cleavage site disposed between each epitope encoded by said nucleic acid.

Embodiment 208: The immunogenic nanoparticle of embodiment 207, wherein said cathepsin cleavage site comprises the amino acid sequence PMGLP (SEQ ID NO:99).

Embodiment 209: The immunogenic nanoparticle of embodiment 170, wherein said cathepsin cleavage sequence is encoded by the nucleic acid sequence ccgatgggcctgccg (SEQ ID NO:100).

Embodiment 210: The immunogenic nanoparticle according to any one of embodiments 183-209, wherein said nucleic acid sequence comprise a start codon.

Embodiment 211: The immunogenic nanoparticle of embodiment 210, wherein said nucleic acid sequence comprise a start codon having the sequence ATG.

Embodiment 212: The immunogenic nanoparticle according to any one of embodiments 183-211, wherein said nucleic acid sequence comprise a stop codon.

Embodiment 213: The immunogenic nanoparticle of embodiment 212, wherein said nucleic acid sequence comprise a stop codon having the sequence TGA.

Embodiment 214: The immunogenic particle according to any one of embodiments 183-213, wherein said nucleic acid comprises a DNA (e.g., a cDNA).

Embodiment 215: The immunogenic nanoparticle of embodiment 214, wherein said nucleic acid encodes an mRNA comprising a cap, 5′ and 3′ untranslated regions (UTRs), an open-reading frame (ORF) that encodes said plurality of peptide antigens, and a 3′ poly(A) tail.

Embodiment 216: The immunogenic particle according to any one of embodiments 183-213, wherein said nucleic acid comprises an mRNA.

Embodiment 217: The immunogenic particle of embodiment 216, wherein said mRNA comprises a cap, 5′ and 3′ untranslated regions (UTRs), an open-reading frame (ORF) that encodes said plurality of peptide antigens, and a 3′ poly(A) tail.

Embodiment 218: The immunogenic nanoparticle of embodiment 216, wherein said nucleic acid further comprises replication machinery derived from a positive-stranded mRNA virus.

Embodiment 219: The immunogenic nanoparticle of embodiment 214, wherein said nucleic acid encodes replication machinery derived from a positive-stranded mRNA virus.

Embodiment 220: The immunogenic nanoparticle according to any one of embodiment 218-219, wherein said nucleic acid further comprises replication machinery derived from an alphavirus such as Sindbis or Semliki-Forest viruses.

Embodiment 221: The immunogenic nanoparticle according to any one of embodiments 1-220, wherein said nanoparticle is a lipidic nanoparticle (LNP).

Embodiment 222: The immunogenic nanoparticle of embodiment 221, wherein said nanoparticle comprises one or more cationic lipids selected from the group consisting of dilinoleylmethyl-4 dimethyl aminobutyrate (DLin-MC3-DMA), 1,2-dioleoyl-3-dimethylaminopropane (DODAP), didodecyl-dimethylammonium bromide (DDAB), 1,2-dioleoyloxy-3-trimethylammonium propane chloride (DOTAP), (N-[1-(2,3-dioleyloxy)propyl]-N,N,N-trimethylammonium chloride (DOTMA), 1,2-dilinoleyloxy-3-dimethylaminopropane (DLinDMA), 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA), C12-200 (Lipid 5), 3-(dimethylamino) propyl(12Z,15Z)-3-[(9Z,12Z)-octadeca-9,12-dien-1-yl]henicosa-12,15-dienoate (DMAP-BLP), and (2Z)-non-2-en-1-yl 10-[(Z)-(1-methylpiperidin-4 yl)carbonyloxy]nonadecanoate (L101), dicetylphosphate-tetraethylenepentaamine-based polycation lipid, YSK05, YSK12-C4, YSK13-C4, and YSK15-C4.

Embodiment 223: The immunogenic nanoparticle according to any one of embodiments 221-222, wherein said lipidic nanoparticle comprises a helper lipid.

Embodiment 224: The immunogenic nanoparticle of embodiment 223, wherein said helper lipid comprises a lipid selected from the group consisting of 1,2-dioleoyl-sn-glycero-3-phosphoethanolamine (DOPE), 1,2-distearoyl-sn-glycero-3-phosphocholine (DSPC), and 1-Palmitoyl-2-oleoyl-sn-glycero-3-phosphoethanolamine (POPE).

Embodiment 225: The immunogenic nanoparticle according to any one of embodiments 221-224, wherein said lipidic nanoparticle comprises cholesterol.

Embodiment 226: The immunogenic nanoparticle according to any one of embodiments 221-225, wherein said lipidic nanoparticle comprise a PEG-lipid.

Embodiment 227: The immunogenic nanoparticle according to any one of embodiments 221-226, wherein said lipidic nanoparticle comprises an ionizable (e.g., a cationic) lipid, a helper lipid, a PEG-lipid, and cholesterol.

Embodiment 228: The immunogenic nanoparticle of embodiment 227, wherein said lipidic nanoparticle comprises cholesterol, distearoylphosphatidylcholine (DSPC), PEG-Lipid and DODAP.

Embodiment 229: The immunogenic nanoparticle of embodiment 227, wherein said lipidic nanoparticle comprises Lin-DMA, DSPC, cholesterol, PEG2000-C-DMG).

Embodiment 230: The immunogenic nanoparticle of embodiment 227, wherein said lipidic nanoparticle comprises DLin-MC3-DMA, DSPC, cholesterol, and PEG2000-C-DMG).

Embodiment 231: The immunogenic nanoparticle according to any one of embodiments 229-230, where the molar ratio of ionizable cationic lipid:DSPC:Cholesterol:PEG2000-C-DMG is 50:10:39:5:1.5 mol %.

Embodiment 232: The immunogenic nanoparticle of embodiment 227, wherein said lipidic nanoparticle comprises lipidoid 98N12-5(1)4HCl, cholesterol, and mPEG2000-DMG.

Embodiment 233: The immunogenic nanoparticle of embodiment 232, wherein the molar ratio of lipidoid 98N12-5(1)4HCl:cholesterol:mPEG2000-DMG is 42:48:10.

Embodiment 234: The immunogenic nanoparticle of embodiment 227, wherein said lipidic nanoparticle comprises DLin-MC3-DMA, cholesterol, and mPEG2000-DMG.

Embodiment 235: The immunogenic nanoparticle of embodiment 234, wherein the molar ratio of DLin-MC3-DMA:cholesterol:mPEG2000-DMG is 42:48:10.

Embodiment 236: The immunogenic nanoparticle of embodiment 227, wherein said lipidic nanoparticle comprises an ionizable lipid, a helper lipid, cholesterol, and PEG-DMG, where said ionizable lipid is selected from the group consisting of YSK05, YSK12-C4, YSK13-C4, and YSK15-C4.

Embodiment 237: The immunogenic nanoparticle of embodiment 236, wherein said ionizable lipid comprises YSK05.

Embodiment 238: The immunogenic nanoparticle according to any one of embodiments 236-237, wherein said lipid nanoparticle comprises a molar ratio of ionizable lipid:helper lipid:cholesterol:PEG-DMG of 50:10:40:3.

Embodiment 239: The immunogenic nanoparticle according to any one of embodiments 236-237, wherein said lipid nanoparticle comprises a molar ratio of ionizable lipid:cholesterol:PEG-DMG of 70:30:3.

Embodiment 240: The immunogenic nanoparticle according to any one of embodiments 221-227, wherein said immunogenic nanoparticle comprises the cationic lipid, dioleoyl-3-trimethylammonium propane (DOTAP), and the cationic polymer protamine.

Embodiment 241: The immunogenic nanoparticle according to any one of embodiments 3-182, wherein said nanoparticle comprises one or more biocompatible polymer(s).

Embodiment 242: The immunogenic nanoparticle of embodiment 241, wherein said wherein said immunogenic nanoparticle comprises one or more biocompatible polymers selected from the group consisting of poly(lactic-co-glycolic acid) (PLGA), poly(glycolic acid) (PGA), poly(lactic acid) (PLA), poly(caprolactone) (PCL), poly(butylene succinate), poly(trimethylene carbonate), poly(p-dioxanone), Poly(butylene terephthalate), poly(ester amide) (HYBRANE®), polyurethane, poly[(carboxyphenoxy) propane-sebacic acid], poly[bis(hydroxyethyl) terephthalate-ethyl orthophosphorylate/terephthaloyl chloride], poly(β-hydroxyalkanoate), poly(hydroxybutyrate), poly(hydroxybutyrate-co-hydroxyvalerate), Poly-L-lysine (PLL), poly-ethylenimine (PEI), poly[(2-dimethylamino)ethyl methacrylate (pDMAEMA), polyamidoamine (PAMAM) dendrimers, biodegradable poly(β-amino ester) (PBAE) polymer, and a poly(amino-co-ester) (PACE) polymer.

Embodiment 243: The immunogenic nanoparticle according to any one of embodiments 241-242, wherein said biocompatible polymer comprises a cationic polymer.

Embodiment 244: The immunogenic nanoparticle of embodiment 243, wherein said biocompatible polymer comprises a cationic polymer selected from the group consisting of Poly-L-lysine (PLL), poly-ethylenimine (PEI), poly[(2-dimethylamino)ethyl methacrylate (pDMAEMA), polyamidoamine (PAMAM) dendrimers, biodegradable poly(β-amino ester) (PBAE) polymer, and poly(amino-co-ester) (PACE) polymer.

Embodiment 245: The immunogenic nanoparticle of embodiment 241-244, wherein said biocompatible polymer comprises poly(lactic-co-glycolic acid) (PLGA).

Embodiment 246: The immunogenic nanoparticle of embodiment 245, wherein said nanoparticle comprise a PLGA polymer that is coated with chitosan (a linear polysaccharide, composed of randomly distributed D-glucosamine plus N-acetyl-D-glucosamine), to provide a particle surface that allows nucleic acid binding.

Embodiment 247: The immunogenic nanoparticle of embodiment 245-246, wherein said PLGA comprises a lactide/glycolide molar ratio of about 50:50.

Embodiment 248: The immunogenic nanoparticle according to any one of embodiments 245-247, wherein said PLGA includes a content ranging from about 8% up to about 20% of ˜5 kDa PEG.

Embodiment 249: The immunogenic nanoparticle according to any one of embodiments 241-248, wherein said immunogenic nanoparticle further comprises one or more lipids capable of activating a danger signal and/or binding a nucleic acid.

Embodiment 250: The immunogenic nanoparticle of embodiment 249, wherein said one or more lipids comprises one or more cationic lipids independently selected from the group consisting of dilinoleylmethyl-4 dimethyl aminobutyrate (DLin-MC3-DMA), 1,2-dioleoyl-3-dimethylaminopropane (DODAP), didodecyl-dimethylammonium bromide (DDAB), 1,2-dioleoyloxy-3-trimethylammonium propane chloride (DOTAP), (N-[1-(2,3-dioleyloxy)propyl]-N,N,N-trimethylammonium chloride (DOTMA), 1,2-dilinoleyloxy-3-dimethylaminopropane (DLinDMA), 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA), C12-200 (Lipid 5), 3-(dimethylamino) propyl(12Z,15Z)-3-[(9Z,12Z)-octadeca-9,12-dien-1-yl]henicosa-12,15-dienoate (DMAP-BLP), and (2Z)-non-2-en-1-yl 10-[(Z)-(1-methylpiperidin-4 yl)carbonyloxy]nonadecanoate (L101), dicetylphosphate-tetraethylenepentaamine-based polycation lipid, YSK05, YSK12-C4, YSK13-C4, and YSK15-C4.

Embodiment 251: The immunogenic nanoparticle according to any one of embodiments 249-250, wherein said immunogenic nanoparticle comprises a helper lipid.

Embodiment 252: The immunogenic nanoparticle of embodiment 251, wherein said helper lipid comprises a lipid selected from the group consisting of 1,2-dioleoyl-sn-glycero-3-phosphoethanolamine (DOPE), 1,2-distearoyl-sn-glycero-3-phosphocholine (DSPC), and 1-Palmitoyl-2-oleoyl-sn-glycero-3-phosphoethanolamine (POPE).

Embodiment 253: The immunogenic nanoparticle according to any one of embodiments 249-252, wherein said immunogenic nanoparticle comprises cholesterol.

Embodiment 254: The immunogenic nanoparticle according to any one of embodiments 249-253, wherein said immunogenic nanoparticle comprise a PEG-lipid.

Embodiment 255: The immunogenic nanoparticle according to any one of embodiments 249-254, wherein said lipid comprises up to 25%, (molar percentage) of said nanoparticle.

Embodiment 256: The immunogenic nanoparticle of embodiment 255, wherein said lipid comprises about 1%, or about 2%, or about 3%, or about 4%, or about 5%, or about 6%, or about 7% up to about 25%, or up to about 20%, or up to about 15%, or up to about 10% (molar percentage) of said nanoparticle.

Embodiment 257: The immunogenic nanoparticle according to any one of embodiments 1-256, wherein said nanoparticle further comprises an adjuvant.

Embodiment 258: The immunogenic nanoparticle of embodiment 257, wherein said adjuvant comprises a lipid-based adjuvant.

Embodiment 259: The immunogenic nanoparticle of embodiment 258, wherein said adjuvant comprises a lipid-based adjuvant selected from the group consisting of telratolimod, monophosphoryl lipid A, and KRN7000.

Embodiment 260: The immunogenic nanoparticle of embodiment 259, wherein said adjuvant comprises telratolimod or 3M-052.

Embodiment 261: The immunogenic nanoparticle of embodiment 257, wherein said adjuvant comprises a TH1 biased adjuvant.

Embodiment 262: The immunogenic nanoparticle according to any one of embodiments 257, wherein said adjuvant is conjugated to said nucleic acid.

Embodiment 263: The immunogenic nanoparticle according to any one of embodiments 257-262, wherein said adjuvant comprise a moiety selected from the group consisting of a Toll Like Receptor agonist (TLR agonist and a Stimulator of Interferon Genes (STING) agonist.

Embodiment 264: The immunogenic nanoparticle of embodiment 263, wherein said adjuvant comprises a TLR agonist.

Embodiment 265: The immunogenic nanoparticle according of embodiment 264, wherein adjuvant moiety comprises an agonist of a Toll-Like Receptor selected from the group consisting of TLR1, TLR2, TLR3, TLR4, TLRS, TLR6, TLR7, TLR8, TLR9, TLR10, TLR11, and any combination thereof.

Embodiment 266: The immunogenic nanoparticle of embodiment 265, wherein said adjuvant comprises a palmitic acid-modified TLR7/8 agonist R848 (C16-R848).

Embodiment 267: The immunogenic nanoparticle according to any one of embodiments 265-266, wherein said adjuvant comprises a TLR3 agonist selected from the group consisting of Polyinosine-polycytidylic acid (poly (I:C)), Polyadenylic-polyuridylic acid (poly (A:U), and poly(I)-poly(C12U).

Embodiment 268: The immunogenic nanoparticle according to any one of embodiments 265-267, wherein said adjuvant comprises a TLR4 agonist selected from the group consisting of Lipopolysaccharide (LPS) and Monophosphoryl lipid A (MPLA).

Embodiment 269: The immunogenic nanoparticle according to any one of embodiments 265-268, wherein said adjuvant comprises a TL8 agonist comprising flagellin.

Embodiment 270: The immunogenic nanoparticle according to any one of embodiments 265-269, wherein said adjuvant comprises a TLR9 agonist that is a single strand CpG oligodeoxynucleotides (CpG ODN).

Embodiment 271: The immunogenic nanoparticle according to any one of embodiments 265-270, wherein said adjuvant comprises a TLR7, TLR8, or TLR7/8 agonist selected from the group consisting of Gardiquimod (1-(4-amino-2-ethylaminomethylimidazo[4,5-c]quinolin-1-yl)-2-methylpropan-2-ol), Imiquimod (R837), loxoribine, IRM1 (1-(2-amino-2-methylpropyl)-2-(ethoxymethyl)-1H-imidazo-[4,5-c]quinolin-4-amine), IRM2 (2-methyl-1-[2-(3-pyridin-3-ylpropoxy)ethyl]-1H-imidazo[4,5-c]quinolin-4-amine), IRM3 (N-(2-[2-[4-amino-2-(2-methoxyethyl)-1H-imidazo[4,5-c]quinolin-1-yl]ethox-y]ethyl)-N-methylcyclohexanecarboxamide), CL097 (2-(ethoxymethyl)-1H-imidazo[4,5-c]quinolin-4-amine) (agonist for TLR7/8), CL307 (agonist for TLR7), CL264, Resiquimod, 3M-052/MEDI9197, SD-101 (N-[(4S)-2,5-dioxo-4-imidazolidinyl]-urea), motolimod (2-amino-N,N-dipropyl-8-[4-(pyrrolidine-1-carbonyl)phenyl]-3H-1-benzazepine-4-carboxamide), CL075 (3M002, 2-propylthiazolo[4,5-c]quinolin-4-amine), and TL8-506 (3H-1-benzazepine-4-carboxylic acid, 2-amino-8-(3-cyanophenyl)-ethyl ester).

Embodiment 272: The immunogenic nanoparticle according to any one of embodiments 265-271, wherein said adjuvant comprises a TLR2 agonist selected from the group consisting of N-α-palmitoyl-S-[2,3-bis(palmitoyloxy)-(2RS)-propyl]-L-cysteine, palmitoyl-Cys((RS)-2,3-di(palmitoyloxy)-propyl) (“Pam3Cys”), e.g., Pam3Cys, Pam3Cys-Ser-(Lys)4 (also known as “Pam3Cys-SKKKK” and “Pam3CSK4”), Triacyl lipid A (“OM-174”), Lipoteichoic acid (“LTA”), peptidoglycan, and CL419 (S-(2,3-bis(palmitoyloxy)-(2RS)propyl)-(R)-cysteinyl spermine).

Embodiment 273: The immunogenic nanoparticle according to any one of embodiments 265-272, wherein said adjuvant comprises a TLR2/6 agonist that comprises Pam₂CSK₄ (S-[2,3-bis(palmitoyloxy)-(2RS)-propyl]-[R]-cysteinyl-[S]-seryl-[S]-lysyl-[S]-lysyl-[S]-lysyl-[S]-lysine.

Embodiment 274: The immunogenic nanoparticle according to any one of embodiments 265-273, wherein said adjuvant comprises a TLR2/7 agonist selected from the group consisting of CL572 (S-(2-myristoyloxy ethyl)-(R)-cysteinyl 4-((6-amino-2-(butylamino)-8-hydroxy-9H-purin-9-yl)methyl) aniline), CL413 (S-(2,3-bis(palmitoyloxy)-(2RS)propyl)-(R)-cysteinyl-(S)-seryl-(S)-lysyl-(S)-lysyl-(S)-lysyl-(S)-lysyl 4-((6-amino-2-(butylamino)-8-hydroxy-9H-purin-9-yl)methyl)aniline), and CL401 (S-(2,3-bis(palmitoyloxy)-(2RS)propyl)-(R)-cysteinyl 4-((6-amino-2(butyl amino)-8-hydroxy-9H-purin-9-yl)methyl) aniline).

Embodiment 275: The immunogenic nanoparticle according to any one of embodiments 263-274, wherein said adjuvant comprise a STING agonist.

Embodiment 276: The immunogenic nanoparticle of embodiment 275 wherein said adjuvant comprises one or more STING agonists selected from the group consisting of amidobenzimidazole (diABZI), 3′,5′-Cyclic diadenylic acid sodium salt (c-DI-AMP sodium salt), 3′,5′-Cyclic diguanylic acid sodium salt (c-Di-GMP sodium salt), 2′,3′-Cyclic guanosine monophosphate-adenosine monophosphate sodium salt (2′,3′-cGAMP), 3′,3′-Cyclic guanosine monophosphate-adenosine monophosphate sodium salt (3′,3′-cGAMP), 5,6-Dimethyl-9-oxo-9H-xanthene-4-acetic acid (DMXAA), CMA, MK-1454, CRD5500, cyclic di-nucleotide compounds as described in U.S. Patent Publication No: 2020/0062798 A1, tricyclic heteroaryl compounds as described in U.S. Patent Publication No: 2020/0040009 A1, and heteroaryl amide compounds as described in U.S. Patent Publication No: 2020/0039994 A1.

Embodiment 277: The immunogenic nanoparticle of embodiment 276, wherein said adjuvant comprises amidobenzimidazole (diABZI).

Embodiment 278: The immunogenic nanoparticle according to any one of embodiments 257-277, wherein said adjuvant is selected from the group consisting of a combined aluminum salt and TLR4 agonist, rOv-ASP-1 (recombinant Onchocerca volvulus activation associated protein-1, IC31@(a two-component adjuvant consisting of the artificial antimicrobial cationic peptide KLK acting as a vehicle and the TLR9-stimulatory oligodeoxynucleotide ODN1, SPO1, CPG oligonucleotide, alum-TLR7 agonist based on a TLR7 agonist (SMIP7.10), OprI lipoprotein of Pseudomonas aeruginosa, cathelicidin-derived antimicrobial peptides, delta inulin (β-D-[2-1]poly(fructo-furanosyl)α-D-glucose), and β-defensin.

Embodiment 279: The immunogenic nanoparticle according to any one of embodiments 257-277, wherein said adjuvant comprises one or more adjuvants selected from the group consisting of CpG ODNs (TLR9 agonists), imiquimod-family compounds (TLR7 agonists), lipopolysaccharide-based compounds (TLR4) LPS-like compounds, Flageline, dsRNA-like compounds (e.g., Poly (I:C) and derivatives), poly (I:C) and derivatives, MatrixM™90, AS03, CpG 1018), and Advax.

Embodiment 280: The immunogenic nanoparticle according to any one of embodiments 1-279, wherein said nanoparticles are of a size effective for phagocytic uptake by macrophages and/or dendritic cells.

Embodiment 281: The immunogenic nanoparticle of embodiment 280, wherein said nanoparticle ranges in size from about 50 nm up to about 3 μm.

Embodiment 282: The immunogenic nanoparticle of embodiment 281, wherein said nanoparticle ranges in size from about 50 nm, or from about 75 nm, or from about 100 nm, or from about 125 nm, or from about 150 nm, or from about 200 nm, or from about 250 nm, or from about 300 nm, or from about 350 nm, or from about 400 nm, or from about 450 nm, or from about 500 nm up to about 3 μm, or up to about 2.75 μm, or up to about 2.5 μm, or up to about 2.0 μm, or up to about 1.75 μm, or up to about 1.50 μm, or up to about 1.25 μm, or up to about 1 μm, or up to about 900 nm, or up to about 800 nm.

Embodiment 283: The immunogenic nanoparticle of embodiment 282, wherein said nanoparticle ranges in size from 500 to 800 nm.

Embodiment 284: The immunogenic nanoparticle according to any one of embodiments 1-278, wherein said nucleic acid(s) are encapsulated within said nanoparticle.

Embodiment 285: The immunogenic nanoparticle according to any one of embodiments 241-284, wherein said nucleic acid(s) are attached to the surface of said nanoparticle.

Embodiment 286: The immunogenic nanoparticle of embodiment 285, wherein said one or nucleic acids are directly attached to the surface of said nanoparticle.

Embodiment 287: The immunogenic nanoparticle of embodiment 286, wherein said one or more nucleic acids are attached to the surface of said nanoparticle with a linker.

Embodiment 288: The immunogenic nanoparticle of embodiment 287, wherein said linker comprises a DSPE-PEG-maleimide where said linker is incorporated in or on said biocompatible polymer.

Embodiment 289: The immunogenic nanoparticle according to any one of embodiments 1-288, wherein said nanoparticles have one or more targeting moieties attached to the surface where said targeting moieties bind to and/or facilitate uptake by antigen presenting cells (APCs).

Embodiment 290: The immunogenic nanoparticle of embodiment 289, wherein said antigen presenting cells comprise one or more cells selected from the group consisting of macrophages, dendritic cells, and B cells.

Embodiment 291: The immunogenic nanoparticle of embodiment 289, wherein said targeting moieties comprise mannose, high mannse (Man9), or α-1,2-D-mannobiose.

Embodiment 292: The immunogenic nanoparticle of embodiment 291, wherein said targeting moiety is attached to PEG2 kDa-DSPE.

Embodiment 293: The immunogenic nanoparticle according to any one of embodiments 289-290, wherein said targeting moiety binds to a receptor on a dendritic cell.

Embodiment 294: The immunogenic nanoparticle of embodiment 293, wherein said receptor on a dendritic cell is selected from the group consisting of DEC205, MR, Dectin-1, DC-SIGN, DNGR-1, and FcγR.

Embodiment 295: The immunogenic nanoparticle according to any one of embodiments 293-294, wherein said targeting moiety is selected from the group consisting of SAG-1 (T. gondii), HIV-1 gagP24, Ova, AHc, RSV fusion protein, MUC1, MAA, hCGβ, Ova, Diphtheria toxin (CRM₁₉₇), Ag85B (Mtb), triMN-LPR, Ova, Anti-Clec9A, Ova, MUC1, MUC1-Tn, E75 (HER-2), iFT, gp120_{αgal}/p24, and α-gal.

Embodiment 296: The immunogenic nanoparticle according to any one of embodiments 289-295, wherein said targeting moiety binds to a receptor on a macrophage.

Embodiment 297: The immunogenic nanoparticle of embodiment 296, wherein said targeting moiety binds to a receptor selected from the group consisting of a sialoadhesin receptor, a folate receptor, a galactose receptor, a mannose receptor, a β-glucan receptor, a scavenger receptor, and a tuftsin receptor.

Embodiment 298: The immunogenic nanoparticle of embodiment 297, wherein said targeting moiety is selected from the group consisting of sialic acid, 9-N-(4H-thieno[3,2-c]chromene-2-carbamoyl)-Neu5Acα2-3Galβ1-4GlcNAc (TCCNeu5Ac), folic acid, methotrexate, folate, galactose residue, lactose, low density lipoprotein (LDL), ovalbumin (OVA), lactobionic acid, mannose-rich glycoconjugates, mannose, mannan, mannosylated poly(L-lysine) (MPL), zymosan and other β-glucans, glucan, poly-guanine, apoB protein fragment, and Tufsin tetrapeptide (Thr-Lys-Pro-Arg, (SEQ ID NO:307)).

Embodiment 299: The immunogenic nanoparticle of embodiment 297, wherein said targeting moiety binds to or more scavenger receptors selected from the group consisting of Stabilin 1, Stabilin 2, and mannose receptor.

Embodiment 300: The immunogenic nanoparticle of embodiment 299, wherein said targeting moiety comprises a fragment of apolipoprotein B protein effective to bind to Stabilin 1 and/or Stabilin 2.

Embodiment 301: The immunogenic nanoparticle of embodiment 300, wherein said targeting moiety fragment ranges in length from about 5, or from about 8, or from about 10 up to about 50, or up to about 40, or up to about 30, or up to about 20 amino acids.

Embodiment 302: The immunogenic nanoparticle of embodiment 301, wherein said targeting moiety comprises a fragment of the apoB protein comprising the amino acid sequence RKRGLK (SEQ ID NO:302).

Embodiment 303: The immunogenic nanoparticle of embodiment 302, wherein said first targeting moiety comprises a fragment of the apoB protein comprising the amino acid sequence RLYRKRGLK (SEQ ID NO:303).

Embodiment 304: The immunogenic nanoparticle of embodiment 302, wherein said first targeting moiety comprise or consists of the amino acid sequence

(SEQ ID NO: 304)
CGGKLGRKYRYLR.

Embodiment 305: The immunogenic nanoparticle of embodiment 297, wherein said targeting moiety binds to a mannose receptor.

Embodiment 306: The immunogenic nanoparticle of embodiment 305, wherein said targeting moiety comprises mannan.

Embodiment 307: The immunogenic nanoparticle of embodiment 306, wherein said targeting moiety comprises a mannan having a MW ranging from about 35 to about 60 kDa.

Embodiment 308: The immunogenic nanoparticle according to any one of embodiments 289-307, wherein said targeting moiety targets lymph nodes.

Embodiment 309: The immunogenic nanoparticle of embodiment 308, wherein said targeting moiety targets lymph node germinal centers.

Embodiment 310: The immunogenic nanoparticle of embodiment 309, wherein said surface of said nanoparticle is glycosylated.

Embodiment 311: The immunogenic nanoparticle of embodiment 310, wherein targeting moiety comprises a glycan-rich moiety.

Embodiment 312: The immunogenic nanoparticle of embodiment 311, wherein targeting moiety comprises the glycan-rich bacterial protein, lumazine synthase.

Embodiment 313: The immunogenic nanoparticle of embodiment 308, wherein said targeting moiety is selected from the group consisting of CpG, and a member of the amph-CpG family.

Embodiment 314: The immunogenic nanoparticle according to any one of embodiments 289-313, wherein said second binding moiety is adsorbed to said nanoparticle.

Embodiment 315: The immunogenic nanoparticle according to any one of embodiments 289-313, wherein said second binding moiety is covalently bound to said nanoparticle directly or through a linker.

Embodiment 316: The immunogenic nanoparticle according to any one of embodiments 3-315, wherein said nanoparticle contains a compound that facilitates cytosolic release from the endolysosomal compartment.

Embodiment 317: The immunogenic nanoparticle of embodiment 316, wherein said compound comprises an endo-osmolytic peptide.

Embodiment 318: The immunogenic nanoparticle of embodiment 316, wherein said compound comprises an endo-osmolytic peptide selected from the group consisting of MPG, Pep-1, and PPTG1) that destabilizes the in the endolysosomal membrane, antimicrobial peptide LL-37 with glutamic acid substituting for all basic residues, antimicrobial peptide melittin with glutamic acid substituting for all basic residues, and antimicrobial peptide bombolitin V with glutamic acid substituting for all basic residues.

Embodiment 319: The immunogenic nanoparticle according to any one of embodiments 3-318, wherein said nanoparticle contains a compound that increases immunogenicity.

Embodiment 320: The immunogenic nanoparticle of embodiment 319, wherein said compound is a compound that permits or induce the maturation of dendritic cells (DCs).

Embodiment 321: The immunogenic nanoparticle of embodiment 320, wherein said compound that permit or induce the maturation of dendritic cells (DCs) is selected from the group consisting of a lipopolysaccharide, TNF-alpha, and a CD40 ligand.

Embodiment 322: The immunogenic nanoparticle of embodiment 321, wherein said compound is selected from the group consisting of IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta, TNF-alpha, and hGH.

Embodiment 323: The immunogenic nanoparticle of embodiment 321, wherein said compound is selected from a cytokine shown in Table 46.

Embodiment 324: The immunogenic nanoparticle according to any one of embodiments 3-323, wherein said immunogenic nanoparticle is effective to raise an immune response when administered to a mammal.

Embodiment 325: The immunogenic nanoparticle of embodiment 324, wherein said nanoparticle is effective to raise a humoral immune response when administered to a mammal.

Embodiment 326: The immunogenic nanoparticle according to any one of embodiments 324-325, wherein said nanoparticle is effective to raise a cellular immune response when administered to a mammal.

Embodiment 327: The immunogenic nanoparticle according to any one of embodiments 324-326, wherein said nanoparticle is effective to raise one or more panels of neutralizing antibodies directed against SARS-Cov-2 when administered to a mammal.

Embodiment 328: The immunogenic nanoparticle according to any one of embodiments 324-327, wherein said nanoparticle is effective to raise a protective immune response against directed against SARS-Cov-2 when administered to a mammal.

Embodiment 329: The immunogenic nanoparticle of embodiment 328, wherein said protective immune response is partially protective.

Embodiment 330: The immunogenic nanoparticle of embodiment 328, wherein said protective immune response is fully protective.

Embodiment 331: The immunogenic nanoparticle according to any one of embodiments 324-330, wherein said mammal is a human.

Embodiment 332: The immunogenic nanoparticle according to any one of embodiments 324-330, wherein said mammal is a non-human mammal.

Embodiment 333: A pharmaceutical formulation comprising:
a population of immunogenic nanoparticles according to any one of embodiments 1-332; and
a pharmaceutically acceptable carrier.

Embodiment 334: The pharmaceutical formulation of embodiment 333, wherein said formulation is formulated for administration via a route selected from the group consisting of subcutaneous administration, intramuscular administration, inhalation, topical microneedle administration, and oral administration.

Embodiment 335: A method of inducing an immune response directed against SARS-Cov-2 in a mammal, said method comprising:
administering to said mammal an effective amount of a population of immunogenic nanoparticles according to any one of embodiments 1-332, and/or a pharmaceutical formulation according to any one of embodiments 333-334.

Embodiment 336: The method of embodiment 335, wherein said nanoparticle is effective to raise a humoral immune response when administered to a mammal.

Embodiment 337: The method according to any one of embodiments 335-336, wherein said nanoparticle is effective to raise a cellular immune response when administered to a mammal.

Embodiment 338: The method according to any one of embodiments 335-337, wherein said nanoparticle is effective to raise a neutralizing or protective antibody population directed against SARS-Cov-2 when administered to a mammal.

Embodiment 339: The method according to any one of embodiments 335-338, wherein said nanoparticle is effect to raise a protective innate immune response against SARS-Cov-2 when administered to a mammal.

Embodiment 340: A method for the prophylaxis and/or treatment of a SARS-Cov-2 infection in a mammal, said method comprising:
administering to said mammal an effective amount of a population of immunogenic nanoparticle according to any one of embodiments 1-332, and/or a pharmaceutical formulation according to any one of embodiments 333-334.

Embodiment 341: The method a of embodiment 340, wherein said method is effective to raise a humoral immune response in said mammal.

Embodiment 342: The method according to any one of embodiments 340-341, wherein said method is effective to raise a cellular immune response in said mammal.

Embodiment 343: The method according to any one of embodiments 340-342, wherein said method is effective to raise neutralizing antibody population directed against the virus.

Embodiment 344: The method according to any one of embodiments 340-343, wherein said method is effective to raise a protective immune response against the virus.

Embodiment 345: The method of embodiment 344, wherein said protective immune response is partially protective.

Embodiment 346: The method of embodiment 344, wherein said protective immune response is fully protective.

Embodiment 347: The method according to any one of embodiments 335-346, wherein said mammal is a human.

Embodiment 348: The method according to any one of embodiments 335-346, wherein said mammal is a non-human mammal.

### Definitions

The terms “polypeptide”, “peptide”, and “protein” are used interchangeably herein to refer to a polymer of amino acid residues.

The term “viral protein” refers to a protein that is found in a virus. Illustrative viral proteins include but are not limited to the coronavirus spike protein (S-protein), and nucleocapsid protein (N-protein).

The terms “epitope” and “antigen determinant” as used herein may comprise viral protein fragments having a length ranging from about 6 to about 20 or even more amino acids, e.g., fragments as processed and presented by MHC class I molecules, preferably having a length of about 8 to about 10 amino acids, e.g., 8, 9, or 10, (or even 11, or 12 amino acids), or fragments as processed and presented by MHC class II molecules, preferably having a length of about 13 or more amino acids, e.g., 13, 14, 15, 16, 17, 18, 19, 20 or even more amino acids, wherein these fragments may be selected from any part of the amino acid sequence. These fragments are typically recognized by T cells in the form of a complex consisting of the peptide fragment, docked onto an MHC molecule.

A “B cell epitope” is the antigen portion binding to the immunoglobulin or antibody. These epitopes are typically fragments located on the outer surface of (native) protein or peptide antigens, having from about 5 to about 15 amino acids, or from about 5 to about 12 amino acids, or from about 6 to about 9 amino acids, which may be recognized by antibodies, e.g., in their native form. B-cell epitopes can be structural (e.g., one contiguous stretch of peptide sequence) or conformational (e.g., strung together by different pieces of the allergen that is not necessarily sequential but based on protein folding.

The term “T cell epitope” refers to a peptide derived from a protein that is recognized by the T-cell receptor (TCR) when bound to MHC molecules displayed on the cell surface of antigen-presenting cells (APCs).

The term “B-cell epitope” refers to solvent-exposed portions of an antigen (e.g., viral protein) that binds to secreted and cell-bound immunoglobulins.

An “immune response” refers to a specific reaction of the adaptive immune system to a particular antigen (so-called specific or adaptive immune response) or an unspecific reaction of the innate immune system (so-called unspecific or innate immune response). In certain embodiments the adaptive immune response refers to the core to specific reactions (adaptive immune responses) of the adaptive immune system. Particularly, it relates to adaptive immune responses to infections by viruses like e.g., SARS CoV-2, and MERS coronaviruses. However, this specific response can be supported by an additional unspecific reaction (innate immune response).

The terms “cellular immunity” and “cellular immune response” refer typically to the activation of macrophages, natural killer cells (NK), antigen-specific cytotoxic T-lymphocytes, helper T cells, memory T cells and the release of various cytokines and chemokines in response to an antigen. In a more general way, cellular immunity is not restricted to the role of assisting antibody production but also the activation of cellular elements of the immune system that provide protective immune responses. The cellular immune response includes the role of activating antigen-specific cytotoxic T-lymphocytes that are capable of inducing cytotoxic cell death of cells in the body that display antigenic epitopes on their surface, such as virus-infected cells, cells with intracellular infectious agents, or cancer cells displaying tumor antigens; activating macrophages and natural killer cells, enabling them to destroy pathogens, and stimulating cells to secrete a variety of cytokines that influence the function of other cells involved in adaptive immune responses and innate immune responses. After an acute phase infection, the immune system also allows the development of memory B and T-cell responses, that confer long-term protective immunity that is key to vaccine development.

The terms “humoral immunity” and “humoral immune response” refer typically to antibody production and the accessory processes that may accompany it. A humoral immune response may be typically characterized by a sequence of events that allow precursor B cells to develop into antibody producing cells, with or without the assistance of helper T cells that assist B-cell development, e.g., through cytokine production, germinal center formation, immunoglobulin isotype switching, immunoglobulin affinity maturation and the generation of memory B cells. Humoral immunity also typically may refer to the effector functions of antibodies, which include pathogen and toxin neutralization, classical complement activation, and opsonin promotion of phagocytosis and pathogen elimination.

The term “STING agonist” refers to an agonist of the Stimulator of Interferon Genes receptor also known as transmembrane protein 173 (TMEM173)

The term “about” when used with respect to a numerical value refers to that value ±10%, or ±5%, ±3%, or ±2%, or ±1% of that value. In certain embodiments about refers to ±10% of the value. In certain embodiments about refers to ±5% of the value. In certain embodiments about refers to ±2% of the value.

As used herein, the term “targeting moiety” refers to any moiety that binds to a component of a cell. In some embodiments, the targeting moiety specifically binds to a component of a cell. Such a component can be referred to as a “target”, as a “marker” or as a “receptor”. In various embodiments a targeting moiety may be a polypeptide, glycoprotein, nucleic acid, small molecule, carbohydrate, lipid, aptamer etc. In some embodiments, a targeting moiety is an antibody or characteristic portion thereof. In some embodiments, a targeting moiety is a receptor or characteristic portion thereof. In some embodiments, a targeting moiety is a ligand or characteristic portion thereof. In some embodiments, a targeting moiety is a nucleic acid targeting moiety (e.g., an aptamer) that binds to a cell type specific marker. In some embodiments, a targeting moiety is a small molecule. In certain embodiments, the targeting moiety binds a receptor expressed on the surface of a cell. The targeting moiety, in some embodiments, binds a soluble receptor. In some embodiments, the soluble receptor is a complement protein or a pre-existing antibody. In certain embodiments, the targeting moiety is for delivery of the nanocarrier to antigen-presenting cells, T cells, or B cells. In some embodiments, the antigen-presenting cells are macrophages and dendritic cells. In other embodiments, the macrophages are located in the subcapsular sinus of lymph nodes. In still other embodiments, the antigen-presenting cells are dendritic cells. In some embodiments, the antigen-presenting cells are follicular dendritic cells or Langerhans cells. Specific non-limiting examples of targeting moieties include, but are not limited to, molecules that bind to CD11b, CD169, mannose receptor, DEC-205, CD11c, CD21/CD35, CX3CR1, Fc receptor or a toll-like receptor (TLR). In some embodiments, the molecule that binds any of the foregoing is an antibody or antigen-binding fragment thereof (e.g., an anti-CD169 antibody). In some embodiments, the molecule that binds a Fc receptor is one that comprises the Fc portion of an immunoglobulin (e.g., IgG). In other embodiments, the Fc portion of an immunoglobulin is a human Fc portion. In some embodiments, the molecule that binds CX3CR1 is CX3CL1 (fractalkine). Targeting moieties that bind CD169 include anti-CD169 antibodies and ligands of CD169, e.g., sialylated CD227, CD43, CD206, or portions of these ligands that retain binding function, e.g., soluble portions. In some embodiments, the molecule that may direct a nanoparticles vaccine to the lymph node germinal center may be a fragment of the complement system, which is activated at the surface of the nanoparticle nanocarrier by glycosylated particle components.

The terms “TH1-biased adjuvant”, TH1-preferential adjuvant”, and “TH1-promoting adjuvant” are used interchangeably and refer to an adjuvant that is defined in the literature (see, e.g., Liu et al. (2003) Nature Immunology, 4: 687-693) as an immunomodulator that is able to promote or trigger a TH1 immune response against a given antigen when used together with this antigen. A TH1 immune response is mediated by a T-helper type I CD4+ T-cell that promotes cell-mediated immunity and is required for host defense against intracellular viral and bacterial pathogens. The generation of TH1 immunity by a vaccine adjuvant can be reflected by the production of TH1 cytokines, such as interferon-gamma, IL-2 and tumor necrosis factor-alpha or beta in the supernatant of splenocytes or peripheral blood lymphocytes that are collected from treated subjects and cultured in the presence of an antigen. The TH1 differentiation pathway is controlled by a master transcription factor, T-bet. The control in such a demonstration would be to use a splenocyte population from a TH1 subject or the control group that has not been treated with the antigen and the adjuvant, or a subject or treatment group that was treated with the antigen only. In various embodiments, triggering or promoting a TH1 immune response is defined by the preferential induction of IFN-gamma over IL-4. e.g., as detected by harvesting peripheral blood mononuclear cells or splenocytes of a treated subject to perform an analysis, or assessing the isotype of the antibodies being produced against the vaccinating antigens, e.g., measurement of the IgG2a subclass that reflects immunoglobulin class switching under the influence of TH1 cells (as compared to IgG1a subclass switching under the influence of TH2 cells). The assessment of the induction of this cytokine can be carried out by methods well known to those of skill in the art, for example, the use of ELISA to assess cytokine production in splenocyte supernatants, flow cytometry assessing IFN-gamma/IL-4 or IFN-gamma/IL-10 ratios in permeabilized mononuclear cells or determining IgG subclasses specific antibody titers by ELISA. In certain embodiments, the induction of IFN gamma and/or IgG2a and IgG2b upon stimulation of splenocytes with antigen and an adjuvant indicates that the adjuvant exerts TH1-promoting immunostimulatory effects. In certain embodiments, alternatively or in combination with the first definition of triggering or promoting a TH1 immune response given above, triggering or promoting a TH1 immune response may further be defined by the absence (or the absence of an induction) of a TH2 immune response. A TH2 immune response is characterized by a detectable increase in IL-4, IL-5, IL-10, and IL-13 induction and/or the production of detectable IgG1 immunoglobulins when compared with non-treated splenocytes. The assessment of the induction of IL-4 and/or IL-10 can be carried out by methods well known to those of skill in the art, for example, the use of ELISA or flow cytometry on splenocytes. The assessment of the induction of an IgG1 is preferably carried out by ELISA or Western Blot as described in the example. In certain embodiments, alternatively or in combination with the two above definitions of triggering or promoting a TH1 immune response or promoting a TH immune response may further be defined by the generation of an increase in IFN gamma/IL-10 ratio and/or IFN gamma/IL-4 ratio and/or a decrease in IgG1/IgG2a ratio against a defined antigen. In certain embodiments a change (increase or decrease as indicated above) in any of these ratios of 2 or more indicates that an adjuvant has TH1 properties. The assessment of the induction of each of the mentioned cytokines can be carried out by methods well known to those of skill in the art, for example, by ELISA or performance of RT-PCR on splenocytes.

A vaccine is typically understood to be a prophylactic or therapeutic material providing at least one antigen or antigenic function. The antigen or antigenic function may stimulate the body's cognate immune system to provide an adaptive immune response. It is often necessary that the antigenic portion is presented to the adaptive immune system by components of the innate immune system (antigen-presenting cells such as dendritic cells), and that the vaccine may include an adjuvant that promotes the ability of the innate immune system to perform the antigen-presenting function.

### BRIEF DESCRIPTION OF THE DRAWINGS

FIG. 1 provides an overview of multi-epitope vaccines described herein. secretary peptide is MLLLLLLLLLLALALA is SEQ ID NO:1.

FIG. 2 illustrates the omicron BA.1 spike protein sequence with mutations (see also Table 1 herein). Shown is the overall topology of the SARS-CoV-2 spike monomer, with the following subunits: NTD, N-terminal domain; RBD, receptor-binding domain, RBM, receptor-binding motif (RBM) that interacts directly with ACE2 receptor; SD1, subdomain 1; SD2, subdomain 2; S1/S2 furin cleavage and phospho-regulation site. Omicron is a SARS-CoV-2 variant that was first reported to the World Health Organization (WHO) from South Africa on Nov. 24, 2021. The Omicron BA.1 variant is one of the most mutated SARS-CoV-2 VOC. Its high transmissibility and immune evasion ability have raised global concerns. Many Omicron variants have evolved since, including BA.2, BA.2.12.1, BA.2.75, BA.3, BA.4, BA.5, and BA.XE.

FIG. 3 schematically illustrates one embodiment of a Type I Vaccine, comprised of multi-epitope linkage of conserved CD8+ T-cell epitopes that are routed for MHC I presentation. This allows the T-cell epitope combination to generate a diverse population of cytotoxic virus-clearing, memory and tissue resident CD8+ cell responses. Exogenous antigens are typically processed through the classical pathway of MHC class I presentation, also known as the proteasome-TAP pathway. This involves degradation by proteasomes and the resulting peptides are transported into the endoplasmic reticulum (ER) by the transporter associated with antigen processing (TAP) proteins. In the ER, the peptides are loaded into MHC class I molecules by a number of molecular chaperones. In addition, there is also an alternative pathway, included in this design, for accomplishing MHC I loading by signal sequences that are inserted into the ER. One example is the ppCT signal peptide, which can be N-terminally attached to downstream peptides for antigen presentation. This peptide, ppCT16-25 (VLLQAGSLHA (SEQ ID NO:2)), is derived from the calcitonin-related polypeptide alpha gene and is processed independently of proteasomes and TAP. The alternative pathway is explained in FIG. 4. The schematic also demonstrates that delivery of the multi-epitope mRNA construct into APC by lipid nanoparticles (LNPs) is transcribed into the poly-epitope peptide once released from the endosomes into the cytosol. PolyA sequence AAAAAAAAA (SEQ ID NO:3).

FIG. 4 shows a schematic to explain the classical/traditional and alternative pathways for MHC class I presentation, including the use of alternative peptide sequences (other than ppCT), also referred to as T-cell epitopes associated with impaired peptide processing (TEIPP) (Minev et al. (20000) Eur. J. Immunol. 30: 2115-2124; Durgeau et al. (2018) Nature Com. 9: 5097; Kovjazin & Carmon, Human Vaccines & Immunotherapeutics 10:9, 2733-2740; Hage et al. (2013) Ann. N.Y. Acad. Sci. 1283: 75-80; Oliveira and Hall. Frontiers in Immunology, 2015, Volume 6, Article 298). The classical pathway is explained in FIG. 3. In contrast, MHC I presentation in the alternative pathway is dependent on secretory and membrane proteins containing a signal peptide sequence (SP). SP domains are short 13-50 amino acid-long lipophilic targeting sequences, typically located at the N-terminus of proteins, destined for secretion or integration within cellular membranes (Kovjazin& Carmon, supra.). SPs exhibit a tripartite structure, comprised of a central hydrophobic region and flanking hydrophilic N- and C-terminal regions. Once protein translation is completed, SPs incorporated in the ER membrane are generally removed from the rest of the protein by a dedicated signal peptidase (SPase) protease. However, they can still enter the ER lumen to bind and stabilize MHC class I molecules, either indirectly via the TAP machinery, or directly by a novel TAP-independent pathway reliant on the protease activity of the ER-membrane-associated signal peptide peptidase (SPP). This unique “ER-targeting property” of SP domains has been used to improve ER localization, and, consequently, the immunogenicity of non-SP-linked epitopes (Kovjazin & Carmon, supra.). SP-generated peptides have assumed therapeutic importance in developing cancer immunotherapy vaccination. Moreover, the alternative pathway is frequently used during viral infections that cause degradation or mislocalization of MHC class I molecules, including SARS CoV2 (see, e.g., Zhang et al. (2021) Proc. Natl. Acad. Sci. USA, 118(23): e2024202118).

FIG. 5, panels A-B, illustrates targets of neutralizing antibodies by generated by certain embodiments of Vaccine II illustrated in FIG. 1. In the illustrated embodiment, Vaccine II expresses CD4 positive T-follicular helper epitopes upstream of a compilation of conserved linear B-cell epitopes that are targeted for secretion or cell surface expression. CD4 epitopes are separated from the B-cell epitopes by a self-cleaving linker peptides (T2A) sequence (GSGEGRGSLLTCGDVE ENPG (SEQ ID NO:4)). T2A peptides are 18-22 amino-acid (aa) viral oligopeptides, mediating polypeptide cleavage during translation in eukaryotic cells (see, e.g., Liu et al. (2017) Sci. Rep. 7: 2193). Panel A) The schematic shows that the receptor binding (RBD) and the N-terminal domains (NTD) of the spike protein, the two dominant regions targeted by neutralizing antibodies against SARS-CoV-2 spike protein (Yoshida et al. (2021) Sci. Rep. 11: 5934). The majority of the neutralizing antibodies generated by memory B-cell responses target the RBD, with a smaller number of less potent antibodies recognizing NTD (see, e.g., Kreer et al. (2020) Cell, 182: 843-854.e12.; Liu et al. (2020) Nature, 584: 450-456; Zost et al. (2020) Nature, 584: 443-449. Panel B) The NTD and RDB localities in a crystallographic structure of the spike protein (see, e.g., Guo et al. (2022) Mol. Biomed. 3: 12).

FIG. 6 illustrates the importance of lymph node germinal centers in memory B-cell development (see, e.g., Nel & Miller (2021) ACS Nano, 15: 5793-5818). As illustrated, naïve CD4+ T-cells are activated in the T-cell zone by antigen-presenting dendritic cells (DC). This includes the generation of follicular helper T lymphocytes (Tfh), which upregulate CXC-chemokine receptor 5 (CXCR5) expression before interacting with B-cells at the T-B border. Both cell types migrate to B-cell-follicles to form germinal centers. Active cooperation of Tfh with germinal center B-cells promotes immunoglobulin hypermutation, class switching and affinity maturation. In the process, B-cells differentiate into memory cells and long-lived plasma cells. (FDC: Follicle dendritic cell; BCR: B-cell receptor; MHC: major histocompatibility complex; TCR: T-cell receptor).

FIG. 7 shows a schematic demonstrating an integrated mRNA construct for delivery of CD4+ follicular helper (Tfh) epitopes plus B-cell epitopes. The design takes into consideration the lymph node cooperation between these cell types as explained in FIG. 6. The routing of the CD4 epitopes to MHC II is achieved by upstream signal peptides, including the transferrin receptor (TfR 1-118) or long or short invariant (li) chain sequences. Upstream sequences for directing the secretion or membrane expression of B-cell epitopes is explained in FIG. 8. Integral to this design, is separating the expression of CD4 from CD8 epitopes by a self-cleavable linker peptide (T2A) sequence (GSGEGRGSLLTCGDVEENPG (SEQ ID NO:5), as described in FIG. 5. After, codon optimization, the mRNA construct is delivered by lipid nanoparticles, followed by epitope processing and described above. The role of epitope routing in these cells, is explained in FIG. 8.

FIG. 8 provides a schematic explaining processing of B-cell epitopes by membrane targeting or secretory signal sequences. Although recognition of B-cell epitopes by the B-cell mIg receptor is not dependent on MHC expression, B-cells can act as antigen presenting cells, which present epitopes to CD4+ T cells via MHC-II. This allows T cell help for B-cell differentiation and antibody production. To facilitate epitope interactions with B cells in certain embodiments, the mRNA vaccine is designed to utilize two different but complementary expression mechanisms, namely: (i) epitope expression on the plasma membrane of the antigen presenting cell (APC); or (ii) secretion from the APC to allow extracellular contact with B-cells. Surface membrane expression on the APC can be accomplished by inserting the epitopes downstream of the transferrin receptor sequence (aa 1-118), acting as a leader sequence for surface membrane epitope expression (Diebold et al. (2001) Gene Therapy, 8: 487-493). This endosomal pathway also allows endocytic transport of the antigen into the MHC-II compartment, where processing allows activation of helper CD4+ lymphocytes to enhance antibody production and immunoglobulin class switching (Id.). To accomplish epitope secretion for extracellular interaction with B-cells, we made use of a secretory peptide signal. One example is a 16 aa secretory peptide, MLLLLLLLLLLALALA (SEQ ID NO:1) (Barash et al. (2002) Biochem. Biophys. Res. Comm. 294: 835-842; Giler-Gane et al. (2016) PLoS One, 11(50). An alternative secretory sequence is a peptide obtained from prolactin

(KGSPWKGSLLLLLMNIVSNLLCQSVAPLP (SEQ ID NO: 6)).

FIG. 9 illustrates the design of tandem CD4 and CD8 epitope-expressing constructs (e.g., a type II vaccine encoding CD4+ and CD8+ epitopes rather than CD4+ and B cell epitopes).

FIG. 10 illustrates mRNA lipid nanoparticle delivery, peptide expression and MHC-I and MHC-II presentation in APCs. Multi-epitope mRNA (SEQ ID NO:7) is delivered by lipid nanoparticles (LNPs) to antigen-presenting cells (APCs). After phagocytosis by APCs, the mRNA is released to the cytosol by endosomal escape, followed by transcription into hybrid peptides in the cytosol. The presence of the T2A self-cleavage site leads to ribosomal skipping, allowing the transcription process to generate two sets of hybrid peptide sequences, one for CD4 and the other four CD8 epitopes. CD4 epitopes are targeted to the MHC-II compartment by the transferrin receptor 1-118 or invariant chain 1-80 endosomal targeting sequences. The role of TFR₁₋₁₁₈ is to provide hybrid peptide trafficking to the plasma membrane, where its transmembrane domain allows incorporation in the surface membrane, before endocytosis of that membrane domain towards the MHC-II compartment. In contrast, Ii₁₋₈₀ is directly transported to the MHC-II compartment. In the case of CD8 epitopes, the expressed hybrid peptide is processed by the 26S proteasome, allowing the cleaved epitopes to be transported into the ER and Golgi by TAP transporters. The addition of a ppCT signal peptide enhances MHC-I presentation.

FIG. 11 illustrates the design of a mRNA construct that is delivered to the APC by cationic lipid nanoparticles (LNP) to allow expression of epitopes, allowing for B-cell activation and antibody production. To do so, the multi-epitope mRNA construct is designed through conjugation to one of two signal peptides linked to the B-cell epitopes. One signal peptide is comprised of the transferrin receptor domain (TFR1-118), which as lead sequence, allows insertion of the hybrid peptide into the APC plasma membrane, where conformational and non-confirmational epitopes can be recognized by the B-cell membrane immunoglobulin receptor (BCR). The alternative is conjugation of a secretory signal peptide to the B-cell epitopes, allowing their export from the APC to make extracellular contact with BCR expressing cells. An example of a secretory sequence is the 16 aa sequence MLLLLLLLLLLALALA (SEQ ID NO:1). It is also important to consider that B cells themselves can act as APCs, which can use antigen binding to their BCR for endocytosis and antigen presentation on surface expressed MHC-II molecules. In this setting, the interaction of B-cells with helper T-cells delivers additional activation stimuli, culminating in immunoglobulin class switching, increased immunoglobulin production, and B-cell differentiation into plasma cells.

FIG. 12, panels A-B, show the characterization of cationic lipid nanoparticles (LNPs), which are specifically constructed to allow mRNA encapsulation and delivery.

FIG. 13, panels A-B, depict confocal microscopy images to show the expression of green fluorescent protein (GFP) in liver sinusoidal endothelial (panel A) and Kup5 cells (panel B) that were incubated with LNPs, encapsulating GFP mRNA.

FIG. 14, schematically illustrates certain ionizable lipids for inclusion in hybrid biocompatible polymer/lipid immunogenic nanoparticles and/or for formulation of immunogenic lipid nanoparticles.

FIG. 15 illustrates some representative lipid-based adjuvants that can be incorporated into the immunogenic nanoparticles described herein.

FIG. 16 shows a schematic explaining the use of a mannose targeting ligand in promoting lipid nanoparticle uptake in lymph nodes by targeting of the type II mannose receptor, DC-SIGN, on the surface of dendritic cells. The construction of mannosylated particles with sugar units (that increase avidity by mannose receptors) and co-delivery of danger signals (that upregulate the expression of mannose receptors in immune cells) have been shown as ways of improving vaccine efficacy. The number of saccharide subunits in the ligand determines binding avidity and efficacy.,

FIG. 17. Development of a liver targeting cationic lipid nanoparticles (LNP), delivering mRNA for the expression of green fluorescence protein (GFP). For proof-of-principle purposes, we selected a GFP RNA construct to develop in-house LNPs, with and without mannose surface decoration. GFP-mRNA LNPs were synthesized through the mixing of the ionizable cationic lipid, i.e. DLin-MC3-DMA, with DSPC, cholesterol and DMG-PEG-2k or DSPE-PEG2000-mannose, dissolved in ethanol in the molar ratios of 50, 10, 38.5, 1.5, while the mRNA (GFP-mRNA) components were diluted in Rnase-free 100 mM Sodium Acetate Buffer (pH 4.0). Briefly, the lipid compositions of DLin-MC3-DMA, DSPC, cholesterol and DMG-PEG-2k/DSPE-PEG2000-mannose were dissolved in ethanol stock solutions at 10, 20, 10 and 20 mg/mL, respectively. To prepare a 2.2 mL LNP batch, an appropriate amount of each of the lipid stock solution was mixed in the ethanol phase to provide a 0.55 mL lipid mix, designated as the Organic Phase. The Aqueous Phase was prepared by dissolving 0.2 mg GFP-mRNA in 1.4438 mL sodium acetate buffer. The final concentration of RNA in the Aqueous Phase was calculated to be 0.12 mg/mL; the final concentration of lipid mix was calculated to be 8.72 mM. The chemical structures of the four lipids shown in the figure. Following preparation of the Aqueous and Organic Phases, a microfluidics mixer (NanoAssemblr Benchtop, Precision NanoSystems Inc.) was used to synthesize a batch of GFP mRNA delivering LNPs. After initiating sample mixing, the flow through samples were collected and purified by ultracentrifugation to remove the excess ethanol. The LNP samples were purified by filtration of the 40×diluted sample using an Amicon® Ultra-15 centrifugal filtration tube. The size and the surface potential of the purified LNP samples were determined by dynamic light scattering (DLS), and the results are shown in FIG. 43. The encapsulation efficiency (EE %) of mRNA were determined using a RIBOGREEN®. The encapsulation efficiency (EE %) of GFP-mRNA LNPs were >97%. Subsequently, the LNPs samples were imaged by cryogenic electron microscopy (Cryo-EM) to investigate a nano-precipitated core structure.

FIG. 18. Schematic to show physicochemical characterization of unmodified and mannose decorated LNP.

FIG. 19, panels A-C, illustrates a flow cytometry approach to show enhanced uptake of decorated versus non-decorated LPN in an Antigen Presenting Cell. Panel A: Schematic diagram to show the approach for assessing and comparing LNP cellular uptake and GFP expression in antigen presenting cells, using non-decorated and mannose-decorated particles. 5×105 cells were inoculated separately in each well of a 6-well plate. After 24 h incubation, the cells were treated with mGFP/LNP or mGFP/LNP-Man (RNA concentration: 0.25 μg/mL or 0.5 μg/mL) for another 24 h. The cells were digested and collected for flow cytometry test. Panel B: EGF expression was increased by both particle types, with a significant increase in the fluorescence signal for mGFP/LNP-mannose compared to mGFP/LNP-mannose. Panel C: Mean fluorescence intensity of LSECs after treatment with mGFP/LNP or mGFP/LNP-Man. (n=3, * p<0.05, ** p<0.01). We also demonstrated in subsequent experiments that the increased uptake and expression of decorated nanoparticles could be outcompeted by the ligand or an antibody to the mannose receptor.

FIG. 20, panels A-B, illustrates co-delivery of a TLR agonist enhance DC-SIGN expression and targeting. Panel A: Schematic to show how the expression of DC-SIGN on dendritic cells can be increased by delivery of danger signals, leading to enhanced particle uptake (Li et al. (2019) Front. Chem. 2019, Volume 7, Article 650). Panel B: Illustrates how the incorporation of a lipid-tailed TLR7 agonist, 3M-052, in the particle surface could deliver a danger signal that promotes the immunogenic effects of nanoparticles, as demonstrated in the context of cancer immunotherapy (Lou et al. (2022) ACS Nano, 16: 13168-13182).

FIG. 21, panels A-B, illustrates alternative approaches for targeting DC-SIGN, using more complex ligands. Panel A: An alternative DC-SIGN targeting strategy is to use larger and more complex oligomannosides with intrinsic multivalence. Various oligosaccharides have been explored for DC-SIGN binding (McIntosh et al. (2015) Chem. Sci. 6: 4636-4642), a prime example being the “high mannose” structure Man9. This ligand has been used to deliver cancer antigens to DCs to improve immunotherapy (Buskas et al., 2005; Moyle et al., 2007; Srinivas et al., 2007; McIntosh et al., 2015; Glaffig et al., 2018). Panel B) This DC-targeting strategy takes advantage of DC-SIGN binding α1,2-mannobiose, which is present at terminal ends of oligosaccharides in certain viruses, bacteria, and other pathogens Pappalardo et al. (2021) Mol. Pharmaceutics, 18: 2540-2555. The oxidative conjugation of α1′,2-mannobiose to NH2-PEG2 kDa-DSPE allows us to use the conjugate for surface display of the ligand in the same way as explained in FIGS. 17 and 18.

### DETAILED DESCRIPTION

While neutralizing antibodies will remain the cornerstone of broadly protective Covid-19 vaccines, we believe the induction of virus-specific CD8+ T-cells can greatly augment antibody mediated protection. For instance, vaccine-induced generation of tissue-resident memory (TRM) CD8+ T-cells at the infection site could trigger improved protection by inducing early onset immune effector function by accelerating the recruitment of circulatory immune cells (see, e.g., Steinbach et al. (2018) Frontiers Immunol, 9: 2827 doi.org/10.3389/fimmu.2018.02827). Even individuals with mild or asymptomatic coronavirus disease (COVID-19) have increased clonal expansion of CD8+ T-cells in bronchoalveolar lavage fluid (Liao et al. (2020) Nat. Med., 26: 842-844), robust CD8+ T-cell reactivity to SARS-CoV-2 epitopes (Peng et al. (2020) Nat. Immunol., 21: 1336-1345; Sekine et al. (2020) Cell, 183: 158-168.e14), and rapid CD8+ T-cell-mediated viral clearance (Tan et al. (2021) Cell Rep. 34: 108728). In addition, it has been observed that patients with X-linked agammaglobulinemia who lack circulating B cells or individuals treated with rituximab (to suppress autoimmune disease activity) display functional T-cells that assist in the development of mild to moderate COVID-19 disease (Soresina et al. (2020) Pediatr. Allergy Immunol., 31: 565-569). Moreover antibody-mediated depletion of CD8+ T-cells from convalescent macaques partially decreases protective immunity (McMahan et al. (2021) Nature 590(7847): 630-634).

From the perspective of a broad-based coronavirus vaccine, CD8+ T-cells developing in response to SARS-CoV-1 infection exhibit long-lasting immune memory (Ng et al. (2016) Vaccine, 34: 2008-2014), and vaccine-induced CD8+ T-cells protect against lethal SARS-CoV-1 challenge in mice (Channappanavar et al. (2014) J. Virol. 88: 11034-11044). Further, in contrast to the generation of neutralizing antibody responses to spike protein, CD8+ T-cells target regions across the SARS-CoV-2 proteome (structural and nonstructural proteins), including to viral domains that are constrained from mutation, even in the current circulating variants of concern. This feature is of particular relevance given the emerging evidence that SARS-CoV-2 can evade cellular immunity through mutation of HLA-class-I-restricted epitopes (Agerer et al. (2021) Sci. Immunol., 6: eabg6461).

Accordingly, in various embodiments immunogenic nanoparticles are provided that have the capability of inducing an immune response directed against SAR-Cov-2 viral proteins through the generation of robust CD8+ T-cell responses. Methods of use of the nanoparticles are provided. In certain embodiments the nanoparticles can inhibit or prevent infection by SARS-Cov-2 virus, or reduce the severity of infection, and/or reduce or eliminate the need for hospitalization of an infected subject. Accordingly, various methods of immunization or vaccination using the nanoparticles are provided.

In certain embodiments the immunogenic nanoparticles (e.g., nanoparticle-based vaccines) provide for the delivery of one or more mRNAs that each encode a plurality of SARS-Cov-2 antigens (e.g., epitopes) to generate potent cellular and/or humoral immunity against the virus. In certain embodiments the immunogenic nanoparticles described herein induce and/or boost an immune response against SARS-Cov-2, and potentially other coronaviruses, through the activation of CD4 and CD8 T cells as well as B lymphocytes. It is believed the multi-epitope nanoparticles described herein are able to provide protective vaccine or booster responses against COVID-19.

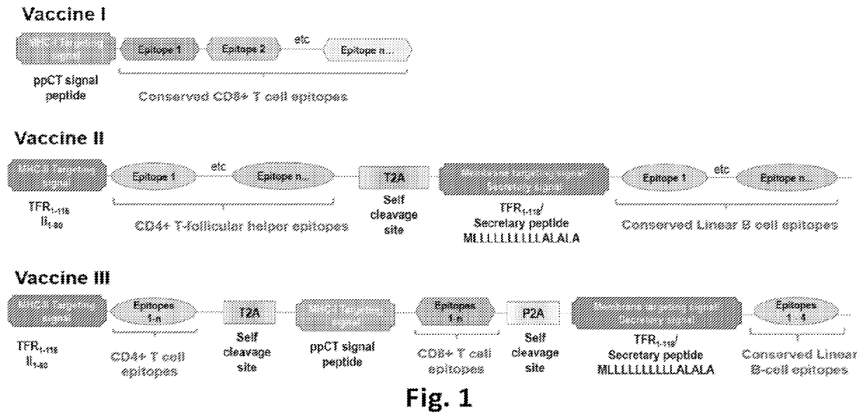

FIG. 1 provides an overview of multi-epitope vaccines described herein. Vaccine I is aimed at the delivery of a series of conserved CD8 positive T-cell epitopes, with a view to promote activation of T cells that can engage in the cytotoxic killing and clearance of viral infected cells, including infection by a range of SARS-CoV-2 variants of concern as well as other sarbecoviruses. The vaccine generates a number of CD8 positive T-cell subsets that exhibit cytotoxic activity, T-effector memory (TEM) and T-resident memory (TRM) activities. Epitopes selected after IEDB search, with refinement, to allow HLA binding, are integrated into a codon-optimized mRNA strand for incorporating into lipid nanoparticles or other nanoparticles. Nanoparticle uptake into antigen presenting cells are translated into multi-epitope peptides, designed for MHC I targeting and CD8 T-cell activation. Vaccine II is constructed by combining conserved CD4 positive epitopes with conserved B-cell epitopes for the purpose of raising neutralizing antibody production in lymph node germinal centers, where the cooperation of T-follicular helper cells (CD4+) with B-cell precursors are responsible for antibody production, including by promoting immunoglobulin class switching, hypermutation and generation of memory B cells. The mRNA construct is premised on an upstream targeting sequence that directs the CD4+ epitopes to the MHC II compartment in APCs. Linkage of the CD4+ multi-epitope sequence to the B-cell sequence is done by a T2A self-cleavage peptide that allows their independent processing after cellular translation. B-cell epitopes are routed for membrane expression or secretion from the APC. Vaccine III combines all three modalities, which, in certain embodiments, is illustrated by combining epitope selections in Vaccines I and II, as well as for conserved sequences other than spike protein epitopes, also widely expressed in other coronaviruses, including to construct pan-coronavirus vaccines

In various embodiments, nanoparticles formed from a biocompatible polymer provide the basic platform for delivering the mRNA(s) encoding SARS-Cov-2 antigens. One illustrative, but non-limiting biocompatible polymer is poly (lactic-co-glycolic acid) (PLGA) copolymer. This biocompatible polymer has been approved for a host of therapeutic applications by the Food and Drug Administration (FDA) because of high biodegradability and biocompatibility. The biocompatible nanoparticles provide a versatile platform that can be custom designed to incorporate nucleic acids. In certain embodiments the nanoparticles comprise one or more lipid(s) and/or one or more biocompatible polymer(s). In certain embodiments the nanoparticles can exclude biocompatible polymers and thereby provide nanoparticles comprising substantially or all lipids. Such nanoparticle may be designated lipid (or lipidic, or lipidoic) nanoparticles (LNPs). In certain embodiments the nanoparticles comprise one or more biocompatible polymers and optionally one or more lipids (e.g., cationic lipids) to, e.g., facilitate nucleic acid encapsulation. In various embodiments the biopolymer nanoparticles, lipidic nanoparticles and combined lipid/biocompatible polymer nanoparticles can be modified by the choice of the combination of copolymers as well as inclusion of additional polymeric or lipid components that can promote or alter the self-assembly of the particle components. Moreover, the particle surface can be functionalized with ligands or surface functionalities that target specific cell types.

### Nucleic Acid Constructs for the Delivery of SARs-Cov-2 Epitopes.

In certain embodiments the immunogenic nanoparticles described herein that comprise one or more nucleic acids that encode a plurality of SARS-Cov-2 epitopes selected to elicit a potent immune response (see, e.g., Tables 5, 2, 33, and the like shown below. In certain embodiments the nucleic acid comprises a DNA. In certain embodiments the nucleic acid comprises a mRNA.

The core principle behind mRNA as a technology for vaccination is to deliver the transcript of interest, encoding one or more immunogen(s), into the host cell cytoplasm where expression generates protein(s) or hybrid peptides that are intracellularly located with the possibility of being proteolytically digested (before further processing), transported to the surface membrane, or secreted. In certain embodiments the mRNA comprises one of two categories of mRNA constructs: 1) Non-replicating mRNA (NRM); and 2) Self-amplifying mRNA (SAM) constructs. NRM and SAM constructs typically comprise a cap structure, 5′ and 3′ untranslated regions (UTRs), an open-reading frame (ORF) encoding the antigen(s) of interest, and a 3′ poly(A) tail (see, e.g., Pardi (2018) Nat. Rev. Drug Dis. 17: 261-279). SAMs differ from NRM constructs by additionally including genetic replication machinery derived from positive-stranded mRNA viruses, most commonly from alphaviruses such as Sindbis and Semliki-Forest viruses (see, e.g., Cheng et al. (2001) J. Virol. 75: 2368-2376; Ljungberg & Liljestrom (2015) Exp. Rev. Vacc. 14: 177-194; and the like). Generally, the ORF encoding viral structural proteins is replaced by the selected transcript of interest, and the viral RNA-dependent RNA polymerase is retained to direct cytoplasmic amplification of the replicon construct.

Once delivered to the cytosol, NRM constructs are translated by ribosomes to produce the protein of interest (e.g., proteins/peptides comprising the antigen(s)/epitope(s)), that can undergo post-translational modification. Similarly, SAM constructs can also be translated by ribosomes to produce the replicase machinery necessary for self-amplification of the mRNA. Self-amplified mRNA constructs are translated by ribosomes to produce the protein of interest, which can undergo subsequent post-translational modification. The innate and adaptive immune responses detect the protein of interest and generate the desired immune response.

In various embodiments, mRNA vaccine manufacturing begins with the generation of a plasmid DNA (pDNA) containing a DNA-dependent RNA polymerase promoter, such as T7 (see, e.g., Rong et al. (1998) Proc. Natl. Acad. Sci. USA, 95: 515-519) and the corresponding sequence for the mRNA construct. The pDNA is linearized to serve as a template for the DNA-dependent RNA polymerase to transcribe the mRNA, and subsequently degraded by a DNase process step. In various illustrative, but non-limiting embodiments the addition of the 5′ cap and the 3′ poly(A) tail can be achieved during the in vitro transcription step (see, e.g., Stepinski et al. (2001) RNA, 7: 1486-1495; Grudzien-Nogalska et al. (2007) Meth. Enzym. Ch. 431: 203-227; and the like) or enzymatically after transcription (see, e.g., Martin & Moss (1975) J. Bio Chem. 250: 9330-9335). Enzymatic addition of the cap can be accomplished by using, for example, guanylyl transferase and 2′-O-methyltransferase to yield a Cap 0 (^{N7Me}GpppN) or Cap 1 (^{N7Me}GpppN^{2′-OMe}) structure, respectively, while the poly-A tail can be provided through enzymatic addition via poly-A polymerase.

In various embodiments purification or the mRNA construct can be achieved with the application of high-pressure liquid chromatography (HPLC). The resultant drug substance is then formulated into the drug nanoparticle delivery system described herein.

The nanoparticles described herein are formulated so that the mRNA construct, once released into the cytoplasm of a cell, effectively engages the transcriptional machinery of the host cell to generate a sufficient quantity of the encoded immunogen for presentation to the immune system. In this regard a number of parameters can readily be optimized for any particular nucleic acid construct. These include but are not limited to amount/concentration of the nucleic acid in the delivery particle, 5′ capping efficiency and structure; UTR structure, length, and regulatory elements; modification of coding sequence; and poly-A-tail properties.

Thus, for example, the length of the 3′ UTR, 5′ UTR structures, and regulatory elements in both UTRs can impact transcription efficiency. Third, the 5′ 7-methylguanosine (m7G) cap of the mRNA molecule, linked via a triphosphate bridge to the first transcribed nucleotide, facilitates efficient translation, and blocks 5′-3′ exonuclease-mediated degradation. The specific cap structure can play an important role in both protein production and immunogenicity, with incomplete capping (5′ triphosphate) and Cap 0 structures shown to stimulate RIG-1 (see, e.g., Devarkar et al. (2016) Proc. Natl. Acad. Sci. USA, 113: 596-601; Xu et al. (2018) Protein Cell, 9: 246-253; Lassig & Hopfner (2017) J. Biol. Chem. 292: 9000-9009; and the like). Additionally, 2-0′-unmethylated capped RNA can be sequestered by cellular IFN-induced proteins with tetratricopeptide repeats (IFIT1) that prevent the initiation of translation or detected by the cytoplasmic RNA sensor MDA5. Typically, the choice of enzyme and reaction conditions can be optimized to catalyze the highest percentage of cap formation. Additionally, the poly (A) tail and its properties such as length, can be optimized for translation and protection of the mRNA molecule (see, e.g., Goldstrohm & Wickens (2008) Nat. Rev. Mol. Cell Biol. 9: 337-344; Azoubel Lima et al. (2017) Nat. Struct. Mol. Biol. 24: 1057-1064; and the like)).

In certain embodiments codon optimization and modification of nucleotides can contribute to translation efficiency. For example, optimization of guanine and cytosine (GC) content can have a significant impact (see, e.g., Kudla et al. (2016) Plos Biol. 4: e180). Innate immune activation by mRNA can influence its utility as a delivery system. The use of modified nucleosides, such as pseudouridine or N-1-methylpseudouridine to remove intracellular signaling triggers for protein kinase R (PKR) activation, provides enhanced antigen expression and adaptive immune responses (see, e.g., Andersonet al. (2010) Nucleic Acids Res. 38: 5884-5892; Andries et al. (2015) J. Contr. Rel. 217: 337-344; Pardi et al. (2018) J. Exp. Med. 215: 1571-1588; and the like).

It will be appreciated that the foregoing nucleic acid constructs and manufacturing methods are illustrative and non-limiting and using the teaching provided herein, numerous other constructs and manufacturing methods will be available to one of skill in the art.

Development of specific mRNAs that encode a plurality of SARS-Cov-2 epitopes for inducing a potent immune response against SARS-Cov-2 is described below.

Development of mRNA(s) that Encode Multiple SARS-Cov-2 Epitopes.

### Identification of Epitopes.

Our approach has been to construct a series of multi-epitope vaccines that are premised on epitope analysis in Omicron viral variants to assist selection of conserved epitopes in key SARS CoV2 regions that are structurally constrained, free from mutations. To accomplish this, we performed an initial analysis on Omicron BA.1 to identify mutations that impact B-cell and T-cell epitopes in the Spike protein, using this analysis to search for conserved epitopes that also appear in other SARS-CoV-2 VOC and sarbecoviruses. The refinement took into consideration the quantification of interconnectivity between amino acids within each SARS CoV2 protein, using a so-called network approach for providing a topological network score for each residue (McMichael & Carrington (2019) Science, 364: 6439). The network score reflects constraints on mutation, which affects viral fitness and the ability of the immune system to clear the mutated virus. An example of how this technology was used recently for network analysis of structural and non-structural SARS CoV2 and other sarbecovirus protein sequences has been provided by Nathan et al. (2021) Cell, 184: 4401-4413). For further analysis, we also took into consideration the screening of CD8+ T-cell epitopes in human blood samples from SARS CoV2 infected people for assessment of recall T cell responses to stimulation by mega peptide pools (see, e.g., Tarke et al. (2022) Cell, 185: 847-859; Peng et al. (2020) Nature Immunol. 1336: 21). Finally, we also did (i): comparative analysis of conserved linear B-cell epitopes identified in serum blood samples from humans following SARS CoV2 infection or vaccination (see, e.g., Yoshida et al. (2021) Sci Rep. 11: 5934; Amrun et al. (2020) EBioMedicine, 58: 102911102919; Guo et al. (2022) Mol. Biomed. 3: 12; Prakash et al. (2021) J. Immunol. 206: 2566-2582; Wang et al. (2022) Immunity, 55: 998-1012), and (ii) expression of CD4 T-cell epitopes that support antibody production and development of memory B cells (see, e.g., Lu et al. (2021) J. Exp. Med. 218(12): e20211327; Tarke et al. (2022) Cell, 185: 847-859),

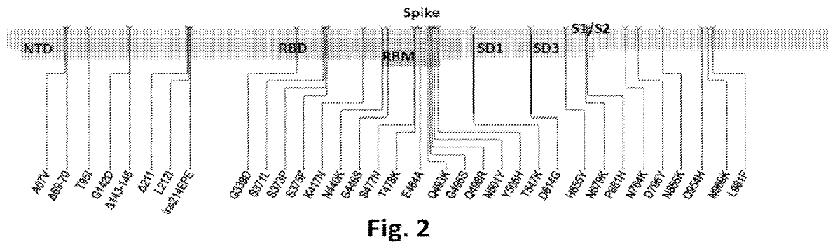

Our first approach was to compare the hotspots of mutation in the Omicron BA.1 variant in relation to CD8+ T-cell epitopes for provisional identification of conserved regions that can be included into a series of multi-epitope vaccines, as depicted in FIG. 1. In order to find a potential series of conserved epitopes for inclusion into a multi-epitope vaccine that induces CD8 T-cell responses (Vaccine I), our approach was to use the Omicron BA.1 spike protein sequence (see, e.g., FIG. 2 Table 1), obtained from the Stanford University Coronavirus Antiviral & Resistance Database (//covdb.stanford.edu/variants/omicron), to search for CD8+ T-cell epitopes, using the software and tools in the NIH Immune Epitope Database (IEDB) (//tools.iedb.org/mhci/). This included the use of the consensus tool, NetMHCpan BA4.0 (//tools.iedb.org/mhci/).

Table 1. Spike protein with mutations. Bold/underline=mutations. Italics/double underline=deletions. Italics/small caps=insertions. The Omicron BA.1 variant is one of the most mutated SARS-CoV-2 VOC. Its high transmissibility and immune evasion ability have raised global concerns. Many Omicron variants have evolved since, including BA.2, BA.2.12.1, BA.2.75, BA.3, BA.4, BA.5, and BA.XE. BA.1 exhibits 30 mutations, 3 deletions, 1 insertion. Most mutations (15) occur in the RBD region, among them, with 10 impacting the RBM, which directly bind to the ACE2 uptake receptor. This explains the enhanced binding between RBD and ACE2 by the conformational changes, resulting from N501Y, Q493K/R, and T478K mutations (Kim et al., bioRxiv [Preprint]. 2022 January 25:2022.01.24.477633). The NTD region has 4 mutations, 3 deletions and 1 insertion, and provides another major target for neutralizing antibodies in addition to RBD. Among the Omicron variants, BA.1 and BA.2 share many common mutations, but also express unique mutations. For instance, BA.2 has an additional 8 unique mutations not seen in BA.1 and lacks 13 of the mutations seen in BA.1. BA.4 and BA.5 spike proteins are most closely related to BA.2. BA.2.75 was independently derived from BA.2, with significant differences from BA.4 and BA.5 (Cao et al., bioRxiv 2022.07.18.500332;). Overall, the combination of novel mutations in Omicron has resulted in variants with higher infectivity than the Wuhan-Hu-1 and Delta variants, as well as significant antibody escape. This gave rise to a 8-127 fold efficacy reduction in the protective effects of current vaccines against Omicron (Shrestha (2022) Rev Med Virol. 2022: e2381). Since the large number of mutations on B cell epitopes are likely responsible for the reduced vaccine efficacy, we used these sets of variants for finally conserved B-cell epitopes.

MFVFLVLLPLVSSQCVNLTTRTQSPPAYTNSFTRGVYYPDKVFRSSVLHSTQDLFLPFFSNVTW
FHAIHVSGTNGTKRFDNPVLPFNDGVYFASIEKSNIIRGWIFGTTLDSKTQSLLIVNNATNVVI
KVCEFQFCNDPFLDVYYHKNNKSWMESEFRVYSSANNCTFEYVSQPFLMDLEGKQGNFKNLREF
VFKNIDGYFKIYSKHTPINLGRDLPQGFSALEPLVDLPIGINITRFQTLLALHRSYLTPGDSSS
GWTAGAAAYYVGYLQPRTFLLKYNENGTITDAVDCALDPLSETKCTLKSFTVEKGIYQTSNFRV
QPTESIVRFPNITNLCPFDEVFNATRFASVYAWNRKRISNCVADYSVLYNFAPFFAFKCYGVSP
TKLNDLCFTNVYADSFVIRGNEVSQIAPGQTGNIADYNYKLPDDFTGCVIAWNSNKLDSKVGGN
YNYLYRLFRKSNLKPFERDISTEIYQAGNKPCNGVAGFNCYFPLRSYGFRPTYGVGHQPYRVVV
LSFELLHAPATVCGPKKSTNLVKNKCVNFNFNGLTGTGVLTESNKKFLPFQQFGRDIADTTDAV
RDPQTLEILDITPCSFGGVSVITPGTNTSNQVAVLYQGVNCTEVPVAIHADQLTPTWRVYSTGS
NVFQTRAGCLIGAEYVNNSYECDIPIGAGICASYQTQTKSHRRARSVASQSIIAYTMSLGAENS
VAYSNNSIAIPTNFTISVTTEILPVSMTKTSVDCTMYICGDSTECSNLLLQYGSFCTQLKRALT
GIAVEQDKNTQEVFAQVKQTYKTPPIKYFGGFNFSQILPDPSKPSKRSFIEDLLFNKVTLADAG
FIKQYGDCLGDIAARDLICAQKFNGLTVLPPLLTDEMIAQYTSALLAGTITSGWTFGAGAALQI
PFAMQMAYRFNGIGVTQNVLYENQKLIANQFNSAIGKIQDSLSSTASALGKLQDVVNHNAQALN
TLVKQLSSKFGAISSVLNDILSRLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYIKWPWYI
WLGFIAGLIAIVMVTIMLCCMTSCCSCLKGCCSCGSCCKFDEDDSEPVLKGVKLHYT
(SEQ ID NO: 8)

To provide broad population coverage, we selected the following HLA alleles: HLA-A*01:01, HLA-B*07:02, HLA-C*01:02, HLA-E*01:01, HLA-G*01:02). Twelve peptide candidates were selected based on percentile score and affinity ranking. Percentile ranking was based on comparing the IC50 of potential epitopes against a random set of peptides from the SWISSPROT database. According to this convention, the epitope percentile score is expressed as an IC50 value (in nM), which is reflective of peptide affinity binding to the selected HLA alleles. As a rough guideline, peptides with IC50 values <50 nM are ranked as high affinity, while values of <500 nM is considered intermediate or low at <5000 nM. This yielded a list of 12 potential CD8+ epitopes with affinity rankings in the high or intermediate-range, which could be used for further consideration (Table 2).

Table 2. The IEDB search identified T cell epitopes in Spike protein of SARS-CoV-2 Omicron BA.1. A search to find conserved CD8+ T-cell epitopes to construct Vaccine I was initiated using the Omicron BA.1 spike protein sequence (see, Table 1). The Immune Epitope Database (IEDB) Consensus tool as well as NetMHCpan BA4.0 were used (//tools.iedb.org/mhci/) in the search. To obtain broad population coverage, we selected HLA-A*01:01, HLA-B*07:02, HLA-C*01:02, HLA-E*01:01, HLA-G*01:02). 12 candidates selected based on affinity ranking were scored for epitope IC50 against the peptides from SWISSPROT database. A lower percentile score is indicative of high affinity and vice versa. Binding affinity is expressed as IC50 (nM). As a rough guideline, peptides with IC50 values <50 nM are considered high affinity, <500 nM intermediate affinity and <5000 nM low affinity. Most of our selected epitopes and high or intermediate affinity scores.

| | SEQ ID | AA | IEDB | NetMHCIIpan |
|---|---|---|---|---|
| Amino acid sequence | NO | coordinates | ranking | BA4.0, IC50 |
| LSFELLHAPATVCGP | 9 | 513-527 | 0.03% | 4.65 |
| VNFNFNGLKGTGVLT | 10 | 539-553 | 0.73% | .31 |
| PLLTDEMIAQYTSAL | 11 | 863-877 | 1.6% | 10.49 |
| KQLSSNFGAISSVLN | 12 | 964-978 | 5.1% | 25.95 |
| FAMQMAYRFNGIGVT | 13 | 898-912 | 11% | 5.95 |
| PTNFTISVTTEILPV | 14 | 715-729 | 11% | 34.68 |
| FCGKGYHLMSFPQSA | 15 | 1,042-1,056 | 13% | 47.54 |
| KLNDLCFTNVY | 16 | 386-396 | 14% | 61.32 |
| TSNFRVQPTESIVRF | 17 | 315-329 | 16% | 63.65 |
| NEVAVLYQDVNCTEV | 18 | 606-620 | 28% | 11.38 |
| RGVYYPDKVFRSSV | 19 | 37-50 | 33% | 46.91 |
| GVFVSNGTHW | 20 | 1,093-1,102 | 37% | 89.54 |

Inhibition concentration: IC50. The predicted output is given as nM units for IC50, where a lower number indicates higher affinity. As a rough guideline, peptides with IC50 values <50 nM are considered high affinity, <500 nM intermediate affinity and <5000 nM low affinity. Most known epitopes have high or intermediate affinity.

Table 3 outlines the epitope distribution in relation to the BA.1 spike protein sequence, with mutations indicated as per the Stanford database. This demonstrates that only 2 out of 12 epitopes were impacted, without signifying these mutations are necessarily less immunogenic.

Table 3. Omicron BA. 1: Spike protein mutations (green bold double underline) in conserved CD8+ T-cell epitopes (bold underline) as per the IEDB search. The CD8 T cell epitopes identified in Table 2 are identified in the Omicron BA.1 sequence including mutations sites. This demonstrates that among the 12 epitopes, only 2 contain mutations, while others are unaffected or conserved. While single point mutations in SARS-CoV-2 can abolish functional responses of individual T cell clones, it is unlikely that across-population mutations will substantially abolish cellular immune deficiency (Moss (2022) Nat. Immunol. 23: 186-193). This is in keeping with the finding that SARS-CoV-2 mRNA vaccines induce a broad range of T cell responses, recognizing SARS CoV2 variants as well as common corona cold viruses (Woldemeskel et al. (2021) J. Clin. Invest. 131:

MFVFLVLLPLVSSQCVNLTTRTQSPPAYTNSFTRGVYYPDKVFRSSVLHSTQDLFLPFFSNVTW
FHAIHVSGTNGTKRFDNPVLPFNDGVYFASTEKSNIIRGWIFGTTLDSKTQSLLIVNNATNVVI
KVCEFQFCNDPFLDVYYHKNNKSWMESEFRVYSSANNCTFEYVSQPFLMDLEGKQGNFKNLREF
VFKNIDGYFKIYSKHTPINLGRDLPQGFSALEPLVDLPIGINITRFQTLLALHRSYLTPGDSSS
GWTAGAAAYYVGYLQPRTFLLKYNENGTITDAVDCALDPLSETKCTLKSFTVEKGIYQTSNFRV
QPTESIVRFPNITNLCPFDEVFNATRFASVYAWNRKRISNCVADYSVLYNFAPFFAFKCYGVSP
TKLNDLCFTNVYADSFVIRGNEVSQIAPGQTGNIADYNYKLPDDFTGCVIAWNSNKLDSKVGGN
YNYLYRLFRKSNLKPFERDISTEIYQAGNKPCNGVAGFNCYFPLRSYGFRPTYGVGHQPYRVVV
LSFELLHAPATVCGPKKSTNLVKNKCVNFNFNGLTGTGVLTESNKKFLPFQQFGRDIADTTDAV
RDPQTLEILDITPCSFGGVSVITPGTNTSNQVAVLYQGVNCTEVPVAIHADQLTPTWRVYSTGS
NVFQTRAGCLIGAEYVNNSYECDIPIGAGICASYQTQTKSHRRARSVASQSIIAYTMSLGAENS
VAYSNNSIAIPTNFTISVTTEILPVSMTKTSVDCTMYICGDSTECSNLLLQYGSFCTQLKRALT
GIAVEQDKNTQEVFAQVKQIYKTPPIKYFGGFNFSQILPDPSKPSKRSFIEDLLFNKVTLADAG
FIKQYGDCLGDIAARDLICAQKFNGLTVLPPLLTDEMIAQYTSALLAGTITSGWTFGAGAALQI
PFAMQMAYRFNGIGVTQNVLYENQKLIANQFNSAIGKIQDSLSSTASALGKLQDVVNHNAQALN
TLVKQLSSKFGAISSVLNDILSRLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYIKWPWYI
WLGFIAGLIAIVMVTIMLCCMTSCCSCLKGCCSCGSCCKFDEDDSEPVLKGVKLHYT
(SEQ ID NO: 21)

Importantly, comparison of all Omicron variants did not deviate from the results obtained with the BA.1 spike protein, showing epitope conservation for 10/12 sequences (Table 4).

TABLE 4
Conserved CD8 T cell epitopes in Spike protein of all Omicron variants
identified by IEDB. Shown is a comparison of the IEDB identified CD8 T cell epitopes
among five Omicron variants. Of the 12 identified epitopes, the same two epitopes
showed mutations in each sub-variant, the rest being mutation free.

| Spike Location | BA.1 | BA.2 | BA.4 |
|---|---|---|---|
| 513-526 | LSFELLHAPATVCG | LSFELLHAPATVCG | LSFELLHAPATVCG |
| (SEQ ID | | | |
| NO: 22) | | | |
| 539-552 | VNFNFNGL | VNFNFNGL | VNFNFNGL |
| (SEQ ID | | | |
| NO: 23) | | | |
| 863-876 | PLLTDEMIAQYTSA | PLLTDEMIAQYTSA | PLLTDEMIAQYTSA |
| (SEQ ID | | | |
| NO: 24) | | | |
| 964-977 | KQLSSKFGAISSVL | KQLSSKFGAISSVL | KQLSSKFGAISSVL |
| (SEQ ID | | | |
| NO: 25) | | | |
| 898-911 | FAMQMAYRFNGIGV | FAMQMAYRFNGIGV | FAMQMAYRFNGIGV |
| (SEQ ID | | | |
| NO: 26) | | | |
| 715-728 | PTNFTISVTTEILP | PTNFTISVTTEILP | PTNFTISVTTEILP |
| (SEQ ID | | | |
| NO: 27) | | | |
| 1,042-1,055 | FCGKGYHLMSFPQS | FCGKGYHLMSFPQS | FCGKGYHLMSFPQS |
| (SEQ ID | | | |
| NO: 28) | | | |
| 386-396 | KLNDLCFTNVY | KLNDLCFTNVY | KLNDLCFTNVY |
| (SEQ ID | | | |
| NO: 29) | | | |
| 315-328 | TSNFRVQPTESIVR | TSNFRVQPTESIVR | TSNFRVQPTESIVR |
| (SEQ ID | | | |
| NO: 30) | | | |
| 606-619 | NQVAVLYQGVNCTE | NQVAVLYQGVNCTE | NQVAVLYQGVNCTE |
| (SEQ ID | | | |
| NO: 31) | | | |
| 37-50 | RGVYYPDKVFRSSV | RGVYYPDKVFRSSV | RGVYYPDKVFRSSV |
| (SEQ ID | | | |
| NO: 32) | | | |
| 1,093-1,102 | GVFVSNGTHW | GVFVSNGTHW | GVFVSNGTHW |
| (SEQ ID | | | |
| NO: 33) | | | |
| Spike | BA.5 | BA.2.75 | |
| 513-526 | LSFELLHAPATVCG | LSFELLHAPATVCG | |
| (SEQ ID NO22) | | | |
| 539-552 | VNFNFNGL | VNFNFNGL | |
| (SEQ ID | | | |
| NO:23) | | | |
| 863-876 | PLLTDEMIAQYTSA | PLLTDEMIAQYTSA | |
| (SEQ ID | | | |
| NO: 24) | | | |
| 964-977 | KQLSSKFGAISSVL | KQLSSKFGAISSVL | |
| (SEQ ID | | | |
| NO: 25) | | | |
| 898-911 | FAMQMAYRFNGIGV | FAMQMAYRFNGIGV | |
| (SEQ ID | | | |
| NO:26) | | | |
| 715-728 | PTNFTISVTTEILP | PTNFTISVTTEILP | |
| (SEQ ID | | | |
| NO: 27) | | | |
| 1,042-1,055 | FCGKGYHLMSFPQS | FCGKGYHLMSFPQS | |
| (SEQ ID | | | |
| NO: 28) | | | |
| 386-396 | KLNDLCFTNVY | KLNDLCFTNVY | |
| (SEQ ID | | | |
| NO: 29) | | | |
| 315-328 | TSNFRVQPTESIVR | TSNFRVQPTESIVR | |
| (SEQ ID | | | |
| NO: 30) | | | |
| 606-619 | NQVAVLYQGVNCTE | NQVAVLYQGVNCTE | |
| (SEQ ID | | | |
| NO: 31) | | | |
| 37-50 | RGVYYPDKVFRSSV | RGVYYPDKVFRSSV | |
| (SEQ ID | | | |
| NO: 32) | | | |
| 1,093-1,102 | GVFVSNGTHW | GVFVSNGTHW | |
| (SEQ ID | | | |
| NO: 33) | | | |

A number of technological approaches and in silico prediction-making software have now become available to determine sequence homology between SARS CoV2 sequences and other sarbecoviruses, in addition to the ability to assign specific epitopes to the activation select T-cell subsets in human blood samples, e.g., cellular activation by epitope megapools (see, e.g. Grifoni et al. (2020) Cell Host Microbe, 27(4): 671-680; Tarke et al. (2021) Cell Rep. Med. 2: 100204; Tarke et al. (2022) Cell, 185: 847-859). This allowed demonstration that T cell responses induced by different vaccine platforms (mRNA-1273, BNT162b2, Ad26.COV2S and NVX-CoV2373) cross-react with SARS-CoV-2 variants. In addition, T cell responses to early variants were preserved across vaccine platforms, in stark contrast to the to the decline of memory B responses, as reflected by the level of neutralizing antibodies (Tarke et al. (2022) Cell, 185: 847-859). Six months post-vaccination, 90% and 87% of CD4+ and CD8+ memory T-cell responses, respectively, were preserved against VOC (including Omicron), as determined by activation-induced marker assays. However, the decline in B-cell responses was considerably more in the Omicron than other variants (Tarke et al. (2022) Cell 185: 847-859). These results are also in good agreement with conservation of T-cell memory function in COVID-19 patients by Peng et al. (2020) Nat. Immunol. 21: 1336-1345), who also used assessment of functional assays in response to a pool of peptides.

Not only is the preservation of T-cell epitopes congruent with the analysis in Table 2, but structure-based proteome analysis can be used to shed light on conserved peptide sequences to develop broadly protective T-cell vaccines. Potentially useful in this consideration, is the network analysis performed by Nathan et al. (2021) Cell, 184: 4401-4413), who pinpointed structurally conserved SARS-CoV-2 amino acid sequences (“highly networked”) that place a constraint on viral mutation and interference in fitness. The mutational resistance of highly networked residues was also confirmed by performing site-directed mutagenesis and assessing the impact on the biological function of the SARS-CoV-2 spike protein. The data were also correlated to viral entropy and the development rate of sequence disorder among a wide range of sarbeco-viruses. An HLA class I peptide stability assay (Kaseke et al. (2021) Cell Rep. 36: 109378) was used to corroborate affinity binding of the CD8+ T-cell epitopes to globally prevalent HLA class I alleles, using the NetMHCpan 4.1 epitope prediction tools (www.cbs.dtu.dk/services/NetMHCpan/). This allowed peptide binding affinity to be established for 18 HLA class I alleles (A*0101, A*0201, A*0301, A*2402, B*0702, B*0801, B*1402, B*1501, B*2705, B*3501, B*3901, B*4001, B*4402, B*5201, B*5701, B*5801, B*8101, and Cw*0701) that cover >99% of the global allele pool (Sette & Sidney (1999) Immunogenetics, 50: 201-212; Sidney et al., (2008) BMC Immunol., 9: 1). Nathan et al. also assessed epitope mutational resistance by performing an analysis of SARS-CoV-2 primary isolates and their immunogenicity in individuals recovering from natural infection or vaccinated with an mRNA-based vaccine. Noteworthy, the analysis culminated in finding 15 highly networked peptide sequences, capable of stabilizing HLA molecules (Table 5).

Table 5. Highly networked regions within SARS-CoV-2 structural and accessory proteins that contain stabilizing CD8+ T-cell epitopes with global HLA coverage (Nathan et al. (2021) Cell, 184: 4401-4413). Nathan et al. identified mutationally constrained regions in the SARS-CoV-2 proteome, using structure-based network analysis to define areas of structural conservation that preserve viral fitness. Based on available high-quality structural data, they were able to calculate amino acid network scores for open and closed spike protein conformations as well as 14 additional proteins that make up 44% of the viral proteome. Residue network scores were binned (<0, 0-2, 2-4, >4) and compared with sequence entropy values from SARS-CoV-2, sarbecoviruses (SARS-CoV-1/bat CoV), and MERS-CoV. This revealed a strong inverse relationship between network measures of topological importance and mutational frequencies. Network scores calculated using structural data for SARS-CoV-1 and MERS-CoV were also highly correlated with SARS-CoV-2 scores, indicating that highly networked residues are likely to be conserved. Bold/underline shows amino acids in Nathan et al. that overlap with our NIEDB search

| | | | AA | SEQ ID |
|---|---|---|---|---|
| Amino acid sequence | Protein | Domain | coordinates | NO: |
| RGVYYPDKVFRSSV | spike | N-terminal S1 | 34-47 | 34 |
| | | domain | | |
| KGIYQTSNFRVQPTESIVR | spike | S1-RBD hinge | 310-329 | 35 |
| F | | domain | | |
| KLNDLCFTNVY | spike | RBD | 386-396 | 36 |
| FELLHAPATV | spike | RBD | 515-524 | 37 |
| TSNEVAVLYQDVNCTEV | spike | C-terminal S1 | 604-620 | 38 |
| | | domain | | |
| TEILPVSMTKTSVDCTMY | spike | N-terminal S2 | 724-741 | 39 |
| | | domain | | |
| PLLTDEMIAQYTSAL | spike | N-terminal S2 | 863-877 | 40 |
| | | domain | | |
| YRFNGIGV | spike | N-terminal S2 | 904-911 | 41 |
| | | domain | | |
| ALNTLVKQLSSNFGAISSV | spike | S2 HR1 domain | 958-984 | 42 |
| LNDILSRL | | | | |
| KRVDFCGKGYHLMSFPQ | spike | S2 CD domain | 1,038-1,062 | 43 |
| SAPHGVVF | | | | |
| GVFVSNGTHW | spike | C-terminal S2 | 1,093-1,102 | 44 |
| | | domains | | |
| NPLLYDANYFLCWHTNCY | ORF3A | domains IV-VI | 137-167 | 45 |
| DYCIPYNSVTSSI | | | | |
| RLFARTRSMWSFNPETNIL | membrane | C-terminal | 101-156 | 46 |
| LNVPLHGTILTR | | domain | | |
| PLLESELVIGAVILRGHLRI | | | | |
| AGHHL | | | | |
| NSSPDDQIGYY | nucleocapsid | RNA-binding | 78-88 | 47 |
| | | domain | | |
| RRGPEQTQGNFGDQELIRQ | nucleocapsid | C-terminal | 277-318 | 48 |
| GTDYKHWPQI | | dimerization | | |
| AQFAPSASAFFGM | | domain | | |

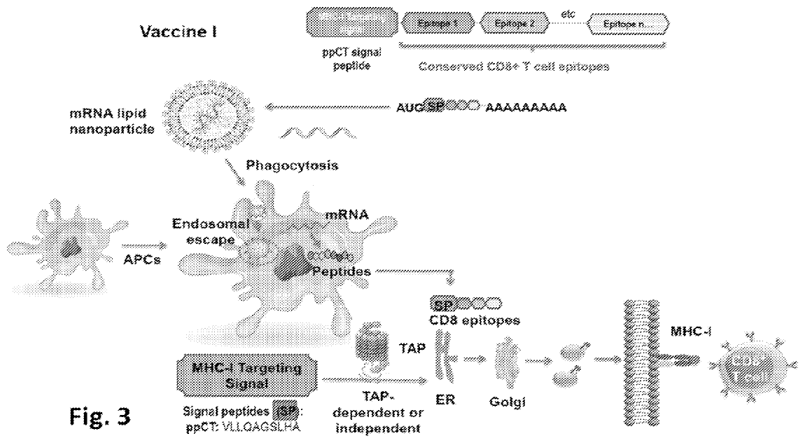

Comparison of the network sequences in Table 5 to our in-house IEDB-derived spike sequences (Table 2), show significant overlap, indicated by bold underline in Table 5. Moreover, display of Nathan et al.'s networked spike protein sequences in relation to the mutational sites in Omicron BA 0.1, confirm mutational alteration of the same sequences (FIG. 3) shown for IEDB analysis in Table 3. All considered, the immunogenicity of these overlapping regions, including functional relevance under natural infectious conditions, makes these viable candidates for developing broadly protective T-cell-based vaccines.

### Type I Vaccine Construction:

As described herein, we illustrate the design of mRNA multi-epitope vaccines that can be used independently or in combination with other covid-19 vaccines (e.g., the spike protein mRNA vaccines) to invoke strong CD8⁺ T-cell responses. This includes the possibility of generating cytotoxic T-cells engaged in viral clearance, memory T-cells that recognize a wide range of corona and sarbecoviruses, as well as tissue-resident memory T-cells that play an early defense role in the mucosal immune system to assist neutralizing antibodies. Vaccine I is premised, inter alia, on a rational combination of well-conserved T-cell epitopes identified in Omicron, showing overlap with other corona and sarbecoviruses (see, e.g., FIG. 1). The epitopes identified, allow vaccine construction based on using spike protein CD8 epitopes (see Table 2), as well as the use of an overlapping set of epitopes that also include conserved sequences from non-spike proteins (see Table 5, supra.). We demonstrate the construction of mRNA-based vaccines for each of the combinations shown in Tables 2 and 5.

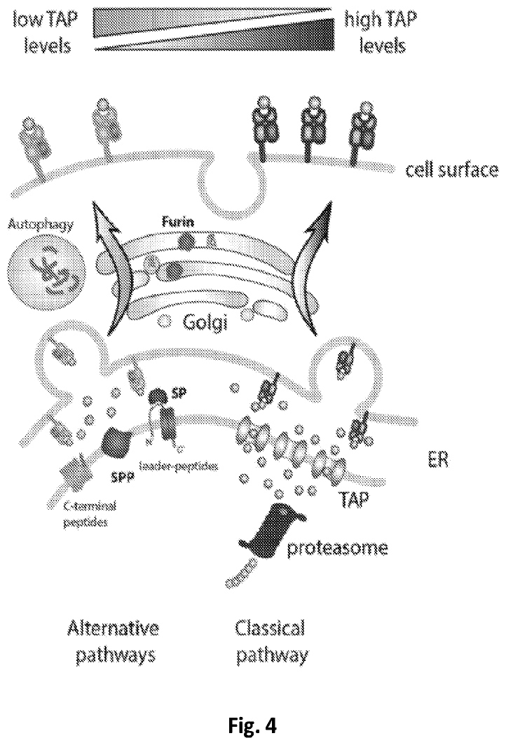

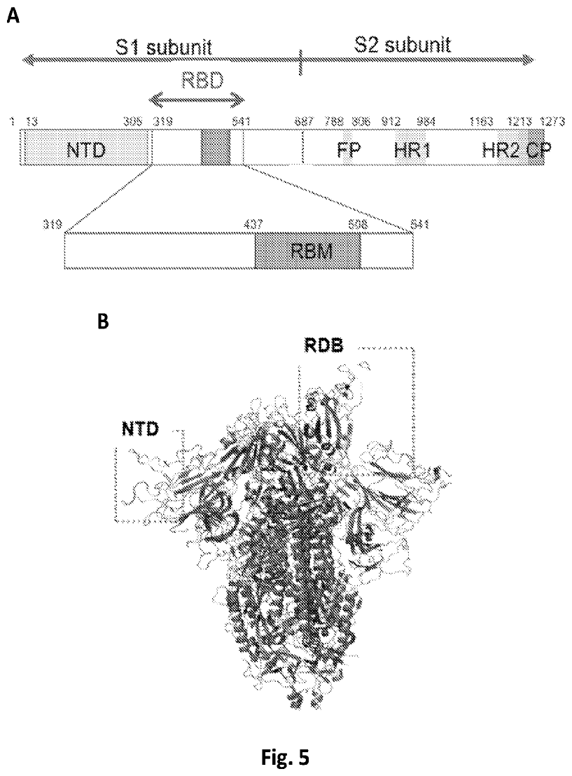

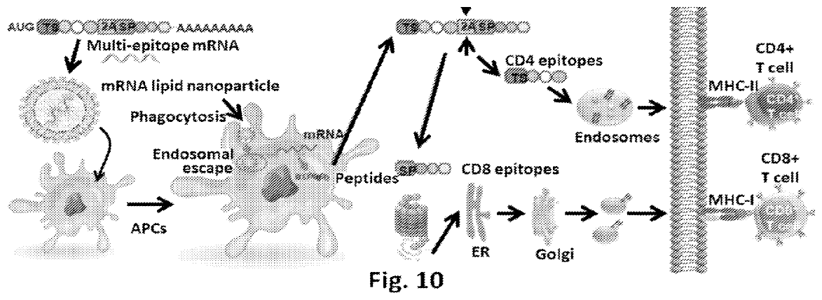

Both Type I vaccine constructs rely on peptide expression in antigen presenting cells (APC), following uptake of lipid nanoparticles delivering a codon-optimized mRNA strand (FIG. 3). The mRNA strand, also includes the instructions for splicing and routing of CD8 plus epitopes to the MHC I complex for T-cell activation (FIG. 3). The nucleic acid construct was developed, using reverse translated epitope sequences for sequential linkage, making use of intermediary flanking or splicing sequences to allow epitope access to MHC I, using classical or alternative MHC I loading pathways, explained in FIGS. 3 and 4. While the classical or proteasome-TAP pathway involves APC degradation in the immuno-proteasome, with epitope import into the endoplasmic reticulum (ER) by transporter associated with antigen processing (TAP) proteins, the alternative pathway involves the use of signaling peptide sequences that can be added upstream of peptide sequences or epitopes. These hybrid peptides are inserted into the ER membrane, where they are processed and released independent of proteasomes and TAP (see, e.g., Minev et al. (2000) Eur. J. Immunol. 30: 2115-2124; Durgeau et al. (2018) Nature Com. 9: 5097; Kovjazin & Cannon, supra.; Hage et al. (2013) Ann. N. Y. Acad. Sci. 2013; 1283: 75-80). While it is possible to achieve MHC 1 expression of exogenous antigens by both pathways, in the current example, we will use the inclusion of upstream leader signal peptide sequence, VLLQAGSLHA (SEQ ID NO:49), derived from the human pre-procalcitonin (ppCT) signal peptide (Durgeau et al. (2018) Nature Com. 9: 5097). This signal peptide represents one of several leader sequences that can facilitate epitope access and presentation by the MHC-I complex for the activation of naïve CD8+ T-cells (FIG. 10). ppCT16-25 (VLLQAGSLHA (SEQ ID NO: 49) is encoded by the calcitonin-related polypeptide alpha gene and allows incorporation of transcribed hybrid peptide sequence into the ER membrane, from where it is removed by a signal peptidase (SPase) protease, as explained in FIG. 4. In addition to SEQ ID NO: 49, MHC I sorting can also be accomplished through the use of alternative lead sequences, shown in Table 6.

TABLE 6
Alternative pathway TEIPP antigens.

| Peptide | SEQ ID | MHC | | |
|---|---|---|---|---|
| Sequence | NO. | Class I | Source Protein | Presented upon |
| MCLRMTAVM | 50 | H2-D | Trh4 | Tap deficiency |
| CVNKTERAY | 51 | H2-K | Unknown | Tap deficiency |
| FAPLPRLPTL | 52 | Qa-1 | Acyl carrier protein | Tap deficiency |
| FYAEATPML | 53 | Qa-1 | Fam49b | ERAAP deficiency |
| VLLQAGSLHA | 54 | HLA- | Pre-procalcitonin | Tap deficiency |
| | | A2*o201 | | |
| AAGIGILTV | 55 | HLA- | MART-1 | Tap-deficient and Tap- |
| | | A2.1 | | expressing cells |

Use of the Highly Networked SARS-CoV-2 Structural and Accessory Protein Domains that Contain Stabilizing CD8+ T-Cell Epitopes for the Construction of mRNA Vaccines

The observation that mRNA-based vaccine recipients solicit modest CD8+ T-cell responsiveness to highly networked spike epitopes present the opportunity to improve vaccine-induced immunity against SARS-CoV-2 and its variants of concern. Given that highly networked regions from structural and accessory proteins (which harbor 53 epitopes for 18 HLA class I alleles) can be combined into collective set of sequences that encompass 315 amino acids (see, e.g., Table 5, supra.), this establishes an immunogen repertoire that can be incorporated by a number of vaccine delivery platforms. We believe this networked T-cell immunogen can be delivered alongside a spike-based vaccine as a tandem molecule in the same nanocarrier or as a separate co-delivered physical entity. This would ensure the induction of de novo responses to highly networked CD8+ T-cell epitopes, which would assist viral clearance and protection in concert with generating neutralizing antibodies.

The detailed design of a mRNA multi-epitope vaccine follows rational principles for construction of a nucleic acid vaccine. Utilizing the compilation of topologically important SARS-CoV-2 viral protein epitopes for CD8 T-cell activation, identified by structure-based network analysis, we designed a cDNA construct to prepare the mRNA strand for the vaccine. Briefly, this was accomplished by reverse translation of the peptide into cDNA nucleotide sequences. These sequences are then linked together to create a linear cDNA construct through joining sequences (see below). In addition, the cDNA construct can be provided with targeting sequences that enhance antigen access to the MHC-I endosomal compartment for peptide presentation to CD8+ T-cells (or for MHC-II targeting to activate CD4+ T cells, as will be described later). The next design step is to use of codon optimization for the integrated cDNA construct, which allows optimal gene expression in accordance with the tRNA pool for human versus non-human cells. The Codon Optimization tool from GenSmart™ (www.genscript.com/gensmart-free-gene-codon-optimization.html) uses a newly-designed algorithm based on the “Population Immune Algorithm”, which takes advantage of population genetics as well as immunology theory to maximize the gene expression. The optimized cDNA sequence is then inserted into a bacterial plasmid vector, such as pTNT (Promega). These vectors typically contain tandem T7 promoters for transcription of mRNA, a 5′ UTR from rabbit 0-globin, and a synthetic poly(A) tail, which are important for efficient protein expression. Following plasmid amplification and purification, the plasmid(s) is/are linearized by restriction enzyme cutting, to facilitate in vitro translation into mRNA constructs through T7 polymerase. A 7-methyl-guanosine cap is added to the 5′ end of RNA using a vaccinia virus capping enzyme. The cap is essential for mRNA stability and efficient translation. The mRNA coding sequence is further modified by a non-natural RNA nucleobase N1-methylpseudouridine (m1Ψ) to reduce the likelihood that the nucleic acid construct triggers robust immunological danger signals (e.g., through interaction with toll-like receptors). Following the construction of the integrated nucleic acid strand, the mRNA is encapsulated in polymer and/or lipid nanoparticles, that are used for delivery and protection against RNA degradation after vaccine administration.

One important consideration in preparing the cDNA construct is to incorporate our customized approach for splicing the nucleotide sequences together, allowing efficient epitope spacing and subsequent splicing in the cytosol or the MHC-I endosomal compartment. This is accomplished by the use of flanking sequences to provide epitope spacing, allow splicing and MHC-I presentation. What follows is an illustrative example of developing a multi-epitope mRNA vaccine can for vaccination against highly networked SARS-CoV-2 domains for generation of robust CD8+ T-cell responses. We will follow that description by a discussion of how the design can be used for the formulation of additional multi-epitope mRNA vaccines.

Reverse Translation and cDNA Design of a Multi-Epitope Vaccine for Generating Cytotoxic T-Cell Responses

Table 7, shows the amino acid sequences of the epitopes, to which we added flanking sequences before reverse translation of peptide sequences into cDNA. The underlined flanking sequences in Table 7 have been introduced for demarcation of the epitope boundaries and represent natural flanking amino acids serving as recognition sites for further immune processing. Additionally, or alternatively, one or more lysines can be added as spacers. Lysine has a positive charge, which can facilitate epitope binding to the MHC-1 peptide groove, which has a closed structure. This leads to epitope tucking into the groove without overhang, in contrast to the open binding pocket of MHC-II, where 15-20 amino acid peptides are allowed to protrude and even hang over the end of the groove. Peptide binding to the MHC-I groove allows the TCR t contact with the entire peptide sequence. However, the addition of a positively charged lysine residue to the C-terminus allows opening of the “K end” of the MHC-J groove, facilitating non-canonical epitope presentation as shown by Remesh et al. (2017) J. Biol. Chem. 292(13): 5262-5270).

TABLE 7
Reverse translation of epitopes shown in Table 5 with flanking sequences.
Flanking sequence shown as bold/underline.

| AA | | |
|---|---|---|
| coordinates | Amino acid sequence | cDNA sequence |
| 34-47 | FTRGVYYPDKVFRSSVLH | tttacccgcggcgtgtattatccggataaa |
| | (SEQ ID NO: 56) | gtgtttcgcagcagcgtgctgcat |
| | | (SEQ ID NO: 57) |
| 310-329 | VEKGIYQTSNFRVQPTESIVRFPN | gtggaaaaaggcatttatcagaccagcaac |
| | (SEQ ID NO: 58) | tttcgcgtgcagccgaccgaaagcattgtg |
| | | cgctttccgaac |
| | | (SEQ ID NO: 59) |
| 386-396 | PTKLNDLCFTNVYAD | ccgaccaaactgaacgatctgtgctttacc |
| | (SEQ ID NO: 60) | aacgtgtatgcggat |
| | | (SEQ ID NO: 61) |
| 515-524 | LSFELLHAPATVCG | ctgagctttgaactgctgcatgcgccggcg |
| | (SEQ ID NO: 62) | accgtgtgcggc |
| | | (SEQ ID NO: 63) |
| 604-620 | TNTSNEVAVLYQDVNCTEVPV | Accaacaccagcaacgaagtggcggtgctg |
| | (SEQ ID NO: 64) | tatcaggatgtgaactgcaccgaagtgccg |
| | | gtg (SEQ ID NO: 65) |
| 724-741 | VTTEILPVSMTKTSVDCTMYIC | Gtgaccaccgaaattctgccggtgagcatg |
| | (SEQ ID NO: 66) | accaaaaccagcgtggattgcaccatgtat |
| | | atttgc (SEQ ID NO: 67) |
| 863-877 | LPPLLTDEMIAQYTSALLA | Ctgccgccgctgctgaccgatgaaatgatt |
| | (SEQ ID NO: 68) | gcgcagtataccagcgcgctgctggcg |
| | | (SEQ ID NO: 69) |
| 904-911 | MAYRFNGIGVTQ | Atggcgtatcgctttaacggcattggcgtg |
| | (SEQ ID NO: 70) | acccag (SEQ ID NO: 71) |
| 958-984 | AQALNTLVKQLSSNFGAISSVLNDIL | Gcgcaggcgctgaacaccctggtgaaacag |
| | SRLDK | ctgagcagcaactttggcgcgattagcagc |
| | (SEQ ID NO: 72) | gtgctgaacgatattctgagccgcctggat |
| | | aaa (SEQ ID NO: 73) |
| 1,038-1,062 | QSKRVDFCGKGYHLMSFPQSAPHGVV | Cagagcaaacgcgtggatttttgcggcaaa |
| | FLH | ggctatcatctgatgagctttccgcagagc |
| | (SEQ ID NO: 74) | gcgccgcatggcgtggtgtttctgcat |
| | | (SEQ ID NO: 75) |
| 1,093-1,102 | REGVFVSNGTHWFV | Cgcgaaggcgtgtttgtgagcaacggcacc |
| | (SEQ ID NO: 76) | cattggtttgtg (SEQ ID NO: 77) |
| 137-167 | SKNPLLYDANYFLCWHTNCYDYCIPY | Agcaaaaacccgctgctgtatgatgcgaac |
| | NSVTSSIVI | tattttctgtgctggcataccaactgctat |
| | (SEQ ID NO: 78) | gattattgcattccgtataacagcgtgacc |
| | | agcagcattgtgatt (SEQ ID |
| | | NO: 79) |
| 101-156 | SFRLFARTRSMWSFNPETNILLNVPL | agctttcgcctgtttgcgcgcacccgcagc |
| | HGTILTRPLLESELVIGAVILRGHLR | atgtggagctttaacccggaaaccaacatt |
| | IAGHHLGR | ctgctgaacgtgccgctgcatggcaccatt |
| | (SEQ ID NO: 80) | ctgacccgcccgctgctggaaagcgaactg |
| | | gtgattggcgcggtgattctgcgcggccat |
| | | ctgcgcattgcgggccatcatctgggccgc |
| | | (SEQ ID NO: 81) |
| 78-88 | NTNSSPDDQIGYYRR | Aacaccaacagcagcccggatgatcagatt |
| | (SEQ ID NO: 82) | ggctattatcgccgc (SEQ ID |
| | | NO: 83) |
| 277-318 | FGRRGPEQTQGNFGDQELIRQGTDYK | Tttggccgccgcggcccggaacagacccag |
| | HWPQIAQFAPSASAFFGMSR | ggcaactttggcgatcaggaactgattcgc |
| | (SEQ ID NO: 84) | cagggcaccgattataaacattggccgcag |
| | | attgcgcagtttgcgccgagcgcgagcgcg |
| | | ttttttggcatgagccgc (SEQ ID |
| | | NO: 85) |

Table 8 shows the combined cDNA sequence for the series of peptides with flanking regions, as called out in Table 2. Three different colors are used for demonstrating the cDNA sequence for each of the peptides, shown in Table 2.

TABLE 8
illustrates the combined amino acid sequences with flanking regions (SEQ ID
NO:86) and the ″combined″ cDNA sequence (SEQ ID NO: 87) encoding the amino acid
sequence. Top row - Protein sequence: underline = flanking sequences. Bottom row -
DNA sequence: Small caps = natural flanking sequences. Single underline - T cell
epitopes, bold/italic = T cell epitopes, double underline = T cell epitopes. Single
underline, double underline and bold/italic delineate the position of individual reverse
translated peptides in Table 7, with maintenance of the same sequence as shown in Table

FTRGVYYPDKVFRSSVLHVEKGIYQTSNFRVQPTESIVRFPNPTKLNDLCFTNVYADLSFELLH
APATVCGTNTSNEVAVLYQDVNCTEVPVVTTEILPVSMTKTSVDCTMYICLPPLLTDEMIAQYT
SALLAMAYRFNGIGVTQAQALNTLVKQLSSNFGAISSVLNDILSRLDKQSKRVDFCGKGYHLMS
FPQSAPHGVVFLHREGVFVSNGTHWFVSKNPLLYDANYFLCWHTNCYDYCIPYNSVTSSIVISF
RLFARTRSMWSFNPETNILLNVPLHGTILTRPLLESELVIGAVILRGHLRIAGHHLGRNTNSSP
DDQIGYYRRFGRRGPEQTQGNFGDQELIRQGTDYKHWPQIAQFAPSASAFFGMSR
(SEQ ID NO: 86)
TTACccgcggcgtgtattatccggataaagtgtttcgcagcagcgtgCTGCATGTGGAAaaaggcatt
tatcagaccagcaactttcgcgtgcagccgaccgaaagcattgtgcgctttcCGAACCCGACcaaac
tgaacgatctgtgctttaccaacgtgtATGCGGATCTGAGCTttgaactgctgcatgcgccggcgac
cgtgTGCGGCACCAACaccagcaacgaagtggcggtgctgtatcaggatgtgaactgcaccgaagtg
ccggtggtgaccaccgaaattctgccggtgagcatgaccaaaaccagcgtggattgcaccatgt
atATTTGCCTGCCGCcgctgctgaccgatgaaatgattgcgcagtataccagcgcgctgCTGGCGATGG
cGtatcgctttaacggcattggcgtgACCCAGGCGCAGgcgctgaacaccctggtgaaacagctgag
cagcaactttggcgcgattagcagcgtgctgaacgatattctgagccgcctgGATAAACAGAGCaaa
cgcgtggatttttgcggcaaaggctatcatctgatgagctttccgcagagcgcgccgcatggcg
tggtgtttCTGCATCGCGAAggcgtgtttgtgagcaacggcacccattggTTTGTGAGCAAAaacccgC
tgctgtatgatgcgaactattttctgtgctggcataccaactgctatgattattgcattccgta
taacagcgtgaccagcagcattGTGATTAGCTTTCgcctgtttgcgcgcacccgcagcatgtggagc
tttaacccggaaaccaacattctgctgaacgtgccgctgcatggcaccattctgacccgcccgc
tgctggaaagcgaactggtgattggcgcggtgattctgcgcggccatctgcgcattgcgggcca
tcatctgGGCCGCAACACcaacagcagcccggatgatcagattggctattatCGCCGCTTTGGCCgCCg
cggcccggaacagacccagggcaactttggcgatcaggaactgattcgccagggcaccgattat
aaacattggccgcagattgcgcagtttgcgccgagcg
(SEQ ID NO: 87)

### Incorporation of an Endosomal Targeting Sequence, Start Codon, Stop Codon

Start and stop codons are necessary for cytosolic expression of the nucleotide construct. The start codon marks the site at which translation into the protein sequence begins, and the stop codon marks the site at which translation ends. We use an AUG sequence (ATG in the corresponding DNA sequence) as start codon (methionine) together with UAA, UAG, or UGA as stop codons. The stop codons encode for a release factor that causes translation to cease. It will be recognized that in various embodiments, any start and stop codon known to those of skill in the art can be utilized.

Table 9 shows the start (ATG) and stop (TAA) codons added to the nucleic acid sequence shown in Table 8.

TABLE 9
Nucleic acid sequence of Table 8 with added start (ATG) and stop (TAA)
codons. Single underline-T cell epitopes, bold/italic = T cell epitopes,
double underline = T cell epitopes.

ATGtttacccgcggcgtgtattatccggataaagtgtttcgcagcagcgtgctgcatgtggaaa
aaggcatttatcagaccagcaactttcgcgtgcagccgaccgaaagcattgtgcgctttccgaa
cccgaccaaactgaacgatctgtgctttaccaacgtgtatgcggatctgagctttgaactgctg
catgcgccggcgaccgtgtgcggcaccaacaccagcaacgaagtggcggtgctgtatcaggatg
tgaactgcaccgaagtgccggtggtgaccaccgaaattctgccggtgagcatgaccaaaaccag
cgtggattgcaccatgtatatttgcctgccgccgctgctgaccgatgaaatgattgcgcagtat
accagcgcgctgctggcgatggcgtatcgctttaacggcattggcgtgacccaggcgcaggcgc
tgaacaccctggtgaaacagctgagcagcaactttggcgcgattagcagcgtgctgaacgatat
tctgagccgcctggataaacagagcaaacgcgtggatttttgcggcaaaggctatcatctgatg
agctttccgcagagcgcgccgcatggcgtggtgtttctgcatcgcgaaggcgtgtttgtgagca
acggcacccattggtttgtgagcaaaaacccgctgctgtatgatgcgaactattttctgtgctg
gcataccaactgctatgattattgcattccgtataacagcgtgaccagcagcattgtgattagc
tttcgcctgtttgcgcgcacccgcagcatgtggagctttaacccggaaaccaacattctgctga
acgtgccgctgcatggcaccattctgacccgcccgctgctggaaagcgaactggtgattggcgc
ggtgattctgcgcggccatctgcgcattgcgggccatcatctgggccgcaacaccaacagcagc
ccggatgatcagattggctattatcgccgctttggccgccgcggcccggaacagacccagggca
actttggcgatcaggaactgattcgccagggcaccgattataaacattggccgcagattgcgca
gtttgcgccgagcgcgagcgcgttttttggcatgagccgcTAA (SEQ ID NO: 88)

Following design of the integrated cDNA construct, codon optimization was performed. Codon optimization is a gene engineering approach for improving gene expression by changing synonymous codons based on an organism's codon bias. For the purpose of designing a nanocarrier for optimal gene expression in humans, we used the codon optimization tool from GENSMART™ Codon Optimization (www.genscript.com/gensmart-free-gene-codon-optimization.html). This is a readily accessible and user-friendly platform that uses a newly designed algorithm based on the “Population Immune Algorithm”. This algorithm takes advantage of population genetics as well as immunology theory. The paradigm also considers more than 200 factors involved in gene expression, including GC content, codon usage and content index, RNase splicing sites, as well as screening and validating cis-acting mRNA destabilizing motifs. Instead of applying a single-factor simulation for computing, a multifactor approach was employed to ensure that all key factors in our target gene sequences carry the appropriate weight. Consequently, each gene optimization was fully customized to maximize the chance of obtaining a functional and active protein. All of the parameters are integrated into the algorithm and require no user input, other than adding our gene sequence and the desired species.

It will be recognized, however, that numerous other codon optimization tools are known to those of skill in the art can readily be utilized to optimize the nucleic acid sequence for the target organism and/or cell type.

Upon entry of the codon sequence shown in Table 9, we obtained an codon construct by GENSMART™ Codon Optimization with a GC content of 56.23%, as shown in Table 10.

TABLE 10
Codon optimized cDNA construct (SEQ ID NO: 89). Average GC content:
56.23%. Single underline, bold/italic, and double underline represent the same epitopes,
as shown in Table 9.

ATGttcacccggggggtgtactatcctgataaggtgttcagatccagcgtgctgcacgtggaaaagggcatctaccagac
cagcaatttccgcgtgcagcctaccgaatctatgtgcggttccccaaccccaccaagctgaacgatctgtgttttaccaatgt
gtacgccgacctgtctttcgagctgctgcacgcccctgccaccgtgtgtggcacaaacacaagcaacgaggtggccgtgct
gtaccaggacgtgaactgtacagaggttcctgtggttacaaccgagatcctccccgtgtccatgacaaaaacctccgtcgact
gcaccatgtacatctgcctgccacctctgctgacagatgagatgatcgcccaatacacctccgccctgctggccatggcttaca
gattcaatggcattggagtgacccaggcccaggccctgaataccctggtgaagcagctgagctctaatttcggcgccatctct
agcgtgctgaacgacatcctgagccggctggacaaacagagcaagcgggtggatttctgcggcaagggatatcacctgatg
agcttccctcaaagcgccccccacggcgtggtgttcctgcatagggaaggcgtctttgtgtctaacggcacccactggttcgt
gtccaagaaccccctgctgtacgacgccaactacttcctgtgctggcacaccaactgctacgactactgcatcccctacaacag
cgtgaccagcagcatcgtgattagctttagactgttcgccagaaccagaagtatgtggagctttaacccagagacaaacatcct
gctgaacgtgcctctgcacggcacaatccttacaagacctctgctggaaagcgagctggtcatcggcgctgtgatcctgcggg
gacacctgagaatcgccggccaccacctgggcagaaacaccaacagcagccctgacgaccagatcggctactaccgga
gattcggcagaagaggtcctgaacagacccaaggcaacttcggcgatcaggagctcatcagacagggcaccgactacaag
cactggcctcagatcgcccagttcgcacctagcgcttctgcttttttcgaatgagcagaTGA (SEQ ID NO: 89)

In the above example the cytosolic mRNA expression product is designed to engage the proteolytic machinery for peptide cleavage in the cytosol, leading to the import of the epitopes into the MHC class I endosomal pathway by TAP transporters. Following endosomal trafficking to the antigen presenting cell surface, this allows epitope presentation to T-cells (see, e.g., Oliveira & van Hall (2015) Front. Immunol. 6: 298). The addition of the flanking epitope sequences allows the 26S proteosome to perform proteolytic cleavage of the expressed polypeptide sequence through sequence recognition; peptide cleavage releases the MHC-I interactive peptides (Id.). It is also important to take into consideration the existence of a number of alternative processing pathways that can lead to antigen presentation and that may prove useful for epitope presentation (Id.). This includes a number of viral and endogenous TAP-independent pathways that can facilitate peptide binding to the MHC class I groove (Hage et al. (2008) Proc. Natl. Acad. Sci. USA, 105: 10119-10124). Some of these peptides may not be displayed in TAP-positive cells and also being referred to as TEIPP, short for T-cell epitopes associated with impaired peptide processing. For example, the peptide sequence VLLQAGSLHA (SEQ ID NO:49) is derived from human pre-procalcitonin (ppCT) signal peptide and is generated in the endoplasmic reticulum by signal peptidase and signal peptide peptidase. This sequence can be used as a leader sequence to provide alternative means of accomplishing MHC-I epitope presentation to CD8 T-cells (Durgeau et al. (2018) Nat. Commun. 9: 5097). Reverse translation of VLLQAGSLHAKK (SEQ ID NO:90) (KK was added as a flanking sequence to enhance MHC-I presentation to CD8+ T-cells) yields gtgctgctgcaggcgggcagcctgcatgcg (SEQ ID NO:91). Table 11 shows a nucleic acid construct further encoding the VLLQAGSLHAKK (SEQ ID NO:90) leader plus spacer sequence (SEQ ID NO:90).

TABLE 11
illustrates the nucleic acid construct further encoding the VLLQAGSLHAKK
(SEQ ID NO: 90) leader plus spacer sequence shown in capitals (SEQ ID
NO: 90). The sequence includes the ATG start codon and the TAA stop codon.
Single underline-T cell epitopes, bold/italic = T cell epitopes, double
underline = T cell epitopes.

atgGTGCTGCTGCAGGCGGGCAGCCTGCATGCGAAAAAAtttacccgcggcgtgtattatccgg
ataaagtgtttcgcagcagcgtgctgcatgtggaaaaaggcatttatcagaccagcaactttcg
cgtgcagccgaccgaaagcattgtgcgctttccgaacccgaccaaactgaacgatctgtgcttt
accaacgtgtatgcggatctgagctttgaactgctgcatgcgccggcgaccgtgtgcggcacca
acaccagcaacgaagtggcggtgctgtatcaggatgtgaactgcaccgaagtgccggtggtgac
caccgaaattctgccggtgagcatgaccaaaaccagcgtggattgcaccatgtatatttgcctg
ccgccgctgctgaccgatgaaatgattgcgcagtataccagcgcgctgctggcgatggcgtatc
gctttaacggcattggcgtgacccaggcgcaggcgctgaacaccctggtgaaacagctgagcag
caactttggcgcgattagcagcgtgctgaacgatattctgagccgcctggataaacagagcaaa
cgcgtggatttttgcggcaaaggctatcatctgatgagctttccgcagagcgcgccgcatggcg
tggtgtttctgcatcgcgaaggcgtgtttgtgagcaacggcacccattggtttgtgagcaaaaa
cccgctgctgtatgatgcgaactattttctgtgctggcataccaactgctatgattattgcatt
ccgtataacagcgtgaccagcagcattgtgattagctttcgcctgtttgcgcgcacccgcagca
tgtggagctttaacccggaaaccaacattctgctgaacgtgccgctgcatggcaccattctgac
ccgcccgctgctggaaagcgaactggtgattggcgcggtgattctgcgcggccatctgcgcatt
gcgggccatcatctgggccgcaacaccaacagcagcccggatgatcagattggctattatcgcc
gctttggccgccgcggcccggaacagacccagggcaactttggcgatcaggaactgattcgcca
gggcaccgattataaacattggccgcagattgcgcagtttgcgccgagcgcgagcgcgtttttt
ggcatgagccgctaa (SEQ ID NO: 92)

A codon-optimized version of this construct is shown in Table 12.

TABLE 12
Codon-optimized construct of Table 11. Average GC content: 56.38%. The
leader plus spacer sequence is shown in capitals. The sequence includes the
ATG start codon and the TAA stop codon. Single underline - T cell epitopes,
bold/italic = T cell epitopes, double underline = T cell epitopes.

atgGTGCTGCTGCAGGCCGGCAGCCTCCACGCCAAGAAGttcaccagaggcgtgtattaccccg
acaaggtgtttagaagcagcgtgctgcacgtggaaaagggcatctaccaaacaagcaacttccg
ggtgcagcctaccgaaagcatcgtgcggtttccaaaccctacaaaactgaacgacctgtgtttt
accaatgtgtacgccgacctgagcttcgagctgctgcatgctcctgccacagtgtgcggcacca
acacatctaacgaggtggccgtgctgtaccaggacgtgaactgcacagaagtgcctgtggtgac
aaccgagatcctgcctgtgagcatgaccaagaccagcgtggattgcaccatgtacatctgcctg
ccccccctgctcaccgacgagatgatcgcccagtacacctctgccctgctggccatggcctaca
gattcaacggaatcggcgttacacaggcccaggccctgaacaccctggtgaaacagctgagcag
caacttcggcgctatctcttccgtgctgaatgacattctgagcagactggacaagcagagcaag
cgggtggatttctgcggcaaaggctaccacctgatgtctttcccccaaagcgccccccacggcg
tcgtgttcctgcatagagagggcgtgttcgtgtccaacggcacacactggttcgtgagcaagaa
ccctctgctttacgatgccaactacttcctgtgttggcacaccaactgctacgactactgtatc
ccttataacagcgtgacaagctccatcgttatttcttttcggctgttcgctagaacccggagca
tgtggtccttcaatccagaaaccaacatcctgctgaacgtccccctgcacgggaccatcctgac
cagacctctgctggaaagcgagctggttatcggcgccgtgatcctgcgcggccacctgagaatc
gccggccaccaccttggacgaaacaccaatagcagccctgatgaccagatcggctactaccgga
gattcggcagaaggggacctgagcagacccagggcaattttggagatcaggagctgatcagaca
gggaacagactacaagcactggcctcaaatcgcccagttcgccccttctgcatctgccttcttc
ggcatgagcagataa (SEQ ID NO: 93)

It will be recognized that in various other embodiments other codon-optimizations may be utilized.

Endowing the Multi-Epitope Linkage with Embedded Proteolytic Cleavage Sequences.

Liberation of MHC-I interactive peptides can also occur through the action of proteolytic enzymes in the trans-Golgi network, independent of the cytosol-localized proteosome. This pathway constitutes an alternative and specific peptide release mechanism should flanking sequences prove to be ineffective. One of the principal proteolytic enzymes involved is furin, a known protease located in the trans-Golgi network (Oliveira & van Hall (2015) Front. Immunol. 6: 298). This proteolytic enzyme is required for the maturation of secreted proteins (e.g., growth factors and neurotransmitters) by cleaving at precise stretches of three to four basic amino acid residues (see insert). Furin is part of a family of proprotein convertases that are comprised of 9 members. Three members (PC5/6, PACE4, and PC7), including furin, are widely expressed and cooperate in a variety of proteolytic processes occurring in the trans-Golgi network, cell surface, or endosomes. This includes a role for furin in the secretory pathway where the cleavage of antigens leads to peptide release for MHC class I presentation on the cell surface (Id.). As such, this could lead to the generation of CD8 T-cell responses in vivo in a TAP-independent fashion. Furin is capable of processing a wide variety of precursor proteins after the C-terminal arginine residue in the preferred consensus motif, Arg-X-Lys/Arg-Arg (SEQ ID NO:94). The use of valine as amino acid X, provides the target sequence, RVRR (SEQ ID NO:95). Accordingly, we used this sequence to insert the corresponding nucleotide sequence (cgcgtgcgccgc (SEQ ID NO:96) into the cDNA construct (see, e.g., Table 13).

TABLE 13
Combined cDNA sequence (SEQ ID NO: 97) with nucleotide sequences of Furin
cleavage sites (shown in small caps). The furin cleavable sequence (small caps)
were inserted in between the T cell epitopes shown by single underline, double
underline, and bold/italic.

atgGTGCTGCTGCAGGCGGGCAGCCTGCATGCGAAAAAAtttacccgcggcgtgtattatccgg
ataaagtgtttcgcagcagcgtgctgcatCGCGTGCGCCGCgtggaaaaaggcatttatcagaccag
caactttcgcgtgcagccgaccgaaagcattgtgcgctttccgaacCGCGTGCGCCGCccgaccaaa
ctgaacgatctgtgctttaccaacgtgtatgcggatCGCGTGCGCCGCctgagctttgaactgctgc
atgcgccggcgaccgtgtgcggcCGCGTGCGCCGCaccaacaccagcaacgaagtggcggtgctgta
tcaggatgtgaactgcaccgaagtgccggtgCGCGTGCGCCGCgtgaccaccgaaattctgccggtg
agcatgaccaaaaccagcgtggattgcaccatgtatatttgcCGCGTGCGCCGCctgccgccgctgc
tgaccgatgaaatgattgcgcagtataccagcgcgctgctggcgcGCGTGCGCCGCatggcgtatcg
ctttaacggcattggcgtgacccagCGCGTGCGCCGCgcgcaggcgctgaacaccctggtgaaacag
ctgagcagcaactttggcgcgattagcagcgtgctgaacgatattctgagccgcctggataaaC
GCGTGCGCCGCcagagcaaacgcgtggatttttgcggcaaaggctatcatctgatgagctttccgc
agagcgcgccgcatggcgtggtgtttctgcatCGCGTGCGCCGCcgcgaaggcgtgtttgtgagcaa
cggcacccattggtttgtgCGCGTGCGCCGCagcaaaaacccgctgctgtatgatgcgaactatttt
ctgtgctggcataccaactgctatgattattgcattccgtataacagcgtgaccagcagcattg
tgattCGCGTGCGCCGCagctttcgcctgtttgcgcgcacccgcagcatgtggagctttaacccgga
aaccaacattctgctgaacgtgccgctgcatggcaccattctgacccgcccgctgctggaaagc
gaactggtgattggcgcggtgattctgcgcggccatctgcgcattgcgggccatcatctgggcc
gcCGCGTGCGCCGCaacaccaacagcagcccggatgatcagattggctattatcgccgcCGCGTGCGCC
GCtttggccgccgcggcccggaacagacccagggcaactttggcgatcaggaactgattcgcca
gggcaccgattataaacattggccgcagattgcgcagtttgcgccgagcgcgagcgcgtttttt
ggcatgagccgctaa (SEQ ID NO: 97)

The corresponding optimized version of the sequence in Table 13 is shown in Table 14.

TABLE 14
Codon optimized sequence of Table 13. Average GC content: 57.00%.
Sequence is shown with ATG start codon and TGA stop codon. Leader
sequence is shown in capitals. Furin cleavage sequences are shown in small
caps. T cell epitopes ares hown in underline, double underline, and
italics/bold and correspond to the epitopes shown above.

atgGTACTGCTGCAGGCTGGCAGCCTGCATGCCAAGAAAttcaccagaggggttt
actatcctgacaaggtgttcagaagcagcgtcctgcacAGAGTGAGACGCgtggaaa
agggaatctaccagacaagcaacttccgggtgcagcccactgagagcatcgtgag
attcccaaacAGAGTGAGAAGAcctaccaaactgaatgatctgtgttttacaaatgt
gtacgccgatCGGGTGAGAAGActgagcttcgagctgctgcacgccccagccacagt
ctgcggcCGAGTGCGCAGGaccaatacctctaacgaggtggccgtgctgtaccagga
cgtgaactgtacagaggtgcccgtgAGAGTGAGACGGgtgaccacagaaatcctgcc
tgtgtctatgaccaagacaagcgtggactgcaccatgtacatctgtAGAGTGAGAAG
Actgcctcctctgctgacagacgagatgatcgctcaatacacctctgctctgctg
gccAGAGTTCGTAGAatggcctaccggtttaacggcatcggcgtgacccagAGAGTGCG
CCGGgcccaggccctgaacacactggtcaagcagctgagcagcaacttcggagcca
tcagctctgtgctgaacgacatcctcagcagactggacaagCGGGTGAGACGCcaga
gcaagagggtggacttctgcggcaagggctaccacctcatgagcttcccccagtc
tgctcctcacggcgtggtgttcctgcatAGAGTGAGAAGAcgggaaggcgtgtttgt
gtccaacggcacccactggttcgtgCGGGTACGGCGGagcaaaaaccccctgctgta
cgacgccaactacttcctgtgctggcacaccaactgctacgactactgcatcccc
tacaacagcgtcaccagttctattgtgatcCGGGTCCGGCGTtcctttagactgttc
gccaggacccggtccatgtggtccttcaatcctgaaaccaatatcctgctgaacg
tgccactgcacggcaccatcctgaccagacccctgctggaatccgagctggtgat
cggcgccgtgatccttagaggacacctgcggatcgccggccaccacctgggaaga
CGGGTGCGGAGAaacaccaacagcagccctgatgatcagattggatattacagaagaA
GAGTGAGACGGttcggcagacggggccctgagcagacacagggcaacttcggcgatc
aggagctgatcagacaaggcaccgactacaagcactggcctcagatcgcccaatt
tgctcctagcgccagcgccttcttcggcatgagcagatga (SEQ ID NO: 98)

In addition to furin, the cysteine protease, cathepsin S (CS), is known to play active roles in endosomal proteolytic cleavage, generating peptides for TAP-Independent MHC Class I cross-presentation in vivo (Shen et al. (2004) Immunity, 21: 155-165). Antigen cleavage by these proteases release peptides for MHC class I epitope presentation. Cathepsin S recognizes the peptide sequence PMGLP (SEQ ID NO:99), which is cleaved between Glycine and Lysine. Reverse translation of the cleavage site yields a ccgatgggcctgccg (SEQ ID NO: 100) sequence. It would be straightforward to swap out the furin cleavage site with an alternative cathepsin recognition sites. It will be recognized that in various other embodiments, nucleic acid sequences encoding any or a number of protease recognition sites known to those of skill in the art can be introduced into the nucleic acid constructs.

### Type I Vaccine Design Using IEDB Analysis.

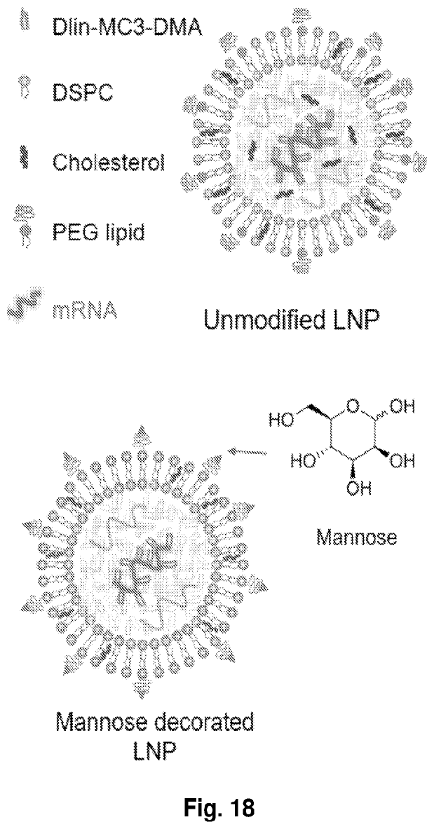

We also constructed another Type I multi-epitope vaccine to activate protective CD8+ T-cell responses, premised on using the IEDB discovery, described in Table 2, supra. Reverse translation of those peptide sequences yielded 12 nucleotide sequences (see, Table 15) that were integrated into another Vaccine I embodiment (FIG. 18).

TABLE 15
IEDB search T cell epitopes in Spike protein of SARS-CoV-2 Omicron BA.1.

| | | Amino | | |
|---|---|---|---|---|
| | | Acid | | Nucleic |
| Spike AA | Amino acid | SEQ ID | | Acid SEQ |
| coordinates | sequence | NOs | Reverse translation | ID NOs. |
| 513-527 | LSFELLHAPAT | 101 | ctgagctttgaactgctgcatgcgccggc | 102 |
| | VCGP | | gaccgtgtgcggcccg | |
| 863-877 | PLLTDEMIAQY | 103 | ccgctgctgaccgatgaaatgattgcgca | 104 |
| | TSAL | | gtataccagcgcgctg | |
| 964-978 | KQLSSNFGAIS | 105 | aaacagctgagcagcaactttggcgcgat | 106 |
| | SVLN | | tagcagcgtgctgaac | |
| 898-912 | FAMQMAYRFN | 107 | tttgcgatgcagatggcgtatcgctttaac | 108 |
| | GIGVT | | ggcattggcgtgacc | |
| 715-729 | PTNFTISVTTEI | 109 | ccgaccaactttaccattagcgtgaccacc | 110 |
| | LPV | | gaaattctgccggtg | |
| 1,042-1,056 | FCGKGYHLMS | 111 | ttttgcggcaaaggctatcatctgatgagct | 112 |
| | FPQSA | | ttccgcagagcgcg | |
| 386-396 | KLNDLCFTNV | 113 | aaactgaacgatctgtgctttaccaacgtg | 114 |
| | Y | | tat | |
| 315-329 | TSNFRVQPTES | 115 | accagcaactttcgcgtgcagccgaccg | 116 |
| | IVRF | | aaagcattgtgcgcttt | |
| 606-620 | NEVAVLYQDV | 117 | aacgaagtggcggtgctgtatcaggatgt | 118 |
| | NCTEV | | gaactgcaccgaagtg | |
| 37-50 | RGVYYPDKVF | 119 | cgcggcgtgtattatccggataaagtgttt | 120 |
| | RSSV | | cgcagcagcgtg | |
| 1,093-1,102 | GVFVSNGTHW | 121 | ggcgtgtttgtgagcaacggcacccattg | 122 |
| | | | g | |
| 539-553 | VNFNFNGLKG | 123 | gtgaactttaactttaacggcctgaaaggc | 124 |
| | TGVLT | | accggcgtgctgacc | |

In contradistinction to the network-based embodiment, this version only contains spike proteins, including the nucleotide sequence for amino acids 539-546 (VNFNFNGL (SEQ ID NO:23). This epitope has been shown by Pardieck et al. (2022) Nature Com. 13: 3966) to induce T-effector memory (TEM), and T-resident memory (TRM) responses in the lungs of K18-hACE2 transgenic mice, without impacting CD4+ T cell responses or raising antibodies. Tissue-resident memory T cells are capable of mounting an early defense in the lung, which is protective in nature, much like the action of antibodies. Zhi et al. (2005) Virology, 335: 34-45) have also shown that this epitope mounts strong vaccine responses against SARS-CoV1, confirming its conserved efficacy.

Similar to the design of cDNA constructs of epitopes identified by Nathan et al., we used the epitopes identified in our IEDB search to assemble a mRNA construct. The epitope sequences were reversed translated into nucleotide sequences.

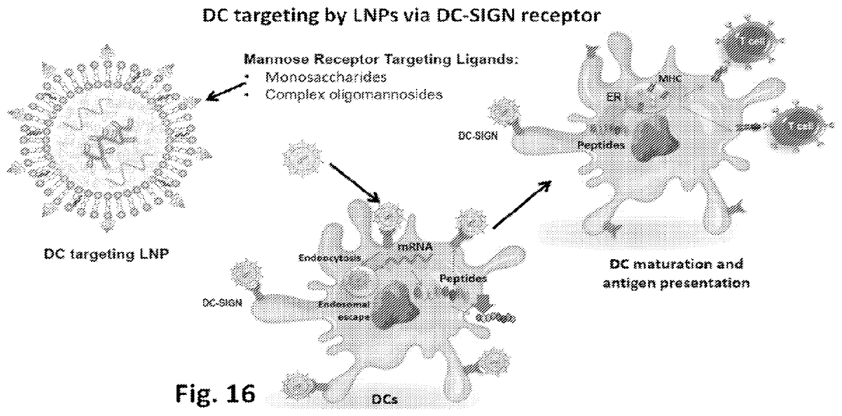

FIG. 16 shows the combined cDNA sequence encoding the epitopes shown in 15. The sequence is illustrated with an ATG start codon and a TAA stop codon.

TABLE 16
The reverse translated IEDB conserved T-cell epitopes are integrated by
using natural flanking sequences (2 aa on each side of each epitope) as
described in FIG. 8. Single underline = T cell epitopes, double
underline = T cell epitopes, bold/italic = T cell epitopes.

atggtggtgctgagctttgaactgctgcatgcgccggcgaccgtgtgcggcccgaaaaaaaaat
gcgtgaactttaactttaacggcctgaaaggcaccggcgtgctgaccgaaagcctgccgccgct
gctgaccgatgaaatgattgcgcagtataccagcgcgctggcgggcctggtgaaacagctgagc
agcaactttggcgcgattagcagcgtgctgaacgatattattccgtttgcgatgcagatggcgt
atcgctttaacggcattggcgtgacccagaacgcgattccgaccaactttaccattagcgtgac
caccgaaattctgccggtgagcatggtggatttttgcggcaaaggctatcatctgatgagcttt
ccgcagagcgcgccgcatccgaccaaactgaacgatctgtgctttaccaacgtgtatgcggatt
atcagaccagcaactttcgcgtgcagccgaccgaaagcattgtgcgctttccgaacaccagcaa
ccaggtggcggtgctgtatcaggatgtgaactgcaccgaagtgccggtgtttacccgcggcgtg
tattatccggataaagtgtttcgcagcagcgtgctgcatcgcgaaggcgtgtttgtgagcaacg
gcacccattggtttgtgtaa (SEQ ID NO: 125)

Table 17 shows the sequence in Table 16 with the reverse translated ppCT signal peptide sequence.

TABLE 17
The reverse translated ppCT signal peptide sequence was added to the
construct shown in Table 16. Capitals = sequence encoding ppCT signal peptide.
Single underline = T cell epitopes, double underline = T cell epitopes,
bold/italic = T cell epitopes. The sequence is shown with an ATG start codon
and a TAA stop codon.

atgGTGCTGCTGCAGGCGGGCAGCCTGCATGCGAAAAAAgtggtgctgagctttgaactgctgc
atgcgccggcgaccgtgtgcggcccgaaaaaaaaatgcgtgaactttaactttaacggcctgaa
aggcaccggcgtgctgaccgaaagcctgccgccgctgctgaccgatgaaatgattgcgcagtat
accagcgcgctggcgggcctggtgaaacagctgagcagcaactttggcgcgattagcagcgtgc
tgaacgatattattccgtttgcgatgcagatggcgtatcgctttaacggcattggcgtgaccca
gaacgcgattccgaccaactttaccattagcgtgaccaccgaaattctgccggtgagcatggtg
gatttttgcggcaaaggctatcatctgatgagctttccgcagagcgcgccgcatccgaccaaac
tgaacgatctgtgctttaccaacgtgtatgcggattatcagaccagcaactttcgcgtgcagcc
gaccgaaagcattgtgcgctttccgaacaccagcaaccaggtggcggtgctgtatcaggatgtg
aactgcaccgaagtgccggtgtttacccgcggcgtgtattatccggataaagtgtttcgcagca
gcgtgctgcatcgcgaaggcgtgtttgtgagcaacggcacccattggtttgtgTAA
(SEQ ID NO: 126)

As an alternative to natural flanking sequences, furin cleavage sites were inserted between the T cell epitopes for MHC 1 processing as described in FIG. 16 and illustrated below in Table 18.

TABLE 18
Combined cDNA sequence with nucleotide sequences of Furin cleavage sites.
Small caps = sequence encoding furin cleavage sites. Capitals = sequence
encoding ppCT signal peptide. Single underline = T cell epitopes, double
underline = T cell epitopes,b old/italic = T cell epitopes. The sequence is
shown with an ATG start codon and a TAA stop codon.

atgGTGCTGCTGCAGGCGGGCAGCCTGCATGCGAAAAAAgtggtgctgagctttgaactgctgc
atgcgccggcgaccgtgtgcggcccgaaaaaacGCGTGCGCCGCaaatgcgtgaactttaactttaa
cggcctgaaaggcaccggcgtgctgaccgaaagcCGCGTGCGCCGCctgccgccgctgctgaccgat
gaaatgattgcgcagtataccagcgcgctggcgggcCGCGTGCGCCgcctggtgaaacagctgagc
agcaactttggcgcgattagcagcgtgctgaacgatattCGCGTGCGCCGCattccgtttgcgatgc
agatggcgtatcgctttaacggcattggcgtgacccagaacCGCGTGCGCCGCgcgattccgaccaa
ctttaccattagcgtgaccaccgaaattctgccggtgagcatgCGCGTGCGCCGCgtggatttttgc
ggcaaaggctatcatctgatgagctttccgcagagcgcgccgcatCGCGTGCGCCGCccgaccaaac
tgaacgatctgtgctttaccaacgtgtatgcggatCGCGTGCGCCGctatcagaccagcaactttc
gcgtgcagccgaccgaaagcattgtgcgctttccgaacCGCGTGCGCCGCaccagcaaccaggtggc
ggtgctgtatcaggatgtgaactgcaccgaagtgccggtgCGCGTGCGCCGCtttacccgcggcgtg
tattatccggataaagtgtttcgcagcagcgtgctgcatCGCGTGCGCCGCcgcgaaggcgtgtttg
tgagcaacggcacccattggtttgtg taa (SEQ ID NO: 127)

Table 19 shows the sequence of a codon optimized version of the sequence shown in Table 19. Codon optimization was performed as described with respect to Table 10.

TABLE 19
Codon optimized version of the sequence shown in Table 18. SmallCaps =
sequence encoding furin cleavage sites. Capitals = sequence encoding ppCT signal
peptide. Single underline = T cell epitopes, double underline = T cell epitopes,
bold/italic = T cell epitopes.

atggTGCTGCTGCAGGCCGGCAGCCTGCACGCCAAGAAAgtggtgctctccttcgagctgctgc
acgcccctgctacagtgtgtggacctaagaaaAGAGTGAGAAGGaagtgcgtgaacttcaactttaa
cggcctgaagggaaccggcgtgctgacagaatctAGAGTGAGAAGActgccccccctgctgacagat
gagatgatcgcccagtacaccagcgctctggccggcAGAGTCCGGAGActggtcaagcagctgagca
gcaacttcggcgctattagcagcgtgctgaacgacatcCGGGTCCGAAGAatccctttcgccatgca
gatggcctatagatttaatggcatcggagtgacccagaacAGAGTGAGAAGAgccatccctacaaac
ttcaccatctccgtgaccaccgaaatcctgcctgtgtccatgCGGGTTCGGCGCgtggacttctgcg
gcaagggctaccacctgatgagcttcccccagagcgcccctcacAGAGTGCGGCGGccaaccaagct
gaatgatctgtgcttcaccaacgtgtacgccgacAGAGTGCGGAGAtaccagaccagcaattttcgg
gtgcaacctaccgagtctatcgtgcggttccctaacAGAGTGAGAAGAaccagcaaccaggtggccg
tgctgtaccaagacgtgaactgtaccgaggtgcccgtgAGAGTCAGACGGtttacaagaggcgtgta
ctacccagacaaggtgttcagaagctctgtgctgcatAGAGTGCGGCGCagggaaggcgttttcgtg
tctaacggcacacactggttcgtgtaa (SEQ ID NO: 128)

### Type II Vaccine Construction.

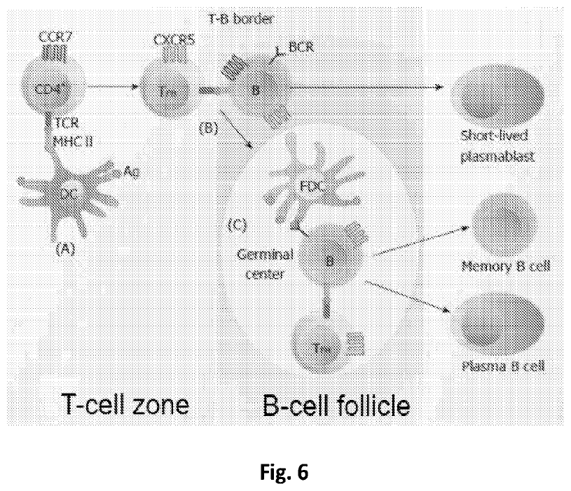

There is an urgent need to identify broadly neutralizing B-cell epitopes that are conserved among SARS-CoV-2 variants, especially those that are heavily mutated, like Omicron. Conserved epitopes provide desirable targets to develop what we call a Type II multi-epitope vaccine. The rationale for constructing such a vaccine is to raise broadly neutralizing antibodies to conserved SARS CoV2 spike protein B-cell epitopes, and to also make use of epitopes for generating CD4+ helper T-cells that assist antibody production in lymph node germinal centers (FIG. 6). The focus on the spike protein, particularly the receptor binding domain (RDB), is the effect of these antibodies in providing early protection, in addition to the antibody response agreeing with generation of spike-specific CD4+ T cell responses. Moreover, the objective of raising broadly neutralizing antibodies is prevention of immune escape by VOC, where recent experience for Delta, Mu, and Omicron has shown a dramatic decline in neutralizing antibody efficacy in response to current vaccines, in addition to escape from the majority of therapeutic monoclonal antibodies in clinical use (see, e.g., Cameroni et al. (2022) Nature, 602: 664-670; Planas et al. (2021) Nature, 596: 276). In considering multi-epitope selections for generating broadly neutralizing antibodies, it is important to keep in mind that the major spike protein B-cell epitopes are localized in the receptor-binding domain (RBD), with a smaller number of lesser potency antibodies directed against the N-terminal domain (NTD).

It is important to discern between continuous epitopes (CPs) and discontinuous B-cell epitopes (DPs) for the purpose of nucleic acid design, namely reverse translation is only possible for CPs. Most continuous epitopes are located on the surfaces of the spike protein trimers, which have to be assessable for Fab subunits that can reach the flexible up or down RBD conformations displayed in these trimers. The ability to cross-link RBDs on the same or adjacent subunits, constitutes the basis for antibody neutralization (see, e.g., Barnes et al. (2020) Nature, 588: 682; Cohen et al. (2022) Science 377: 677: 318). This is confirmed by the cryo-EM structures obtained by Barnes et al., investigating all the possible Fab-spike trimer complexes for antibodies reaching the up or down conformations. High avidity interactions often result from the interaction of 2-3 Fabs per trimer. Further analysis looking at approach angles of Fab interactions with RBDs allow prediction making to improve IgG neutralizing potency, increasing antibody avidity that also serves to select antibodies that are more resistant to spike mutations. The attainment of this structural information allowed the Bjorkman group to evaluate RBD mutants occurring in circulating viral isolates as well as in mutants generated by in vitro selection (Barnes et al. (2020) Nature, 588: 682; Cohen et al. (2022) Science 377(677): 318). This allowed the Bjorkman group to classify structure-activity relationships of anti-SARS CoV2 antibodies into four categories, namely: (i) neutralizing antibodies that block ACE2 and bind only ‘up’ RBDs; (ii) ACE2-blocking neutralizing antibodies that bind both up and ‘down’ RBDs with the ability to contact adjacent RBDs; (iii) neutralizing antibodies that bind outside the ACE2 site and recognize both up and down RBDs; and (iv) antibodies that do not block ACE2, while only binding to up RBDs. The identification of structure activity relationships now allows avidity rulemaking and identification of RBD-targeting antibodies, capable of engaging broadly neutralizing affects, including selection of monoclonal antibodies for clinical use. Our approach is to identify conserved linear B-cell epitopes that are not impacted by the mutations occurring in Omicron BA 0.1 and other subtypes.

The Omicron BA.1 variant exhibits more than 30 mutations in the spike protein, in addition to events in other viral proteins (//covariants.org/variants/21K). It is noted that a large number of spike protein mutations are localized in the RDB domain at sites making contact with the ACE the receptor to enhance infectivity, as explained in FIG. 2. Of key importance is another category of mutations, impacting spike protein epitopes that are recognized by the Fab ends of crosslinking neutralizing antibodies. This leads to loss of crosslinking of the RDB, which increases antibody avidity. Moreover, Shah et al. (Comput & Struct Biotech Journal, 2020; 18: 3402-3414) demonstrated that inability of a therapeutic monoclonal antibody (CR3022) to cross-link adjacent spike proteins, leads to reduced antibody avidity and neutralizing efficacy.

Due to the strong likelihood that several spike mutations are be linked to the decline in antibody neutralizing efficacy and failure of trimer crosslinking, we launched an IEDB search for linear spike epitopes in Omicron BA.1.

This analysis was performed using the BepiPred-2.0: Sequential B-Cell Epitope Predictor tool which forecasts B-cell epitopes in protein sequences, using a Random Forest algorithm trained by using epitope and non-epitope amino acid sequence crystal structures. Sequential prediction smoothing is performed afterwards. Residues with scores above the threshold default value of 0.5 are regarded to be more likely to constitute an actual epitope. Using this prediction tool and the ability to derive structural biological statistics, led to the identification of a series of 15 continuous B-cell epitopes in the Omicron BA.1 spike protein (Table 20).

TABLE 20
IEDB search for linear B-cell epitopes in the spike protein: Another IEDB
search was carried out to identify linear B cell epitopes, using BepiPred 2.0 (Jespersen et
al. (2017) Nucleic Acids Res, doi: 10.1093/nar/gkx3462017). BepiPred-2.0 predicts the
presence of B-cell epitopes in a protein sequence, using a Random Forest algorithm,
trained to evaluate the crystal structures of epitope and non-epitope amino acids. This is
followed by sequential prediction smoothing. The residues with scores above the
threshold value of 0.5 are predicted to be part of an epitope. We identified 15 B-cell
epitopes in the spike protein of Omicron BA.1 (left columns below). We compared our
IEDB results to conserved linear B-cell epitope analysis in the literature. We observed
several IEDB predicted B cell epitopes, to show overlap with conserved epitope sequences
(left columns below) reported by Yoshida et al. (2021) Sci. Rep., 11: 5934; Prakash et al.
(2021) J. Immunol. 206: 2566-2582; Ansori et al. (2021) J. Pharm. Pharmacog. Res. 9(6):
767; Guo et al. (2022) Mol. Biomed. 3: 12; Amrun et al. (2020) EBioMedicine, 58:
102911-102919; Moody et al. (2021), Volume 1: article 709533; Wang et al. (2022) Emer
Microbes & Infections, doi.org/10.1080/22221751.2022.2109515). The overlap is shown
in the table as bold/underline.

| | IEDB search identifying linear | | Linear SARS-CoV-2 B-cell | |
|---|---|---|---|---|
| | Omicron BA.1 B cell epitopes | | epitopes in the literature | |
| | Sequence | Position | Sequence | Position |
| 1 | TRTQSPPAPPAYTNS | 20-34 | SQCVNLTTRTQLPPAYTNSFT | 13-37 |
| | (SEQ ID NO: 129) | | RGVY (SEQ ID NO: 130) | |
| | | | (Prakash) | |
| 2 | GTNGTKRFD (SEQ ID | 73-81 | FSNVTWFHAIHVSGTNGTKRF | 59-81 |
| | NO: 131) | | DN (SEQ ID NO: 132) | |
| | | | (Prakash) | |
| 3 | DPFLDVYYHKNNKSWME | 139-155 | HKNNKSWMESEFRVYSSANN | 146-164 |
| | (SEQ ID NO: 133) | | (SEQ ID NO: 134) | |
| | | | (Yoshida) | |
| 4 | LTPGDSSSGWTAG | 249-261 | LTPGDSSSGWTAG (SEQ | 249-261 |
| | (SEQ ID NO: 135) | | ID NO: 136) | |
| | | | (Moody) | |
| 5 | FPNITNLCPFDEVFNATRF | 329-390 | RFASVYAWNRKRISNCVADY | 346-365 |
| | ASVYAWNRKRISNCVADYSV | | (SEQ ID NO: 138) | |
| | LYNFAPFFAFKCYGVSPTKL | | (Yoshida) | |
| | NDL (SEQ ID NO: 137) | | | |
| 6 | RGNEVSQIAPGQTGNIAD | 403-420 | GQTGNIADYNYKLPDDFTG | 413-432 |
| | (SEQ ID NO: 139) | | (SEQ ID NO: 140) | |
| | | | (Yoshida, Ansori) | |
| 7 | KLDSKVGGNYNY (SEQ | 440-451 | DSKVGGNYNYLYRLFRKSNL | 442-460 |
| | ID NO: 141) | | (SEQ ID NO: 142) | |
| | | | (Yoshida) | |
| 8 | YQAGNKPCN (SEQ ID | 473-480 | YQAGS (SEQ ID | 473-477 |
| | NO: 143) | | NO: 144) | |
| | | | (Guo) | |
| 9 | YGFRPTYGV (SEQ ID | 495-503 | PLRSYGFRPTYGVGHQPYRV | 491-509 |
| | NO: 145) | | (SEQ ID NO: 146) | |
| | | | (Yoshida) | |
| 10 | FELLHAPATVCGPKKSTNL | 515-534 | LHAPATVCGPKKSTNLVKNK | 518-537 |
| | V (SEQ ID NO: 147) | | (SEQ ID NO: 148) | |
| | | | (Yoshida) | |
| 11 | RDIADTTDAVRDP | 567-579 | VCGPKKSTNLVKNKCVNFNFN | 524-598 |
| | (SEQ ID NO: 149) | | GLTGTGVLTESNKKFLPFQQF | |
| | | | GRDIADTTDAVRDPQTLEILD | |
| | | | ITPCSFGGVSVI (SEQ ID | |
| | | | NO: 150) | |
| | | | (Prakash) | |
| 12 | VITPGTNTSN (SEQ ID | 597-606 | GTNTSNQVAVLYQDVNCTEVP | 601-640 |
| | NO: 151) | | VAIHADQL TPTWRVYSTGS | |
| | | | (SEQ ID NO: 152) | |
| | | | (Prakash, Moody) | |
| 13 | ASYQTQTKSHRRARSVASQ | 672-690 | CASYQTQTKSHRRARSVASQ | 671-690 |
| | (SEQ ID NO: 153) | | (SEQ ID NO: 154) | |
| | | | (Yoshida) | |
| 14 | LPDPSKPSKR (SEQ ID | 806-815 | ILPDPSKPSKRS (SEQ ID | 805-816 |
| | NO: 155) | | NO: 156) | |
| | | | (Moody) | |
| 15 | VNNTVYDPLQPELDSFKEE | 1133-1161 | DSFKEELDKYFKNHTSPDVD | 1146-1164 |
| | LDKYFKNHTSPDVDLGDISG | | (SEQ ID NO: 158) | |
| | I (SEQ ID NO: 157) | | (Yoshida) | |

Subsequent display of all the spike protein mutations (green), in relation to the linear epitopes (red), demonstrated that a number of the epitopes, particularly in the locality of the receptor binding motive (RBM, are impacted. However, a number of epitopes were not impacted and therefore considered as prime candidates for vaccine development (Table 21).

TABLE 21
B- cell epitope mutations in Omicron. The B-cell epitopes identified in
Table 20 were highlighted in relation to the Omicron BA.1 sequence,
including demonstration of the occurrence of mutations in those epitopes.
This demonstrates that among 15 epitopes, 9 epitopes exhibit mutations,
while 6 were unaffected or conserved. Spike protein Linear B cell
epitopes (IEDB) (underline) vs Omicron B.1 mutations (uppercase bold).

mfvflvllplvssqcvnlttrtqSPPAppaytnsftrgvyypdkvfrssvlhstqdlflpffsn
vtwfhaisgtngtkrfdnpvlpfndgvyfasIeksniirgwifgttldsktqsllivnnatnvv
ikvcefqfcndpflDvyyhknnkswmesefrvyssannctfeyvsqpflmdlegkqgnfknlre
fvfknidgyfkiyskhtpilGrdlpqgfsaleplvdlpiginitrfqtllalhrsyltpgdsss
gwtagaaayyvgylqprtfllkynengtitdavdcaldplsetkctlksftvekgiyqtsnfrv
qptesivrfpnitnlcpfDevfnatrfasvyawnrkrisncvadysvlynFaPfFAfkcygvsp
tklndlcftnvyadsfvirgNevSqiapgqtgNiadynyklpddftgcviawnsnKldskvSgn
ynylyrlfrksnlkpferdisteiyqagNKpcngvAgfncyfplRsygfRptYgvgHqpyrvvv
lsfellhapatvcgpkkstnlvknkcvnfnfngltgtgvltesnkkflpfqqfgrdiadttdav
rdpqtleilditpcsfggvsvitpgtntsnqvavlyqGvnctevpvaihadqltptwrvystgs
nvfqtragcligaeYvnnsyecdipigagicasyqtqtKsHrrarsvasqsiiaytmslgaens
vaysnnsiaiptnftisvtteilpvsmtktsvdctmyicgdstecsnlllqygsfctqlKralt
giaveqdkntqevfaqvkqiyktppikYfggfnfsqilpdpskpskrsfiedllfnkvtladag
fikqygdclgdiaardlicaqkfngltvlpplltdemiaqytsallagtitsgwtfgagaalqi
pfamqmayrfngigvtqnvlyenqklianqfnsaigkiqdslsstasalgklqdvvnHnaqaln
tlvkqlssKfgaissvlndilsrldkveaevqidrlitgrlqslqtyvtqqliraaeirasanl
aatkmsecvlgqskrvdfcgkgyhlmsfpqsaphgvvflhvtyvpaqeknfttapaichdgkah
fpregvfvsngthwfvtqrnfyepqiittdntfvsgncdvvigivnntvydplqpeldsfkeel
dkyfknhtspdvdlgdisginasvvniqkeidrlnevaknlneslidlqelgkyegyikwpwyi
wlgfiagliaivmvtimlccmtsccsclkgccscgscckfdeddsepvlkgvklhyt
(SEQ ID NO: 159)

Before epitope incorporation in our vaccine, it was important to compare our selection to conserved linear B-cell epitope identified in the literature, including showing the relation of these vaccines to generating neutralizing antibodies in humans. A summary of these comparative findings appear in Table 20, The synopsis in Table 20 demonstrates that we could indeed find some overlap with the epitopes identified by other investigators. Moreover, analysis of neutralizing antibodies in the serum of human subjects, raised in the course of natural infections or through vaccination, could be shown to recognize many of the overlapping epitope sequences, as discovered in our IEDB search (Table 20). For instance, the collection of serum from Covid 19 patients in an intensive care unit by Yoshida, et al., demonstrated good correlation between neutralizing antibody levels and interaction with epitopes 3, 6-10, 13 and 15, as assessed by ELISA and performing neutralizing antibody essays (Yoshida et al. (2021) Sci. Rep. 11: 5934). Prakash et al. (2021) J. Immunol. 206: 2566-2582) identified several human B cell epitopes (including epitopes 1, 2, 11, and 12 in Table 20) that are highly conserved in over 81,000 SARS-CoV-2 genome sequences identified in 190 countries on 6 continents, also appearing in six circulating corona cold viruses, SL-CoVs isolated from bats; SL-CoV isolated from pangolins; SL-CoVs isolated from civet cats; and MERS strains isolated from camels. Moreover, it was possible to demonstrate the recall responses, using B cells derived from the blood of Covid patients. Gao et al. supra. also performed molecular dynamic simulations of wildtype and mutant spike protein conformations, demonstrating maintenance of protein structure in spite of regional mutations. Moreover, our own linear epitope analysis showed that sequences escaping mutation in five different Omicron variants are conserved, allowing these the non-mutated sequences to be included as lead vaccine candidates. These peptide sequences and reverse-translated nucleic acid sequences are shown in Table 22.

TABLE 22
Conserved B cell epitopes and reverse-translated nucleic acid sequences.

| | Conserved B | SEQ | | SEQ |
|---|---|---|---|---|
| Spike | cell epitopes | ID NO | Reverse translation | ID NO |
| 73-81 | GTNGTKRFD | 131 | ggcaccaacggcaccaaacgctttgat | 160 |
| 515-534 | FELLHAPATVCGP | 147 | tttgaactgctgcatgcgccggcgaccg | 161 |
| | KKSTNLV | | tgtgcggcccgaaaaaaagcaccaacct | |
| | | | ggtg | |
| 567-579 | RDIADTTDAVRDP | 149 | cgcgatattgcggataccaccgatgcgg | 162 |
| | | | tgcgcgatccg | |
| 597-606 | VITPGTNTSN | 151 | gtgattaccccgggcaccaacaccagca | 163 |
| | | | ac | |
| 806-815 | LPDPSKPSKR | 155 | ctgccggatccgagcaaaccgagcaaac | 164 |
| | | | gc | |
| 1146-1164 | VNNTVYDPLQPEL | 157 | gtgaacaacaccgtgtatgatccgctgc | 165 |
| | DSFKEELDKYFKN | | agccggaactggatagctttaaagaaga | |
| | HTSPDVDLGDISG | | actggataaatattttaaaaaccatacc | |
| | I | | agcccggatgtggatctgggcgatatta | |
| | | | gcggcatt | |

Before integrating the conserved B-cell epitopes, in Table 22 into Vaccine II, an IEDB search was conducted to find CD4 positive T-cell epitopes that can be included to assist antibody production in lymph node germinal centers (Table 23).

TABLE 23
IEDB identified CD4+ spike T cell epitopes. To find spike protein epitopes
that can be used to activate Tfh, we first performed an IEDB search for CD4+ epitopes.
This search was performed with the assistance of the IEDB recommended 2.22 software to
maximize human population coverage. We included the full available HLA reference set
(27 alleles), including HLA-DRB1, DRB3, DRB4, DRB5; HLA-DQA,, DQB; HLA-DPA,
DPB. The top six ranking (<5%) epitopes were selected for listing together with their
corresponding HLA.

| | | | SEQ ID | |
|---|---|---|---|---|
| Ranking | Position | Sequence | NO | HLA specificity |
| 0.49% | 344-358 | ATRFASVYAWNRKRI | 166 | HLA-DRB5*01:01 |
| 3% | 556-570 | ESNKKFLPFQQFGRD | 167 | HLA- |
| | | | | DPA1*02:01/DPB1*05:01 |
| 0.58% | 753-767 | CSNLLLQYGSFCTQL | 168 | HLA-DRB1*15:01 |
| 4.2% | 825-839 | LFNKVTLADAGFIKQ | 169 | HLA-DRB3*02:02 |
| 0.2% | 856-870 | QKGLTVLPPLLTDEM | 170 | HLA- |
| | | | | DPA1*02:01/DPB1*14:01 |
| 1.22% | 959-973 | AQALNTLVKQLSSKF | 171 | HLA-DRB1*12:01 |

This search was influenced by considering the importance of lymph node germinal centers in memory B-cell development (Nel & Miller (2021) ACS Nano, 15: 5793-5818). Thus, early activation of naïve CD4+ cells by dendritic cells in the lymph node T-cell zone, generates a subset of helper T cells, known as T-follicular helper lymphocytes (Tfh). The CD4 positive T cells are characterized by the expression of the CXC-chemokine receptor 5 (CXCR5), in addition to other activation markers, e.g., CD200, PDCD1, ICOS, CXCL13, CD40LG, and CXCR5 (Lu et al. (2021) J. Exp. Med. 218 No. 12 e20211327). This allows Tfh cell migration to B-cell follicles, where the interaction with B cells facilitates immunoglobulin hypermutation, class switching and affinity maturation. In the process, B-cells differentiate into memory cells and long-lived plasma cells. Plasma cells are end-stage cells selected based on affinity, and home to the bone marrow where they can reside for variable periods of time. Memory cells are found in the circulation and in lymphoid organs. Noteworthy, inefficient induction of Tfh cells is correlated with more severe and fatal COVID-19.

Our IEDB analysis was carried out to identify conserved CD4 epitopes, not impacted by mutations in VOC such as Omicron. The search was for MHC-II binding epitopes with maximal coverage in the human population. This was accomplished by including the full HLA reference set (27 alleles) in the IEDB recommended 2.22 tool, including HLA-DRB1, DRB3, DRB4, DRB5; HLA-DQA, DQB; HLA-DPA, DPB. Among several recovered sequences, the top ranking (<5%) epitopes and their corresponding HLA alleles are outlined in Table 23. We further showed that the sequences were conserved in Omicron variants, without mutation. It was not possible, however, with the IEDB search tool to identify the relationship to specific T-cell subsets. Moreover, the identification of Tfh activating epitopes in SARS CoV2 has only recently become possible by clonotypic identification of CD4+ lymphocytes in SARS CoV2 blood samples, subjected to peptide activation plus analysis of T-cell antigen receptor (TCR) clonotypes (Shomuradova et al., 2020; Dykema et al., 2021). This approach has been shown to be successful for finding public Tfh clonotypes among CD4 positive T cells from COVID-19 patients (Lu et al. (2021) J. Exp. Med. Vol. 218 No. 12 e20211327). The discovery included identification of crystallized TCR constructs that recognize conserved spike epitopes in circulating Tfh (cTfh) clonotypes, retrieved from patients with mild COVID-19 infection. These clonotypes ae also conserved in VOC. Importantly, the most prevalent cTfh clonotype reacted with the Spike 864-882 (LLTDEMIAQYTSALLAGTI (SEQ ID NO:172)) epitope, which can be presented by multiple HLA genotypes, suggesting its role as a universal T cell epitope useful that can activate a variety of Thf clonotypes. Noteworthy, comparison of our in-house IEDB sequences with S864-882 and other public clonotypes described by Lu et al., showed significant overlap, demonstrated by the bold underlining in Table 24.

TABLE 24
T-follicular helper (Tfh) CD4+ T cell epitopes. To determine if the
epitopes in Table 23 s are functionally linked to Tfh activity, we
compared our lineup with T-follicular helper (Tfh) CD4+ T cell
identification by Lu et al. (2021) J. Exp. Med. 218 No. 12 e20211327).

| Epitopes | Positions | Sequence | SEQ ID NO |
|---|---|---|---|
| Spike protein | 353-367 | WNRKRISNCVADYSV | 173 |
| Spike protein | 557-567 | KKFLPFQQFGR | 174 |
| Spike protein | 753-759 | LLQYGSF | 175 |
| Spike protein | 833-843 | FIKQYGDCLGD | 176 |
| Spike protein* | 864-882 | LLTDEMIAQYTSALLAGTI | 177 |
| Spike protein | 957-967 | QALNTLVKQLS | 178 |

A homolog of the 5864-882 sequence received the highest-ranking order in our IEDB analysis, i.e. 5856-870, showing a six amino acid (LLTDEM (SEQ ID NO: 179)) overlap. This discovery, including of the promiscuous S864-882 epitope, led to the consideration of using the Lu et a. epitope lineup to design a type II vaccine prototype. The reverse translated nucleotide sequences, with addition of flanking amino acids, are shown in Table 25.

TABLE 25
To build a mRNA construct using the Lu et al. epitopes, flanking sequences
(bold/underline) were used for linkage of the Tfh epitopes, as shown in Table 24.
Natural flanking sequences (2 aa on each side of the epitope) were selected and
reverse translated into nucleic acid sequences. The lower panel highlights Tfh
epitope expression in the BA.1 spike protein, including their ability to escape
mutations in this variant. An alternative to this multi-epitope lineup, is to use
our IEDB epitopes, which show considerable overlap with those of Lu et al (Table 23).

| Spike | | |
|---|---|---|
| Position | Sequence | Reverse Translation |
| 353-367 | YAWNRKRISNCVADYSVLY | tatgcgtggaaccgcaaacgcattagcaactg |
| | | cgtggcggattatagcgtgctgtat |
| 557-567 | SNKKFLPFQQFGRDI | agcaacaaaaaatttctgccgtttcagcagtt |
| | | tggccgcgatatt |
| 753-759 | NLLLQYGSFCT | aacctgctgctgcagtatggcagcttttgcac |
| | | c |
| 833-843 | AGFIKQYGDCLGDIA | gcgggctttattaaacagtatggcgattgcct |
| | | gggcgatattgcg |
| 864-882 | PPLLTDEMIAQYTSALLAGTI | ccgccgctgctgaccgatgaaatgattgcgca |
| | TS | gtataccagcgcgctgctggcgggcacc |
| | | attaccagc |
| 957-967 | NAQALNTLVKQLSSK | aacgcgcaggcgctgaacaccctggtgaaaca |
| | | gctgagcagcaaa |

Importantly, it has also been shown that S864-882 as a single cryptic epitope is capable of raising antibodies to linear B cell epitopes, suggesting it could be of significance in its own right to be used as an alternative vaccine component, in combination with B-cell epitopes from our IEDB search. Moreover, there is also a commercially available pan-HLA DR-binding epitope (PADRE) sequence, AKFVAAWTLKAAA (SEQ ID NO:180), which can serve as a universal epitope for the activation of antigen specific-CD4+ T cells. PADRE has been used as an adjuvant in immunotherapeutic vaccine development. For instance, PADRE coupling to linear CoV2 B cell epitopes, N- or C-terminally, contributed to the improvement of Spike-specific IgG antibody responses (Pardieck et al. (2022) Nat. Com. 13: 3966).

Following the selection of Thf and conserved B-cell epitopes, an integrated mRNA construct was developed along the lines described for Vaccine L. As a first step, the reverse translated oligonucleotide sequences for the two sets of epitopes were combined, with the addition of flanking sequences, as shown in FIG. 33.

TABLE 26
Lineup of Tfh and B-cell epitopes that can be used in type II vaccine. Small
caps = flanking sequences. Single underline, double underline, bold/italic = epitopes.
Sequences are shown with ATG start codon and TAA stop codon.
T-follicular helper (Tfh) CD4+ T cell epitopes (Lu et al.. J.
Exp. Med. 2021, 218)
atgTATGCGtggaaccgcaaacgcattagcaactgcgtggcggattatagcgtgCTGTATAGCAACaa
aaaatttctgccgtttcagcagtttggccgcGATATTAACCTGctgctgcagtatggcagctttTGC
ACCGCGGGCtttattaaacagtatggcgattgcctgggcgatATTGCGCCGCCGCtgctgaccgatga
aatgattgcgcagtataccagcgcgctgctggcgggcaccattACCAGCAACGCGCaggcgctgaac
accctggtgaaacgctgagcagcaaataa (SEQ ID NO: 181)
Conserved B cell epitopes (IEDB analysis)
atgattagcggcaccaacggcaccaaacgctttgataacccgctgagctttgaactgctgcatg
cgccggcgaccgtgtgcggcccgaaaaaaagcaccaacctggtgaaaaactttggccgcgatat
tgcggataccaccgatgcggtgcgcgatccgcagaccgtgagcgtgattaccccgggcaccaac
accagcaaccaggtgcagattctgccggatccgagcaaaccgagcaaacgcagctttggcattg
tgaacaacaccgtgtatgatccgctgcagccggaactggatagctttaaagaagaactggataa
atattttaaaaaccataccagcccggatgtggatctgggcgatattagcggcattaacgcgtaa
(SEQ ID NO: 182)

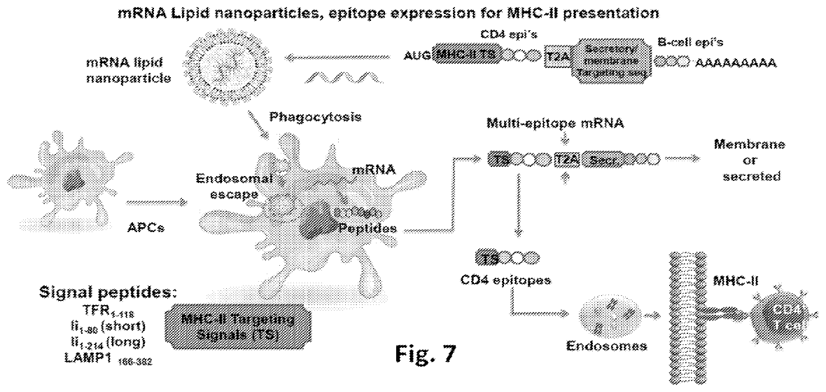

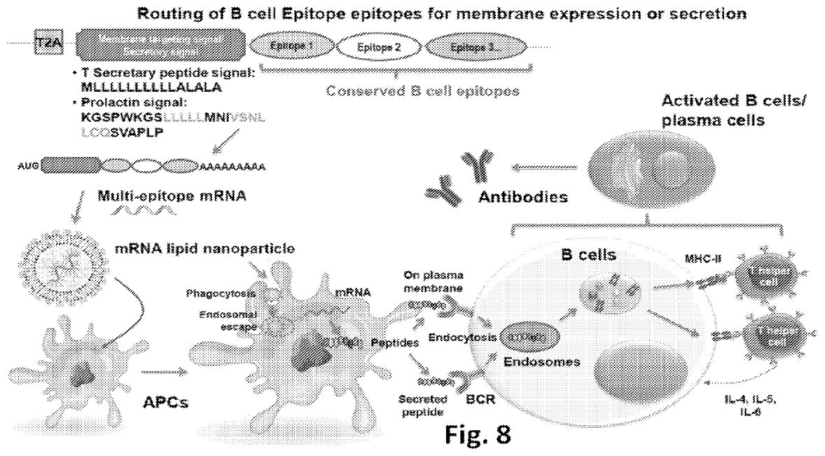

After insertion of start and stop codons, a signaling peptide sequence was inserted that directs CD4 epitopes to MHC-II (FIG. 7), while getting B-cell epitopes excreted or attached to the cell surface for recognition by the B-cell antigen receptor (FIG. 8). CD4 epitopes are targeted to the MHC-II compartment by the transferrin receptor (TRF) 1-118 or invariant (li) chain aa 1-80 endosomal targeting sequence (FIG. 7). The role of TFR1-118 is to provide hybrid peptide trafficking to the plasma membrane, where its transmembrane domain allows membrane incorporation, prior to endocytosis of that membrane segment to the MHC-II compartment. In contrast, Ii1-80 is directly transported to the endoplasmic reticulum membrane surrounding the MHC-II compartment.

Table 27 demonstrates the insertion of the reverse transcribed nucleotide sequence, representing the TFR1-118 targeting sequence, upstream of the T-follicular helper (Tfh) CD4+ T cell epitope compilation. In addition to TFR1-118, it is also possible to accomplish MHC-II targeting by using short (80 amino acids) or long (214 amino acid sequences of the Ii 1-214 protein or a LAMP1 sequence (amino acids 166-382)). This table also shows insertion of a prolactin signal was inserted upstream of B cell epitopes for secretion and extracellular recognition by the B-cell immunoglobulin receptor.

TABLE 27
Top row: The invariant 1i chain 1-80 was inserted upstream of the integrated
Tfh T cell epitope sequence for epitope routing to MHC class II. Single underline, double
underline, and bold/italic = epitopes. Capitals = Li11-80 invariant chain. Bottom row: A
prolactin signal was inserted upstream of B cell epitopes for secretion and extracellular
recognition by the B-cell immunoglobulin receptor. The prolactin secretary signal
exhibits a KGSPWKGSLLLLLMNIVSNLLCQSVAPLP (SEQ ID NO: 183)sequence,
which upon reverse translation yields: aaaggcagcccgtggaaaggcagcctgctgctgctgctg
atgaacattgtgagcaacctgctgtgccagagc gtggcgccgctgccg (SEQ ID NO: 184). Single underline,
double underline, and bold/italic = epitopes. Capitals/single underline = prolactin
sequence. Both sequences are shown with ATG start codons and TAA stop codons.
Insertion of Ii1-80 TS upstream of T-follicular helper (Tfh) CD4+ T cell epitope
compilation
atgATGGATGATCAGCGCGATCTGATTAGCAACCATGAACAGCTGCCGATTCTGGGCAACCGCC
CGCGCGAACCGGAACGCTGCAGCCGCGGCGCGCTGTATACCGGCGTGAGCGTGCTGGTGGCGCT
GCTGCTGGCGGGCCAGGCGACCACCGCGTATTTTCTGTATCAGCAGCAGGGCCGCCTGGATAAA
CTGACCATTACCAGCCAGAACCTGCAGCTGGAAAGCCTGCGCATGAAACTGtatgcgtggaacc
gcaaacgcattagcaactgcgtggcggattatagcgtgctgtatagcaacaaaaaatttctgcc
gtttcagcagtttggccgcgatattaacctgctgctgcagtatggcagcttttgcaccgcgggc
tttattaaacagtatggcgattgcctgggcgatattgcgccgccgctgctgaccgatgaaatga
ttgcgcagtataccagcgcgctgctggcgggcaccattaccagcaacgcgcaggcgctgaacac
cctggtgaaacagctgagcagcaaataa (SEQ ID NO: 185)
Insertion of a Prolactin Peptide signal upstream of Concerned B-cell epitope
Compilation
atgAAAGGCAGCCCGTGGAAAGGCAGCCTGCTGCTGCTGCTGATGAACATTGTGAGCAACCTGC
TGCTGTGCCAGAGCGTGGCGCCGCTGCCGgtggtgctgagctttgaactgctgcatgcgccggc
gaccgtgtgcggcccgaaaaaaaaatgcgtgaactttaactttaacggcctgaaaggcaccggc
gtgctgaccgaaagcctgccgccgctgctgaccgatgaaatgattgcgcagtataccagcgcgc
tggcgggcctggtgaaacagctgagcagcaactttggcgcgattagcagcgtgctgaacgatat
tattccgtttgcgatgcagatggcgtatcgctttaacggcattggcgtgacccagaacgcgatt
ccgaccaactttaccattagcgtgaccaccgaaattctgccggtgagcatggtggatttttgcg
gcaaaggctatcatctgatgagctttccgcagagcgcgccgcatccgaccaaactgaacgatct
gtgctttaccaacgtgtatgcggattatcagaccagcaactttcgcgtgcagccgaccgaaagc
attgtgcgctttccgaacaccagcaaccaggtggcggtgctgtatcaggatgtgaactgcaccg
aagtgccggtgtttacccgcggcgtgtattatccggataaagtgtttcgcagcagcgtgctgca
tcgcgaaggcgtgtttgtgagcaacggcacccattggtttgtgtaa (SEQ ID NO: 186)

Routing of B cell Epitope epitopes for membrane expression or secretion from antigen presenting cells for extracellular interaction with the B-cell antigen receptor (BCR), can be accomplished by the 16 aa secretory peptide signal, MLLLLLLLLLLALALA (SEQ ID NO: 1), which if reverse translated yields: atgctgctgctgctgctgctgctgctgctgctggc gctggcgctggcg (SEQ ID NO:187). The addition of this nucleotide sequence enables efficient secretion of expressed fusion proteins in mammalian cells (Barash et al. (2002) Biochem. Biophys. Res. Comm. 294: 835-842; Güler-Gane et al. (2016) PLoS One, 11(5): e0155340). Another alternative, is to use of a prolactin peptide signal, KGSPWKGSLLLLLMNIV SNLLCQSVAPLP (SEQ ID NO:188), which if reverse translated yields: aaaggcagcccgtggaaaggcagcctgctgctgctgctgatgaacattgtgagcaacctgctgctgtgccagagcgtggcgcc gctgccg (SEQ ID NO:189) (Kanekiyo et al. Cell, 2015; 162, 1090-1100). Table 27, bottom row, demonstrates the insertion of the prolactin signal upstream of the concerned B-cell epitope compilation from FIG. 26. In addition to the role of B cells as antigen responsive cells, they can also function as APCs utilizing their antigen receptors for the endocytosis of antigens. These antigens can be proteolytically processed and expressed by MHC-II molecules on the B-cell surface. This arrangement allows the interaction of B-cells with helper T-cells, which introduces additional cytokine co-stimulatory signals to promote immunoglobulin class switching, enhanced immunoglobulin production, and B-cell differentiation into plasma cells (FIG. 8).

In order to integrate the two sets of multi-epitope sequences in Table 27, we made use of a viral T2A sequence, which mediates self-cleavage, leading to a ribosomal skipping action for translation into two sets of peptide sequences, up- and downstream of T2A in eukaryotic cells (Liu et al. (2017) Sci. Rep. 7: 2193). Reverse translation of this peptide sequence GSGEGRGSLLTCGDVEENPG (SEQ ID NO:190), yields: ggcagcggcgaaggccgcggcagcctgctgacctgcggcgatgtggaagaaaacccgggcccg (SEQ ID NO:191), which was also inserted into the integrated mRNA strand to separate Tfh from multi-epitope B-cell sequences (Table 28).

TABLE 28
Separation of dual epitope subsets by T2A. The two constructs described in
Table 27 a were linked by a ribosomal skipping T2A sequence. CD4 are routed to the ER
by Ii₁₋₈₀ (upper case) to accomplish MHC class II loading, while B cell epitopes are
earmarked for secretion by a prolactin sequence (upper case/single underline), as
described in Table 27. Single underline, double underline, bold/italics = epitopes.
Uppercase (capitals) = 1i1-28 sequence. Uppercase (capitals)/underline = prolactin
sequence. Small caps = nucleic acid encoding T2A (GSGEGRGSLLTCGDVEENPGP
(SEQ ID NO: 192)) sequence. Sequence is shown with ATG start codon and TAA stop
codon.

atgATGGATGATCAGCGCGATCTGATTAGCAACCATGAACAGCTGCCGATTCTGGGCAACCGCC
CGCGCGAACCGGAACGCTGCAGCCGCGGCGCGCTGTATACCGGCGTGAGCGTGCTGGTGGCGCT
GCTGCTGGCGGGCCAGGCGACCACCGCGTATTTTCTGTATCAGCAGCAGGGCCGCCTGGATAAA
CTGACCATTACCAGCCAGAACCTGCAGCTGGAAAGCCTGCGCATGAAACTGtatgcgtggaacc
gcaaacgcattagcaactgcgtggcggattatagcgtgctgtatagcaacaaaaaatttctgcc
gtttcagcagtttggccgcgatattaacctgctgctgcagtatggcagcttttgcaccgcgggc
tttattaaacagtatggcgattgcctgggcgatattgcgccgccgctgctgaccgatgaaatga
ttgcgcagtataccagcgcgctgctggcgggcaccattaccagcaacgcgcaggcgctgaacac
cctggtgaaacagctgagcagcaaaGGCAGCGGCGAAGGCCGCGGCAGCCTGCTGACCTGCGGCGATGTGGAAG
AAAACCCGGGCCCGAAAGGCAGCCCGTGGAAAGGCAGCCTGCTGCTGCTGCTGATGAACATTGTGAG
CAACCTGCTGCTGTGCCAGAGCGTGGCGCCGCTGCGgtggtgctgagctttgaactgctgcatg
cgccggcgaccgtgtgcggcccgaaaaaaaaatgcgtgaactttaactttaacggcctgaaagg
caccggcgtgctgaccgaaagcctgccgccgctgctgaccgatgaaatgattgcgcagtatacc
agcgcgctggcgggcctggtgaaacagctgagcagcaactttggcgcgattagcagcgtgctga
acgatattattccgtttgcgatgcagatggcgtatcgctttaacggcattggcgtgacccagaa
cgcgattccgaccaactttaccattagcgtgaccaccgaaattctgccggtgagcatggtggat
ttttgcggcaaaggctatcatctgatgagctttccgcagagcgcgccgcatccgaccaaactga
acgatctgtgctttaccaacgtgtatgcggattatcagaccagcaactttcgcgtgcagccgac
cgaaagcattgtgcgctttccgaacaccagcaaccaggtggcggtgctgtatcaggatgtgaac
tgcaccgaagtgccggtgtttacccgcggcgtgtattatccggataaagtgtttcgcagcagcg
tgctgcatcgcgaaggcgtgtttgtgagcaacggcacccattggtttgtgtaa (SEQ ID
NO: 193)

Codon optimization of the integrated construct yields a mRNA strand for particle delivery (Table 29).

TABLE 29
Codon optimization for the cDNA construct in Table 28. Single underline,
double underline, bold/italics = epitopes. Uppercase (capitals) = li1-28 sequence.
Uppercase (capitals)/underline = prolactin sequence. Small caps = nucleic acid encoding
T2A sequence. Sequence is shown with ATG start codon and TAA stop codon.

atgATGGACGACCAGAGAGATCTGATCAGCAATCACGAGCAGCTGCCAATCCTGGGCAACCGGC
CTAGAGAACCTGAGAGATGCAGCAGAGGCGCTCTGTACACCGGCGTGAGCGTGCTGGTGGCTCT
GTTGTTGGCTGGCCAGGCCACAACCGCCTACTTCCTGTACCAGCAGCAGGGCAGACTGGACAAG
CTGACCATCACCAGCCAGAACCTGCAGCTGGAAAGCCTCAGAATGAAACTGtacgcctggaaca
ggaagcggatcagcaattgcgtggctgattacagcgtcctgtactccaacaagaaattcctgcc
ttttcagcaatttggccgggacatcaacctcctgctgcagtacggctctttttgcaccgccggc
ttcatcaagcaatacggcgactgcctgggcgatatcgcccctccactgctgaccgacgagatga
tcgcccagtacacctctgccctgctggccggcaccatcacctccaacgctcaagctctgaacac
cctggtcaagcaactgagcagcaagGGCAGCGGCGAGGGCCGGGGCAGCCTGCTGACCTGCGGCGACGTGGAAG
AGAACCCCGGCCCCAAAGGTAGCCCTTGGAAGGGAAGCCTGCTGCTGCTGCTCATGAACATCGTGTC
TAATCTGCTGCTGTGTCAGTCTGTGGCCCCTCTGCCTgtggtgctgtctttcgagctgctgcac
gccccagccaccgtgtgtggacctaagaagaagtgcgtgaacttcaacttcaacggcctgaagg
gaaccggcgtgctgaccgaaagcctgcctcctctgctgaccgatgagatgatcgcccagtatac
atctgccctggccggcctggtgaaacagctgagcagcaacttcggtgccataagcagcgtgctt
aatgatattatccctttcgccatgcagatggcctatagatttaacggcatcggcgtgacacaga
acgccatccccacaaacttcaccatctccgtcacaacagaaatcctgcccgtgagcatggtgga
cttctgcggcaagggctaccacctgatgagcttcccccagagcgcccctcaccccaccaagctg
aacgacctgtgcttcacgaacgtgtacgccgactaccagaccagcaacttccgcgtgcagccta
ccgagagcatcgtgcggttcccaaacacatctaaccaggtggccgtgctgtaccaggacgttaa
ttgtacagaagtgcccgtgttcacaagaggagtgtactaccctgataaggtgttcagaagctct
gtgctgcatagagagggcgtttttgtgtccaatggaacacactggttcgtgtaa (SEQ ID
NO: 194)

The above type II vaccines are illustrative and non-limiting. Using the teachings provided herein numerous other type II vaccine sequences will be available to one of skill in the art.

### Type III Vaccine Construction.

It is possible that future COVID-like outbreaks, caused by a spillover from bat SL-CoV sources will have the capacity to generate another COVID-like pandemic with global health, social, and economic impacts in years to come. Since it is very difficult to predict which viral strain may cause the next coronavirus pandemic, the development of a pan-coronavirus vaccine should be considered for protection against a wide range of human and animal coronavirus strains. The integration of three epitope modalities (CD4, CD8 and linear B-cell epitopes) into Vaccine III provides one approach for developing such a vaccine (FIG. 1, illustration of type III vaccine). The use of human B cell, CD4+ and CD8+ T cell epitopes that are conserved among human and animal coronaviruses is an important consideration, as illustrated by the epitope choices made for Vaccines I and IL. One approach would be to combine these epitopes in a Vaccine III, making use of the identified CD4 and CD8 T-cell epitopes, interspaced by a T2A self-cleaving sequence, placed upstream of the conserved B-cell epitopes, also using T2A (FIG. 1, illustration of type III vaccine). Such a type III vaccine version is expected to be helpful dealing with current SARS CoV2 variants, which continuously to evolve and necessitate frequent use of booster shots. The fact that a third SARS CoV2 booster affords improved vaccination responses, above and beyond the second booster, is indicative of the adaptability of the immune system, which to some extent, can benefit from ft boosting updates. However, the availability of a multi-epitope vaccine, structured around conserved spike protein sequences for T- and B-cells may help to build longer-lasting immune protection.

Accordingly, in various embodiments in addition to the illustrative examples provided for type I and type II vaccines, similar design principles can be used to produce multi-epitope mRNA vaccines that incorporates a combination of CD8, CD4 and B-cell epitopes for recognition by T-cell antigen receptors well as the membrane immunoglobulin receptor of B-lymphocytes. Thus, while epitope recognition by the B-cell membrane immunoglobulin receptor constitutes a direct binding event without the requirement of antigen processing for epitope presentation (i.e., no need for MHC binding), the activation of CD4 and CD8 T-cells require consideration of HLA allele frequency to attain widespread population coverage. Moreover, inclusion of CD4 and CD8 epitopes into a single mRNA strand requires epitopes to be routed to MHC-II and -I, respectively, in addition to providing clues for epitope splicing.

To illustrate an integrated epitope design, one can use the multi-epitope predictions by Fatoba et al. (2021) Vaccine, 39: 1111-1121), who used informatics-based predictions to delineate overlapping CD8+ T-cell, IFN-gamma and IL-4 inducer CD4+ T-cell and linear B-cell epitopes. This study explored potential epitopes-based vaccine candidates by retrieving 600 SARS-CoV-2 genomes from the viPR database to identify CD8+ T-cell, CD4+ T-cell and linear B-cell epitopes. These sequences were further refined by screening for antigenicity, immunogenicity and non-allergenicity. This resulted in delineating 18 promising candidate CD8+ T-cell epitopes that overlap with B-cell epitopes (see Tables 30-32 below). In addition, 19 CD4+ T-cell epitopes were identified through their ability to induce IFN-gamma and IL-4 cytokines. Moreover, the T-cell epitope analysis was further refined to obtain population coverage for MHC-I and MHC-II, in addition to mapping these epitopes to the complete COVID-19 genome. The majority of dominant epitopes were localized on the CoV-2 surface (S) and membrane (M) glycoproteins. All considered, these epitopes can serve as candidates to design multi-epitope vaccines, which can accomplish diverse involvement and activation of cytotoxic T-cells, helper T cells, memory T cells and antibody producing B-cell precursors.

TABLE 30
18 CD8+ T-cells epitopes in SARS-COV-2 that are immunogenic, bind to MHC
class I HLA-alleles, and overlap with CD4+ T-cell epitopes (Fatoba et al. supra.).

| | SEQ | | | | | |
|---|---|---|---|---|---|---|
| CD8* T-cells | ID | Antigenicity | | | | Conservancy |
| epitopes | NO | score | Immunogenicity | MHC-I alleles | Position | scores |
| FLWLLWPV | 195 | 1.4835 | 0.3785 | HLA-A*02:01, | 80 | 100% |
| | | | | HLA-A*02:06 | | |
| RNRFLY1IK | 196 | 0.8364 | 0.3104 | HLA- | 102 | 100% |
| | | | | A*3O:O1,HLA- | | |
| | | | | A*31:01 | | |
| ATRRIRGGD | 197 | 0.6108 | 0.2956 | HLA-A′30:01 | 57 | 100% |
| LWLLWPVTL | 198 | 1.1590 | 0.2480 | HLA-A′23:01 | 276 | 100% |
| ENAFLPFAM | 199 | 1.0278 | 0.2309 | HLA-A′68:02, | 151 | 100% |
| | | | | HLA-B*35:01 | | |
| ATATIPIQA | 200 | 0.7881 | 0.2155 | HLA-A′68:02 | 54 | 100% |
| LAAVYRINW | 201 | 1.1825 | 0.2079 | HLA-B*58:01. | 170 | 100% |
| | | | | HLA-B′53:01. | | |
| | | | | HLA-B′57:01 | | |
| LACFVLAAV | 202 | 1.1343 | 0.1859 | HLA-A*02:06, | 171 | 100% |
| | | | | HLA-A*02:03 | | |
| FAMGIIAMS | 203 | 1.1005 | 0.1624 | HLA-A*68:02 | 160 | 100% |
| NLLLLFVTV | 204 | 0.6912 | 0.1421 | HLA- | 326 | 100% |
| | | | | A*02:06, HLA- | | |
| | | | | A*02:01, HLA- | | |
| | | | | A*02:03 | | |
| TLACFVLAA | 205 | 0.5781 | 0.1248 | HLA- | 240 | 100% |
| | | | | A*02:03, HLA- | | |
| | | | | A*02:01, HLA- | | |
| | | | | A′02:06 | | |
| VTLACFVLA | 206 | 0.5358 | 0.1218 | HLA-A′02:06 | 80 | 100% |
| ELLHAPATV | 207 | 1.0476 | 0.1123 | HLA-A′02:03 | 149 | 100% |
| RWVLSFEL | 208 | 0.6783 | 0.0457 | HLA-A′02:06 | 125 | 100% |
| YIIKLIFLW | 209 | 0.5220 | 0.0330 | HLA- | 130 | 100% |
| | | | | B′58:01, HLA- | | |
| | | | | B′53:01, HLA- | | |
| | | | | A′02:01, HLA- | | |
| | | | | A′02:06, HLA- | | |
| | | | | B′57:01 | | |
| GIAIAMACL | 210 | 0.5505 | 0.0209 | HLA-A*02:03 | 13 | 100% |
| AMACLVGL | 211 | 0.9558 | 0.0086 | HLA- | 31 | 100% |
| M | | | | A*02:03.HLA- | | |
| | | | | B′15:01 | | |
| AFLPFAMGI | 212 | 0.9095 | 0.0076 | HLA-A*02:06 | 17 | 100% |

TABLE 31
19 1 CD4+ T-cells epitopes in SARS-COV-2 that are antigenic, induce IFN-γ and
IL-4, interact with HLA class II -alleles and overlap with CD8+ T-cell epitopes (Fatoba et
al).

| Overlapping CD4 T-cells | SEQ ID | Antigenicity | | Conservancy |
|---|---|---|---|---|
| Epitopes | NO | scores | MHC-II alleles | (%) |
| ACFVLAAVYRINWIT | 213 | 1.1115 | HLA- | 100% |
| | | | DRB4*01:01 | |
| AFLPFAMGI1AMSAF | 214 | 1.0499 | HLA-DQA1′01: | 100% |
| | | | 01 /DQB105: | |
| | | | 01 | |
| FFLYENAFLPFAMGI | 215 | 0.8191 | HLA- | 100% |
| | | | DPA1′02:01/ | |
| | | | DPB1′05:01 | |
| FLPFAMGIIAMSAFA | 216 | 1.0725 | HLA- | 100% |
| | | | DRB4′01:01 | |
| GIAIAMACLVGLMWL | 217 | 0.9155 | HLA- | 100% |
| | | | DRB4′01:01 | |
| IGYYRRATRRIRGGD | 218 | 0.6649 | HLA-DPA102: | 100% |
| | | | 01/DPB1*01: | |
| | | | 01 | |
| IKLIFLWLLWPVTLA | 219 | 0.8704 | HLA-DQA101: | 100% |
| | | | 01 /DQB1′05: | |
| | | | 01 | |
| KLIFLWLLWPVTLAC | 220 | 0.7344 | HLA-DRB1′08: | 100% |
| | | | 02 | |
| LPFAMGIIAMSAFAM | 221 | 1.0879 | HLA- | 100% |
| | | | DRB1′01:01 | |
| NAFLPFAMGHAMSA | 222 | 0.9681 | HLA- | 100% |
| | | | DPAr03:01/ | |
| | | | DPB1′04:02 | |
| PSDFVRATATIPIQA | 223 | 0.5466 | HLA-DPA1′03: | 100% |
| | | | 01 / | |
| | | | DPB104:02 | |
| PVTLACFVLAAVYRI | 224 | 0.9703 | HLA-DPA1′03: | 100% |
| | | | 01 / | |
| | | | DPB1′04:02 | |
| RNRFLYI1KLIFLWL | 225 | 0.5114 | HLA-DPA1′02: | 100% |
| | | | 01/DPB1′01: | |
| | | | 01 | |
| RWVLSFELLHAPAT | 226 | 0.7485 | HLA-DQA101: | 100% |
| | | | 01 / | |
| | | | DQB1′05:01 | |
| SDFVRATATIPIQAS | 227 | 0.5411 | HLA-DQA1′01: | 100% |
| | | | 01 / | |
| | | | DQB1′05:01 | |
| T LAC FV LA AVY RI | 228 | 1.3949 | HLA-DQA1′01: | 100% |
| NW | | | 01 / | |
| | | | DQB1′05:01 | |
| VHFVCNLLLLFVTVY | 229 | 1.3132 | HLA- | 100% |
| | | | DPA1′03:01/ | |
| | | | DPBT04:02 | |
| WLSFELLHAPATVC | 230 | 0.8835 | HLA-DRBT11: | 100% |
| | | | 01 | |
| YRWVLSFELLHAPA | 231 | 0.9711 | HLA-DPA1′01: | 100% |
| | | | 03/ | |
| | | | DPB1′02:01 | |

All 18 CD8+ T-cell epitopes are antigenic as well as immunogenic. All 19 CD4+ T-cell epitopes are able to induce IFN-γ and IL-4 in CD4+ T cells. The conservancy analysis for all CD4+ and CD8+ T-cell epitopes (Table 31) were 100%. The analysis also included identification of 8 B-cell epitopes in SARS-CoV-2 (Table 32), which are antigenic and capable of stimulating violent neutralizing antibodies. All of the above epitopes were demonstrated by informatics to be non-allergic in nature.

TABLE 32
Putative linear B-cell epitopes of SARS-COV-2 with their antigenicity and
allergenicity.

| | SEQ ID | ABCpred | | Vaxijen | Conservancy | |
|---|---|---|---|---|---|---|
| B-cell epitopes | NO | score | Position | score | (%) | Allergenicity |
| GIAIAMACLVG | 232 | 0.74 | 1206 | 0.9132 | 100% | Non-allergen |
| LMWLS | | | | | | |
| GKNWITGG1A1 | 233 | 0.78 | 1199 | 0.6906 | 100% | Non-allergen |
| AMACL | | | | | | |
| LWPVTLACFVL | 234 | 0.59 | 1086 | 0.9305 | 100% | Non-allergen |
| AAVYN | | | | | | |
| LWPVTLACFVL | 235 | 0.56 | 1727 | 0.8961 | 100% | Non-allergen |
| AAVYR | | | | | | |
| RINWITGGIAIA | 236 | 0.77 | 1401 | 1.2392 | 100% | Non-allergen |
| MACL | | | | | | |
| WLSFELLHAPA | 237 | 0.66 | 560 | 0.6184 | 100% | Non-allergen |
| TVCG | | | | | | |
| YENAFLPFAMG | 238 | 0.67 | 1607 | 1.1003 | 100% | Non-allergen |
| IIAMS | | | | | | |
| YFLCWHTNCY | 239 | 0.84 | 773 | 0.8340 | 100% | Non-allergen |
| DYCIPY | | | | | | |

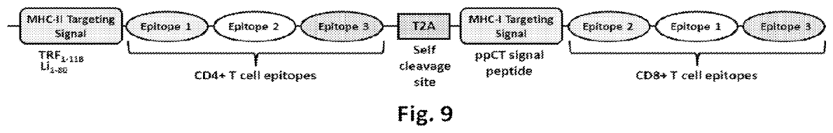

Peptide expression by a mRNA strand is capable of leading to antigen presentation, provided that the expressed epitopes are appropriately processed. To accomplish the construction of a single mRNA strand that can express and deliver native CD4 and CD8 epitopes to the appropriate endosomal compartments, it was necessary for us to arrange these epitopes to allow their processing prior to gaining access to the respective endosomal compartments (FIG. 9). While processing of CD8+ epitopes by the cytosolic 26S proteosome may allow entry into the MHC-I compartment by TAP transporters, it is necessary for CD4 epitopes to be directed towards the MHC-II endosomal compartment by different mechanisms. For CD4 epitopes this can be accomplished by expressing these sequences downstream of a transferrin receptor (TfR 1-118) or invariant (li) chain sequence (1-80), both of which allowing access to the MHC class II compartment. To accomplish this arrangement, our first design was to separate CD4 from CD8 epitopes through the addition of a self-cleaving linker peptides (T2A) sequence (GSGEGRGSLLTCGDVEENPG, (SEQ ID NO:240)). T2A, when reverse translated yields: ggcagcggcgaaggccgcggcagcctgct gacctgcggcga tgtggaagaaaacccgggcccg, (SEQ ID NO:241). T2A peptides are 18-22 amino-acid (aa) viral oligopeptides that mediate “cleavage” of polypeptides during translation in eukaryotic cells (Liu et al. (2017) Sci. Rep. 7: 2193). The mechanism of 2A-mediated “self-cleavage” involves a ribosomal skipping action, which separates the CD4 upstream from the downstream CD8 epitopes (earmarked for cytosolic processing to gain MHC-1 access). In addition to using this compartmental segregation mechanism, it is also possible to add a leader signal peptide sequence, VLLQAGSLHA (SEQ ID NO:49), derived from the human pre-procalcitonin (ppCT) signal peptide (Durgeau et al. (2018) Nature Commun. 9: 5097). This signal peptide provides an alternative means of facilitating MHC-I epitope presentation to CD8 T-cells.

The cDNA construct is provided with stop and stop codons, that can be codon optimized as described above. The untranslated region, 5′ UTR and 3′ UTR region are pre-existing in commercially available plasmids for mRNA preparation, e.g., pTNT from Promega or CleanCap plasmid from TriLink. 5′ cap and poly(A) tail are added post-transcriptionally. The cDNA synthesis, plasmid construction and mRNA preparation can be out sourced to TriLink (Carlsbad, San Diego). FIG. 10 summarizes epitope sorting to MHC-I and MHC-II after delivery of multi-epitope mRNA cationic lipid nanoparticles to APC.

In addition to epitope delineation by Fatoba et al., supra., a large number of CD4 and CD8 epitopes from other sources are listed in our previous disclosure; the reverse translated cDNA sequences can be used to construct mRNA vaccines. For example, CD8+ T-cell epitopes listed by Nathan et al. (2021) Cell, 184: 4401-4413) in Table 30 could be combined with CD4+ T-cell epitopes from Table 31 above, as well as a variety of other sources (see, e.g., Nelde et al. (2021) Nat. Immunol. 22: 74-85; Staquicini et al. (2021) Proc. Natl. Acad. Sci. USA, 118(30): e2105739118). It is also possible to use the design of multi-protein/peptide vaccines to design corresponding mRNA vaccines. For instance, a recent study by COVAXX showed a novel SARS-CoV-2 multi-epitope protein/peptide vaccine candidate that is highly immunogenic and prevents lung infection in an adeno associated virus human angiotensin-converting enzyme 2 (AAV hACE2) mouse model (Guirakhoo et al. (2020) bioRxiv, doi: https://doi.org/10.1101/2020.11.30.399154). This vaccine was synthesized by fusing the S1-RBD-sFc fusion protein to six synthetic peptides (one universal peptide and five SARS-CoV-2-derived peptides). Their approach is multi-step, multi-component, and time-consuming enterprise, which could be more rapidly accomplished by a mRNA construct, such as described herein.

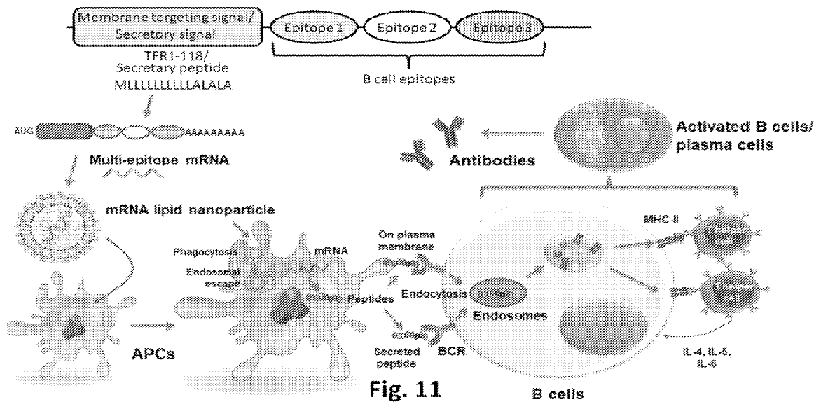

In order for B-cell epitopes to be recognized by B-cell mIg receptors, it is not necessary to achieve MHC expression, but MHC-II binding interactions can be used by antigen presenting B-cells for epitope display to CD4 helper T-cells that assist antibody production. To facilitate epitope recognition by antigen responsive B-cells, the mRNA vaccine can be designed using two different but complementary expression mechanisms, namely: (i) epitope expression on the plasma membrane of the antigen presenting cell (APC); or (ii) secretion from the APC to allow contact with B-cells, extracellularly (FIG. 11). Surface expression on the APC is be accomplished by inserting the epitopes downstream of the transferrin receptor domain (aa 1-118), which acts as a leader sequence that transports the expressed peptide to the cell surface (Diebold et al. (2001) Gene Therapy, 8: 487-493). This endosomal pathway also allows secondary endocytic transport to the MHC-II compartment, where epitope processing allows helper CD4+ T-cell activation that can enhance antibody production and immunoglobulin class switching (Id.). For the secretion of expressed epitopes, allowing extracellular B-cell antigen recognition, the mRNA strand can be endowed with a secretory peptide signal. An example of a 16 aa secretory peptide is MLLLLLLLLLLALALA (SEQ ID NO:1), which upon reverse translation yields the cDNA sequence: atgctgctgctgctgctgctgctgctgctgctg gcgctggcgctggcg (SEQ ID NO:242). The addition of this nucleotide sequence has been demonstrated to enable the efficient secretion of expressed fusion proteins in mammalian cells (see, e.g., Barash et al. (2002) Biochem. Biophys. Res. Comm. 294: 835-842), as well as Giler-Gane et al. (2016) PLoS One, 11(5): e0155340).

In a further iteration of mRNA multi-epitope vaccine design, B-cell epitopes can be combined with CD8+ and CD4 T cell epitopes in various combinations. All of these design modifications can further be supplemented by designing the mRNA constructs to enhance Th1 skewing of the immune response, which is important for preventing antibody-dependent enhancement and generation of Th2-mediated eosinophilic lung damage (Nel & Miller (2021) ACS Nano, 15, 5793-5818). It is also possible to achieve this outcome by adding adjuvants that can be added to the nanoparticles, such as cPG oligonucleotide sequences or the list of adjuvants included in our previous disclosure.

Another example of a further iteration of mRNA multi-epitope vaccine design comes from Prakash et al. (2021) J Immunol. 206: 2566-2582), who have proposed a preemptive multi-epitope pan-coronavirus vaccine, which extends beyond a focus on spike protein epitopes to include highly conserved human B and CD4+ and CD8+ T cell epitopes, derived identified from other viral protein sequences, including proteins from other coronavirus species that infect animals. By employing several immunoinformatics and sequence alignment tools, Prakash et al. identified several conserved human B cell as well as CD4+ and CD8+ T-cell epitopes in over 81,000 SARS-CoV-2 genome sequences identified on six continents. Moreover, this lineup also includes epitopes identified in common cold CoVs, SL-CoVs isolated from bats, SL-CoV isolated from pangolins, SL-CoVs isolated from civet cats; and MERS strains isolated from camels. Refinement of those epitope selections, culminated in collecting three sets of epitope selections for multi-epitope design of a vaccine for pandemic prevention, (Table 33).

TABLE 33
Example of preventative pan-corona vaccine T-cell and B-cell epitope choices.
Prakash et al. (2021) J. Immunol. 206: 2566-2582 evaluated 81,963 SARSCoV-2 genome
sequences from around the globe, using a 100% conservancy threshold to identify
candidate CD8+ T cell, CD4+ T cell, and B cell epitopes in the human genome. The
selections also show s overlap with sequences from CoVs causing common cold outbreaks
in humans, as well as CoVs isolated from bats, pangolins, civet cats or camels. The
sequence selection allowed for population coverage, as determined by Population
Coverage software in the IEDB platform. Altogether, they identified 27 highly conserved
CD8+ T cell interactive sequences, subsequently refined to 15 sequences, mostly
expressed in non-spike proteins. Similar identification of CD4 interactive sequences were
obtained using multiple databases and algorithms, including the SYFPEITHI (SEQ ID
NO: 243) MHC-II binding predictions, TepiTool, and TEPITOPEpan. This led to the
demonstration of 16 highly conserved CD4+ T cell epitopes present in 9 of 12 available
open reading frames (ORF's), including ORF1 for S, E, M, ORF6, ORF7a, ORF7b, ORF8,
and nucleoprotein. Six non-spike protein epitopes were identified on the basis of recall
memory T-cell responses in Covid-19 patients as well as asymptomatic individuals
(presumably previously infected with corona cold viruses). Reverse translations of the
epitope sequences are also shown.
Human CD8+ T cell epitopes

| Viral | | | SEQ | | SEQ |
|---|---|---|---|---|---|
| protein | Position | Sequence | ID NO | Reverse translation | ID NO |
| ORF1ab | 1675-1683 | YLATALLTL | 244 | tatctggcgaccgcgctg | 245 |
| | | | | ctgaccctg | |
| ORF1ab | 2363-2371 | WLMWLIINL | 246 | tggctgatgtggctgattat | 247 |
| | | | | taacctg | |
| ORFIab | 3013-3021 | SLPGVFCGV | 248 | agcctgccgggcgtgttttg | 249 |
| | | | | cggcgtg | |
| ORFIab | 5470-5478 | KLSYGIATV | 250 | aaactgagctatggcattgc | 251 |
| | | | | gaccgtg | |
| ORFIab | 6749-6757 | LLLDDFVEI | 252 | ctgctgctggatgattttgt | 253 |
| | | | | ggaaatt | |
| Spike | 2-10 | FVFLVLLPL | 254 | tttgtgtttctggtgctgct | 255 |
| | | | | gccgctg | |
| Spike | 958-966 | ALNTLVKQL | 256 | gcgctgaacaccctggtgaa | 257 |
| | | | | acagctg | |
| Spike | 1000-1008 | RLQSLQTYV | 258 | cgcctgcagagcctgcagac | 259 |
| | | | | ctatgtg | |
| Spike | 1220-1228 | FIAGLIAIV | 260 | tttattgcgggcctgattgc | 261 |
| | | | | gattgtg | |
| Envelope | 20-28 | FLAFVVFLL | 262 | tttctggcgtttgtggtgtt | 263 |
| protein | | | | tctgctg | |
| Envelope | 26-34 | FLLNKEMYL | 264 | tttctgctgaacaaagaaat | 265 |
| protein | | | | gtatctg | |
| ORF7b | 26-34 | IIFWFSLEL | 266 | attattttttggtttagcct | 267 |
| | | | | ggaactg | |
| ORF8a | 73-81 | YIDIGNYTV | 268 | tatattgatattggcaacta | 269 |
| | | | | taccgtg | |
| ORF | 103-11 | YINVFAFPF | 270 | tatattaacgtgtttgcgtt | 271 |
| | | | | tccgttt | |
| ORF | 105-13 | NVFAFPFTI | 272 | aacgtgtttgcgtttccgtt | 273 |
| | | | | taccatt | |

Human CD4+ T cell epitopes

| Viral | | | | | |
|---|---|---|---|---|---|
| protein | Position | Sequence | | Reverse translation | |
| ORF1a | 1350-1365 | KSAFYILPSIIS | 274 | aaaagcgcgttttatattct | 275 |
| | | NEK | | gccgagcattattagcaacg | |
| | | | | aaaaa | |
| ORF1ab | 5019-5033 | PNMLRIMASLVL | 276 | ccgaacatgctgcgcattat | 277 |
| | | ARK | | ggcgagcctggtgctggcgc | |
| | | | | gcaaa | |
| Membrane | 76-190 | LSYYKLGASQRV | 278 | ctgagctattataaactggg | 279 |
| glycoprot | | AGD | | cgcgagccagcgcgtggcgg | |
| 1 | | | | gcgat | |
| ORF6 | 12-26 | AEILLIIMRTFK | 280 | gcggaaattctgctgattat | 281 |
| | | VSI | | tatgcgcacctttaaagtga | |
| | | | | gcatt | |
| ORF7b | 8-22 | DFYLCFLAFLLF | 282 | gatttttatctgtgctttct | 283 |
| | | LVL | | ggcgtttctgctgtttctgg | |
| | | | | tgctg | |
| ORF8b | 1-15 | MKFLVFLGIITT | 284 | atgaaatttctggtgtttct | 285 |
| | | VAA | | gggcattattaccaccgtgg | |
| | | | | cggcg | |

TABLE 34
Similarly, Prakash et al. carried out linear B cell epitope predictions for SARS
CoV2 spike protein, using the BepiPred 2.0 algorithm hosted on the IEDB platform. An
epitope probability score was calculated for each amino acid. Potential B cell epitopes
were predicted using a cutoff of 0.55 (corresponding to a specificity >0.81 and sensitivity
<0.3). This screening originally resulted in 28 B-cell peptides, from which 4 highly
conserved immunogenic and antigenic human B cell sequences, recognized by IgG Abs in
the serum of COVID-19 patients as well as unexposed healthy individuals. While the
identification of epitopes present in spike protein is of immediate relevance for shaping the
immune response to SARS COV2 variants of concern, the use of epitopes from non-spike
proteins, can be included in this lineup, if needed. Reverse translations of the epitope
sequences are also shown.

| Spike | | SEQ | | SEQ |
|---|---|---|---|---|
| Protein | | ID | | ID |
| Position | Sequence | NO | Reverse Translation | NO |
| 13-37 | SQCVNLTTRTQLPP | 286 | agccagtgcgtgaacctgaccacccgcacc | 287 |
| | AYTNSFTRGVY | | cagctgccgccggcgtataccaacagcttt | |
| | | | acccgcggcgtgtat | |
| 218-317 | DAVDCALDPLSETK | 288 | gatgcggtggattgcgcgctggatccgctg | 289 |
| | CTLKSFTVEKGIYQ | | agcgaaaccaaatgcaccctgaaaagcttt | |
| | T SN | | accgtggaaaaaggcatttatcagaccagc | |
| | | | aac | |
| 601-640 | GTNTSNQVAVLYQD | 290 | ggcaccaacaccagcaaccaggtggcggtg | 291 |
| | VNCTEVPVAIHADQ | | ctgtatcaggatgtgaactgcaccgaagtg | |
| | LTPTWRVYSTGS | | ccggtggcgattcatgcggatcagctgacc | |
| | | | ccgacctggcgcgtgtatagcaccggcagc | |
| 524-598 | VCGPKKSTNLVKNK | 292 | gtgtgcggcccgaaaaaaagcaccaacctg | |
| | CVNFNFNGLTGTG | | gtgaaaaacaaatgcgtgaactttaacttt | |
| | VLTESNKKFLPFQQ | | aacggcctgaccggcaccggcgtgctgacc | |
| | FGRDIADTTDAVRD | | gaaagcaacaaaaaatttctgccgtttcag | 293 |
| | PQTLEILDITPCSF | | cagtttggccgcgatattgcggataccacc | |
| | GGVSVI | | gatgcggtgcgcgatccgcagaccctggaa | |
| | | | attctggatattaccccgtgcagctttggc | |
| | | | ggcgtgagcgtgatt | |

Importantly, the identified epitopes were able to recall B-as well as CD4+ and CD8+ T cell responses in blood cells from COVID-19 patients and healthy individuals, never previously exposed to SARS-CoV-2. Most of the T cell epitopes appeared in nonstructural proteins, with ORF1a/b being the most frequently targeted antigen. The work also demonstrated that the selected epitopes are capable of inducing strong B cell and T cell responses in humanized HLA-DR1/HLA-A*02:01 double-transgenic mice (Prakash et al.; J Immunol 2021; 206:2566-2582). As an illustrative example of how to design a pandemic prevention type III nucleic acid vaccine, we used the reverse translated nucleotide sequences, representative of the epitope selections in Tables 33 and 34, for generating an integrated multi-epitope mRNA vaccine that includes CD4 epitopes (Table 35), CD8 epitopes (Table 36), and B-cell epitopes (Table 37).

TABLE 35
The CD4 T cell epitopes were linked together downstream of the invariant Li
(1-80) sequence, as shown in Table 33. Uppercase (capital) = Li1-80 leader. Single
underline, double underline, bold/italics = CD4 epitopes.

ATGGACGACCAGAGAGATCTGATCAGCAATCACGAGCAGCTGCCAATCCTGGGCAACCGGCCTA
GAGAACCTGAGAGATGCAGCAGAGGCGCTCTGTACACCGGCGTGAGCGTGCTGGTGGCTCTGTT
GTTGGCTGGCCAGGCCACAACCGCCTACTTCCTGTACCAGCAGCAGGGCAGACTGGACAAGCTG
ACCATCACCAGCCAGAACCTGCAGCTGGAAAGCCTCAGAATGAAACTGggcccgggcccgggca
aaagcgcgttttatattctgccgagcattattagcaacgaaaaaggcccgggcccgggcccgaa
catgctgcgcattatggcgagcctggtgctggcgcgcaaaggcccgggcccgggcctgagctat
tataaactgggcgcgagccagcgcgtggcgggcgatggcccgggcccgggcgcggaaattctgc
tgattattatgcgcacctttaaagtgagcattggcccgggcccgggcgatttttatctgtgctt
tctggcgtttctgctgtttctggtgctgggcccgggcccgggcatgaaatttctggtgtttctg
ggcattattaccaccgtggcggcg (SEQ ID NO: 294)

TABLE 36
CD8 T cell epitopes were linked with the ppCT signal sequence inserted
upstream of the epitope selection shown in Table 33. Uppercase (capital) = ppCT signal
sequence. Single underline, double underline, bold/italics = CD8 epitopes.

GTGCTGCTGCAGGCCGGCAGCCTGCACGCCAAGAAAgaagcggcggcgaaatatctggcgaccg
cgctgctgaccctggcggcgtattggctgatgtggctgattattaacctggcggcgtatagcct
gccgggcgtgttttgcggcgtggcggcgtataaactgagctatggcattgcgaccgtggcggcg
tatctgctgctggatgattttgtggaaattgcggcgtattttgtgtttctggtgctgctgccgc
tggcggcgtatgcgctgaacaccctggtgaaacagctggcggcgtatcgcctgcagagcctgca
gacctatgtggcggcgtattttattgcgggcctgattgcgattgtggcggcgtattttctggcg
tttgtggtgtttctgctggcggcgtattttctgctgaacaaagaaatgtatctggcggcgtata
ttattttttggtttagcctggaactggcggcgtattatattgatattggcaactataccgtggc
ggcgtattatattaacgtgtttgcgtttccgtttgcggcgtataacgtgtttgcgtttccgttt
accatt (SEQ ID NO: 295)

TABLE 37
The B-cell epitopes identified in Table 34 were linked together with upstream
insertion of a prolactin secretory signal. Uppercase (capital) = prolactin secretory signal.
Single underline, double underline, bold/italics = CD8 epitopes.

GTGCTGCTGCAGGCCGGCAGCCTGCACGCCAAGAAAgaagcggcggcgaaatatctggcgaccg
cgctgctgaccctggcggcgtattggctgatgtggctgattattaacctggcggcgtatagcct
gccgggcgtgttttgcggcgtggcggcgtataaactgagctatggcattgcgaccgtggcggcg
tatctgctgctggatgattttgtggaaattgcggcgtattttgtgtttctggtgctgctgccgc
tggcggcgtatgcgctgaacaccctggtgaaacagctggcggcgtatcgcctgcagagcctgca
gacctatgtggcggcgtattttattgcgggcctgattgcgattgtggcggcgtattttctggcg
tttgtggtgtttctgctggcggcgtattttctgctgaacaaagaaatgtatctggcggcgtata
ttattttttggtttagcctggaactggcggcgtattatattgatattggcaactataccgtggc
ggcgtattatattaacgtgtttgcgtttccgtttgcggcgtataacgtgtttgcgtttccgttt
accatt (SEQ ID NO: 296)

The CD4, CD8, and B cell epitope compilations shown in Tables 35-37 above were linked together, using insertion of a T2A cleavable linker and a P2A cleavable linker.

Table 38 shows the integrated construct.

TABLE 38
integrated construct comprising CD4, CD8 and B cell epitopes. While regions
are shown in different rows, the sequence is a single contiguous sequence (SEQ ID
NO: 297). Note upper case (capitals) = leader sequences as identified. SmallCaps =
cleavage sequence. Single underline, double underline, bold/italics = epitopes. Sequence
is shown with ATG start codon and TAA stop codon.

| Sequence | Domain |
|---|---|
| atgTGGACGACCAGAGAGATCTGATCAGCAATCACGAGCAGCTGCCAATCCTGGGC | Li1-80 + |
| AACCGGCCTAGAGAACCTGAGAGATGCAGCAGAGGCGCTCTGTACACCGGCGTGAG | CD4 |
| CGTGCTGGTGGCTCTGTTGTTGGCTGGCCAGGCCACAACCGCCTACTTCCTGTACC | epitopes |
| AGCAGCAGGGCAGACTGGACAAGCTGACCATCACCAGCCAGAACCTGCAGCTGGAA | |
| AGCCTCAGAATGAAACTGggcccgggcccgggcaaaagcgcgttttatattctgcc | |
| gagcattattagcaacgaaaaaggcccgggcccgggcccgaacatgctgcgcatta | |
| tggcgagcctggtgctggcgcgcaaaggcccgggcccgggcctgagctattataaa | |
| ctgggcgcgagccagcgcgtggcgggcgatggcccgggcccgggcgcggaaattct | |
| gctgattattatgcgcacctttaaagtgagcattggcccgggcccgggcgattttt | |
| atctgtgctttctggcgtttctgctgtttctggtgctgggcccgggcccgggcatg | |
| aaatttctggtgtttctgggcattattaccaccgtggcggcg | |
| GGCAGCGGCGAAGGCCGCGGCAGCCTGCTGACCTGCGGCGATGTGGAAGAAAACCCGGGCCCG | T2A |
| GTGCTGCTGCAGGCCGGCAGCCTGCACGCCAAGAAAgaagcggcggcgaaatatct | ppCT |
| ggcgaccgcgctgctgaccctggcggcgtattggctgatgtggctgattattaacc | leader + |
| tggcggcgtatagcctgccgggcgtgttttgcggcgtggcggcgtataaactgagc | CD8 |
| tatggcattgcgaccgtggcggcgtatctgctgctggatgattttgtggaaattgc | epitopes |
| ggcgtattttgtgtttctggtgctgctgccgctggcggcgtatgcgctgaacaccc | |
| tggtgaaacagctggcggcgtatcgcctgcagagcctgcagacctatgtggcggcg | |
| tattttattgcgggcctgattgcgattgtggcggcgtattttctggcgtttgtggt | |
| gtttctgctggcggcgtattttctgctgaacaaagaaatgtatctggcggcgtata | |
| ttattttttggtttagcctggaactggcggcgtattatattgatattggcaactat | |
| accgtggcggcgtattatattaacgtgtttgcgtttccgtttgcggcgtataacgt | |
| gtttgcgtttccgtttaccatt | |
| GGCAGCGGCGCGACCAACTTTAGCCTGCTGAAACAGGCGGGCGATGTGGAAGAAAACCCGGGCCCG | P2A |
| GTGCTGCTGCAGGCCGGCAGCCTGCACGCCAAGAAAgaagcggcggcgaaatatct | Prolactin |
| ggcgaccgcgctgctgaccctggcggcgtattggctgatgtggctgattattaacc | + B cel |
| tggcggcgtatagcctgccgggcgtgttttgcggcgtggcggcgtataaactgagc | epitopes |
| tatggcattgcgaccgtggcggcgtatctgctgctggatgattttgtggaaattgc | |
| ggcgtattttgtgtttctggtgctgctgccgctggcggcgtatgcgctgaacaccc | |
| tggtgaaacagctggcggcgtatcgcctgcagagcctgcagacctatgtggcggcg | |
| tattttattgcgggcctgattgcgattgtggcggcgtattttctggcgtttgtggt | |
| gtttctgctggcggcgtattttctgctgaacaaagaaatgtatctggcggcgtata | |
| ttattttttggtttagcctggaactggcggcgtattatattgatattggcaactat | |
| accgtggcggcgtattatattaacgtgtttgcgtttccgtttgcggcgtataacgt | |
| gtttgcgtttccgtttaccatttaa | |

Table 39 shows the codon optimized version of the sequence in Table 38.

TABLE 39
Codon optimized sequence version of Table 38 (SEQ ID NO: 298). While
regions are shown in different rows, the sequence is a single contiguous sequence (SEQ ID
NO: 299). Note upper case (capitals) = leader sequences as identified. Small caps =
cleavage sequence. Single underline, double underline, bold/italics = epitopes. Sequence
is shown with ATG start codon and TAA stop codon.

| Sequence | Domain |
|---|---|
| atgATGGACGACCAGCGCGATCTGATCAGCAACCACGAGCAGCTGCCTATCCTG | Li1-80 + |
| GGCAATAGACCTAGAGAGCCTGAGCGGTGCAGCAGGGGCGCTCTGTACACTGG | CD4 |
| AGTGTCCGTGCTGGTGGCTCTGCTGCTGGCCGGCCAGGCCACCACCGCCTACTT | epitopes |
| CCTGTACCAGCAGCAGGGCAGACTGGATAAGCTGACCATCACCAGCCAGAATCT | |
| GCAACTCGAGAGCCTCAGAATGAAGCTGggcccaggccctggaaagtctgccttctacatcc | |
| tgcctagcatcatcagcaatgagaagggccccggccccggacccaacatgctgagaatcatggccagcc | |
| tggtactggcccggaagggccccggcccaggcctgagctactacaagctgggcgctagccagagagtgg | |
| ccggcgacggccccggccccggcgccgagattctgctgatcatcatgcggacattcaaggtgtccatcgg | |
| ccccggccctggagacttctacctgtgcttcctggccttcctgctgtttctggtgctgggcccaggcccgggc | |
| atgaaattcctggtgtttctgggcatcatcacaacagtggccgct | |
| GGCTCTGGCGAGGGCAGAGGCAGCCTGCTGACATGCGGGGACGTGGAAGAAAACCCCGGACCT | T2A |
| GTGCTGCTGCAGGCCGGCAGCCTGCACGCCAAGAAAgaggccgccgccaagtacctgg | ppCT |
| caaccgccctgctgaccctggctgcctactggctgatgtggctgatcatcaacctggccgcctatagcctgc | leader + |
| caggagtgttctgtggagtggccgcttacaaactgagctacggcatcgccacagtggccgcttacctgctg | CD8 |
| ttagatgatttcgtggaaatcgctgcctacttcgtgttcctggtgctgctgcctctggctgcatacgccctga | epitopes |
| acaccctggtcaagcaactggccgcttatagactgcagagcctgcagacctacgtggccgcttacttcatc | |
| gccggcctgatcgccattgtggccgcctactttctggcctttgtggtgtttctgctggccgcctacttcctgct | |
| caacaaggaaatgtacctggccgcttacatcatcttctggttcagcctcgagctggccgcctactacatcg | |
| acatcggcaactacacggttgctgcctattacatcaacgtgttcgcctttcctttcgccgcttacaacgtgtt | |
| cgccttccccttcaccatc | |
| GGCAGCGGCGCCACAAACTTCAGCCTGCTCAAGCAGGCCGGAGATGTGGAAGAGAACCCTGGACCT | P2A |
| AAGGGCAGCCCTTGGAAGGGATCTCTGCTGCTGCTGCTGATGAACATCGTCAGC | prolactin + |
| AATCTGCTGCTTTGTCAGTCTGTGGCCCCTCTGCCTaagaagtctcagtgcgtgaacctga | b cel |
| caaccagaacccagctgccacctgcctataccaacagctttaccagaggggtctacaagaaagacgccg | epitopes |
| tggactgcgccctggatcctctgtccgagacaaagtgcaccctgaaatccttcaccgtggaaaagggaat | |
| ctaccagacaagcaacaaaaagggcacaaacacatctaaccaggtggccgtgctctatcaggacgttaa | |
| ctgcaccgaggtgcctgtggcgatccacgccgaccagctgacacctacctggcgggtgtacagcaccggc | |
| tctaagaaagtgtgcggccctaagaagtccaccaacctggtgaagaataagtgtgtgaatttcaacttca | |
| acggcctgaccggcaccggtgttctgacggaaagcaacaagaagttcctgcctttccagcaatttggcag | |
| agacatcgccgatacaaccgacgccgtgcgggacccccagaccctggaaatcctggacattaccccttgt | |
| tctttcggcggcgtgagcgtgatc | |

This example serves as one of many emerging demonstrations of the use of bioinformatics tools to design multi-epitope vaccines, including the use of artificial intelligence approaches (Yazdani et al. (2020) Infection and Drug Resistance, 13 3007-3022; Dong et al. (2020) Front. Immunol. 11: 1784; Kathwate (2022) Int. J. Peptide Res. & Therapeutics 28: 37). However, it is believed that none of these technologies have been previously linked to the ability to perform multi-epitope delivery by a mRNA design.

The foregoing example of a type II vaccine is illustrative and non-limiting. Using the teachings provided herein numerous type III vaccines incorporating the epitopes identified above and other epitopes will be available to one of skill in the art.

While the foregoing constructs are described with reference to mRNA(s). It will be recognized that in certain embodiments, DNA constructs can be provided that encode the mRNAs discussed above, and cellular transcription machinery can be exploited to transcribe the desired mRNA(s).

Nanoparticles for Carrying Nucleic Acid(s) that Encode for One or More Peptide Epitope(s)

In certain embodiments, nanoparticles for the delivery of one or more nucleic acid(s) (e.g., DNA or mRNA) encoding multiple peptide epitopes as described herein are provided. In certain embodiments the nanoparticles comprise one or more lipid(s) and/or one or more biocompatible polymer(s). In certain embodiments the nanoparticles can exclude biocompatible polymers and thereby provide nanoparticles comprising substantially or all lipids. Such nanoparticle may be designated lipid (or lipidic, or lipidoic) nanoparticles (LNPs). In certain embodiments the nanoparticles comprise one or more biocompatible polymers and optionally one or more lipids (e.g., cationic lipids) to, e.g., facilitate nucleic acid encapsulation. In certain embodiments the nanoparticles comprise a single nucleic acid sequence that encodes multiple epitopes. In certain embodiments the nanoparticle comprises a plurality of nucleic acid sequences that individually encode a plurality of epitopes described herein, or that in combination encode a plurality of epitopes described herein.

### Lipidic Nanoparticles (LNPs).

Lipid nanoparticles (LNPs) have emerged as the most promising non-viral delivery vehicle for exogenous mRNA in therapeutic treatments. The LNP is a complex nano-structured entity that serves to protect the delicate RNA molecule from the harshly degrading nuclease environment in vivo while facilitating intracellular delivery (see, e.g., Thomas et al. (2021) N. Engl. J. Med. doi: 10.1056/NEJMoa2110345).

In particular, lipid-based nucleic acid (e.g., mRNA) nanoparticles comprising ionizable lipids are gaining increasing attention as versatile technologies for delivering nucleic acids for a variety of applications. Such lipid-based nanoparticles can be fine-tuned towards a given use and have proven potential for successful development up to clinical practice (see, e.g., Dammes & Peer (2020) Trends in Pharmacological Sci., 41: 665-775; Uebbing et al. (2020) Langmuir, 36: 13331-13341; Siewert et al. (2020) Cells, 9: 2034; Kranz et al. (2016) Nature, 534: 396). In certain illustrative, but non-limiting embodiments, the LNPs comprise several lipid components, including, for example, an ionizable lipid that plays a central role in mRNA delivery efficacy, helper lipids (distearoylphosphatidylcholine, DSPC, and cholesterol), and a poly(ethylene glycol) (PEG) lipid.

It has been demonstrated that the transfection efficacy of RNA lipid nanoparticles depends on the pKa value of the ionizable lipid used for particle formation (see, e.g., Uebbing et al. (2020) Langmuir, 36: 13331-13341; Kauffman et al. (2015) Nano Lett. 15: 7300-7306). More specifically, the cationic charge that these particles develop under acidic pH conditions is important for endosomal processing and mRNA release (Nel & Miller (2021) ACS Nano 15: 5793-5818). In addition to pKa, the fusogenicity of different lipids, which results from their ability to form the reversed hexagonal phase H_{II}, has an influence in facilitating endosomal escape (see, e.g., Cullis et al. (2-017) Mol. Ther. 25: 1467-1475; Heyes et al. (2005) J. Control Release, 107: 276-287). Both of these lipid properties, which are a result of the headgroup structure and lipid tail saturation, are important factors to be kept in mind when choosing lipids for mRNA lipoplex formulation.

Due to the biocompatible and nontoxic characteristics of lipid-based delivery systems, they have shown the most advances for delivery of nucleic acids (e.g., gene therapy) in human clinical trials. Recently, the development of siRNA lipid nanoparticles (LNPs) has experienced a significant breakthrough, which is reflected by the FDA approval of ONPATTRO®, the first LNP delivering siRNA for treating a liver-based rare genetic disease, TTR-mediated amyloidosis.

The composition of a lipid nanoparticles (LNP) plays a major role in particle size, surface properties, encapsulation efficiency and intracellular release. Additionally, it influences the uptake of the LNP cargo to the target cell and eventually a specific cellular compartment.

Among these, lipid-based mRNA nanoparticles comprising ionizable lipids are gaining increased attention as versatile technologies for fine-tuning towards a given application, with proven potential for successful development up to clinical practice (see, e.g., Dammes & Peer (2020) Trends in Pharmacological Sci. 41: 665-775; Uebbing et al. (2020) Langmuir, 36: 13331-13341; Siewert et al. (2020) Cells, 9: 2034; Kranz et al. (2016) Nature, 534: 396). It has now been demonstrated that the transfection efficacy of RNA lipid nanoparticles depends on the pKa value of the ionizable lipid used for particle formation (see, e.g., Uebbing et al. (2020) Langmuir, 36: 13331-13341; Kauffman et al. (2015) Nano Lett. 15: 7300-7306). More specifically, the cationic charge that these particles develop under acidic pH conditions is important for endosomal processing and mRNA release (Nel & Miller (2021) ACS Nano 15: 5793-5818). In addition to pKa, the fusogenicity of different lipids, which results from their ability to form a reverse hexagonal phase H_{II}, has had an influence in facilitating endosomal escape (see, e.g., Cullis et al. (2-017) Mol. Ther. 25: 1467-1475; Heyes et al. (2005) J. Control Release, 107: 276-287). Both of these lipid properties, which are a result of the headgroup structure and lipid tail saturation, are important factors to be kept in mind when choosing lipids for mRNA lipoplex formulation.

As noted above, in certain illustrative, but non-limiting embodiments LNP formulations for delivery of nucleic acids are comprised of an ionizable amino lipid, helper lipids, PEG-lipid and cholesterol. The most important component is often the ionizable cationic lipid, e.g., often with a pKa ˜6.5. When formulating nucleic acids with lipids into the LNPs in an acidic condition (e.g., pH ˜4), the ionizable lipid is positively charged to interact with the negatively charged nucleic acids to promote loading into the LNPs. The pH of the formed LNPs can then be brought up to, e.g., pH 7.4, and in normal physiological conditions, the LNPs become neutral for reduced non-specific interaction with cells and blood components. When the LNPs are internalized by the target cell and enter into the acidic endo/lysosomal compartment (e.g., pH ˜5), the lipid reverts to a positive charge, mediating binding to the negatively charged endolysosomal membrane (Maier et al. (2013) Mol. Ther. 21: 1570-1578). This interaction leads to endolysosomal disruption and cytosolic release of the nucleic acid.

The incorporation of an ionizable aminolipid to deliver nucleic acids was first reported with 1,2-dioleoyl-3-dimethylaminopropane (DODAP) LNPs (Maurer et al. (2001) Biophys. J. 80: 2310-2326). This formulation composed of cholesterol, distearoyl phosphatidylcholine (DSPC), PEG-Lipid and DODAP (pKa 6.6), encapsulated up to 70% of the oligonucleotide into 100 nm-sized LNPs (Semple et al. (2001) Bba-Biomembranes, 1510: 152-166).

The lipid structure and pKa of the ionizable lipids were demonstrated to have a significant impact on the efficient delivery of siRNA to the target cells (Semple et al. (2010) Nat. Biotechnol. 28: 172-176). Semple et al. (Id.) evaluated the effect of ester-, alkoxy-, and ketal-linkers, between the head group and alkyl chain of 1,2-dilinoleoyl-3-dimethylaminopropane, 1,2-dilinoleyloxy-3-dimethyl aminopropane (DLin-DMA), and 2,2-dilinoleyl-4-dimethylaminomethyl-[1,3]-dioxolane (DLin-K-DMA), respectively. The observed potency of DLin-K-DMA lipid on the in vivo silencing of factor VII was 2.5-fold higher than the other tested lipids, indicating the superior effect of the ketal linker (Jayaraman et al. (2012) Angew. Chem. Int. Ed. Eng. 51: 8529-8533). A further modification to the ketal linker by incorporating a single methylene group (2,2-dilinoleyl-4-(2-dimethylaminoethyl)-[1,3]-dioxolane, DLin-KC2-DMA) has shown a 4-fold higher activity over DLin-K-DMA. Additionally, the pKa of the ionizable lipid was found to be a key factor for the potency of siRNA-LNPs. Ultimately, the most potent LNP that is used in the ONPATTRO® formulation is based on (6Z,9Z,28Z,31Z)-heptatriaconta-6,9,28,31-tetraen-19-yl-4-(dimethylamino) butanoate (DLin-MC3-DMA) lipid with a pKa of 6.44. This lipid showed 10-fold higher potency than DLin-KC2-DMA (Jayaraman et al. (2012) Angew. Chem. Int. Ed. Eng. 51: 8529-8533; Lin et al. (2014) Clin. Lipidol. 9: 317-331). There have been other reports focusing on developing new ionizable lipids to improve nucleic acid delivery. For instance, multiple ionizable groups were introduced in these novel ionizable lipids to enhance the gene delivery efficiency including dicetylphosphate-tetraethylenepentaamine-based polycation liposomes for systemic siRNA delivery (Asai et al. (2100) Bioconjug. Chem. 22: 429-435) and alkenyl amino alcohols LNPs for mRNA delivery (Fenton et al. (2016) Adv. Mater. 28: 2939-2943). Additionally, chemical modification of ionizable lipids such as biodegradable linkers can also improve the overall gene delivery (Fenton et al. (2017) Adv. Mater. 29(33). doi: 10.1002/adma.201606944).

In addition to an ionizable lipid, in various embodiments helper lipids can be incorporated into the LNPs to improve their stability. 1,2-dioleoyl-sn-glycero-3-phosphoethanolamine (DOPE) was one of the first phospholipids used to promote membrane fusion. Due to the unsaturated acyl chains and small head group, DOPE can adapt the hexagonal phase and interact with the endosomal membrane. This interaction facilitates the release of payload to the cytosol by disrupting the endosomal bilayer (Hafez et al. (2001) Adv. Drug Deliv. Rev. 47: 139-148). DSPC is regarded as the most efficient helper lipid in the LNP formulation for siRNA delivery. In contrast to DOPE, DSPC has two saturated acyl chains and a large head group which results in a cylindrical geometry that plays a major role in maintaining the stability and structure of the LNPs outer layer (Kulkarni et al. (2018) ACS Nano, 12: 4787-4795). Another major component of the LNPs is cholesterol that influences membrane permeability and fluidity. Incorporation of cholesterol in the LNP formulation can result in tight packaging, leading to enhanced stability (Coderch et al. (2000) J. Control. Release, 68: 85-95).

In certain embodiments a PEG-lipid is included in the LNP formulation to reduce aggregation during the particle formation, to prolong the storage stability (Bao et al. (2013) Pharm. Res. 30: 342-351) and can be important for the effective delivery of nucleic acid into target cells. Mui et al. (2013) Mol. Ther. 2: e139, studied the effect of the PEG-lipid acyl chain length on the in vivo pharmacokinetics and delivery efficiency of siRNA. The short acyl chain PEG-C14 was found to dissociate rapidly at a rate of 45% per hour, whereas the longer chain PEG-lipids, PEG-C16 and PEG-C18 diffused out of the LNPs at slower rates of 1.3 and 0.2% per hour, respectively.

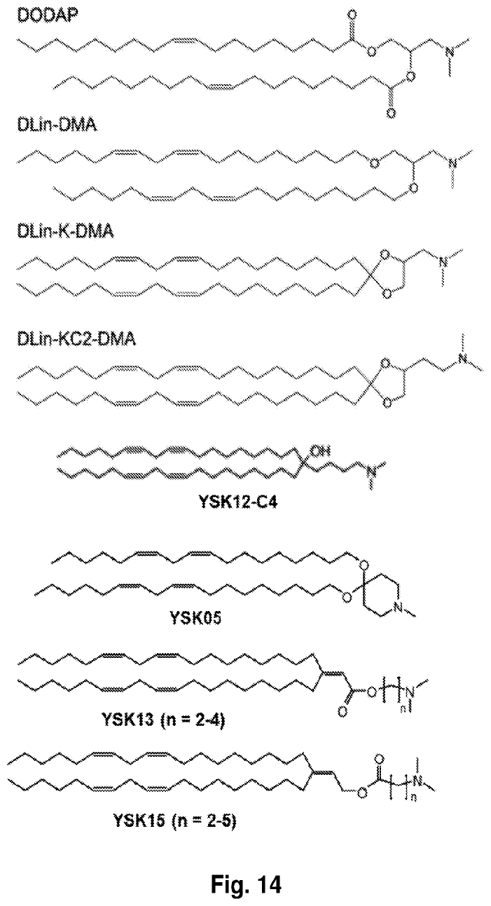

ONPATTRO® (Alnylam), for treating TTR-mediated amyloidosis, is the most clinically advanced siRNA-LNP product. Mutation in the TTR gene results in a build-up of amyloidosis protein in tissues and organs that can eventually cause cardiomyopathy or neuropathy. Alnylam has produced two generations of TTR-LNPs which involve the incorporation of different ionizable lipids. The formulation consisting of DSPC, cholesterol, PEG-1,2-Dimyristoyl-sn-glycerol (DMG) and either DLin-DMA (see, e.g., FIG. 14) in the first generation (ALN-TTR01) or DLin-MC3-DMA (see, e.g., FIG. 14) in the second generation (ALN-TTR02) (Wan et al. (2014) Drug Deliv. Transl. Res. 4: 74-83). ALN-TTR02 showed improved efficacy in human patients, inducing up to 70% suppression of TTR at a dose of 0.15-0.5 mg/kg (Lin et al. (2014) Clin. Lipidol. 9: 317-331; Coelho et al. (2013) N. Engl. J. Med. 369: 819-829) (see, e.g., Lin et al. (2014) Clin. Lipidol. 9: 317-331; Coelho et al. (2013) N. Engl. J. Med. 369: 819-829). In August 2018, ALN-TTR02 known as ONPATTRO® was approved by the FDA as a treatment for both mutant and non-mutant forms of TTR (Coelho et al. (2013) N. Engl. J. Med. 369: 819-829; Garber (2018) Nat. Biotechnol. 36: 777-778). ONPATTRO® is also the first approved siRNA therapeutics and the first approved non-viral gene delivery system.

ALN-PCS is the latest development of siRNA-LNPs by Alnylam and has reached clinical trials in 2011. This formulation was developed to treat hypercholesterolemia by targeting proprotein convertase subtilisin/kexin type 9 (PCSK9). The ALN-PCS01 formulation is composed of lipidoid 98N12-5(1)4HCl, cholesterol, and mPEG2000-DMG at a molar ratio of 42:48:10. The phase I trial reported a dose-dependent silencing effect of PCSK9 with reductions of 84% PCSK9 protein and 50% LDL-cholesterol at the highest dose of 0.4 mg/kg (Id.). In the ALN-PCS02 formulation, lipidoid was replaced with DLin-MC3-DMA and an increase in potency in a phase I trial was reported with a 50% reduction of LDL-cholesterol at a low dose of 0.25 mg/kg (Alabi et al. (2012) Curr. Opin. Pharmacol. 12: 427-433).

Sato et al. (2012) J. Control. Release, 163: 267-276, developed a multifunctional envelope-type nanodevice (MEND) composed of a novel pH-sensitive cationic lipid for siRNA delivery. At first, MEND was incorporated into a peptide (PPD, GALA and shGALA)-based functionalizing device, which was later replaced with an ionizable lipid for improved gene silencing activity. YSK05 (see, e.g., FIG. 14) is one of their earlier synthesized ionizable lipids with one tertiary amine and two unsaturated acyl chains. This discovery was followed by the synthesis of various pH-sensitive lipids that showed liver-selective accumulation and efficient cytosolic delivery. MENDs were initially prepared using an ionizable lipid, helper lipid (DSPC, DOPE, 1,2-dioleoyl-sn-glycero-3-phosphocholine or 1-Palmitoyl-2-oleoyl-sn-glycero-3-phosphoethanolamine), cholesterol and PEG-DMG at a molar ratio of 50:10:40:3, and then optimized to a formulation of ionizable lipid:cholesterol:PEG-DMG at 70:30:3 (Sato et al. (2012) J. Control. Release, 163: 267-276; Watanabe et al. (2014) Sci. Rep. 4: 4750). The authors reported that the optimized formulation with no helper lipid and a higher ratio of the ionizable lipid resulted in enhanced endosomal escape due to an enhanced fusogenic property, and subsequently higher transfection efficiency (Hatakeyama et al. (2014) J. Control. Release, 173: 43-50).

Sato et al. (2016) Mol. Ther. 24: 788-795 investigated the effect of the lipid pKa on the NLP performance. YSK05 and YSK13-C3 with pKa of 6.50 and 6.45, respectively, have shown significant delivery to target cells, and the YSK-13-C3 formulation showed four-fold higher gene silencing activity compared to YSK05 (Watanabe et al. (2014) Sci. Rep. 4: 4750; Hatakeyama et al. (2014) J. Control. Release, 173: 43-50; Yamamoto et al. (2016) J. Hepatol. 64: 547-555). Lipids with a higher pKa (YSK13-C4 and YSK15-C4 with pKa 6.80 and 7.10, respectively) have also been tested. Lipase-resistant YSK05 (pKa 6.50) and YSK012-C4 (pKa 8.0) have been used to prepare nanoparticles with a pKa of 7.15 (Shobaki et al. (2018) Int. J. Nanomedicine, 13: 8395-8410; Warashina et al. (2016) J. Control. Release, 225: 183-191).

The same group has also developed an ionizable SS-cleavable and pH-activated lipid-like material (ssPalmM) that contains a cleavable di-sulfide bond for liver-targeted delivery (Ukawa et al. (2014) Adv. Healthc. Mater. 3: 1222-1229). This synthesized com-pound contains two myristoyl lipid chains and two ionizable tertiary amines linked through a disulfide bond. The ionizable amino groups are to promote nucleic acid loading and endosomal disruption and the disulfide bond is cleaved in the intracellular environment, which is reductive and GSH-rich, to facilitate nucleic acid release in the cytosol. Ukawa et al. (2014) Adv. Healthc. Mater. 3: 1222-1229, reported a lipid envelope-type nanoparticle encapsulating pDNA for liver delivery, composed of ssPalmM, 1-octadecanoyl-2-(9Z-octadecenoyl)-sn-glycero-3-phosphocholine, cholesterol and distearoyl-rac-glycerol-PEG. Akita et al. (2015) ACS Biomater. Sci. Eng. 1: 834-844, replaced the two myristoyl lipid chains in the ssPalmM with vitamin E (ssPalmE) and demonstrated improved gene silencing activity in mice by approximately 6-fold. The group reported that the incorporation of a highly fat-soluble vitamin E as a hydrophobic scaffold enhanced the hepatic delivery of siRNA.

To date, we have prepared mRNA encapsulating nanoparticles by using lipid film, ethanol injection, and microfluidic mixing methods, as described in protocols developed for the NanoAssemblr® Benchtop. For the lipid film method, we dissolved the lipids in required ratios in chloroform, followed by pipetting into glass vials. A thin film forms when the chloroform is evaporated in a rotary evaporator. The nucleic acid (e.g., mRNA) is diluted to the required concentration in 10 mM HEPES/EDTA buffer and then added to the lipid films. After vortexing the samples are left at room temperature overnight, before further vortexing and collecting the particles. For lipoplexes, we use an ethanol injection method. The lipids are dissolved in the required ratios in pure ethanol, and then injected into a glass vial containing mRNA diluted to the appropriate concentrations, using a handheld mixer. Microfluidic preparation is carried out in NanoAssemblr® Benchtop, using the mRNA Lipid Nanoparticles Robust low-volume production protocol from the manufacturer (Precision Nanosystems).

In one illustrative, but non-limiting embodiment, by using a microfluidic apparatus (NanoAssemblr) developed by Precision NanoSystems Inc., we have synthesized mRNA delivering nanoparticles, including GFP-mRNA, which was used for a proof-of-principle purposes. GFP-mRNA encapsulating LNPs (GFP-mRNA LNPs) were developed in our laboratory through the mixing of the ionizable cationic lipid species, DLin-MC3-DMA, with DSPC, cholesterol and PEG2k-DMG, which are dissolved in ethanol at the molar ratios of 50, 10, 38.5, 1.5, while the mRNA (GFP-mRNA) is diluted in RNase free sodium acetate buffer (pH 4.0). Poly A encapsulated LNPs (Poly A LNPs) were synthesized in the illustrative example shown below as a negative control for GFP expression. The aqueous and ethanol solution phases were mixed in a 3:1 volume ratio in the NanoAssemblr at a mixing rate of 12 mL/min. After synthesis, LNPs were purified by filtration of the 40×diluted sample in sterile Ca2+- and Mg2+-free PBS using an Amicon® Ultra-15 centrifugal filtration tube at 2000×g for 30 minutes at 20° C. Table 40, illustrates various physical properties of the lipidic nanoparticles.

Table 40 shows the hydrodynamic size, PDI, Zeta potential and encapsulation efficiency of illustrative mRNA-LNPs.

| | GFP mRNA-LNP | Poly A-LNP |
|---|---|---|
| Size (nm) in PBS | 137.5 ± 2.1 | 112.5 ± 0.9 |
| PDI | 0.113 ± 0.054 | 0.120 ± 0.028 |
| ζ-potential (mV) | −5.54 ± 4.19 | −11.43 ± 3.94 |
| Encapsulation Efficiency | 92.0 ± 0.06 | 96.6 ± 0.05 |
| (%) | | |

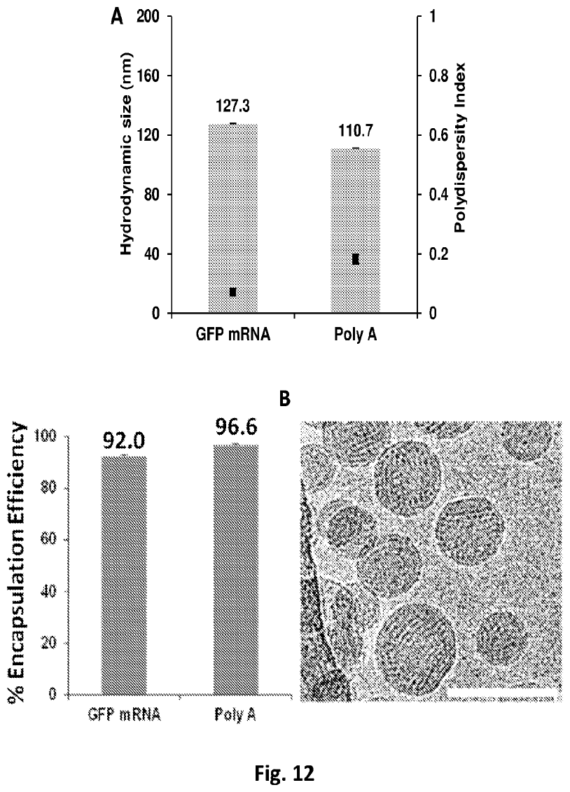

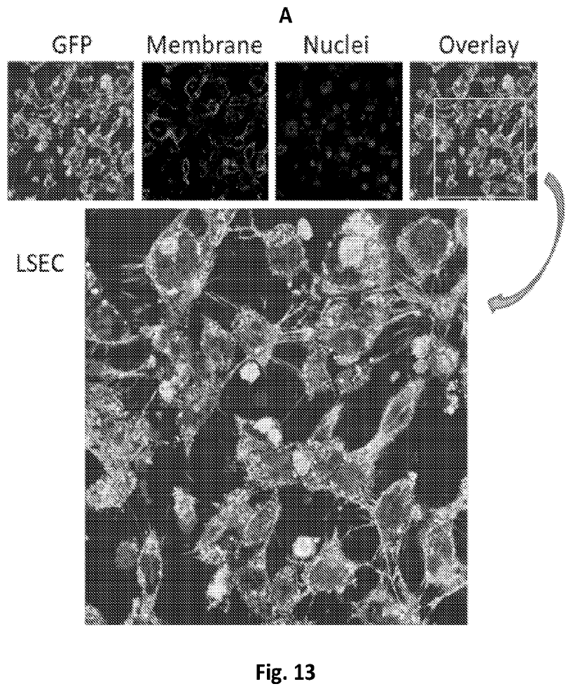

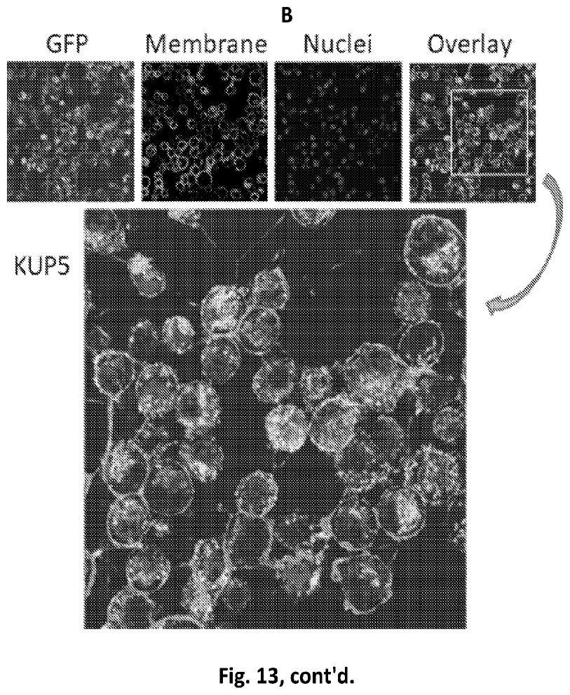

The size of LNPs was determined by DLS measurements, using a Zetasizer Nano ZS from Malvern Instruments Ltd. The encapsulation efficiency (EE %) of mRNA were determined using the RIBOGREEN® assay. RIBOGREEN® is a dye that exhibits fluorescence when bound to single stranded mRNA but cannot enter the LNPs. The encapsulation efficiency (EE %) of GFP-mRNA LNPs and Poly A LNPs was 92.0 and 96.6%, respectively. This serves to demonstrate that GFP mRNA or Poly A is located inside the LNPs. Following it synthesis, the LNPs samples were imaged by cryogenic electron microscopy (Cryo-EM) to investigate its nano-precipitated core structure. These cryo-EM images clearly demonstrate the encapsulation of mRNA in what appears to be a LNP, exhibiting a multilayered core structure (FIG. 12). Taken together, the encapsulation efficiency (EE %) and cryo-TEM evidence show that the mRNA-LNPs form nanoparticles with encapsulated mRNA that is protected from the external medium. Subsequent confocal imaging was performed by incubating the particles with liver sinusoidal endothelial cells (LSECs) and Kupffer (Kup5) cells (FIG. 13). Confocal imaging showed the expression of GFP in both cell types. No fluorescent imaging was obtained with polyA nanoparticles (not shown). We have also demonstrated that intravenous administration of the GFP mRNA leads to effective expression of a fluorescent protein in the liver and lung of mice that received intravenous injection of the nanoparticles. Approximately 60% of the particles were delivered in the liver, 25% in the lung and the rest localizing in the spleen and other sites.

### Nanoparticle Comprising Biocompatible Polymers.

In addition to the use of LNPs, it is also known that mRNA can be delivered by cationic polymers, which, in certain embodiments, can also be combined with cationic lipids for mRNA nanoparticle construction (see, e.g., Siewert et al. (2020) Cells, 9: 2034). Methods of making polymeric nanoparticles are well known to those of skill in the art (see, e.g., Marin et al. (2013) Int. J. Nanomed., 8: 3071-3091, and references therein), and can be adapted to deliver DNA or mRNA nucleic acid constructs. In various embodiments any of a number of biocompatible polymers can be used to form the immunogenic nanoparticles described herein. Such biocompatible polymers are well known to those of skill in the art and include but are not limited to polyesters (e.g., poly(lactic-co-glycolic acid), poly(glycolic acid), poly(lactic acid), poly(caprolactone), poly(butylene succinate), poly(trimethylene carbonate), poly(p-dioxanone), poly(butylene terephthalate), and the like), poly(ester amide)s (PEA) (see, e.g., Guerrero et al. (2015) J. Control Release., 211: 105-117), polyurethanes and polyurethane copolymers (see, e.g., Cherng et al. (2013) Int. J. Pharmaceutics, 450(1-2): 145-162), poly-anhydrides (e.g., poly [bis(p-carboxyphenoxy)methane], poly[bis(hydroxyethyl) terephthalate-ethylortho-phosphorylate/terephthaloyl chloride], see, e.g., Chang et al. (1983) Biomaterials, 4(2): 131-133), poly(ortho esters) (see, e.g., Nair et al. (2006) Adv. Biochem. Eng. Biotechnol. 102: 47-90; Park et al. (2005) Molecules, 10: 146-161), polyphosphoesters (e.g., polyphosphoesters poly[bis(hydroxyethyl) terephthalate-ethyl orthophosphorylate/terephthaloyl chloride]), poly(alkyl cyanoacrylates) (e.g., poly(butyl cyanoacrylate), poly(β-hydroxyalkanoate)s, poly(hydroxybutyrate), poly(hydroxybutyrate-co-hydroxyvalerate, collagen, albumin, gluten, chitosan, hyaluronate, cellulose, alignate, and starch.

In certain embodiments the biocompatible polymer comprises one or more cationic polymers. It is noted that such cationic polymers are particularly well suited for delivery of a nucleic acid (e.g., DNA or mRNA). Illustrative cationic polymers include, but are not limited to poly-L-lysine (PLL), poly-ethylenimine (PEI; a branched cationic polymer, assisting in endosomal delivery), poly[(2-dimethylamino) ethyl methacrylate](pDMAEMA), polyamidoamine (PAMAM) dendrimers, biodegradable poly(β-amino ester) (PBAE) polymers, poly(amino-co-ester) (PACE)-based polymers (branched amino acids, assisting in endosomal delivery), and the like (see, e.g., Wahane et al. (2020) Molecules, 25: 2866).

In certain embodiments the biocompatible polymer comprises one or more polymers selected from the group consisting of poly(lactic-co-glycolic acid) (PLGA), poly(glycolic acid) (PGA), poly(lactic acid) (PLA), poly(caprolactone) (PCL), poly(butylene succinate), poly(trimethylene carbonate), poly(p-dioxanone), poly(butylene terephthalate), poly(ester amide) (HYBRANE®), polyurethane, poly[(carboxyphenoxy) propane-sebacic acid], poly[bis(hydroxyethyl) terephthalate-ethyl orthophosphorylate/terephthaloyl chloride], poly(β-hydroxyalkanoate), poly(hydroxybutyrate), and poly(hydroxybutyrate-co-hydroxyvalerate). In certain embodiments the biocompatible polymer comprises poly(lactic-co-glycolic acid) (PLGA).

In certain embodiments the nanoparticle may be entirely composed of a biocompatible polymer (e.g., PLGA), while in other embodiments, the nanoparticle may comprise one or more lipids, including cationic lipids. We have achieved success with the method of Siewert et al. (2020) Cells, 9: 2034), who demonstrated that the combination of two complexing ingredients, the cationic lipid, dioleoyl-3-trimethylammonium propane (DOTAP), and at the cationic polymer, protamine, could result in improved mRNA transfection in comparison to single complexing agents. In certain embodiments, the lipid(s) that can be included to bind to the nucleic acid, includes (but are not limited to) didodecyl-dimethylammonium bromide (DDAB), and 1,2-dioleoyloxy-3-trimethylammonium propane chloride (DOTAP). DOTAP is cationic lipid that can bind mRNA and nuclei acids, in addition to delivering danger signals. Other suitable lipids include DOTMA (N-[1-(2,3-dioleyloxy)propyl]-N,N,N-trimethylammonium chloride), 1,2-dilinoleyloxy-3-dimethylaminopropane (DLinDMA), dilinoleylmethyl-4 dimethyl aminobutyrate (DLin-MC3-DMA), C12-200 (Lipid 5), 3-(dimethylamino) propyl(12Z,15Z)-3-[(9Z,12Z)-octadeca-9,12-dien-1-yl]henicosa-12,15-dienoate (DMAP-BLP), (2Z)-non-2-en-1-yl 10-[(Z)-(1-methylpiperidin-4 yl)carbonyloxy]nonadecanoate (L101), and the like. In certain embodiments, inclusion of lipid can also be used to fine-tune the particle size and/or charge.

In certain embodiments the lipid comprises up to 25%, [molar percentage (excluding nucleic acid(s)) of the nanoparticle. In certain embodiments the lipid comprises about 1%, or about 2%, or about 3%, or about 4%, or about 5%, or about 6%, or about 7% up to about 25%, or up to about 20%, or up to about 15%, or up to about 10% (molar percentage (excluding nucleic acid(s)) of the nanoparticle.

Methods of making polymeric nanoparticles are well known to those of skill in the art (see, e.g., Marin et al. (2013) Int. J. Nanomed., 8: 3071-3091, and references therein).

In certain embodiments the nucleic acid(s) are encapsulated within the nanoparticle, and this can readily be accomplished by combining the nucleic acid(s) with the biocompatible polymer during nanoparticle synthesis. In certain embodiments the nucleic acid(s) are attached to the surface of the nanoparticle, e.g., by adsorption or by coupling directly or using a linker.

### Adjuvants.

It has been demonstrated that adjuvant-pulsed mRNA vaccine nanoparticles (NPs) comprise an mRNA and a palmitic acid-modified TLR7/8 agonist R848 (C16-R848), coated with a lipid-polyethylene glycol (lipid-PEG) shell retain the adjuvant activity of encapsulated C16-R848 and markedly improved the transfection efficacy of the mRNA (>95%) and subsequent MHC class I presentation (see, e.g., Islam et al. (2021) Biomaterials, 266: 120431). The C16-R848 adjuvant-pulsed mRNA vaccine NP approach induced an effective adaptive immune response by significantly improving the expansion of mRNA encoded antigen-specific CD8+ T cells relative to the mRNA vaccine NP without adjuvant.

Accordingly, in various embodiments, the immunogenic nanoparticles described herein may contain one or more adjuvants (e.g., a TLR7/8 agonist such as R848 (C16-R848), a lipid-based adjuvant, and the like.

Typically, the adjuvant(s) are added in order to enhance the immunostimulatory properties of the immunogenic nanoparticles. In this context, an adjuvant may be understood as any compound, that is suitable for incorporation in the immunogenic nanoparticles described herein that, without being bound to a particular theory, initiates, and/or increases an innate immune response (e.g., a viral protein and/or protein fragment) as defined herein. Such innate immune responses strongly influence the adaptive immune response.

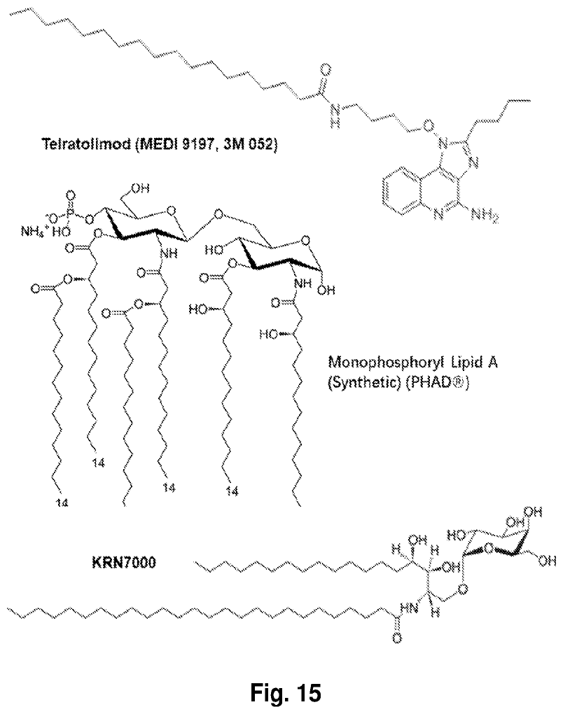

In certain embodiments lipid-based adjuvants are readily incorporated into the immunogenic nanoparticles described herein. In our own nanoparticle vaccination studies, we have recently used Telratolimod (MEDI 9197, 3M 052) for incorporation into lipid-based nanoparticles, including into a liposome. Telratolimod is a novel, injectable, tissue retained TLR7/8 agonist that forms a tissue depot with gradual, sustained release, allowing local TLR triggering activity without systemic cytokine release (see, e.g., FIG. 15). This agonist induces innate immune activation as well as systemic, antigen-specific CD8+ T cell responses

Other lipid-based agonists are known to those of skill in the art. For example, another lipid-based TLR agonist for incorporation into lipid nanoparticles is Monophosphoryl Lipid A (Synthetic) (PHAD®). PHAD® is a synthetic structural analog of monophosphoryl Lipid A (MPLA) (see, e.g., FIG. 15) that has been shown to boost the immune system through activation of the toll-like receptor 4 (TLR4) resulting in production of proinflammatory cytokines and antigen-specific effector CD4+ and memory CD8+ T cells. Also referred to as GLA, this adjuvant has been administered to well over 1000 human subjects without serious adverse events. PHAD® is available in bulk quantities for vaccine development and commercial manufacturing. Still another illustrative and non-limiting lipid adjuvant is KRN7000 (see, e.g., FIG. 15). KRN7000 is a synthetic analog of α-galactosylceramide and the marine natural product agelasphin. This lipid-based argument is a specific ligand for human and mouse NKT cells and remains the best studied ligand of the lipid-binding MHC class I-like protein CD1d. KRN7000 has been incorporated into lipid-based nanoparticles and display potent antitumor activity in various in vivo models (see, e.g., Nakagawa et al. (1998) Cancer Res. 58: 1202-1207). Other lipid-based agonists are known to those of skill in the art and, in view of the teachings provided herein are readily incorporated into the immunogenic nanoparticles described herein.

In certain embodiments the adjuvant is an adjuvant that elicits a TH1-biased immune response. Without being bound to a particular theory, in certain embodiments, the use of TH1 preferential adjuvants may prevent vaccine-related immunopathology. Such pathology has appeared in previous treatment attempts with SARS-CoV-1, and is thought to originate, inter alia, from the use of TH2-promoting adjuvants. It is noted that in various embodiments, TH1-biased adjuvants can be used for numerous other anti-viral immunogenic nanoparticles.

Without being bound to a particular theory, it is believed the TH1 adjuvant, e.g., beta-inulin or STING, is able to allow the development of a balanced immune response, that reduces the deleterious effects of TH2 skewing. TH2 skewing can lead to eosinophilic lung damage and a type of antibody (IgG1) that enhances rather than neutralizes the viral effect.

In this regard it has been shown that when compared in a murine model a range of recombinant spike protein or inactivated whole-virus vaccine candidates alone or adjuvanted with either alum, CpG, or Advax (a delta inulin-based polysaccharide adjuvant), all vaccines protected against lethal infection. The addition of an adjuvant significantly increased serum neutralizing-antibody titers and reduced lung virus titers on day 3 post-challenge (Okubo et al. (2015) J. Virol., 89(6): 2995-3007). Unadjuvanted or alum-formulated vaccines were associated with significantly increased lung eosinophilic immunopathology on day 6 post-challenge. This was not observed in mice immunized with vaccines formulated with delta inulin adjuvant. Protection against eosinophilic immunopathology by vaccines containing delta inulin adjuvants correlated better with enhanced T-cell gamma interferon (IFN-γ) recall responses rather than reduced interleukin-4 (IL-4) responses, suggesting that immunopathology predominantly reflects an inadequate vaccine-induced TH1 response (Id.).

In view of these and other observations, it has been proposed that immunization with SARS antigens alone or formulated with alum fails to induce a sufficient number of IFN-γ secreting memory T cells and this lack of IFN-γ is further exacerbated by active TH1 pathway downregulation by the SARS-CoV itself. This enables a cycle of ever-increasing TH2-polarization of the anti-coronavirus response to become established. In view of this it is proposed that the ideal coronavirus vaccine needs to induce not only neutralizing antibodies or, at a minimum, memory B cells capable of rapidly producing neutralizing antibody upon virus exposure but also a robust long-lived T-cell IFN-γ response, thereby preventing any risk of lung eosinophilic immunopathology (Id.). It is believed this is the vaccine function that can be imparted by formulation of the antigen(s) with a TH1-biased adjuvant (e.g., delta inulin, STING, and the like).

Accordingly, as noted above, in various embodiments the immunogenic nanoparticles described herein comprise one or more TH1-biased adjuvants. TH1-biased (TH1-preferential) adjuvants are well known to those of skill in the art. Illustrative TH1-biased adjuvants include, but are not limited to: a combined aluminum salt and TLR4 agonist, rOv-ASP-1 (recombinant Onchocerca volvulus activation associated protein-1 (see, e.g., He et al. (2009) J. Immunol., 182(7): 4005-4016), IC31@(a two-component adjuvant consisting of the artificial antimicrobial cationic peptide KLK acting as a vehicle and the TLR9-stimulatory oligodeoxynucleotide ODN1 (see, e.g., Schellack et al. (2006) Vaccine, 24: 5461-5472), SPO1 (Yu et al. (2012) Vaccine, 30(36): 5425-5436), CPG oligonucleotide, alum-TLR7 agonist based on a TLR7 agonist (SMIP7.10), selected from a benzonaphthyridines series of TLR7 agonists, adsorbed to Aluminium Hydroxid TLR7 (see, e.g., Buonsanti et al. (2016) Scientific Reports, 6: Article number: 29063), OprI lipoprotein of Pseudomonas aeruginosa (see, e.g., U.S. Patent Pub. No: 2003/0059439 A1), cathelicidin-derived antimicrobial peptides (see, e.g., PCT Patent Pub. No: WO 02/013857), delta inulin (β-D-[2-1]poly(fructo-furanosyl)α-D-glucose), e.g., Advax-1 and Advax-2, and STING agonists.

In certain embodiments the TH1-promoting adjuvant comprises one or more Stimulator of Interferon Genes (STING) agonists. STING agonists are well known to those of skill and include, but are not limited to amidobenzimidazole (diABZI), 3′,5′-Cyclic diadenylic acid sodium salt (c-DI-AMP sodium salt), 3′,5′-Cyclic diguanylic acid sodium salt (c-Di-GMP sodium salt), 2′,3′-Cyclic guanosine monophosphate-adenosine monophosphate sodium salt (2′,3′-cGAMP), 3′,3′-Cyclic guanosine monophosphate-adenosine monophosphate sodium salt (3′,3′-cGAMP), 5,6-Dimethyl-9-oxo-9H-xanthene-4-acetic acid (DMXAA), (DMXAA), CMA, MK-1454, CRD5500, cyclic di-nucleotide compounds as described in U.S. Patent Publication No: 2020/0062798 A1, which is incorporated herein by reference for the STING agonists described therein, tricyclic heteroaryl compounds as described in U.S. Patent Publication No: 2020/0040009 A1, which is incorporated herein by reference for the STING agonists described therein, heteroaryl amide compounds as described in U.S. Patent Publication No: 2020/0039994 A1, which is incorporated herein by reference for the STING agonists described therein, and the like.

In certain embodiments, amidobenzimidazole (diABZI) is a preferred STING (stimulator of interferon genes) agonist for the immunogenic nanoparticles.

In certain embodiments the adjuvant comprises one or more TLR agonists. TLR agonists are well known to those of skill in the art.

TLRs are type-I transmembrane proteins that are responsible for initiation of innate immune responses in vertebrates. TLRs elicit overlapping yet distinct biological responses due to differences in cellular expression and in the signaling pathways that they initiate. Once engaged (e.g., by a natural stimulus or a synthetic TLR agonist) TLRs initiate a signal transduction cascade leading to activation of NF-κB via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-κB inhibitor I-κB. As a result, NF-κB enters the cell nucleus and initiates transcription of genes whose promoters contain NF-κB binding sites, such as cytokines. Additional modes of regulation for TLR signaling include TIR-domain containing adapter-inducing interferon-β (TRIF)-dependent induction of TRAF6 and activation of MyD88 independent pathways via TRIF and TRAF3, leading to the phosphorylation of interferon response factor three (IRF3). Similarly, the MyD88 dependent pathway also activates several IRF family members, including IRF5 and IRF7 whereas the TRIF dependent pathway also activates the NF-κB pathway.

Illustrative TLR agonists for use in the immunogenic nanoparticles described herein include, but are not limited to agonists of TLR1, TLR2, TLR3, TLR4, TLRS, TLR6, TLR7, TLR8, TLR9, TLR10, TLR11, or any combination thereof (e.g., TLR7/8 agonists).

Examples of TLR3 agonists include, but are not limited to Polyinosine-polycytidylic acid (poly (I:C)), Polyadenylic-polyuridylic acid (poly (A:U), and poly(I)-poly(C12U).

Examples of TLR4 agonists include but are not limited to Lipopolysaccharide (LPS) and Monophosphoryl lipid A (MPLA).

On example of a TLR8 agonist is Flagellin.

Examples of TLR9 agonists include single strand CpG oligodeoxynucleotides (CpG ODN). Three major classes of stimulatory CpG ODNs have been identified based on structural characteristics and activity on human peripheral blood mononuclear cells (PBMCs), in particular B cells and plasmacytoid dendritic cells (pDCs). These three classes are Class A (Type D), Class B (Type K), and Class C.

Examples of TLR7 agonists and TLR8 agonists are described, e.g., by Vacchelli, et al. (2013) Oncolmmunology, 2: 8, e25238, DOI: 10.4161/onci.25238 (2013)) and Carson et al. (U.S. Patent Application Publication 2013/0165455, which is hereby incorporated by reference in its entirety). Examples of TLR7, TLR8 or TLR7/8 agonists include but are not limited to Gardiquimod (1-(4-amino-2-ethylaminomethylimidazo[4,5-c]quinolin-1-yl)-2-methylpropan-2-ol), Imiquimod (R837) (agonist for TLR7), loxoribine (agonist for TLR7), IRM1 (1-(2-amino-2-methylpropyl)-2-(ethoxymethyl)-1H-imidazo-[4,5-c]quinolin-4-amine), IRM2 (2-methyl-1-[2-(3-pyridin-3-ylpropoxy)ethyl]-1H-imidazo[4,5-c]quinolin-4-amine) (agonist for TLR8), IRM3 (N-(2-[2-[4-amino-2-(2-methoxyethyl)-1H-imidazo[4,5-c]quinolin-1-yl]ethox-y]ethyl)-N-methylcyclohexanecarboxamide) (agonist for TLR8), CL097 (2-(ethoxymethyl)-1H-imidazo[4,5-c]quinolin-4-amine) (agonist for TLR7/8), CL307 (agonist for TLR7), CL264 (agonist for TLR7), Resiquimod (agonist for TLR7/8), 3M-052/MEDI9197 (agonist for TLR7/8), SD-101 (N-[(4S)-2,5-dioxo-4-imidazolidinyl]-urea) (agonist for TLR7/8), motolimod (2-amino-N,N-dipropyl-8-[4-(pyrrolidine-1-carbonyl)phenyl]-3H-1-benzazepine-4-carboxamide) (agonist for TLR8), CL075 (3M002, 2-propylthiazolo[4,5-c]quinolin-4-amine) (agonist for TLR7/8), and TL8-506 (3H-1-benzazepine-4-carboxylic acid, 2-amino-8-(3-cyanophenyl)-ethyl ester) (agonist for TLR8).

Examples of TLR2 agonists include but are not limited to an agent comprising N-α-palmitoyl-S-[2,3-bis(palmitoyloxy)-(2RS)-propyl]-L-cysteine, palmitoyl-Cys((RS)-2,3-di(palmitoyloxy)-propyl) (“Pam3Cys”), e.g., Pam3Cys, Pam3Cys-Ser-(Lys)4 (also known as “Pam3Cys-SKKKK” and “Pam3CSK4”), Triacyl lipid A (“OM-174”), Lipoteichoic acid (“LTA”), peptidoglycan, and CL419 (S-(2,3-bis(palmitoyloxy)-(2RS)propyl)-(R)-cysteinyl spermine).

An example of a TLR2/6 agonist is Pam₂CSK₄ (S-[2,3-bis(palmitoyloxy)-(2RS)-propyl]-[R]-cysteinyl-[S]-seryl-[S]-lysyl-[S]-lysyl-[S]-lysyl-[S]-lysine×3 CF₃COOH).

Examples of TLR2/7 agonists include CL572 (S-(2-myristoyloxy ethyl)-(R)-cysteinyl 4-((6-amino-2-(butylamino)-8-hydroxy-9H-purin-9-yl)methyl) aniline), CL413 (S-(2,3-bis(palmitoyloxy)-(2RS)propyl)-(R)-cysteinyl-(S)-seryl-(S)-lysyl-(S)-lysyl-(S)-lysyl-(S)-lysyl 4-((6-amino-2-(butylamino)-8-hydroxy-9H-purin-9-yl)methyl)aniline), and CL401 (S-(2,3-bis(palmitoyloxy)-(2RS)propyl)-(R)-cysteinyl 4-((6-amino-2(butyl amino)-8-hydroxy-9H-purin-9-yl)methyl) aniline).

There are a large number of other adjuvants available, many of which can be co-encapsulated with the viral protein and/or protein fragments (antigen) in the biocompatible polymer (e.g., PLGA) nanoparticles. Examples for which there are strong experimental support as vaccine components include bisphosphonates (e.g., alendronate), CpG ODNs (TLR9 agonists) (see, e.g., Bode et al. (2011) Expert Rev. Vaccines. 10(4): 499-511), imiquimod-family compounds (TLR7 agonists) (see, e.g., Zhang et al. (2015) Clin. Vacc. Immunol, 21(4): 5709-579)), lipopolysaccharide-based compounds (TLR4) (see, e.g., Patil et al. (2014) J. Control Release, 174: 51-62). Other possible choices include but are not limited to LPS-like compounds (e.g., MPLA, AS01, AS02, AS04, etc.), Flagellin, dsRNA-like compounds (e.g., Poly (I:C) and derivatives). It is also possible to target retinoic acid-inducible gene I (RIG-I)-like receptors (RLRs) using poly (I:C) and derivatives.

In certain embodiments the incorporation of any other adjuvant known to a skilled person and suitable for use in combination with one or more of the viral protein(s) or protein fragment(s) in the immunogenic nanoparticles is contemplated. Illustrative adjuvants include, but are not limited to, TDM, MDP, muramyl dipeptide, pluronics, alum solution, aluminum hydroxide, ADJUMER™ (polyphosphazene), aluminium phosphate gel, glucans from algae, algammulin, aluminium hydroxide gel (alum), highly protein-adsorbing aluminium hydroxide gel, low viscosity aluminium hydroxide gel, AF or SPT (emulsion of squalane (5%), Tween 80 (0.2%), Pluronic L121 (1.25%), phosphate-buffered saline, pH 7.4), AVRIDINE™ (propanediamine), BAY R1005™ ((N-(2-deoxy-2-L-leucylamino-b-D-glucopyranosyl)-N-oc-tadecy 1-dodecanoy I-amide hydroacetate), CALCITRI O LTM (1-alpha,25-dihydroxy-vitamin D3), calcium phosphate gel, CAP™ (calcium phosphate nanoparticles), cholera holo-toxin, cholera-toxin-A1-protein-A-D-fragment fusion protein, sub-unit B of the cholera toxin, CRL 1005 (block copolymer P1205), cytokine-containing liposomes, DDA (dimethyldioctadecylammonium bromide), DHEA (dehydroepiandrosterone), DMPC (dimyristoylphosphatidylcholine), DMPG (dimyristoylphosphatidylglycerol), DOC/alum complex (deoxycholic acid sodium salt), Freund's complete adjuvant, Freund's incomplete adjuvant, gamma inulin, Gerbu adjuvant (mixture of: i) N-acetylglucosaminyl-(Pl-4) N-acetylmuramyl-L-alanyl-D-glutamine (GMDP), ii) dimethyldioctadecylammonium chloride (DDA), iii) zinc-L-proline salt complex (ZnPro-8), GM-CSF), GMDP (N-acety lglucosaminyl-(b 1-4)-N-acety lmuramy 1-L-alany 1-D-isoglutamine), imiquimod (1-(2-methypropyl)-1H-imidazo[4,5-c]quinoline-4-amine), ImmTher™ (N-acetylglucosaminyl-N-acetylmuramyl-L-Ala-D-isoGlu-L-Ala-glycerol dipalmitate), DRVs (immunoliposomes prepared from dehydration-rehydration vesicles), interferon-gamma, interleukin-I beta, interleukin-2, interleukin-7, interleukin-12, ISCOMS™, ISCOPREP 7.0.3.™, liposomes, LOXORIBINE™ (7-allyl-8-oxoguanosine), LT oral adjuvant (E. coli labile enterotoxin-protoxin), MF59™, (squalene-water emulsion), MONTANIDE ISA 51™ (purified incomplete Freund's adjuvant), MONTANIDE ISA 720™ (metabolisable oil adjuvant), MPL™ (3-Q-desacyl-4′-monophosphoryl lipid A), MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt), MURAMETIDE™ (Nac-Mur-L-Ala-D-Gln-OCH3), MURAPALMITINE™ and D-MURAPALMITINE™ (Nac-Mur-L-Thr-D-isoGln-sn-glyceroldipalmitoyl), NAGO (neuraminidase-galactose oxidase), protein cochleates (Avanti Polar Lipids, Inc., Alabaster, Ala.), STIMULON™ (QS-21), Quil-A (Quil-A saponin), S-28463 (4-amino-otec-dimethyl-2-ethoxymethyl-1H-imidazo[4,5c]quinoline-1-ethanol), SAF-1™ (“Syntex adjuvant formulation”), ROBANE® (2,6,10,15,19,23-hexamethyltetracosan and 2,6,10,15,19,23-hexamethyl-2,6,10,14,18,22-tetracosahexane), stearyltyrosine (octadecyltyrosine hydrochloride), THERAMID® (N-acetylglucosaminyl-N-acetylmuramyl-L-Ala-D-isoGlu-L-Ala-dipalmitoxypropylamide), Theronyl-MD P (TERMURTIDE™ or [thr 1]-MDP, N-acetylmuramyl-L-threo-nyl-D-isoglutamine), Ty particles (Ty-VLPs or virus-like particles), plant derived adjuvants, including QS21, Quil A, Iscomatrix, ISCOM, adjuvants suitable for costimulation including ‘Iomatine, Inulin, microbe derived adjuvants, including Romurtide, DETOX, MPL, CWS, Mannose, CpG nucleic acid sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP, and adjuvants suitable as antagonists including CGRP neuropeptide.

Other illustrative, but non-limiting adjuvants include MATRIX™ (Magnusson et al. (2018) Immunol. Res. 66: 224-233) which consists of 40 nm honeycomb-like nanoparticles derived from plant saponins, mixed with cholesterol and a phospholipid, the GSK adjuvant (AS03) which is comprised of α-squalene and polysorbate 80 in an oil-in water emulsion (Cohet et al. (2019) Vaccine, 37: 3006-3021), the adjuvant (CpG 1018) (Campbell (2017) Meth. Mol. Biol. 1494: 15-27) which consists of a 22-mer oligonucleotide that interacts with TLR9, and the Vaxine adjuvant (Advax) which is a microparticle comprised of delta-inulin polysaccharides (Hayashi et al. (2017) EBioMedicine, 15: 127-136).

In still another illustrative, but non limiting embodiment, β-defensin (GIINTLQKYYCRVRGG RCAVLSCLPKEEQIGKC STRGRKCCRRKK (SEQ ID NO:300) can be added as an adjuvant to, for example, the amino terminus of the polypeptide using linker (e.g., EAAAK linker, SEQ ID NO:301).

The foregoing adjuvants are illustrative and non-limiting. Using the teaching provided herein, numerous other adjuvants will be available to one of skill in the art in the formulation of the immunogenic nanoparticles.

### Targeting Moieties.

In certain embodiments the immunogenic nanoparticles described herein have one or more targeting moieties attached to the surface where the targeting moieties bind to and/or facilitate uptake by antigen-presenting cells (APCs) (e.g., macrophages, dendritic cells, and B cells).

It is well-known that transmembrane mannose receptors, which allow particulate matter endocytosis, can assist antigen capture and presentation by dendritic cells, including for purposes of vaccine development (Irache et al. (2008) Expert Opin. Drug Deliv. 5(6): 703-724). Noteworthy, mannosylated particles are capable of increasing antigen uptake by dendritic cells (DC) a 100-fold over antigen uptake from a fluid phase (Engering et al. (1997) Eur. J. Immunol. 27: 2417-2425; Gijzen et al. (2007) J. Leukoc. Biol. 81: 729-740). Mannose targeting is accomplished by DC expression of a Type II mannose membrane receptor (MMR), DC-SIGN, which is present in low abundance on the DC surface in the absence of a danger signal. This introduces the possibility of decorating lipid nanoparticle (LPN) surfaces with surface mannose ligands to improve nucleic acid uptake in lymph node DC (Chen et al. (2022) Proc. Natl. Acad. Sci. USA, 119:34 e220784111; Nakamura et al. (2020) Adv. Drug Deliv. Rev. 167: 78-88) (FIG. 16). For mRNA vaccines, targeted delivery to the lymph node is predicted to reduce side effects and increase the immune response (Chen et al., supra.). This is compatible with the pharmacokinetics data provided by Pfizer to the European Medicines Agency (EMA) for their COVID-19 mRNA vaccine, BNT162b2, which in spite of IM injection could be seen to distribute mainly to the liver and injected site, with the possibility of reversible hepatic changes in animals (Alden et al. (2022) Curr. Iss. Mol. Biol. 44:1115-1126. Moreover, the BNT162b2 mRNA could be rapidly reverse-transcribed intracellularly into DNA in a human liver cell line (Huh7), which could pose significant risk to human health. Therefore, the targeting expression of mRNA in vivo can minimize side effects and improve efficacy, a key consideration of the next-generation of LNPs (Loughrey & Dahlman (2021) Acc. Chem. Res. 55: 13-23; Van der Jeught et al. (2018) ACS Nano, 12: 9815-9829: Sahin et al. (2020) Nature, 585: 107-112).

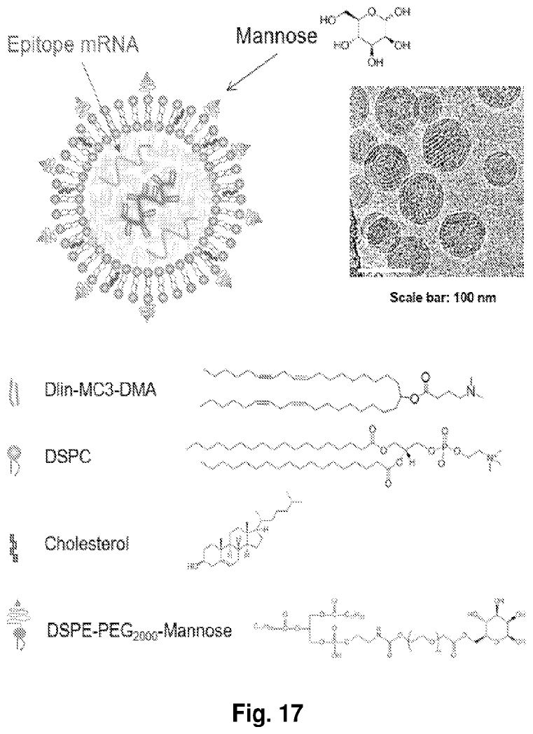

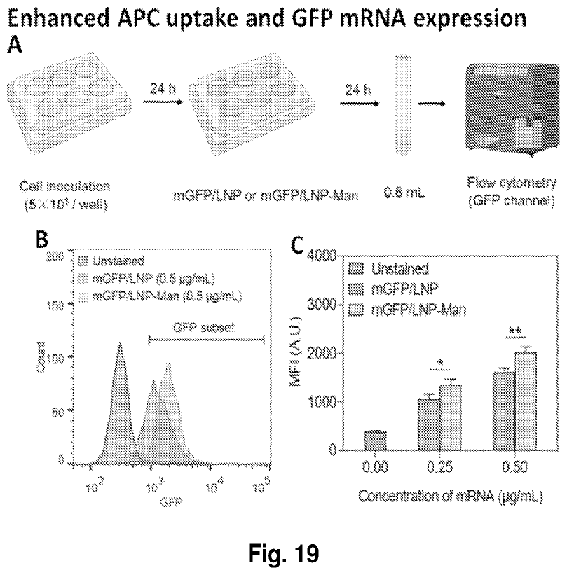

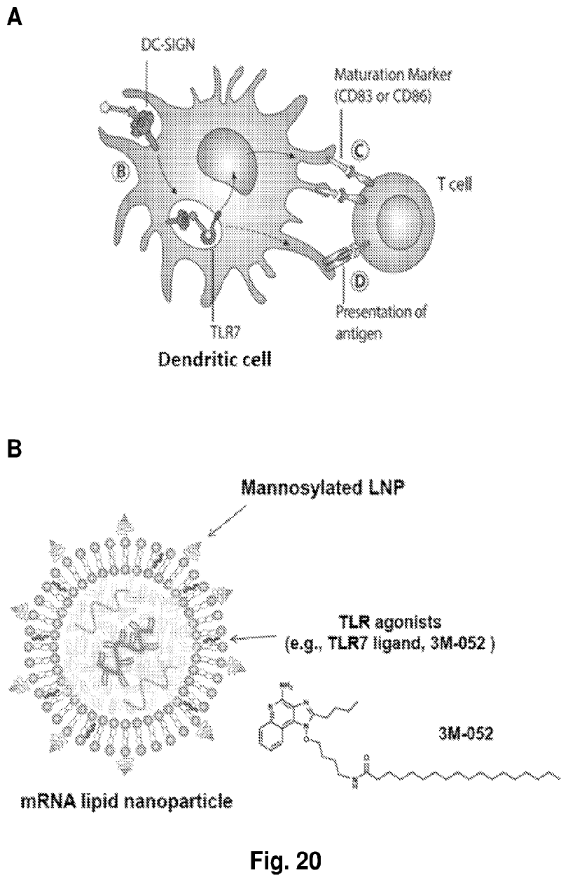

To develop lymph node targeting to improve multi-epitope nucleic acid delivery, our first approach was to use a mannose-lipid conjugate that can be used during microfluidics mixing (Nanoassemblr) to construct a cationic lipid nanoparticle, wherein the DSPE-PEG2000-mannose is included in the lipid suspension before mixing with an aqueous mRNA phase. For proof-of-principle, we selected a GFP RNA construct to develop in-house LNPs, with and without surface decoration with mannose (FIG. 17). GFP-mRNA LNPs were synthesized through the mixing of the ionizable cationic lipid, i.e., DLin-MC3-DMA, with DSPC, cholesterol and DMG-PEG-2k or DSPE-PEG2000-mannose. Briefly, the lipid compositions of DLin-MC3-DMA, DSPC, cholesterol and DMG-PEG-2k/DSPE-PEG2000-mannose were dissolved in ethanol stock solutions at 10, 20, 10 and 20 mg/mL, respectively. The aqueous phase was prepared by dissolving 0.2 mg GFP-mRNA in sodium acetate buffer. Following preparation of the aqueous and organic phases, a microfluidics mixer (NanoAssemblr Benchtop, Precision NanoSystems Inc.) was used to synthesize two batches of GFP mRNA delivering LNPs, which were characterized by CryoEM (FIG. 17) and DLS (FIG. 18). A subsequent flow cytometry experiment demonstrated increased uptake of the fluorescent protein when delivered by mGFP/LNP-Man compared to mGFP/LNP in an antigen presenting cell (FIG. 19). We could also demonstrate interference in the increased uptake of the decorated LNP by an excess of mannose or antibody interference with the mannose receptor. Another iteration of using this monosaccharide-decorated nanoparticle, is the incorporation of the lipid tail of a TLR7 agonist, 3M-052, on the particle surface, with a view to increasing DC SIGN expression, thereby further improving mRNA uptake and epitope presentation (FIG. 20). This is in agreement with observation by Li et al (Frontiers in Chemistry, 2019, Volume 7, Article 650), using multivalent conjugation of an antigenic peptide epitope to a TLR7 agonist plus one of a libraryof mannosides to improve dendritic cell activation through increased DC SIGN, expression (FIG. 20).

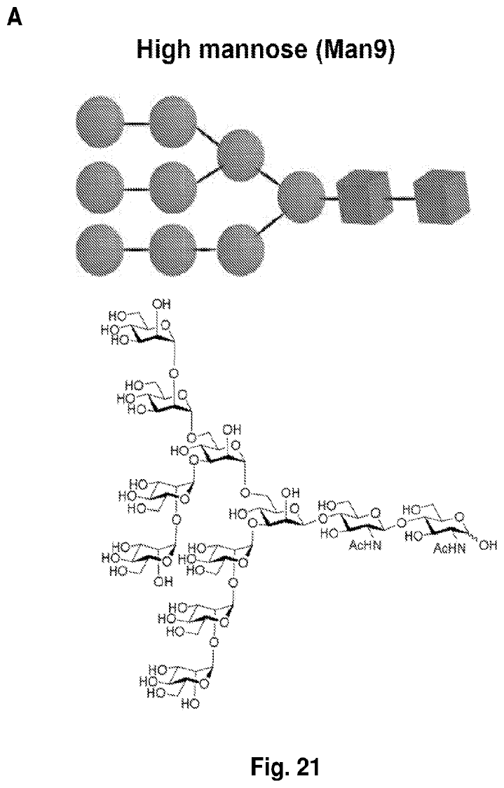

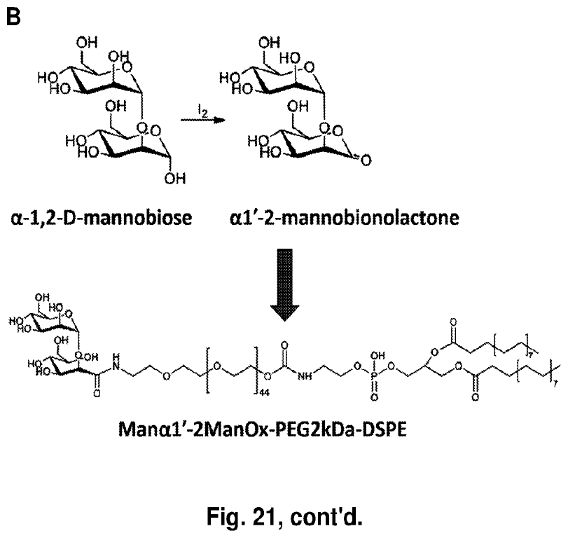

The structure of the mannosylated ligand is another variable determining the interaction with DC SIGN, and the quality of the immune response. This is illustrated by the work of Li et al. (2019) Front. Chem. Volume 7, Article 650), who synthesized a library of well-defined mannosides based on the knowledge that multivalent nanostructures improve DC-SIGN binding. Thus, instead of using a monosaccharide, we are constructing lipid nanoparticles that include more complex oligomannosides (Van Liempt et al. (2006) FEBS Lett. 2006; 580: 6123-6131; Le Moignic et al. (2018) 278:110-121). In addition to the library of materials available in the work of Li et al., a number of larger and more complex oligomannosides with intrinsic multivalence are available, including the oligomannose N-linked oligosaccharide, Nan 9, which displays 9 mannosyl residues (Ni et al., 2006; McIntosh et al., 2015) (FIG. 21). An alternative approach is the use of α1′-2-mannobionolactone (2-O-α-d-mannopyranosyl-d-mannonolactone), which can be prepared from α1′-2-mannobiose, for conjugation to DSPE-PEG, as described by Pappalardo et al. (2021) Mol. Pharmaceutics, 18: 2540-2555). This conjugate is used in a similar way as described for the monosaccharide conjugate, demonstrated in FIGS. 17 and 18.

The LNP composition is shown below in Table 41 and nanoassembly setting are shown in Table 42.

TABLE 41
LNP and Man-LNP composition.

| Composition | LNP | LNP-MAN |
|---|---|---|
| Dlin-MC3-DMA | 50 | 50 |
| DSPC | 10 | 10 |
| Cholesterol | 38.5 | 38.5 |
| DMG-PEG₂₀₀₀ | 1.5 | / |
| DSPE-PEG₂₀₀₀-Man | / | 1.5 |

| | |
|---|---|
| mRNA | PolyA or mGFP or mAra H2 |
| N:P | 4:1 |

TABLE 42
Nanoassemblr setting and parameters.

| Settings | Parameters | |
|---|---|---|
| Volume | 2.2 | mL |
| Flow Rate Ratio | 3:1 (aq:org) | |
| Total Flow Rate | 12 | mL/min |
| Left syringe size (aqueous) | 1.65 | mL |
| Right syringe size (organic) | 0.55 | mL |

The size and charge of mannose-LNPs is shown below in Table 43.

TABLE 43
Size and charge of LNPs and Mannose-LNPs.

| Characterization | LNP | LNP-Man |
|---|---|---|
| Size (nm) | 100.6 ± 0.5 | 153.2 ± 1.1 |
| Zet (mV) | −3.70 | −5.37 |
| Polydispersity | 0.170 ± 0.005 | 0.177 ± 0.023 |
| EE % | 90.2% | 92% |

It is of interest to note that highly glycosylated or mannosylated particulates promote antigen access to lymph node germinal centers, where T-follicular helper cells assist B-cell antibody production (Nel and Miller, ACS Nano, 2021; 15:5793-5818).

In certain embodiments the nanoparticles comprise targeting moieties that bind to dendritic cells or other myeloid or lymphoid antigen-presenting cells. Illustrative dendritic cell receptors that are readily targeted include but are not limited to, DEC205, MR, Dectin-1, DC-SIGN, DNGR-1, FcγR, and the like. An illustrative, but non-limiting list of targeting moieties that bind to these receptors is shown in Table 44.

TABLE 44
Receptors that can be targeted on dendritic
cells and illustrative targeting moieties.

| Receptors | Targeting moieties |
|---|---|
| DEC205 | SAG-1 (T. gondii) |
| | HIV-1 gagP24 |
| | Ova |
| | AHc |
| | RSV fusion protein |
| MR | MUC1 |
| | MAA |
| | hCGβ |
| Dectin-1 | Ova |
| | Diphtheria toxin (CRM₁₉₇) |
| DC-SIGN | Ag85B (Mtb) |
| | triMN-LPR |
| DNGR-1 | Ova |
| | Anti-Clec9A |
| | Ova |
| | MUC1 |
| FcγR | MUC1-Tn |
| | E75 (HER-2) |
| | iFT |
| | gp120_{αgal}/p24 |
| | α-gal |

Abbreviations: AHc = recombinant Hc of Clostridium botulinum neurotoxin serotype A, MAA = melanoma-associated antigens, iFT = inactivated Francisella tularensis, CTB = cholera toxin B subunit.

In certain embodiments the nanoparticles comprise targeting moieties that bind to macrophages. Illustrative macrophage receptors that are readily targeted include but are not limited to sialoadhesin receptors, folate receptors, galactose receptors, mannose receptors, P3-glucan receptors, scavenger receptors, tuftsin receptors, and the like. Illustrative, but non-limiting list of targeting moieties that bind to these receptors is shown in Table 45.

TABLE 45
Receptors that can be targeted on macrophages
and illustrative targeting moieties.

| Receptors | Targeting moieties |
|---|---|
| sialoadhesin receptors | sialic acid |
| | 9-N-(4H-thieno[3,2-c]chromene-2-carbamoyl)- |
| | Neu5Acα2-3Galβ1-4GlcNAc (TCCNeu5Ac) |
| folate receptors | folic acid and methotrexate or dendrimers thereof |
| | folate |
| galactose receptors | galactose residues |
| | lactose |
| | low density lipoprotein (LDL) |
| | ovalbumin (OVA) |
| | lactobionic acid |
| mannose receptors | mannose-rich glycoconjugates |
| | mannose |
| | mannan |
| | mannosylated poly(L-lysine) (MPL) |
| β-glucan receptors | zymosan and other β-glucans |
| | glucan |
| scavenger receptors | poly-guanine |
| | apoB protein fragment |
| tuftsin receptors | Tufsin tetrapeptide (Thr-Lys-Pro-Arg, (SEQ ID |
| | NO: 302)) |

With respect to scavenger receptors, it is noted that scavenger receptors are receptors on macrophages and other cells that bind to numerous ligands, such as bacterial PP19T cell-wall components, and remove them from the blood. Illustrative scavenger receptors on macrophages include, but are not limited to stabilin-1, stabilin-2, SCARA1 or MSR1, SCARA2 or MARCO, SCARA3, SCARA4 or COLEC12, SCARA5, SCARB1, SCARB2, SCARB3 or CD36, and the like. In certain embodiments the targeting moiety comprises a moiety (e.g., a peptide) that binds to stabilin-1 and/or to stabilin-2.

Ligands that bind to scavenger receptors are well known to those of skill in the art and as noted above, can readily be incorporated as a targeting moiety into the immunogenic nanoparticles described herein. In certain embodiments, for example, suitable ligand that binds to stabilin-1 and/or to stabilin-2 comprises a fragment of the apoB protein. In certain embodiments, the fragment ranges in length from about 5 up to about 50, or up to about 40, or up to about 30, or up to about 20 amino acids. In certain embodiments the fragment ranges in length from about 5 up to about 20, or up to about 10 amino acids. In certain embodiments the first targeting moiety is a peptide comprising the amino acid sequence RKRGLK (SEQ ID NO:303). In certain embodiments the first targeting moiety is a peptide comprising the amino acid sequence RLYRKRGLK (SEQ ID NO:304). In certain embodiments the first targeting moiety is a peptide comprising the amino acid sequence CGGKLGRKRYLR (SEQ ID NO:305)). In certain embodiments the surface can be coated by sugars such as mannan as well as targeting moieties such as aptamers and the like. Aptamers are oligonucleotide molecules that exhibit 3D structure to allow them to bind to specific target molecules. Aptamers are usually created by selecting them from a random sequence pool but natural aptamers also exist.

The mannose receptor (cluster of differentiation 206, CD206) is a C-type lectin primarily present on the surface of macrophages, immature dendritic cells and liver sinusoidal endothelial cells. Ligands that bind to the mannose receptor are well known to those of skill in the art and can also readily be incorporated as a targeting moiety into the immunogenic nanoparticles described herein. Illustrative, but non-limiting examples of ligands for the mannose receptor include, but are not limited to mannan, mannose, N-acetylglucosamine, and fucose. In certain embodiments the targeting moiety can comprise a mannan (e.g., a mannan ranging from about 35 to about 30 kDa).

In certain embodiments the targeting moiety can target lymph nodes. Illustrative moieties that target lymph nodes include, but are not limited to CpG (see, e.g., Thomas, et al. (2014) Biomaterials, 35(2): 814-824), and the novel amph-CpG family. The introduction of glycosylated components on the particle surface, has also been shown to allow interaction mannose-binding protein, which activates the complement system and can be used, to promote nanoparticle access to germinal centers, where they promote memory B-cell responses (see Tokatlian, et al. (2019). Science, 363, 649-654).

In certain embodiments the targeting moiety can be attached to the nanoparticle by simple adsorption or by non-covalent linkages. Useful non-covalent linkages include, but are not limited to, affinity binding pairs, such as biotin-streptavidin and immunoaffinity, having sufficiently high affinity to maintain the linkage during use. Such non-covalent linkers/linkages are well known to those of skill in the art.

In certain embodiments the targeting moiety can be covalently coupled to the nanoparticle directly or through a linker. The art is also replete with conjugation chemistries useful for covalently linking a targeting moiety to a second moiety (e.g., a biocompatible polymer). Art-recognized covalent coupling techniques are disclosed, for instance, in U.S. Pat. Nos. 5,416,016, 6,335,435, 6,528,631, 6,861,514, 6,919,439, and the like. Other conjugation chemistries are disclosed in U.S. Patent Publication No. 2004/0249178. Still other conjugation chemistries include: p-hydroxy-benzoic acid linkers (see, e.g., Chang-Po et al. (2002) Bioconjugate Chem. 13(3): 525-529), native ligation (see, e.g., Stetsenko et al. (2000) J. Org. Chem. 65: 4900-4908), disulfide bridge conjugates (see, e.g., Oehlke et al. (2002) Eur. J. Biochem. 269: 4025-4032; Rogers et al. (2004) Nucl. Acids Res. 32(22): 6595-6604), maleimide linkers (see, e.g., Zhu et al. (1993) Antisense Res Dev. 3: 265-275), thioester linkers (see, e.g., Ede et al. (1994) Bioconjug. Chem. 5: 373-378), Diels-Alder cycloaddition (see, e.g., Marchan et al. (2006) Nucl. Acids Res. 34(3): e24); U.S. Pat. No. 6,656,730 and the like). For reviews of conjugation chemistries, see also Tung et al. (2000) Bioconjugate Chem. 11: 605-618, Zatsepin et al. (2005) Curr. Pharm. Des. 11(28): 3639-3654, Juliano (2005) Curr. Opin. Mol. Ther. 7(2): 132-136, and the like. While certain of the foregoing chemistries are utilized for nucleic acid-peptide conjugation one of skill will recognize that they can readily be modified for attachment of first and/or second targeting moieties to the nanoparticles.

Tom, should we also add a short section about the fact that the cationic ionizable lipid component and the lipid composition of of LNPs can have a determining effect on systemic biodistribution

The foregoing targeting moieties and methods of attachment to the nanoparticle(s) are illustrative and non-limiting. Using the teaching provided herein numerous other targeting moieties and attachment chemistries will be available to one of skill in the art.

### Auxiliary Components.

In certain embodiments the immunogenic nanoparticles described herein can additionally include one or more auxiliary substances in order to facilitate cytosolic release of the cargo from the endolysosomal compartment and/or to increase the immunogenicity or immunostimulatory capacity of the nanoparticle.

Thus, in certain embodiments the nanoparticles described herein can additionally include substances that facilitate cytosolic release of the cargo (e.g., mRNA) from the endolysosomal compartment. Such substances include but are not limited to endo-osmolytic peptides (e.g., MPG, Pep-1 and PPTG1) that destabilizes the endolysosomal membrane. Other endo-osmolytic peptides include peptides derived from antimicrobial peptides (e.g., LL-37, melittin, and bombolitin V, with glutamic acid substituting for all basic residues, see, e.g., Ahmad et al. (2015) Biochimica et Biophysica Acta (BBA)—Biomembranes, 1848(2): 544-553), and the like.

Depending on the various types of auxiliary substances, various mechanisms can come into consideration in this respect. For example, compounds that permit or induce the maturation of dendritic cells (DCs), for example lipopolysaccharides, TNF-α or CD40 ligand, form a first illustrative class of suitable auxiliary substances. In general, it is possible to use as auxiliary substance any agent that influences the immune system in the manner of a “danger signal” (LPS, GP96, didodecyldimethylammonium bromide (DDAB) (see, e.g., Liu et al. (2018) Mol. Pharmaceutics, 15: 11: 5227-5235, etc.) or cytokines, such as GM-CSF, which allow an immune response to be enhanced and/or influenced in a targeted manner. Illustrative auxiliary substances include but are not limited to, cytokines, such as monokines, lymphokines, interleukins or chemokines, that further promote the innate immune response, such as IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-α, IFN-β, IFN-γ, GM-CSF, G-CSF, M-CSF, LT-β or TNF-α, growth factors, such as hGH, and the like. An illustrative, but non-limiting list of cytokines that can be included in the immunogenic nanoparticles is shown in Table 46.

TABLE 46
Illustrative CC, CL, CXC, and CX3C cytokines that can be incorporated
into immunogenic nanoparticles described herein.

| Type | Name | Gene | Other Name(s) | Receptor | Uniprot |
|---|---|---|---|---|---|
| CC | CCL1 | Scya1 | I-309, TCA-3 | CCR8 | |
| CC | CCL2 | Scya2 | MCP-1 | CCR2 | P13500 |
| CC | CCL3 | Scya3 | MIP-1a | CCR1 | P10147 |
| CC | CCL4 | Scya4 | MIP-1β | CCR1, CCR5 | P13236 |
| CC | CCL5 | Scya5 | RANTES | CCR5 | P13501 |
| CC | CCL6 | Scya6 | C10, MRP-2 | CCR1 | P27784 |
| CC | CCL7 | Scya7 | MARC, | CCR2 | P80098 |
| | | | MCP-3 | | |
| CC | CCL8 | Scya8 | MCP-2 | CCR1, CCR2B, | P80075 |
| | | | | CCR5 | |
| CC | CCL9/CCL10 | Scya9 | MRP-2, | CCR1 | P51670 |
| | | | CCF18, MIP-1 | | |
| CC | CCL11 | Scya11 | Eotaxin | CCR2, CCR3, | P51671 |
| | | | | CCR5 | |
| CC | CCL12 | Scya12 | MCP-5 | | Q62401 |
| CC | CCL13 | Scya13 | MCP-4, | CCR2, CCR3, | Q99616 |
| | | | NCC-1, | CCR5 | |
| | | | Ckβ10 | | |
| CC | CCL14 | Scya14 | HCC-1, | CCR1 | Q16627 |
| | | | MCIF, Ckβ1, | | |
| | | | NCC-2, CCL | | |
| CC | CCL15 | Scya15 | Leukotactin-1, | CCR1, CCR3 | Q16663 |
| | | | MIP-5, | | |
| | | | HCC-2, | | |
| | | | NCC-3 | | |
| CC | CCL16 | Scya16 | LEC, NCC-4, | CCR1, CCR2, | O15467 |
| | | | LMC, Ckβ12 | CCR5, CCR8 | |
| CC | CCL17 | Scya17 | TARC, | CCR4 | Q92583 |
| | | | dendrokine, | | |
| | | | ABCD-2 | | |
| CC | CCL18 | Scya18 | PARC, DC-CK1, | | P55774 |
| | | | AMAC-1, | | |
| | | | Ckβ7, MIP-4 | | |
| CC | CCL19 | Scya19 | ELC, | CCR7 | Q99731 |
| | | | Exodus-3, | | |
| | | | Ckβ11 | | |
| CC | CCL20 | Scya20 | LARC, | CCR6 | P78556 |
| | | | Exodus-1, | | |
| | | | Ckβ4 | | |
| CC | CCL21 | Scya21 | SLC, 6Ckine, | CCR7 | O00585 |
| | | | Exodus-2, | | |
| | | | Ckβ9, TCA-4 | | |
| CC | CCL22 | Scya22 | MDC, DC/β-CK | CCR4 | O00626 |
| CC | CCL23 | Scya23 | MPIF-1, | CCR1 | P55773 |
| | | | Ckβ8, MIP-3, | | |
| | | | MPIF-1 | | |
| CC | CCL24 | Scya24 | Eotaxin-2, | CCR3 | O00175 |
| | | | MPIF-2, | | |
| | | | Ckβ6 | | |
| CC | CCL25 | Scya25 | TECK, | CCR9 | O15444 |
| | | | Ckβ15 | | |
| CC | CCL26 | Scya26 | Eotaxin-3, | CCR3 | Q9Y258 |
| | | | MIP-4a, | | |
| | | | IMAC, TSC-1 | | |
| CC | CCL27 | Scya27 | CTACK, | CCR10 | Q9Y4X3 |
| | | | ILC, Eskine, | | |
| | | | PESKY, | | |
| | | | skinkine | | |
| CC | CCL28 | Scya28 | MEC | CCR3, CCR10 | Q9NRJ3 |
| CXC | CXCL1 | Scyb1 | Gro-a, | CXCR2 | P09341 |
| | | | GRO1, NAP-3, | | |
| | | | KC | | |
| CXC | CXCL2 | Scyb2 | Gro-β, | CXCR2 | P19875 |
| | | | GRO2, MIP-2a | | |
| CXC | CXCL3 | Scyb3 | GRO3, MIP-2β | CXCR2 | P19876 |
| CXC | CXCL4 | Scyb4 | PF-4 | CXCR3B | P02776 |
| CXC | CXCL5 | Scyb5 | ENA-78 | CXCR2 | P42830 |
| CXC | CXCL6 | Scyb6 | GCP-2 | CXCR1, CXCR2 | P80162 |
| CXC | CXCL7 | Scyb7 | NAP-2, | | P02775 |
| | | | CTAPIII, | | |
| | | | β-Ta, PEP | | |
| CXC | CXCL8 | Scyb8 | IL-8, NAP-1, | CXCR1, CXCR2 | P10145 |
| | | | MDNCF, | | |
| | | | GCP-1 | | |
| CXC | CXCL9 | Scyb9 | MIG, CRG-10 | CXCR3 | Q07325 |
| CXC | CXCL10 | Scyb10 | IP-10, CRG-2 | CXCR3 | P02778 |
| CXC | CXCL11 | Scyb11 | I-TAC, β-R1, | CXCR3, CXCR7 | O14625 |
| | | | IP-9 | | |
| CXC | CXCL12 | Scyb12 | SDF-1, PBSF | CXCR4, CXCR7 | P48061 |
| CXC | CXCL13 | Scyb13 | BCA-1, BLC | CXCR5 | O43927 |
| CXC | CXCL14 | Scyb14 | BRAK, | | O95715 |
| | | | bolekine | | |
| CXC | CXCL15 | Scyb15 | Lungkine, | | Q9WVL7 |
| | | | WECHE | | |
| CXC | CXCL16 | Scyb16 | SRPSOX | CXCR6 | Q9H2A7 |
| CXC | CXCL17 | VCC-1 | DMC, VCC-1 | | Q6UXB2 |
| C | XCL1 | Scyc1 | Lymphotactin | XCR1 | P47992 |
| | | | a, SCM-1a, | | |
| | | | ATAC | | |
| C | XCL2 | Scyc2 | Lymphotactin | XCR1 | Q9UBD3 |
| | | | β, SCM-1β | | |
| CX3C | CX3CL1 | Scyd1 | Fractalkine, | CX3CR1 | P78423 |
| | | | Neurotactin, | | |
| | | | ABCD-3 | | |

The foregoing auxiliary substances are illustrative and non-limiting. Using the teaching provided herein numerous other auxiliary substances known to one of skill in the art can readily be incorporated in the immunogenic nanoparticles described herein.

Pharmaceutical formulations.

In various embodiments pharmaceutical formulations (e.g., formulations suitable for vaccination of a mammal) comprising the immunogenic nanoparticles described herein are provided. Such pharmaceutical formulations can be prepared by any method known or hereafter developed in the art of pharmaceutics. In general, such preparatory methods include the step of bringing the active ingredient (e.g., immunogenic nanoparticles) into association with one or more excipients and/or one or more other accessory ingredients, and then, if necessary and/or desirable, shaping and/or packaging the product into a desired single- or multi-dose unit.

In certain embodiments a pharmaceutical composition may be prepared, packaged, and/or sold in bulk, as a single unit dose, and/or as a plurality of single unit doses. As used herein, a “unit dose” is a discrete amount of the pharmaceutical composition comprising a predetermined amount of the active ingredient (immunogenic nanoparticles). The amount of the active ingredient is generally equal to the dosage of the active ingredient which would be administered to a subject and/or a convenient fraction of such a dosage such as, for example, one-half or one-third of such a dosage.

The relative amounts of the active ingredient (immunogenic nanoparticles), the pharmaceutically acceptable excipient(s), and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient (immunogenic nanoparticles).

In certain embodiments the pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21.sup.st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the pharmaceutical formulations comprising the immunogenic nanoparticles described herein. Excipients such as cocoa butter and suppository waxes, coloring agents, coating agents, sweetening, flavoring, and perfuming agents can be present in the composition, according to the judgment of the formulator.

Illustrative diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Illustrative granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water-insoluble starch, calcium carboxymethyl cellulose, magnesium aluminum silicate (Veegum), sodium lauryl sulfate, quaternary ammonium compounds, etc., and combinations thereof.

Illustrative surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and Veegum [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN20@], polyoxyethylene sorbitan [TWEEN60@], polyoxyethylene sorbitan monooleate [TWEEN80@], sorbitan monopalmitate [SPAN40@], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ®45], polyoxyethylene hydrogenated castor oil, polyethoxylated castor oil, polyoxymethylene stearate, and Solutol), sucrose fatty acid esters, polyethylene glycol fatty acid esters (e.g., CREMOPHOR®), polyoxyethylene ethers, (e.g., polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Illustrative binding agents include, but are not limited to, starch (e.g., cornstarch and starch paste); gelatin; sugars (e.g., sucrose, glucose, dextrose, dextrin, molasses, lactose, lactitol, mannitol,); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Illustrative preservatives may include antioxidants, chelating agents, antimicrobial preservatives, antifungal preservatives, alcohol preservatives, acidic preservatives, and other preservatives. Illustrative antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Illustrative chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Illustrative antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Illustrative antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Illustrative alcohol preservatives include, but are not limited to, ethanol, polyethylene glycol, phenol, phenolic compounds, bisphenol, chlorobutanol, hydroxybenzoate, and phenylethyl alcohol. Illustrative acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, Germall 115, Germaben II, Neolone™, Kathon™, and EUXYL®. In certain embodiments, the preservative is an antioxidant. In other embodiments, the preservative is a chelating agent.

Illustrative buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Illustrative lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Illustrative oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Illustrative oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, vaccine nanoparticles of the invention are mixed with solubilizing agents such as CREMOPHOR®, alcohols, oils, modified oils, glycols, polysorbates, cyclodextrins, polymers, and combinations thereof.

Injectable formulations, for example, sterile injectable aqueous or oleaginous suspensions may be formulated according to the known art using suitable dispersing or wetting agents and suspending agents. A sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable media prior to use.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

In certain embodiments the active ingredients (immunogenic nanoparticles described herein) can be in micro-encapsulated form with one or more excipients as noted above. Solid dosage forms of tablets, dragees, capsules, pills, and granules can be prepared with coatings and shells such as enteric coatings, release controlling coatings and other coatings well known in the pharmaceutical formulating art. In such solid dosage forms, active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

In certain embodiments, dosage forms for topical and/or transdermal administration of vaccine nanoparticles may include ointments, pastes, creams, lotions, gels, powders, solutions, sprays, inhalants and/or patches. Generally, active ingredient is admixed under sterile conditions with a pharmaceutically acceptable excipient and/or any needed preservatives and/or buffers as may be required. Additionally, the use of transdermal patches is also contemplated. Such patches often have the added advantage of providing controlled delivery of an active ingredient to the body. Such dosage forms may be prepared, for example, by dissolving and/or dispensing the active ingredient in the proper medium. Alternatively, or additionally, the rate may be controlled by either providing a rate controlling membrane and/or by dispersing the active ingredient in a polymer matrix and/or gel.

Suitable devices for use in delivering intradermal pharmaceutical compositions described herein include soluble microneedle array patches, e.g., as described in U.S. Patent Publication Nos: US 2019/0358441 A1, US 2019/0240469 A1, US 2019/0151638 A1, US 2019/0151638 A1, US 2018/0001070 A1, US 2017/0209553 A1, US 2016/0263362 A1, US 2017/0196966 A1, US 2016/0213908 A1, US 2016/0015952 A1, US 2015/0112250 A1, US 2012/0150023, and the like. Also contemplated are short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices that limit the effective penetration length of a needle into the skin, such as those described in PCT publication WO 99/34850 and functional equivalents thereof. Jet injection devices that deliver vaccines to the dermis via a liquid jet injector and/or via a needle that pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/037705 and WO 97/013537, and the like. Ballistic powder/particle delivery devices that use compressed gas to accelerate vaccine in powder form through the outer layers of the skin to the dermis are suitable. Alternatively or additionally, conventional syringes may be used in the classical mantoux method of intradermal administration.

In certain embodiments formulations suitable for topical administration include, but are not limited to, liquid and/or semi liquid preparations such as liniments, lotions, oil in water and/or water in oil emulsions such as creams, ointments and/or pastes, and/or solutions and/or suspensions. In certain embodiments, topically-administrable formulations may, for example, comprise from about 1% to about 10% (w/w) active ingredient (immunogenic nanoparticles described herein), although the concentration of the active ingredient may be as high as the solubility limit of the active ingredient in the solvent. In certain embodiments, formulations for topical administration may further comprise one or more of the additional ingredients described herein.

In certain embodiments, a pharmaceutical composition comprising the immunogenic nanoparticles described herein may be prepared, packaged, and/or sold in a formulation suitable for pulmonary administration via the buccal cavity. Such a formulation may, for example, comprise particles that comprise or contain the immunogenic nanoparticles described herein. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container.

Low boiling propellants generally include liquid propellants having a boiling point of below 65° F. at atmospheric pressure. In certain embodiments the propellant may constitute 50% to 99.9% (w/w) of the composition, and the active ingredient (immunogenic nanoparticles described herein) may constitute 0.1% to 20% (w/w) of the composition. The propellant may further comprise additional ingredients such as a liquid non-ionic and/or solid anionic surfactant and/or a solid diluent (which may have a particle size of the same order as particles comprising the active ingredient).

In certain embodiments, pharmaceutical compositions comprising the immunogenic nanoparticles described herein formulated for pulmonary delivery may provide the nanoparticles in the form of droplets of a solution and/or suspension. In certain embodiments, such formulations may be prepared, packaged, and/or sold as aqueous and/or dilute alcoholic solutions and/or suspensions, optionally sterile, comprising the active ingredient, and may conveniently be administered using any nebulization and/or atomization device. In certain embodiments, such formulations may further comprise one or more additional ingredients including, but not limited to, a flavoring agent such as saccharin sodium, a volatile oil, a buffering agent, a surface active agent, and/or a preservative such as methylhydroxybenzoate. In certain embodiments, the droplets provided by this route of administration may have an average diameter in the range from about 0.1 μm up to about 200 μm.

In certain embodiments, the formulations described herein as being useful for pulmonary delivery are useful for intranasal delivery of a pharmaceutical composition of the invention. Another illustrative formulation suitable for intranasal administration is a coarse powder comprising the active ingredient and having an average particle from about 0.2 μm to about 500 μm. Such a formulation can be administered in the manner in which snuff is taken, i.e. by rapid inhalation through the nasal passage from a container of the powder held close to the nares.

In certain embodiments, formulations suitable for nasal administration may, for example, comprise from about as little as 0.1% (w/w) and as much as 100% (w/w) of the immunogenic nanoparticles described herein, and may comprise one or more of the additional ingredients described herein. In certain embodiments, a pharmaceutical composition of immunogenic nanoparticles described herein may be prepared, packaged, and/or sold in a formulation suitable for buccal administration. Such formulations may, for example, be in the form of tablets and/or lozenges made using conventional methods, and may, for example, 0.1% to 20% (w/w) immunogenic nanoparticles, the balance comprising an orally dissolvable and/or degradable composition and, optionally, one or more of the additional ingredients described herein. Alternately, in certain embodiments, formulations suitable for buccal administration may comprise a powder and/or an aerosolized and/or atomized solution and/or suspension comprising the immunogenic nanoparticles. In certain embodiments, such powdered, aerosolized, and/or aerosolized formulations, when dispersed, may have an average particle and/or droplet size in the range from about 0.1 μm to about 200 μm, and may further comprise one or more of the additional ingredients described herein.

General considerations in the formulation and/or manufacture of pharmaceutical agents may be found, for example, in Remington: The Science and Practice of Pharmacy 21.sup.st ed., Lippincott Williams & Wilkins, 2005.

### Administration

In some embodiments, a therapeutically effective amount of an immunogenic nanoparticle vaccine described herein is delivered to a patient and/or animal prior to, simultaneously with, and/or after diagnosis with SARS-Cov-2. In some embodiments, a therapeutic amount of an inventive composition is delivered to a patient and/or animal prior to, simultaneously with, and/or after onset of symptoms the disease. In some embodiments, the amount of a vaccine nanoparticle is sufficient to treat, alleviate, ameliorate, relieve, delay onset of, inhibit progression of, reduce severity of, and/or reduce incidence of one or more symptoms or features of SARS-Cov-2. In some embodiments, the amount of a vaccine nanoparticle is sufficient to elicit a detectable immune response in a subject. In some embodiments, the amount of a vaccine nanoparticle is sufficient to elicit a detectable antibody response in a subject. In some embodiments, the amount of a vaccine nanoparticle is sufficient to elicit a detectable T cell response in a subject. In some embodiments, the amount of a vaccine nanoparticle is sufficient to elicit a detectable antibody and T cell response in a subject. In some embodiments, an advantage of the nanoparticles provided is that the nanoparticles can elicit potent responses with a much lower concentration of antigen than required with a conventional vaccine.

The compositions, according to the methods described herein, can be administered using any amount and any route of administration effective for treatment. The exact amount required will vary from subject to subject, depending on the species, age, and general condition of the subject, the severity of the infection, the particular composition, its mode of administration, its mode of activity, and the like. The compositions comprising the immunogenic nanoparticles described herein are typically formulated in dosage unit form for ease of administration and uniformity of dosage. It will be understood, however, that the total daily usage of the compositions will be decided by the attending physician within the scope of sound medical judgment. The specific therapeutically effective dose (or prophylactically effective dose) level for any particular subject or organism will depend upon a variety of factors including the disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The pharmaceutical compositions comprising the immunogenic nanoparticles described herein may be administered by any route suitable for administering an immunogen. In some embodiments, the pharmaceutical compositions described are administered by a variety of routes, including oral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. Specifically, contemplated routes are oral administration, intravenous injection, intramuscular injection, and/or subcutaneous injection. In some embodiments, the nanoparticles are administered parenterally. In some embodiments, the nanoparticles are administered intravenously. In some embodiments, the nanoparticles are administered orally.

In general the most appropriate route of administration will depend upon a variety of factors including the nature of the vaccine nanoparticle (e.g., its stability in the environment of the gastrointestinal tract), the condition of the subject (e.g., whether the subject is able to tolerate oral administration), etc.

In certain embodiments, the immunogenic nanoparticles described herein may be administered in amounts ranging from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. In certain embodiments, the desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

In some embodiments, the present invention encompasses “therapeutic cocktails” comprising populations immunogenic nanoparticles described herein. In some embodiments, all of the nanoparticles within a population of nanoparticles comprise a single species of targeting moiety which can bind to multiple targets (e.g., can bind to both SCS-Mph and FDCs). In some embodiments, different nanoparticles within a population of nanoparticles comprise different targeting moieties, and all of the different targeting moieties can bind to the same target. In some embodiments, different nanoparticles comprise different targeting moieties, and all of the different targeting moieties can bind to different targets. In some embodiments, such different targets may be associated with the same cell type. In some embodiments, such different targets may be associated with different cell types.

### Combination Therapies

It will be appreciated that immunogenic nanoparticles described herein and pharmaceutical compositions comprising such nanoparticles can be employed in combination therapies. The particular combination of therapies (therapeutics or procedures) to employ in a combination regimen will take into account compatibility of the desired therapeutics and/or procedures and the desired therapeutic effect to be achieved. It will be appreciated that the therapies employed may achieve a desired effect for the same purpose (for example, nanoparticle useful for vaccinating against a particular type of viral infection may be administered concurrently with another agent useful for treating the same viral infection), or they may achieve different effects (e.g., control of any adverse effects attributed to the nanoparticle).

In some embodiments, pharmaceutical compositions comprising the immunogenic nanoparticles described herein may be administered either alone or in combination with one or more other therapeutic or prophylactic agents (e.g., S protein SARS-Cov-2 vaccines). By “in combination with,” it is not intended to imply that the agents must be administered at the same time and/or formulated for delivery together, although these methods of delivery are within the scope of the invention. The compositions can be administered concurrently with, prior to, or subsequent to, one or more other desired therapeutics or medical procedures. In general, each agent will be administered at a dose and/or on a schedule determined for that agent. Additionally, the delivery of the immunogenic nanoparticles in combination with agents that may improve their bioavailability, reduce and/or modify their metabolism, inhibit their excretion, and/or modify their distribution within the body is contemplated.

The particular combination of therapies (therapeutics and/or procedures) to employ in a combination regimen will take into account compatibility of the desired therapeutics and/or procedures and/or the desired therapeutic effect to be achieved. It will be appreciated that the therapies employed may achieve a desired effect for the same disorder (for example, an immunogenic nanoparticle may be administered concurrently with another therapeutic agent used to treat the same disorder), and/or they may achieve different effects (e.g., control of any adverse effects attributed to the vaccine nanoparticle). In some embodiments, immunogenic nanoparticles described herein are administered with a second therapeutic agent that is approved by the U.S. Food and Drug Administration.

It will further be appreciated that therapeutically active agents utilized in combination may be administered together in a single composition or administered separately in different compositions.

In general, it is expected that agents utilized in combination will be utilized at levels that do not exceed the levels at which they are utilized individually. In some embodiments, the levels utilized in combination will be lower than those utilized individually.

In some embodiments, the immunogenic nanoparticles may be administered in combination with an agent, including, for example, therapeutic, diagnostic, and/or prophylactic agents. Illustrative agents to be delivered include, but are not limited to, small molecules, organometallic compounds, nucleic acids, proteins (including multimeric proteins, protein complexes, etc.), peptides, lipids, carbohydrates, hormones, metals, radioactive elements and compounds, drugs, vaccines, immunological agents, etc., and/or combinations thereof.

In certain embodiments, the immunogenic nanoparticles that delay the onset and/or progression of a particular viral infection may be administered in combination with one or more additional therapeutic agents that treat the symptoms of the viral infection. To give but one example, upon exposure to rabies virus, the immunogenic nanoparticles useful for vaccination against rabies virus may be administered in combination with one or more therapeutic agents useful for treatment of symptoms of rabies virus (e.g., antipsychotic agents useful for treatment of paranoia that is symptomatic of rabies virus infection).

In some embodiments, pharmaceutical compositions comprising the immunogenic nanoparticles described herein comprise less than 50% by weight, less than 40% by weight, less than 30% by weight, less than 20% by weight, less than 15% by weight, less than 10% by weight, less than 5% by weight, less than 1% by weight, or less than 0.5% by weight of an agent to be delivered.

In some embodiments, the immunogenic nanoparticles are administered in combination with an agent comprising one or more small molecules and/or organic compounds with pharmaceutical activity. In some embodiments, the agent is a clinically-used drug. In some embodiments, the drug is an antibiotic, and/or anti-viral agent, anti-HIV agent, anti-parasite agent, anti-protozoal agent, anesthetic, anticoagulant, inhibitor of an enzyme, steroidal agent, steroidal or non-steroidal anti-inflammatory agent, antihistamine, immunosuppressant agent, anti-neoplastic agent, antigen, vaccine, antibody, decongestant, sedative, opioid, analgesic, anti-pyretic, birth control agent, hormone, prostaglandin, progestational agent, anti-glaucoma agent, ophthalmic agent, anti-cholinergic, analgesic, anti-depressant, anti-psychotic, neurotoxin, hypnotic, tranquilizer, anti-convulsant, muscle relaxant, anti-spasmodic, muscle contractant, channel blocker, miotic agent, anti-secretory agent, anti-thrombotic agent, anticoagulant, anti-cholinergic, β-adrenergic blocking agent, diuretic, cardiovascular active agent, vasoactive agent, vasodilating agent, anti-hypertensive agent, angiogenic agent, modulators of cell-extracellular matrix interactions (e.g., cell growth inhibitors and anti-adhesion molecules), inhibitors of DNA, RNA, or protein synthesis, etc.

In certain embodiments, a small molecule agent can be any drug. In some embodiments, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460; drugs for veterinary use are listed by the FDA under 21 C.F.R. §§ 500 through 589. All listed drugs are considered acceptable for use in accordance with the present invention.

A more complete listing of classes and specific drugs suitable for use in the present invention may be found in Pharmaceutical Drugs: Syntheses, Patents, Applications by Axel Kleemann and Jurgen Engel, Thieme Medical Publishing, 1999 and the Merck Index: An Encyclopedia of Chemicals, Drugs and Biologicals, Ed. by Budavari et al., CRC Press, 1996, both of which are incorporated herein by reference.

In some embodiments, the immunogenic nanoparticles are administered in combination with one or more nucleic acids (e.g., functional RNAs, functional DNAs, etc.) to a specific location such as a tissue, cell, or subcellular locale. For example, the immunogenic nanoparticles that are used to delay the onset and/or progression of a particular viral infection may be administered in combination with RNAi agents which reduce expression of viral proteins.

In some embodiments, the immunogenic nanoparticles are administered in combination with one or more proteins or peptides. In some embodiments, the agent to be delivered may be a peptide, hormone, erythropoietin, insulin, cytokine, antigen for vaccination, etc. In some embodiments, the agent to be delivered may be an antibody and/or characteristic portion thereof.

In some embodiments, the immunogenic nanoparticles are administered in combination with one or more carbohydrates, such as a carbohydrate that is associated with a protein (e.g., glycoprotein, proteoglycans, etc.). A carbohydrate may be natural or synthetic. A carbohydrate may also be a derivatized natural carbohydrate. In certain embodiments, a carbohydrate may be a simple or complex sugar. In certain embodiments, a carbohydrate is a monosaccharide, including but not limited to glucose, fructose, galactose, and ribose. In certain embodiments, a carbohydrate is a disaccharide, including but not limited to lactose, sucrose, maltose, trehalose, and cellobiose. In certain embodiments, a carbohydrate is a polysaccharide, including but not limited to cellulose, microcrystalline cellulose, hydroxypropyl methylcellulose (HPMC), methylcellulose (MC), dextrose, dextran, glycogen, xanthan gum, gellan gum, starch, and pullulan. In certain embodiments, a carbohydrate is a sugar alcohol, including but not limited to mannitol, sorbitol, xylitol, erythritol, maltitol, and lactitol.

In some embodiments, the immunogenic nanoparticles are administered in combination with one or more lipids, such as a lipid that is associated with a protein (e.g., lipoprotein). Illustrative lipids that may be used include, but are not limited to, oils, fatty acids, saturated fatty acid, unsaturated fatty acids, essential fatty acids, cis fatty acids, trans fatty acids, glycerides, monoglycerides, diglycerides, triglycerides, hormones, steroids (e.g., cholesterol, bile acids), vitamins (e.g., vitamin E), phospholipids, sphingolipids, and lipoproteins.

Those skilled in the art will recognize that this is an illustrative, not comprehensive, list of therapeutic, diagnostic, imaging and/or prophylactic agents that can be delivered in combination with the immunogenic nanoparticles described herein. Any therapeutic, diagnostic, and/or prophylactic agent may be administered with the immunogenic nanoparticles described herein.

### Kits

In various embodiments kits are provided for inducing an immune response directed against a SARS-Cov-2 virus are provided. In various embodiments the kits comprise a container containing one or more of the immunogenic nanoparticles described herein.

In addition, the kits optionally include labeling and/or instructional materials providing directions (e.g., protocols) for the use of the immunogenic nanoparticles described herein, e.g., alone or in with, e.g., other anti-SARS-COV-2 vaccines, and/or various immune stimulants, for the treatment or prophylaxis of SARS-Cov-2, and potentially other coronaviruses.

While the instructional materials in the various kits typically comprise written or printed materials, they are not limited to such. Any medium capable of storing such instructions and communicating them to an end user is contemplated by this invention. Such media include but are not limited to electronic storage media (e.g., magnetic discs, tapes, cartridges, chips), optical media (e.g., CD ROM), and the like. Such media may include addresses to internet sites that provide such instructional materials.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An immunogenic nanoparticle comprising:
a nanoparticle comprising one or more lipid and/or one or more biocompatible polymer; and
a nucleic acid encapsulated within or attached to said nanoparticle where said nucleic acid encodes a plurality of covid-19 peptide antigens, wherein said covid-19 peptide antigens comprise one or more CD8+ T cell epitopes, one or more conserved linear B cell epitopes, and one or more CD4 T cell epitopes.

2.-3. (canceled)

4. The immunogenic nanoparticle of claim 1, wherein said plurality of covid-19 CD8+ epitopes comprise at least 1, or at least 2, or at least 3, or at least 4, or at least 5, or at least 6, or at least 7, or at least 8, or at least 9, or at least 10, or at least 11, or at least 12, or at least 13, or at least 14 amino acid sequences selected from the group consisting of RGVYYPDKVFRSSV (SEQ ID NO:34), KGIYQTSNFRVQPTESIVRF (SEQ ID NO:35), KLNDLCFTNVY (SEQ ID NO:36), FELLHAPATV (SEQ ID NO:37), TSNEVAVLYQDVNCTEV (SEQ ID NO:38), TEILPVSMTKTSVDCTMY (SEQ ID NO:39), PLLTDEMIAQYTSAL (SEQ ID NO:40), YRFNGIGV (SEQ ID NO:41), ALNTLVKQLSSNFGAISSVLNDILSRL (SEQ ID NO:42), KRVDFCGKGYHLMSFPQSAPHGVVF (SEQ ID NO:43), GVFVSNGTHW (SEQ ID NO:44), NPLLYDANYFLCWHTNCYDYCIPYNSVTSSI (SEQ ID NO:45), RLFARTRSMWSFNPETNILLNVPLHGTILTR PLLESELVIGAVILRGHLRIAGHHL (SEQ ID NO:46), NSSPDDQIGYY (SEQ ID NO:47), and RRGPEQTQGNFGDQELIRQGTDYKHWPQI AQFAPSASAFFGM (SEQ ID NO:48).

5. The immunogenic nanoparticle of claim 1, wherein said plurality of covid-19 CD8+ peptide epitopes comprises at least 1, or at least 2, or at least 3, or at least 4, or at least 5, or at least 6, or at least 7, or at least 8, or at least 9, or at least 10, or at least 11, or 12, amino acid sequences independently selected from the group consisting of LSFELLHAPATVCGP (SEQ ID NO:9), VNFNFNGLKGTGVLT (SEQ ID NO:10), PLLTDEMIAQYTSAL (SEQ ID NO: 11), KQLSSNFGAISSVLN (SEQ ID NO:12), FAMQMAYRFNGIGVT (SEQ ID NO:13), PTNFTISVTTEILPV (SEQ ID NO:14), FCGKGYHLMSFPQSA (SEQ ID NO:15), KLNDLCFTNVY (SEQ ID NO:16), TSNFRVQPTESIVRF (SEQ ID NO:17), NEVAVLYQDVNCTEV (SEQ ID NO:18), RGVYYPDKVFRSSV (SEQ ID NO:19), and GVFVSNGTHW (SEQ ID NO:20).

6. The immunogenic nanoparticle of claim 1, wherein said plurality of covid-19 CD8+ peptide epitopes antigens comprises at least 1, or at least 2, or at least 3, or at least 4, or at least 5, or at least 6, or at least 7, or at least 8, or at least 9, or at least 10, or at least 11, or at least 12, or at least 13, or at least 14, or 15 amino acid sequences selected from the group consisting of YLATALLTL (SEQ ID NO:243), WLMWLIINL (SEQ ID NO:245), SLPGVFCGV (SEQ ID NO:247), KLSYGIATV (SEQ ID NO:249), LLLDDFVEI (SEQ ID NO:251), FVFLVLLPL (SEQ ID NO:253), ALNTLVKQL (SEQ ID NO:255), RLQSLQTYV (SEQ ID NO:257), FIAGLIAIV (SEQ ID NO:259), FLAFVVFLL (SEQ ID NO:261), FLLNKEMYL (SEQ ID NO:263), IIFWFSLEL (SEQ ID NO:265), YIDIGNYTV (SEQ ID NO:267), YINVFAFPF (SEQ ID NO:269), and NVFAFPFTI (SEQ ID NO:271).

7.-9. (canceled)

10. The immunogenic nanoparticle of claim 1, wherein said nucleic acid encodes one or more CD8+ peptide epitopes selected from the group consisting of FLWLLWPV (SEQ ID NO:194), RNRFLYIIK (SEQ ID NO:195), ATRRIRGGD (SEQ ID NO:196), LWLLWPVTL (SEQ ID NO: 197), ENAFLPFAM(SEQ ID NO:198), ATATIPIQA (SEQ ID NO:199), LAAVYRINW (SEQ ID NO:200), LACFVLAAV (SEQ ID NO:201), FAMGIIAMS (SEQ ID NO:202), NLLLLFVTV(SEQ ID NO:203), TLACFVLAA (SEQ ID NO:204), VTLACFVLA (SEQ ID NO:205), ELLHAPATV (SEQ ID NO:206), RWVLSFEL (SEQ ID NO:207), YIIKLIFLW (SEQ ID NO:208), GIAIAMACL (SEQ ID NO:209), AMACLVGLM (SEQ ID NO:210), and AFLPFAMGI (SEQ ID NO:211).

11. The immunogenic nanoparticle of claim 1, wherein said nucleic acid encodes a peptide sequence that is an MHC-I targeting sequence, such as a ppCT signal peptide, upstream from said nucleic acid encoding one or more CD8+ T cell epitope.

12.-13. (canceled)

14. The immunogenic nanoparticle of claim 1, wherein said one or more CD4 T cell epitopes are CD4+ T-follicular epitopes.

15. (canceled)

16. The immunogenic nanoparticle of claim 14, wherein said nucleic acid encodes at least one, or at least 2, or at least 3, or at least 4, or at least 5, or at least 6, or at least 7, or at least 8, or at least 9, or at least 10 or at least 11, or 12 CD4+ T-follicular epitopes selected from the group consisting of ATRFASVYAWNRKRI (SEQ ID NO:165), ESNKKFLPFQQFGRD (SEQ ID NO:166), CSNLLLQYGSFCTQL (SEQ ID NO:167), LFNKVTLADAGFIKQ (SEQ ID NO:168), QKGLTVLPPLLTDEM (SEQ ID NO:169), AQALNTLVKQLSSKF (SEQ ID NO:170), WNRKRISNCVADYSV (SEQ ID NO:172), KKFLPFQQFGR (SEQ ID NO:173), LLQYGSF (SEQ ID NO:174), FIKQYGDCLGD (SEQ ID NO:175), LLTDEMIAQYTSALLAGTI (SEQ ID NO:176), and QALNTLVKQLS (SEQ ID NO:177).

17. (canceled)

18. The immunogenic nanoparticle of claim 1, wherein said nucleic acid encodes one or more CD4 epitopes selected from the group consisting of
(SEQ ID NO: 273)
KSAFYILPSIISNEK,
(SEQ ID NO: 275)
PNMLRIMASLVLARK,
(SEQ ID NO: 277)
LSYYKLGASQRVAGD,
(SEQ ID NO: 279)
AEILLIIMRTFKVSI,
(SEQ ID NO: 281)
DFYLCFLAFLLFLVL,
(SEQ ID NO: 283)
MKFLVFLGIITTVAA,
(SEQ ID NO: 212)
ACFVLAAVYRINWIT,
(SEQ ID NO: 213)
AFLPFAMGIIAMSAF,
(SEQ ID NO: 214)
FFLYENAFLPFAMGI,
(SEQ ID NO: 215)
FLPFAMGIIAMSAFA,
(SEQ ID NO: 216)
GIAIAMACLVGLMWL,
(SEQ ID NO: 217)
IGYYRRATRRIRGGD,
(SEQ ID NO: 218)
IKLIFLWLLWPVTLA,
(SEQ ID NO: 219)
KLIFLWLLWPVTLAC,
(SEQ ID NO: 220)
LPFAMGIIAMSAFAM,
(SEQ ID NO: 221)
NAFLPFAMGHAMSA,
(SEQ ID NO: 222)
PSDFVRATATIPIQA,
(SEQ ID NO: 223)
PVTLACFVLAAVYRI,
(SEQ ID NO: 224)
RNRFLYIIKLIFLWL,
(SEQ ID NO: 225)
RWVLSFELLHAPAT,
(SEQ ID NO: 226)
SDFVRATATIPIQAS,
(SEQ ID NO: 227)
T LAC FV LA AVY RI NW,
(SEQ ID NO: 228)
VHFVCNLLLLFVTVY,
(SEQ ID NO: 229)
WLSFELLHAPATVC
and
(SEQ ID NO: 230
YRWVLSFELLHAPA
shown in Table 33 (SEQ ID NOS: 273, 275, 277,
279, 281, and 283) and/or Table 31 (SEQ ID Nos:
212 230).

19. The immunogenic nanoparticle according to claim 1, wherein said nucleic acid encodes an MHC-II targeting peptide, such as TFR1-118 or Lil-80, upstream from said nucleic acid encoding said CD4+ T cell epitopes or CD4+ T-follicular epitopes.

20. (canceled)

21. The immunogenic nanoparticle according to claim 1, wherein said nucleic acid encodes at least 1, or at least 2, or at least 3, or at least 4, or at least 5, or 6 B cell epitopes selected from TRTQSPPAPPAYTNS (SEQ ID NO:128), SQCVNLTTRTQLPPAYTNSFTRGVY (SEQ ID NO:129), GTNGTKRFD (SEQ ID NO:130), FSNVTWFHAIHVSGTNGTKRFDN (SEQ ID NO:131), DPFLDVYYHKNNKSWME (SEQ ID NO:132), HKNNKSWMESEFRVYSSANN (SEQ ID NO:133), LTPGDSSSGWTAG (SEQ ID NO:134), LTPGDSSSGWTAG (SEQ ID NO:135), FPNITNLCPFDEVFNATRFASVYAWNRKRISNCVADYSVLYNFAPFFAFKCYGVSPT KLNDL (SEQ ID NO:136), RFASVYAWNRKRISNCVADY (SEQ ID NO:137), RGNEVSQIAPGQTGNIAD (SEQ ID NO:138), GQTGNIADYNYKLPDDFTG (SEQ ID NO:139), KLDSKVGGNYNY (SEQ ID NO:140), DSKVGGNYNYLYRLFRKSNL (SEQ ID NO:141), YQAGNKPCN (SEQ ID NO:142), YQAGS (SEQ ID NO:143), YGFRPTYGV (SEQ ID NO:144), PLRSYGFRPTYGVGHQPYRV (SEQ ID NO:145), FELLHAPATVCGPKKSTNLV (SEQ ID NO:146), LHAPATVCGPKKSTNLVKNK (SEQ ID NO:147), RDIADTTDAVRDP (SEQ ID NO:148), VCGPKKSTNLVKNKCVNFNFNGLTGTGVLTESNKKFLPFQQFGRDIADTTDAVRDP QTLEILDITPCSFGGVSVI (SEQ ID NO:149), VITPGTNTSN (SEQ ID NO:150), GTNTSNQVAVLYQDVNCTEVPVAIHADQLTPTWRVYSTGS (SEQ ID NO:151), ASYQTQTKSHRRARSVASQ (SEQ ID NO:152), CASYQTQTKSHRRARSVASQ (SEQ ID NO:153), LPDPSKPSKR (SEQ ID NO:154), ILPDPSKPSKRS (SEQ ID NO:155), VNNTVYDPLQPELDSFKEELDKYFKNHTSPDVDLGDISGI (SEQ ID NO:156) and DSFKEELDKYFKNHTSPDVD (SEQ ID NO:157).

22. (canceled)

23. The immunogenic nanoparticle according to claim 1, wherein said nucleic acid encodes a membrane targeting signal/secretory signal, such as MLLLLLLLLLLALALA (SEQ ID NO:1) or the prolactin peptide KGSPWKGSLLLLLMNIVSNLLCQSVAPLP (SEQ ID NO:6), upstream from said B cell epitopes.

24. (canceled)

25. The immunogenic nanoparticle according to claim 11, wherein a first cleavage site such as a T2A or a P2A cleavage site is disposed between said CD4 epitopes and said MHC-I targeting sequence upstream from said CD8+ T cell epitopes.

26. (canceled)

27. The immunogenic nanoparticle according to claim 23, wherein a second cleavage site such as a T2A or a P2A cleavage site is disposed between said CD8+ T cell epitopes and said membrane targeting signal/secretory signal upstream from said B cell epitopes.

28.-29. (canceled)

30. The immunogenic nanoparticle according to claim 1, wherein said nucleic acid encodes a spacer disposed between each of said epitopes wherein said spacer comprises an amino acid or a peptide.

31. The immunogenic nanoparticle of claim 30, wherein the spacer(s) disposed between said epitopes comprise or consist of an amino acid sequence independently selected from the group consisting of one or more lysines, AD, AQ, CG, DK, FG, FT, FV, GR, VI, LA, LH, LH, LP, LS, MA, NT, PN, PT, PV, QS, RE, RR, SF, SK, SR, TN, TQ, VE, VT, IC, LHVE, PNPT, ADLS, CGTN, PVVT, ICLP, LAMA, TQAQ, DKQS, LHRE, FVSK, VISF, GRNT, and RRFG.

32.-36. (canceled)

37. The immunogenic nanoparticle according to claim 1, wherein said nucleic acid encodes a furin cleavage site disposed between each epitope expressed by said nucleic acid sequence.

38. The immunogenic nanoparticle of claim 37, wherein said furin cleavage site comprises the amino acid sequence Arg-X-Lys/Arg-Arg (SEQ ID NO:94) where X is any amino acid.

39. The immunogenic nanoparticle of claim 38, wherein said furin cleavage site comprises the amino acid sequence RVRR (SEQ ID NO:95).

40. (canceled)

41. The immunogenic nanoparticle according to claim 1, wherein said nucleic acid encodes a cathepsin cleavage site disposed between each epitope encoded by said nucleic acid.

42. The immunogenic nanoparticle of claim 41, wherein said cathepsin cleavage site comprises the amino acid sequence PMGLP (SEQ ID NO:99).

43.-45. (canceled)

46. The immunogenic particle according to claim 1, wherein said nucleic acid comprises an mRNA.

47.-50. (canceled)

51. The immunogenic nanoparticle according to claim 1, wherein said nanoparticle is a lipidic nanoparticle (LNP).

52. The immunogenic nanoparticle of claim 51, wherein said nanoparticle comprises one or more cationic lipids selected from the group consisting of dilinoleylmethyl-4 dimethyl aminobutyrate (DLin-MC3-DMA), 1,2-dioleoyl-3-dimethylaminopropane (DODAP), didodecyl-dimethylammonium bromide (DDAB), 1,2-dioleoyloxy-3-trimethylammonium propane chloride (DOTAP), (N-[1-(2,3-dioleyloxy)propyl]-N,N,N-trimethylammonium chloride (DOTMA), 1,2-dilinoleyloxy-3-dimethylaminopropane (DLinDMA), 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA), C12-200 (Lipid 5), 3-(dimethylamino) propyl(12Z,15Z)-3-[(9Z,12Z)-octadeca-9,12-dien-1-yl]henicosa-12,15-dienoate (DMAP-BLP), and (2Z)-non-2-en-1-yl 10-[(Z)-(1-methylpiperidin-4 yl)carbonyloxy]nonadecanoate (L101), dicetylphosphate-tetraethylenepentaamine-based polycation lipid, YSK05, YSK12-C4, YSK13-C4, and YSK15-C4.

53. The immunogenic nanoparticle according to claim 51, wherein said lipidic nanoparticle comprises a helper lipid such as 1,2-dioleoyl-sn-glycero-3-phosphoethanolamine (DOPE), 1,2-distearoyl-sn-glycero-3-phosphocholine (DSPC), or 1-Palmitoyl-2-oleoyl-sn-glycero-3-phosphoethanolamine (POPE).

54. (canceled)

55. The immunogenic nanoparticle according to claim 51, wherein said lipidic nanoparticle comprises cholesterol.

56. The immunogenic nanoparticle according to claim 51, wherein said lipidic nanoparticle comprise a PEG-lipid.

57.-70. (canceled)

71. The immunogenic nanoparticle according to claim 1, wherein said nanoparticle comprises one or more biocompatible polymer(s).

72.-85. (canceled)

86. The immunogenic nanoparticle according to claim 1, wherein said nanoparticle further comprises an adjuvant.

87.-90. (canceled)

91. The immunogenic nanoparticle of claim 1, wherein said nanoparticle ranges in size from about 50 nm up to about 3 μm.

92.-95. (canceled)

96. The immunogenic nanoparticle according to claim 1, wherein said nanoparticles have one or more targeting moieties attached to the surface where said targeting moieties bind to and/or facilitate uptake by antigen presenting cells (APCs).

97. The immunogenic nanoparticle of claim 96, wherein said antigen presenting cells comprise one or more cells selected from the group consisting of macrophages, dendritic cells, and B cells.

98.-100. (canceled)

101. A pharmaceutical formulation comprising:
a population of immunogenic nanoparticles according to claim 1; and
a pharmaceutically acceptable carrier.

102. A method of inducing an immune response directed against SARS-Cov-2 in a mammal, said method comprising:
administering to said mammal an effective amount of the pharmaceutical formulation of claim 101.

103. A method for the prophylaxis or treatment of a SARS-Cov-2 infection in a mammal, said method comprising:
administering to said mammal an effective amount of the pharmaceutical formulation of claim 101.


SEQUENCE LISTING (338 sequences)

SEQ ID NO: 1 - protein, 4 aa, synthetic construct
  REGION 1..4: note = Spacer peptide
LHVE

SEQ ID NO: 2 - protein, 4 aa, synthetic construct
  REGION 1..4: note = Spacer peptide
PNPT

SEQ ID NO: 3 - protein, 4 aa, synthetic construct
  REGION 1..4: note = Spacer peptide
ADLS

SEQ ID NO: 4 - protein, 4 aa, synthetic construct
  REGION 1..4: note = Spacer peptide
CGTN

SEQ ID NO: 5 - protein, 4 aa, synthetic construct
  REGION 1..4: note = Spacer peptide
PVVT

SEQ ID NO: 6 - protein, 4 aa, synthetic construct
  REGION 1..4: note = Spacer peptide
ICLP

SEQ ID NO: 7 - protein, 4 aa, synthetic construct
  REGION 1..4: note = Spacer peptide
LAMA

SEQ ID NO: 8 - protein, 4 aa, synthetic construct
  REGION 1..4: note = Spacer peptide
TQAQ

SEQ ID NO: 9 - protein, 4 aa, synthetic construct
  REGION 1..4: note = Spacer peptide
DKQS

SEQ ID NO: 10 - protein, 4 aa, synthetic construct
  REGION 1..4: note = Spacer peptide
LHRE

SEQ ID NO: 11 - protein, 4 aa, synthetic construct
  REGION 1..4: note = Spacer peptide
FVSK

SEQ ID NO: 12 - protein, 4 aa, synthetic construct
  REGION 1..4: note = Spacer peptide
VISF

SEQ ID NO: 13 - protein, 4 aa, synthetic construct
  REGION 1..4: note = Spacer peptide
GRNT

SEQ ID NO: 14 - protein, 4 aa, synthetic construct
  REGION 1..4: note = Spacer peptide
RRFG

SEQ ID NO: 15 - protein, 4 aa, synthetic construct
  REGION 1..4: note = Spacer peptide
LLLL

SEQ ID NO: 16 - protein, 16 aa, synthetic construct
  REGION 1..16: note = Secretory signal peptide
MLLLLLLLLLLALALA

SEQ ID NO: 17 - protein, 10 aa, synthetic construct
  REGION 1..10: note = ppCT signal peptide
VLLQAGSLHA

SEQ ID NO: 18 - protein, 20 aa, synthetic construct
  REGION 1..20: note = Self cleaving linker peptide
GSGEGRGSLLTCGDVEENPG

SEQ ID NO: 19 - protein, 20 aa, synthetic construct
  REGION 1..20: note = Self cleaving linker peptide
GSGEGRGSLLTCGDVEENPG

SEQ ID NO: 20 - protein, 29 aa, synthetic construct
  REGION 1..29: note = Secretory signal peptide
KGSPWKGSLLLLLMNIVSNLLCQSVAPLP

SEQ ID NO: 21 - protein, 1273 aa, Betacoronovirus SARS-CoV-2
MFVFLVLLPLVSSQCVNLTTRTQSPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - protein, 15 aa, Betacoronovirus SARS-CoV-2
LSFELLHAPATVCGP

SEQ ID NO: 23 - protein, 15 aa, Betacoronovirus SARS-CoV-2
VNFNFNGLKGTGVLT

SEQ ID NO: 24 - protein, 15 aa, Betacoronovirus SARS-CoV-2
PLLTDEMIAQYTSAL

SEQ ID NO: 25 - protein, 15 aa, Betacoronovirus SARS-CoV-2
KQLSSNFGAISSVLN

SEQ ID NO: 26 - protein, 15 aa, Betacoronovirus SARS-CoV-2
FAMQMAYRFNGIGVT

SEQ ID NO: 27 - protein, 15 aa, Betacoronovirus SARS-CoV-2
PTNFTISVTTEILPV

SEQ ID NO: 28 - protein, 15 aa, Betacoronovirus SARS-CoV-2
FCGKGYHLMSFPQSA

SEQ ID NO: 29 - protein, 11 aa, Betacoronovirus SARS-CoV-2
KLNDLCFTNVY

SEQ ID NO: 30 - protein, 15 aa, Betacoronovirus SARS-CoV-2
TSNFRVQPTESIVRF

SEQ ID NO: 31 - protein, 15 aa, Betacoronovirus SARS-CoV-2
NEVAVLYQDVNCTEV

SEQ ID NO: 32 - protein, 14 aa, Betacoronovirus SARS-CoV-2
RGVYYPDKVFRSSV

SEQ ID NO: 33 - protein, 10 aa, Betacoronovirus SARS-CoV-2
GVFVSNGTHW

SEQ ID NO: 34 - protein, 1273 aa, Betacoronovirus SARS-CoV-2
MFVFLVLLPLVSSQCVNLTTRTQSPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 35 - protein, 14 aa, Betacoronovirus SARS-CoV-2
LSFELLHAPATVCG

SEQ ID NO: 36 - protein, 8 aa, Betacoronovirus SARS-CoV-2
VNFNFNGL

SEQ ID NO: 37 - protein, 14 aa, Betacoronovirus SARS-CoV-2
PLLTDEMIAQYTSA

SEQ ID NO: 38 - protein, 14 aa, Betacoronovirus SARS-CoV-2
KQLSSKFGAISSVL

SEQ ID NO: 39 - protein, 14 aa, Betacoronovirus SARS-CoV-2
FAMQMAYRFNGIGV

SEQ ID NO: 40 - protein, 14 aa, Betacoronovirus SARS-CoV-2
PTNFTISVTTEILP

SEQ ID NO: 41 - protein, 14 aa, Betacoronovirus SARS-CoV-2
FCGKGYHLMSFPQS

SEQ ID NO: 42 - protein, 11 aa, Betacoronovirus SARS-CoV-2
KLNDLCFTNVY

SEQ ID NO: 43 - protein, 14 aa, Betacoronovirus SARS-CoV-2
TSNFRVQPTESIVR

SEQ ID NO: 44 - protein, 14 aa, Betacoronovirus SARS-CoV-2
NQVAVLYQGVNCTE

SEQ ID NO: 45 - protein, 14 aa, Betacoronovirus SARS-CoV-2
RGVYYPDKVFRSSV

SEQ ID NO: 46 - protein, 10 aa, Betacoronovirus SARS-CoV-2
GVFVSNGTHW

SEQ ID NO: 47 - protein, 14 aa, Betacoronovirus SARS-CoV-2
RGVYYPDKVFRSSV

SEQ ID NO: 48 - protein, 20 aa, Betacoronovirus SARS-CoV-2
KGIYQTSNFRVQPTESIVRF

SEQ ID NO: 49 - protein, 11 aa, Betacoronovirus SARS-CoV-2
KLNDLCFTNVY

SEQ ID NO: 50 - protein, 10 aa, Betacoronovirus SARS-CoV-2
FELLHAPATV

SEQ ID NO: 51 - protein, 17 aa, Betacoronovirus SARS-CoV-2
TSNEVAVLYQDVNCTEV

SEQ ID NO: 52 - protein, 18 aa, Betacoronovirus SARS-CoV-2
TEILPVSMTKTSVDCTMY

SEQ ID NO: 53 - protein, 15 aa, Betacoronovirus SARS-CoV-2
PLLTDEMIAQYTSAL

SEQ ID NO: 54 - protein, 8 aa, Betacoronovirus SARS-CoV-2
YRFNGIGV

SEQ ID NO: 55 - protein, 27 aa, Betacoronovirus SARS-CoV-2
ALNTLVKQLSSNFGAISSVLNDILSRL

SEQ ID NO: 56 - protein, 25 aa, Betacoronovirus SARS-CoV-2
KRVDFCGKGYHLMSFPQSAPHGVVF

SEQ ID NO: 57 - protein, 10 aa, Betacoronovirus SARS-CoV-2
GVFVSNGTHW

SEQ ID NO: 58 - protein, 31 aa, Betacoronovirus SARS-CoV-2
NPLLYDANYFLCWHTNCYDYCIPYNSVTSSI

SEQ ID NO: 59 - protein, 56 aa, Betacoronovirus SARS-CoV-2
RLFARTRSMWSFNPETNILLNVPLHGTILTRPLLESELVIGAVILRGHLRIAGHHL

SEQ ID NO: 60 - protein, 11 aa, Betacoronovirus SARS-CoV-2
NSSPDDQIGYY

SEQ ID NO: 61 - protein, 42 aa, Betacoronovirus SARS-CoV-2
RRGPEQTQGNFGDQELIRQGTDYKHWPQIAQFAPSASAFFGM

SEQ ID NO: 62 - protein, 10 aa, synthetic construct
  REGION 1..10: note = Leader signal peptide
VLLQAGSLHA

SEQ ID NO: 63 - protein, 9 aa, synthetic construct
  REGION 1..9: note = TEIPP antigen peptide
MCLRMTAVM

SEQ ID NO: 64 - protein, 9 aa, synthetic construct
  REGION 1..9: note = TEIPP antigen peptide
CVNKTERAY

SEQ ID NO: 65 - protein, 10 aa, synthetic construct
  REGION 1..10: note = TEIPP antigen peptide
FAPLPRLPTL

SEQ ID NO: 66 - protein, 9 aa, synthetic construct
  REGION 1..9: note = TEIPP antigen peptide
FYAEATPML

SEQ ID NO: 67 - protein, 10 aa, synthetic construct
  REGION 1..10: note = TEIPP antigen peptide
VLLQAGSLHA

SEQ ID NO: 68 - protein, 9 aa, synthetic construct
  REGION 1..9: note = TEIPP antigen peptide
AAGIGILTV

SEQ ID NO: 69 - protein, 18 aa, synthetic construct
  REGION 1..18: note = SARS Cov-2 antigen with flanking sequences
FTRGVYYPDKVFRSSVLH

SEQ ID NO: 70 - DNA, 54 bp, synthetic construct
  misc_feature 1..54: note = DNA encoding SARS Cov-2 antigen with flanking sequences
  source 1..54: mol_type = other DNA
tttacccgcggcgtgtattatccggataaagtgtttcgcagcagcgtgctgcat

SEQ ID NO: 71 - protein, 24 aa, synthetic construct
  REGION 1..24: note = SARS Cov-2 antigen with flanking sequences
VEKGIYQTSNFRVQPTESIVRFPN

SEQ ID NO: 72 - DNA, 72 bp, synthetic construct
  misc_feature 1..72: note = DNA encoding SARS Cov-2 antigen with flanking sequences
  source 1..72: mol_type = other DNA
gtggaaaaaggcatttatcagaccagcaactttcgcgtgcagccgaccgaaagcattgtgcgctttccgaac

SEQ ID NO: 73 - protein, 15 aa, synthetic construct
  REGION 1..15: note = SARS Cov-2 antigen with flanking sequences
PTKLNDLCFTNVYAD

SEQ ID NO: 74 - DNA, 45 bp, synthetic construct
  misc_feature 1..45: note = DNA encoding SARS Cov-2 antigen with flanking sequences
  source 1..45: mol_type = other DNA
ccgaccaaactgaacgatctgtgctttaccaacgtgtatgcggat

SEQ ID NO: 75 - protein, 14 aa, synthetic construct
  REGION 1..14: note = SARS Cov-2 antigen with flanking sequences
LSFELLHAPATVCG

SEQ ID NO: 76 - DNA, 42 bp, synthetic construct
  misc_feature 1..42: note = DNA encoding SARS Cov-2 antigen with flanking sequences
  source 1..42: mol_type = other DNA
ctgagctttgaactgctgcatgcgccggcgaccgtgtgcggc

SEQ ID NO: 77 - protein, 21 aa, synthetic construct
  REGION 1..21: note = SARS Cov-2 antigen with flanking sequences
TNTSNEVAVLYQDVNCTEVPV

SEQ ID NO: 78 - DNA, 63 bp, synthetic construct
  misc_feature 1..63: note = DNA encoding SARS Cov-2 antigen with flanking sequences
  source 1..63: mol_type = other DNA
accaacaccagcaacgaagtggcggtgctgtatcaggatgtgaactgcaccgaagtgccggtg

SEQ ID NO: 79 - protein, 22 aa, synthetic construct
  REGION 1..22: note = SARS Cov-2 antigen with flanking sequences
VTTEILPVSMTKTSVDCTMYIC

SEQ ID NO: 80 - DNA, 66 bp, synthetic construct
  misc_feature 1..66: note = DNA encoding SARS Cov-2 antigen with flanking sequences
  source 1..66: mol_type = other DNA
gtgaccaccgaaattctgccggtgagcatgaccaaaaccagcgtggattgcaccatgtatatttgc

SEQ ID NO: 81 - protein, 19 aa, synthetic construct
  REGION 1..19: note = SARS Cov-2 antigen with flanking sequences
LPPLLTDEMIAQYTSALLA

SEQ ID NO: 82 - DNA, 57 bp, synthetic construct
  misc_feature 1..57: note = DNA encoding SARS Cov-2 antigen with flanking sequences
  source 1..57: mol_type = other DNA
ctgccgccgctgctgaccgatgaaatgattgcgcagtataccagcgcgctgctggcg

SEQ ID NO: 83 - protein, 12 aa, synthetic construct
  REGION 1..12: note = SARS Cov-2 antigen with flanking sequences
MAYRFNGIGVTQ

SEQ ID NO: 84 - DNA, 36 bp, synthetic construct
  misc_feature 1..36: note = DNA encoding SARS Cov-2 antigen with flanking sequences
  source 1..36: mol_type = other DNA
atggcgtatcgctttaacggcattggcgtgacccag

SEQ ID NO: 85 - protein, 31 aa, synthetic construct
  REGION 1..31: note = SARS Cov-2 antigen with flanking sequences
AQALNTLVKQLSSNFGAISSVLNDILSRLDK

SEQ ID NO: 86 - DNA, 93 bp, synthetic construct
  misc_feature 1..93: note = DNA encoding SARS Cov-2 antigen with flanking sequences
  source 1..93: mol_type = other DNA
gcgcaggcgctgaacaccctggtgaaacagctgagcagcaactttggcgcgattagcagcgtgctgaacgatattctgagccgcctggataaa

SEQ ID NO: 87 - protein, 29 aa, synthetic construct
  REGION 1..29: note = SARS Cov-2 antigen with flanking sequences
QSKRVDFCGKGYHLMSFPQSAPHGVVFLH

SEQ ID NO: 88 - DNA, 87 bp, synthetic construct
  misc_feature 1..87: note = DNA encoding SARS Cov-2 antigen with flanking sequences
  source 1..87: mol_type = other DNA
cagagcaaacgcgtggatttttgcggcaaaggctatcatctgatgagctttccgcagagcgcgccgcatggcgtggtgtttctgcat

SEQ ID NO: 89 - protein, 14 aa, synthetic construct
  REGION 1..14: note = SARS Cov-2 antigen with flanking sequences
REGVFVSNGTHWFV

SEQ ID NO: 90 - DNA, 42 bp, synthetic construct
  misc_feature 1..42: note = DNA encoding SARS Cov-2 antigen with flanking sequences
  source 1..42: mol_type = other DNA
cgcgaaggcgtgtttgtgagcaacggcacccattggtttgtg

SEQ ID NO: 91 - protein, 35 aa, synthetic construct
  REGION 1..35: note = SARS Cov-2 antigen with flanking sequences
SKNPLLYDANYFLCWHTNCYDYCIPYNSVTSSIVI

SEQ ID NO: 92 - DNA, 105 bp, synthetic construct
  misc_feature 1..105: note = DNA encoding SARS Cov-2 antigen with flanking sequences
  source 1..105: mol_type = other DNA
agcaaaaacccgctgctgtatgatgcgaactattttctgtgctggcataccaactgctatgattattgcattccgtataacagcgtgaccagcagcattgtgatt

SEQ ID NO: 93 - protein, 60 aa, synthetic construct
  REGION 1..60: note = SARS Cov-2 antigen with flanking sequences
SFRLFARTRSMWSFNPETNILLNVPLHGTILTRPLLESELVIGAVILRGHLRIAGHHLGR

SEQ ID NO: 94 - DNA, 180 bp, synthetic construct
  misc_feature 1..180: note = DNA encoding SARS Cov-2 antigen with flanking sequences
  source 1..180: mol_type = other DNA
agctttcgcctgtttgcgcgcacccgcagcatgtggagctttaacccggaaaccaacattctgctgaacgtgccgctgcatggcaccattctgacccgcccgctgctggaaagcgaactggtgattggcgcggtgattctgcgcggccatctgcgcattgcgggccatcatctgggccgc

SEQ ID NO: 95 - protein, 15 aa, synthetic construct
  REGION 1..15: note = SARS Cov-2 antigen with flanking sequences
NTNSSPDDQIGYYRR

SEQ ID NO: 96 - DNA, 45 bp, synthetic construct
  misc_feature 1..45: note = DNA encoding SARS Cov-2 antigen with flanking sequences
  source 1..45: mol_type = other DNA
aacaccaacagcagcccggatgatcagattggctattatcgccgc

SEQ ID NO: 97 - protein, 46 aa, synthetic construct
  REGION 1..46: note = SARS Cov-2 antigen with flanking sequences
FGRRGPEQTQGNFGDQELIRQGTDYKHWPQIAQFAPSASAFFGMSR

SEQ ID NO: 98 - DNA, 138 bp, synthetic construct
  misc_feature 1..138: note = DNA encoding SARS Cov-2 antigen with flanking sequences
  source 1..138: mol_type = other DNA
tttggccgccgcggcccggaacagacccagggcaactttggcgatcaggaactgattcgccagggcaccgattataaacattggccgcagattgcgcagtttgcgccgagcgcgagcgcgttttttggcatgagccgc

SEQ ID NO: 99 - protein, 375 aa, synthetic construct
  REGION 1..375: note = Peptide sequence comprising multiple SARS-Cov-2 epitopes
FTRGVYYPDKVFRSSVLHVEKGIYQTSNFRVQPTESIVRFPNPTKLNDLCFTNVYADLSFELLHAPATVCGTNTSNEVAVLYQDVNCTEVPVVTTEILPVSMTKTSVDCTMYICLPPLLTDEMIAQYTSALLAMAYRFNGIGVTQAQALNTLVKQLSSNFGAISSVLNDILSRLDKQSKRVDFCGKGYHLMSFPQSAPHGVVFLHREGVFVSNGTHWFVSKNPLLYDANYFLCWHTNCYDYCIPYNSVTSSIVISFRLFARTRSMWSFNPETNILLNVPLHGTILTRPLLESELVIGAVILRGHLRIAGHHLGRNTNSSPDDQIGYYRRFGRRGPEQTQGNFGDQELIRQGTDYKHWPQIAQFAPSASAFFGMSR

SEQ ID NO: 100 - DNA, 1098 bp, synthetic construct
  misc_feature 1..1098: note = DNA encoding peptide sequence comprising multiple SARS-Cov-2 epitopes
  source 1..1098: mol_type = other DNA
ttacccgcggcgtgtattatccggataaagtgtttcgcagcagcgtgctgcatgtggaaaaaggcatttatcagaccagcaactttcgcgtgcagccgaccgaaagcattgtgcgctttccgaacccgaccaaactgaacgatctgtgctttaccaacgtgtatgcggatctgagctttgaactgctgcatgcgccggcgaccgtgtgcggcaccaacaccagcaacgaagtggcggtgctgtatcaggatgtgaactgcaccgaagtgccggtggtgaccaccgaaattctgccggtgagcatgaccaaaaccagcgtggattgcaccatgtatatttgcctgccgccgctgctgaccgatgaaatgattgcgcagtataccagcgcgctgctggcgatggcgtatcgctttaacggcattggcgtgacccaggcgcaggcgctgaacaccctggtgaaacagctgagcagcaactttggcgcgattagcagcgtgctgaacgatattctgagccgcctggataaacagagcaaacgcgtggatttttgcggcaaaggctatcatctgatgagctttccgcagagcgcgccgcatggcgtggtgtttctgcatcgcgaaggcgtgtttgtgagcaacggcacccattggtttgtgagcaaaaacccgctgctgtatgatgcgaactattttctgtgctggcataccaactgctatgattattgcattccgtataacagcgtgaccagcagcattgtgattagctttcgcctgtttgcgcgcacccgcagcatgtggagctttaacccggaaaccaacattctgctgaacgtgccgctgcatggcaccattctgacccgcccgctgctggaaagcgaactggtgattggcgcggtgattctgcgcggccatctgcgcattgcgggccatcatctgggccgcaacaccaacagcagcccggatgatcagattggctattatcgccgctttggccgccgcggcccggaacagacccagggcaactttggcgatcaggaactgattcgccagggcaccgattataaacattggccgcagattgcgcagtttgcgccgagcg

SEQ ID NO: 101 - DNA, 1131 bp, synthetic construct
  misc_feature 1..1131: note = DNA encoding peptide sequence comprising multiple SARS-Cov-2 epitopes
  source 1..1131: mol_type = other DNA
atgtttacccgcggcgtgtattatccggataaagtgtttcgcagcagcgtgctgcatgtggaaaaaggcatttatcagaccagcaactttcgcgtgcagccgaccgaaagcattgtgcgctttccgaacccgaccaaactgaacgatctgtgctttaccaacgtgtatgcggatctgagctttgaactgctgcatgcgccggcgaccgtgtgcggcaccaacaccagcaacgaagtggcggtgctgtatcaggatgtgaactgcaccgaagtgccggtggtgaccaccgaaattctgccggtgagcatgaccaaaaccagcgtggattgcaccatgtatatttgcctgccgccgctgctgaccgatgaaatgattgcgcagtataccagcgcgctgctggcgatggcgtatcgctttaacggcattggcgtgacccaggcgcaggcgctgaacaccctggtgaaacagctgagcagcaactttggcgcgattagcagcgtgctgaacgatattctgagccgcctggataaacagagcaaacgcgtggatttttgcggcaaaggctatcatctgatgagctttccgcagagcgcgccgcatggcgtggtgtttctgcatcgcgaaggcgtgtttgtgagcaacggcacccattggtttgtgagcaaaaacccgctgctgtatgatgcgaactattttctgtgctggcataccaactgctatgattattgcattccgtataacagcgtgaccagcagcattgtgattagctttcgcctgtttgcgcgcacccgcagcatgtggagctttaacccggaaaccaacattctgctgaacgtgccgctgcatggcaccattctgacccgcccgctgctggaaagcgaactggtgattggcgcggtgattctgcgcggccatctgcgcattgcgggccatcatctgggccgcaacaccaacagcagcccggatgatcagattggctattatcgccgctttggccgccgcggcccggaacagacccagggcaactttggcgatcaggaactgattcgccagggcaccgattataaacattggccgcagattgcgcagtttgcgccgagcgcgagcgcgttttttggcatgagccgctaa

SEQ ID NO: 102 - DNA, 1130 bp, synthetic construct
  misc_feature 1..1130: note = DNA encoding peptide sequence comprising multiple SARS-Cov-2 epitopes
  source 1..1130: mol_type = other DNA
atgttcacccggggggtgtactatcctgataaggtgttcagatccagcgtgctgcacgtggaaaagggcatctaccagaccagcaatttccgcgtgcagcctaccgaatctatcgtgcggttccccaaccccaccaagctgaacgatctgtgttttaccaatgtgtacgccgacctgtctttcgagctgctgcacgcccctgccaccgtgtgtggcacaaacacaagcaacgaggtggccgtgctgtaccaggacgtgaactgtacagaggttcctgtggttacaaccgagatcctccccgtgtccatgacaaaaacctccgtcgactgcaccatgtacatctgcctgccacctctgctgacagatgagatgatcgcccaatacacctccgccctgctggccatggcttacagattcaatggcattggagtgacccaggcccaggccctgaataccctggtgaagcagctgagctctaatttcggcgccatctctagcgtgctgaacgacatcctgagccggctggacaaacagagcaagcgggtggatttctgcggcaagggatatcacctgatgagcttccctcaaagcgccccccacggcgtggtgttcctgcatagggaaggcgtctttgtgtctaacggcacccactggttcgtgtccaagaaccccctgctgtacgacgccaactacttcctgtgctggcacaccaactgctacgactactgcatcccctacaacagcgtgaccagcagcatcgtgattagctttagactgttcgccagaaccagaagtatgtggagctttaacccagagacaaacatcctgctgaacgtgcctctgcacggcacaatccttacaagacctctgctggaaagcgagctggtcatcggcgctgtgatcctgcggggacacctgagaatcgccggccaccacctgggcagaaacaccaacagcagccctgacgaccagatcggctactaccggagattcggcagaagaggtcctgaacagacccaaggcaacttcggcgatcaggagctcatcagacagggcaccgactacaagcactggcctcagatcgcccagttcgcacctagcgcttctgcttttttcgaatgagcagatga

SEQ ID NO: 103 - protein, 12 aa, synthetic construct
  REGION 1..12: note = Peptide leader sequence
VLLQAGSLHAKK

SEQ ID NO: 104 - DNA, 30 bp, synthetic construct
  misc_feature 1..30: note = DNA encoding peptide leader sequence
  source 1..30: mol_type = other DNA
gtgctgctgcaggcgggcagcctgcatgcg

SEQ ID NO: 105 - DNA, 1167 bp, synthetic construct
  misc_feature 1..1167: note = DNA encoding peptide sequence comprising multiple SARS-Cov-2 epitopes
  source 1..1167: mol_type = other DNA
atggtgctgctgcaggcgggcagcctgcatgcgaaaaaatttacccgcggcgtgtattatccggataaagtgtttcgcagcagcgtgctgcatgtggaaaaaggcatttatcagaccagcaactttcgcgtgcagccgaccgaaagcattgtgcgctttccgaacccgaccaaactgaacgatctgtgctttaccaacgtgtatgcggatctgagctttgaactgctgcatgcgccggcgaccgtgtgcggcaccaacaccagcaacgaagtggcggtgctgtatcaggatgtgaactgcaccgaagtgccggtggtgaccaccgaaattctgccggtgagcatgaccaaaaccagcgtggattgcaccatgtatatttgcctgccgccgctgctgaccgatgaaatgattgcgcagtataccagcgcgctgctggcgatggcgtatcgctttaacggcattggcgtgacccaggcgcaggcgctgaacaccctggtgaaacagctgagcagcaactttggcgcgattagcagcgtgctgaacgatattctgagccgcctggataaacagagcaaacgcgtggatttttgcggcaaaggctatcatctgatgagctttccgcagagcgcgccgcatggcgtggtgtttctgcatcgcgaaggcgtgtttgtgagcaacggcacccattggtttgtgagcaaaaacccgctgctgtatgatgcgaactattttctgtgctggcataccaactgctatgattattgcattccgtataacagcgtgaccagcagcattgtgattagctttcgcctgtttgcgcgcacccgcagcatgtggagctttaacccggaaaccaacattctgctgaacgtgccgctgcatggcaccattctgacccgcccgctgctggaaagcgaactggtgattggcgcggtgattctgcgcggccatctgcgcattgcgggccatcatctgggccgcaacaccaacagcagcccggatgatcagattggctattatcgccgctttggccgccgcggcccggaacagacccagggcaactttggcgatcaggaactgattcgccagggcaccgattataaacattggccgcagattgcgcagtttgcgccgagcgcgagcgcgttttttggcatgagccgctaa

SEQ ID NO: 106 - DNA, 1167 bp, synthetic construct
  misc_feature 1..1167: note = DNA encoding peptide sequence comprising multiple SARS-Cov-2 epitopes
  source 1..1167: mol_type = other DNA
atggtgctgctgcaggccggcagcctccacgccaagaagttcaccagaggcgtgtattaccccgacaaggtgtttagaagcagcgtgctgcacgtggaaaagggcatctaccaaacaagcaacttccgggtgcagcctaccgaaagcatcgtgcggtttccaaaccctacaaaactgaacgacctgtgttttaccaatgtgtacgccgacctgagcttcgagctgctgcatgctcctgccacagtgtgcggcaccaacacatctaacgaggtggccgtgctgtaccaggacgtgaactgcacagaagtgcctgtggtgacaaccgagatcctgcctgtgagcatgaccaagaccagcgtggattgcaccatgtacatctgcctgccccccctgctcaccgacgagatgatcgcccagtacacctctgccctgctggccatggcctacagattcaacggaatcggcgttacacaggcccaggccctgaacaccctggtgaaacagctgagcagcaacttcggcgctatctcttccgtgctgaatgacattctgagcagactggacaagcagagcaagcgggtggatttctgcggcaaaggctaccacctgatgtctttcccccaaagcgccccccacggcgtcgtgttcctgcatagagagggcgtgttcgtgtccaacggcacacactggttcgtgagcaagaaccctctgctttacgatgccaactacttcctgtgttggcacaccaactgctacgactactgtatcccttataacagcgtgacaagctccatcgttatttcttttcggctgttcgctagaacccggagcatgtggtccttcaatccagaaaccaacatcctgctgaacgtccccctgcacgggaccatcctgaccagacctctgctggaaagcgagctggttatcggcgccgtgatcctgcgcggccacctgagaatcgccggccaccaccttggacgaaacaccaatagcagccctgatgaccagatcggctactaccggagattcggcagaaggggacctgagcagacccagggcaattttggagatcaggagctgatcagacagggaacagactacaagcactggcctcaaatcgcccagttcgccccttctgcatctgccttcttcggcatgagcagataa

SEQ ID NO: 107 - skipped

SEQ ID NO: 108 - protein, 4 aa, synthetic construct
  REGION 1..4: note = Furin cleavage site
RVRR

SEQ ID NO: 109 - DNA, 12 bp, synthetic construct
  misc_feature 1..12: note = DNA encoding furin cleavage site.
  source 1..12: mol_type = other DNA
cgcgtgcgccgc

SEQ ID NO: 110 - DNA, 1335 bp, synthetic construct
  misc_feature 1..1335: note = DNA encoding peptide sequence comprising multiple SARS-Cov-2 epitopes
  source 1..1335: mol_type = other DNA
atggtgctgctgcaggcgggcagcctgcatgcgaaaaaatttacccgcggcgtgtattatccggataaagtgtttcgcagcagcgtgctgcatcgcgtgcgccgcgtggaaaaaggcatttatcagaccagcaactttcgcgtgcagccgaccgaaagcattgtgcgctttccgaaccgcgtgcgccgcccgaccaaactgaacgatctgtgctttaccaacgtgtatgcggatcgcgtgcgccgcctgagctttgaactgctgcatgcgccggcgaccgtgtgcggccgcgtgcgccgcaccaacaccagcaacgaagtggcggtgctgtatcaggatgtgaactgcaccgaagtgccggtgcgcgtgcgccgcgtgaccaccgaaattctgccggtgagcatgaccaaaaccagcgtggattgcaccatgtatatttgccgcgtgcgccgcctgccgccgctgctgaccgatgaaatgattgcgcagtataccagcgcgctgctggcgcgcgtgcgccgcatggcgtatcgctttaacggcattggcgtgacccagcgcgtgcgccgcgcgcaggcgctgaacaccctggtgaaacagctgagcagcaactttggcgcgattagcagcgtgctgaacgatattctgagccgcctggataaacgcgtgcgccgccagagcaaacgcgtggatttttgcggcaaaggctatcatctgatgagctttccgcagagcgcgccgcatggcgtggtgtttctgcatcgcgtgcgccgccgcgaaggcgtgtttgtgagcaacggcacccattggtttgtgcgcgtgcgccgcagcaaaaacccgctgctgtatgatgcgaactattttctgtgctggcataccaactgctatgattattgcattccgtataacagcgtgaccagcagcattgtgattcgcgtgcgccgcagctttcgcctgtttgcgcgcacccgcagcatgtggagctttaacccggaaaccaacattctgctgaacgtgccgctgcatggcaccattctgacccgcccgctgctggaaagcgaactggtgattggcgcggtgattctgcgcggccatctgcgcattgcgggccatcatctgggccgccgcgtgcgccgcaacaccaacagcagcccggatgatcagattggctattatcgccgccgcgtgcgccgctttggccgccgcggcccggaacagacccagggcaactttggcgatcaggaactgattcgccagggcaccgattataaacattggccgcagattgcgcagtttgcgccgagcgcgagcgcgttttttggcatgagccgctaa

SEQ ID NO: 111 - DNA, 1335 bp, synthetic construct
  misc_feature 1..1335: note = DNA encoding peptide sequence comprising multiple SARS-Cov-2 epitopes
  source 1..1335: mol_type = other DNA
atggtactgctgcaggctggcagcctgcatgccaagaaattcaccagaggggtttactatcctgacaaggtgttcagaagcagcgtcctgcacagagtgagacgcgtggaaaagggaatctaccagacaagcaacttccgggtgcagcccactgagagcatcgtgagattcccaaacagagtgagaagacctaccaaactgaatgatctgtgttttacaaatgtgtacgccgatcgggtgagaagactgagcttcgagctgctgcacgccccagccacagtctgcggccgagtgcgcaggaccaatacctctaacgaggtggccgtgctgtaccaggacgtgaactgtacagaggtgcccgtgagagtgagacgggtgaccacagaaatcctgcctgtgtctatgaccaagacaagcgtggactgcaccatgtacatctgtagagtgagaagactgcctcctctgctgacagacgagatgatcgctcaatacacctctgctctgctggccagagttcgtagaatggcctaccggtttaacggcatcggcgtgacccagagagtgcgccgggcccaggccctgaacacactggtcaagcagctgagcagcaacttcggagccatcagctctgtgctgaacgacatcctcagcagactggacaagcgggtgagacgccagagcaagagggtggacttctgcggcaagggctaccacctcatgagcttcccccagtctgctcctcacggcgtggtgttcctgcatagagtgagaagacgggaaggcgtgtttgtgtccaacggcacccactggttcgtgcgggtacggcggagcaaaaaccccctgctgtacgacgccaactacttcctgtgctggcacaccaactgctacgactactgcatcccctacaacagcgtcaccagttctattgtgatccgggtccggcgttcctttagactgttcgccaggacccggtccatgtggtccttcaatcctgaaaccaatatcctgctgaacgtgccactgcacggcaccatcctgaccagacccctgctggaatccgagctggtgatcggcgccgtgatccttagaggacacctgcggatcgccggccaccacctgggaagacgggtgcggagaaacaccaacagcagccctgatgatcagattggatattacagaagaagagtgagacggttcggcagacggggccctgagcagacacagggcaacttcggcgatcaggagctgatcagacaaggcaccgactacaagcactggcctcagatcgcccaatttgctcctagcgccagcgccttcttcggcatgagcagatga

SEQ ID NO: 112 - protein, 5 aa, synthetic construct
  REGION 1..5: note = Cathepsin S cleavage site
PMGLP

SEQ ID NO: 113 - DNA, 15 bp, synthetic construct
  misc_feature 1..15: note = DNA encoding cathepsin S cleavage site
  source 1..15: mol_type = other DNA
ccgatgggcctgccg

SEQ ID NO: 114 - protein, 15 aa, Betacoronovirus SARS-CoV-2
LSFELLHAPATVCGP

SEQ ID NO: 115 - DNA, 45 bp, Betacoronovirus SARS-CoV-2
  source 1..45: mol_type = other DNA
ctgagctttgaactgctgcatgcgccggcgaccgtgtgcggcccg

SEQ ID NO: 116 - protein, 15 aa, Betacoronovirus SARS-CoV-2
PLLTDEMIAQYTSAL

SEQ ID NO: 117 - DNA, 45 bp, Betacoronovirus SARS-CoV-2
  source 1..45: mol_type = other DNA
ccgctgctgaccgatgaaatgattgcgcagtataccagcgcgctg

SEQ ID NO: 118 - protein, 15 aa, Betacoronovirus SARS-CoV-2
KQLSSNFGAISSVLN

SEQ ID NO: 119 - DNA, 45 bp, Betacoronovirus SARS-CoV-2
  source 1..45: mol_type = other DNA
aaacagctgagcagcaactttggcgcgattagcagcgtgctgaac

SEQ ID NO: 120 - protein, 15 aa, Betacoronovirus SARS-CoV-2
FAMQMAYRFNGIGVT

SEQ ID NO: 121 - DNA, 45 bp, Betacoronovirus SARS-CoV-2
  source 1..45: mol_type = other DNA
tttgcgatgcagatggcgtatcgctttaacggcattggcgtgacc

SEQ ID NO: 122 - protein, 15 aa, Betacoronovirus SARS-CoV-2
PTNFTISVTTEILPV

SEQ ID NO: 123 - DNA, 45 bp, Betacoronovirus SARS-CoV-2
  source 1..45: mol_type = other DNA
ccgaccaactttaccattagcgtgaccaccgaaattctgccggtg

SEQ ID NO: 124 - protein, 15 aa, Betacoronovirus SARS-CoV-2
FCGKGYHLMSFPQSA

SEQ ID NO: 125 - DNA, 45 bp, Betacoronovirus SARS-CoV-2
  source 1..45: mol_type = other DNA
ttttgcggcaaaggctatcatctgatgagctttccgcagagcgcg

SEQ ID NO: 126 - protein, 11 aa, Betacoronovirus SARS-CoV-2
KLNDLCFTNVY

SEQ ID NO: 127 - DNA, 33 bp, Betacoronovirus SARS-CoV-2
  source 1..33: mol_type = other DNA
aaactgaacgatctgtgctttaccaacgtgtat

SEQ ID NO: 128 - protein, 15 aa, Betacoronovirus SARS-CoV-2
TSNFRVQPTESIVRF

SEQ ID NO: 129 - DNA, 45 bp, Betacoronovirus SARS-CoV-2
  source 1..45: mol_type = other DNA
accagcaactttcgcgtgcagccgaccgaaagcattgtgcgcttt

SEQ ID NO: 130 - protein, 15 aa, Betacoronovirus SARS-CoV-2
NEVAVLYQDVNCTEV

SEQ ID NO: 131 - DNA, 45 bp, Betacoronovirus SARS-CoV-2
  source 1..45: mol_type = other DNA
aacgaagtggcggtgctgtatcaggatgtgaactgcaccgaagtg

SEQ ID NO: 132 - protein, 14 aa, Betacoronovirus SARS-CoV-2
RGVYYPDKVFRSSV

SEQ ID NO: 133 - DNA, 42 bp, Betacoronovirus SARS-CoV-2
  source 1..42: mol_type = other DNA
cgcggcgtgtattatccggataaagtgtttcgcagcagcgtg

SEQ ID NO: 134 - protein, 10 aa, Betacoronovirus SARS-CoV-2
GVFVSNGTHW

SEQ ID NO: 135 - DNA, 30 bp, Betacoronovirus SARS-CoV-2
  source 1..30: mol_type = other DNA
ggcgtgtttgtgagcaacggcacccattgg

SEQ ID NO: 136 - protein, 15 aa, Betacoronovirus SARS-CoV-2
VNFNFNGLKGTGVLT

SEQ ID NO: 137 - DNA, 45 bp, Betacoronovirus SARS-CoV-2
  source 1..45: mol_type = other DNA
gtgaactttaactttaacggcctgaaaggcaccggcgtgctgacc

SEQ ID NO: 138 - DNA, 660 bp, synthetic construct
  misc_feature 1..660: note = DNA encoding peptide sequence comprising multiple SARS-Cov-2 epitopes
  source 1..660: mol_type = other DNA
atggtggtgctgagctttgaactgctgcatgcgccggcgaccgtgtgcggcccgaaaaaaaaatgcgtgaactttaactttaacggcctgaaaggcaccggcgtgctgaccgaaagcctgccgccgctgctgaccgatgaaatgattgcgcagtataccagcgcgctggcgggcctggtgaaacagctgagcagcaactttggcgcgattagcagcgtgctgaacgatattattccgtttgcgatgcagatggcgtatcgctttaacggcattggcgtgacccagaacgcgattccgaccaactttaccattagcgtgaccaccgaaattctgccggtgagcatggtggatttttgcggcaaaggctatcatctgatgagctttccgcagagcgcgccgcatccgaccaaactgaacgatctgtgctttaccaacgtgtatgcggattatcagaccagcaactttcgcgtgcagccgaccgaaagcattgtgcgctttccgaacaccagcaaccaggtggcggtgctgtatcaggatgtgaactgcaccgaagtgccggtgtttacccgcggcgtgtattatccggataaagtgtttcgcagcagcgtgctgcatcgcgaaggcgtgtttgtgagcaacggcacccattggtttgtgtaa

SEQ ID NO: 139 - DNA, 696 bp, synthetic construct
  misc_feature 1..696: note = DNA encoding peptide sequence comprising multiple SARS-Cov-2 epitopes
  source 1..696: mol_type = other DNA
atggtgctgctgcaggcgggcagcctgcatgcgaaaaaagtggtgctgagctttgaactgctgcatgcgccggcgaccgtgtgcggcccgaaaaaaaaatgcgtgaactttaactttaacggcctgaaaggcaccggcgtgctgaccgaaagcctgccgccgctgctgaccgatgaaatgattgcgcagtataccagcgcgctggcgggcctggtgaaacagctgagcagcaactttggcgcgattagcagcgtgctgaacgatattattccgtttgcgatgcagatggcgtatcgctttaacggcattggcgtgacccagaacgcgattccgaccaactttaccattagcgtgaccaccgaaattctgccggtgagcatggtggatttttgcggcaaaggctatcatctgatgagctttccgcagagcgcgccgcatccgaccaaactgaacgatctgtgctttaccaacgtgtatgcggattatcagaccagcaactttcgcgtgcagccgaccgaaagcattgtgcgctttccgaacaccagcaaccaggtggcggtgctgtatcaggatgtgaactgcaccgaagtgccggtgtttacccgcggcgtgtattatccggataaagtgtttcgcagcagcgtgctgcatcgcgaaggcgtgtttgtgagcaacggcacccattggtttgtgtaa

SEQ ID NO: 140 - DNA, 828 bp, synthetic construct
  misc_feature 1..828: note = DNA encoding peptide sequence comprising multiple SARS-Cov-2 epitopes
  source 1..828: mol_type = other DNA
atggtgctgctgcaggcgggcagcctgcatgcgaaaaaagtggtgctgagctttgaactgctgcatgcgccggcgaccgtgtgcggcccgaaaaaacgcgtgcgccgcaaatgcgtgaactttaactttaacggcctgaaaggcaccggcgtgctgaccgaaagccgcgtgcgccgcctgccgccgctgctgaccgatgaaatgattgcgcagtataccagcgcgctggcgggccgcgtgcgccgcctggtgaaacagctgagcagcaactttggcgcgattagcagcgtgctgaacgatattcgcgtgcgccgcattccgtttgcgatgcagatggcgtatcgctttaacggcattggcgtgacccagaaccgcgtgcgccgcgcgattccgaccaactttaccattagcgtgaccaccgaaattctgccggtgagcatgcgcgtgcgccgcgtggatttttgcggcaaaggctatcatctgatgagctttccgcagagcgcgccgcatcgcgtgcgccgcccgaccaaactgaacgatctgtgctttaccaacgtgtatgcggatcgcgtgcgccgctatcagaccagcaactttcgcgtgcagccgaccgaaagcattgtgcgctttccgaaccgcgtgcgccgcaccagcaaccaggtggcggtgctgtatcaggatgtgaactgcaccgaagtgccggtgcgcgtgcgccgctttacccgcggcgtgtattatccggataaagtgtttcgcagcagcgtgctgcatcgcgtgcgccgccgcgaaggcgtgtttgtgagcaacggcacccattggtttgtgtaa

SEQ ID NO: 141 - DNA, 828 bp, synthetic construct
  misc_feature 1..828: note = DNA encoding peptide sequence comprising multiple SARS-Cov-2 epitopes
  source 1..828: mol_type = other DNA
atggtgctgctgcaggccggcagcctgcacgccaagaaagtggtgctctccttcgagctgctgcacgcccctgctacagtgtgtggacctaagaaaagagtgagaaggaagtgcgtgaacttcaactttaacggcctgaagggaaccggcgtgctgacagaatctagagtgagaagactgccccccctgctgacagatgagatgatcgcccagtacaccagcgctctggccggcagagtccggagactggtcaagcagctgagcagcaacttcggcgctattagcagcgtgctgaacgacatccgggtccgaagaatccctttcgccatgcagatggcctatagatttaatggcatcggagtgacccagaacagagtgagaagagccatccctacaaacttcaccatctccgtgaccaccgaaatcctgcctgtgtccatgcgggttcggcgcgtggacttctgcggcaagggctaccacctgatgagcttcccccagagcgcccctcacagagtgcggcggccaaccaagctgaatgatctgtgcttcaccaacgtgtacgccgacagagtgcggagataccagaccagcaattttcgggtgcaacctaccgagtctatcgtgcggttccctaacagagtgagaagaaccagcaaccaggtggccgtgctgtaccaagacgtgaactgtaccgaggtgcccgtgagagtcagacggtttacaagaggcgtgtactacccagacaaggtgttcagaagctctgtgctgcatagagtgcggcgcagggaaggcgttttcgtgtctaacggcacacactggttcgtgtaa

SEQ ID NO: 142 - protein, 15 aa, Betacoronovirus SARS-CoV-2
TRTQSPPAPPAYTNS

SEQ ID NO: 143 - protein, 25 aa, Betacoronovirus SARS-CoV-2
SQCVNLTTRTQLPPAYTNSFTRGVY

SEQ ID NO: 144 - protein, 9 aa, Betacoronovirus SARS-CoV-2
GTNGTKRFD

SEQ ID NO: 145 - protein, 23 aa, Betacoronovirus SARS-CoV-2
FSNVTWFHAIHVSGTNGTKRFDN

SEQ ID NO: 146 - protein, 17 aa, Betacoronovirus SARS-CoV-2
DPFLDVYYHKNNKSWME

SEQ ID NO: 147 - protein, 20 aa, Betacoronovirus SARS-CoV-2
HKNNKSWMESEFRVYSSANN

SEQ ID NO: 148 - protein, 13 aa, Betacoronovirus SARS-CoV-2
LTPGDSSSGWTAG

SEQ ID NO: 149 - protein, 13 aa, Betacoronovirus SARS-CoV-2
LTPGDSSSGWTAG

SEQ ID NO: 150 - protein, 62 aa, Betacoronovirus SARS-CoV-2
FPNITNLCPFDEVFNATRFASVYAWNRKRISNCVADYSVLYNFAPFFAFKCYGVSPTKLNDL

SEQ ID NO: 151 - protein, 20 aa, Betacoronovirus SARS-CoV-2
RFASVYAWNRKRISNCVADY

SEQ ID NO: 152 - protein, 18 aa, Betacoronovirus SARS-CoV-2
RGNEVSQIAPGQTGNIAD

SEQ ID NO: 153 - protein, 19 aa, Betacoronovirus SARS-CoV-2
GQTGNIADYNYKLPDDFTG

SEQ ID NO: 154 - protein, 12 aa, Betacoronovirus SARS-CoV-2
KLDSKVGGNYNY

SEQ ID NO: 155 - protein, 20 aa, Betacoronovirus SARS-CoV-2
DSKVGGNYNYLYRLFRKSNL

SEQ ID NO: 156 - protein, 9 aa, Betacoronovirus SARS-CoV-2
YQAGNKPCN

SEQ ID NO: 157 - protein, 5 aa, Betacoronovirus SARS-CoV-2
YQAGS

SEQ ID NO: 158 - protein, 9 aa, Betacoronovirus SARS-CoV-2
YGFRPTYGV

SEQ ID NO: 159 - protein, 20 aa, Betacoronovirus SARS-CoV-2
PLRSYGFRPTYGVGHQPYRV

SEQ ID NO: 160 - protein, 20 aa, Betacoronovirus SARS-CoV-2
FELLHAPATVCGPKKSTNLV

SEQ ID NO: 161 - protein, 20 aa, Betacoronovirus SARS-CoV-2
LHAPATVCGPKKSTNLVKNK

SEQ ID NO: 162 - protein, 13 aa, Betacoronovirus SARS-CoV-2
RDIADTTDAVRDP

SEQ ID NO: 163 - protein, 75 aa, Betacoronovirus SARS-CoV-2
VCGPKKSTNLVKNKCVNFNFNGLTGTGVLTESNKKFLPFQQFGRDIADTTDAVRDPQTLEILDITPCSFGGVSVI

SEQ ID NO: 164 - protein, 10 aa, Betacoronovirus SARS-CoV-2
VITPGTNTSN

SEQ ID NO: 165 - protein, 40 aa, Betacoronovirus SARS-CoV-2
GTNTSNQVAVLYQDVNCTEVPVAIHADQLTPTWRVYSTGS

SEQ ID NO: 166 - protein, 19 aa, Betacoronovirus SARS-CoV-2
ASYQTQTKSHRRARSVASQ

SEQ ID NO: 167 - protein, 20 aa, Betacoronovirus SARS-CoV-2
CASYQTQTKSHRRARSVASQ

SEQ ID NO: 168 - protein, 10 aa, Betacoronovirus SARS-CoV-2
LPDPSKPSKR

SEQ ID NO: 169 - protein, 12 aa, Betacoronovirus SARS-CoV-2
ILPDPSKPSKRS

SEQ ID NO: 170 - protein, 40 aa, Betacoronovirus SARS-CoV-2
VNNTVYDPLQPELDSFKEELDKYFKNHTSPDVDLGDISGI

SEQ ID NO: 171 - protein, 20 aa, Betacoronovirus SARS-CoV-2
DSFKEELDKYFKNHTSPDVD

SEQ ID NO: 172 - protein, 1273 aa, Betacoronovirus SARS-CoV-2
MFVFLVLLPLVSSQCVNLTTRTQSPPAPPAYTNSFTRGVYYPDKVFRSSVLHSTQDLFLPFFSNVTWFHAISGTNGTKRFDNPVLPFNDGVYFASIEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPILGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 173 - DNA, 27 bp, Betacoronovirus SARS-CoV-2
  source 1..27: mol_type = other DNA
ggcaccaacggcaccaaacgctttgat

SEQ ID NO: 174 - DNA, 60 bp, Betacoronovirus SARS-CoV-2
  source 1..60: mol_type = other DNA
tttgaactgctgcatgcgccggcgaccgtgtgcggcccgaaaaaaagcaccaacctggtg

SEQ ID NO: 175 - DNA, 39 bp, Betacoronovirus SARS-CoV-2
  source 1..39: mol_type = other DNA
cgcgatattgcggataccaccgatgcggtgcgcgatccg

SEQ ID NO: 176 - DNA, 30 bp, Betacoronovirus SARS-CoV-2
  source 1..30: mol_type = other DNA
gtgattaccccgggcaccaacaccagcaac

SEQ ID NO: 177 - DNA, 30 bp, Betacoronovirus SARS-CoV-2
  source 1..30: mol_type = other DNA
ctgccggatccgagcaaaccgagcaaacgc

SEQ ID NO: 178 - DNA, 120 bp, Betacoronovirus SARS-CoV-2
  source 1..120: mol_type = other DNA
gtgaacaacaccgtgtatgatccgctgcagccggaactggatagctttaaagaagaactggataaatattttaaaaaccataccagcccggatgtggatctgggcgatattagcggcatt

SEQ ID NO: 179 - protein, 15 aa, Betacoronovirus SARS-CoV-2
ATRFASVYAWNRKRI

SEQ ID NO: 180 - protein, 15 aa, Betacoronovirus SARS-CoV-2
ESNKKFLPFQQFGRD

SEQ ID NO: 181 - protein, 15 aa, Betacoronovirus SARS-CoV-2
CSNLLLQYGSFCTQL

SEQ ID NO: 182 - protein, 15 aa, Betacoronovirus SARS-CoV-2
LFNKVTLADAGFIKQ

SEQ ID NO: 183 - protein, 15 aa, Betacoronovirus SARS-CoV-2
QKGLTVLPPLLTDEM

SEQ ID NO: 184 - protein, 15 aa, Betacoronovirus SARS-CoV-2
AQALNTLVKQLSSKF

SEQ ID NO: 185 - protein, 19 aa, Betacoronovirus SARS-CoV-2
LLTDEMIAQYTSALLAGTI

SEQ ID NO: 186 - protein, 15 aa, Betacoronovirus SARS-CoV-2
WNRKRISNCVADYSV

SEQ ID NO: 187 - protein, 11 aa, Betacoronovirus SARS-CoV-2
KKFLPFQQFGR

SEQ ID NO: 188 - protein, 7 aa, Betacoronovirus SARS-CoV-2
LLQYGSF

SEQ ID NO: 189 - protein, 11 aa, Betacoronovirus SARS-CoV-2
FIKQYGDCLGD

SEQ ID NO: 190 - protein, 19 aa, Betacoronovirus SARS-CoV-2
LLTDEMIAQYTSALLAGTI

SEQ ID NO: 191 - protein, 11 aa, Betacoronovirus SARS-CoV-2
QALNTLVKQLS

SEQ ID NO: 192 - protein, 6 aa, Betacoronovirus SARS-CoV-2
LLTDEM

SEQ ID NO: 193 - protein, 19 aa, Betacoronovirus SARS-CoV-2
YAWNRKRISNCVADYSVLY

SEQ ID NO: 194 - DNA, 57 bp, Betacoronovirus SARS-CoV-2
  source 1..57: mol_type = other DNA
tatgcgtggaaccgcaaacgcattagcaactgcgtggcggattatagcgtgctgtat

SEQ ID NO: 195 - protein, 15 aa, Betacoronovirus SARS-CoV-2
SNKKFLPFQQFGRDI

SEQ ID NO: 196 - DNA, 45 bp, Betacoronovirus SARS-CoV-2
  source 1..45: mol_type = other DNA
agcaacaaaaaatttctgccgtttcagcagtttggccgcgatatt

SEQ ID NO: 197 - protein, 11 aa, Betacoronovirus SARS-CoV-2
NLLLQYGSFCT

SEQ ID NO: 198 - DNA, 33 bp, Betacoronovirus SARS-CoV-2
  source 1..33: mol_type = other DNA
aacctgctgctgcagtatggcagcttttgcacc

SEQ ID NO: 199 - protein, 15 aa, Betacoronovirus SARS-CoV-2
AGFIKQYGDCLGDIA

SEQ ID NO: 200 - DNA, 45 bp, Betacoronovirus SARS-CoV-2
  source 1..45: mol_type = other DNA
gcgggctttattaaacagtatggcgattgcctgggcgatattgcg

SEQ ID NO: 201 - protein, 23 aa, Betacoronovirus SARS-CoV-2
PPLLTDEMIAQYTSALLAGTITS

SEQ ID NO: 202 - DNA, 69 bp, Betacoronovirus SARS-CoV-2
  source 1..69: mol_type = other DNA
ccgccgctgctgaccgatgaaatgattgcgcagtataccagcgcgctgctggcgggcaccattaccagc

SEQ ID NO: 203 - protein, 15 aa, Betacoronovirus SARS-CoV-2
NAQALNTLVKQLSSK

SEQ ID NO: 204 - DNA, 45 bp, Betacoronovirus SARS-CoV-2
  source 1..45: mol_type = other DNA
aacgcgcaggcgctgaacaccctggtgaaacagctgagcagcaaa

SEQ ID NO: 205 - protein, 13 aa, synthetic construct
  REGION 1..13: note = Pan-HLA DR-binding epitope (PADRE) sequence
AKFVAAWTLKAAA

SEQ ID NO: 206 - DNA, 300 bp, synthetic construct
  misc_feature 1..300: note = DNA encoding peptide sequence comprising multiple SARS-Cov-2 epitopes
  source 1..300: mol_type = other DNA
atgtatgcgtggaaccgcaaacgcattagcaactgcgtggcggattatagcgtgctgtatagcaacaaaaaatttctgccgtttcagcagtttggccgcgatattaacctgctgctgcagtatggcagcttttgcaccgcgggctttattaaacagtatggcgattgcctgggcgatattgcgccgccgctgctgaccgatgaaatgattgcgcagtataccagcgcgctgctggcgggcaccattaccagcaacgcgcaggcgctgaacaccctggtgaaacagctgagcagcaaataa

SEQ ID NO: 207 - DNA, 384 bp, synthetic construct
  misc_feature 1..384: note = DNA encoding peptide sequence comprising multiple SARS-Cov-2 epitopes
  source 1..384: mol_type = other DNA
atgattagcggcaccaacggcaccaaacgctttgataacccgctgagctttgaactgctgcatgcgccggcgaccgtgtgcggcccgaaaaaaagcaccaacctggtgaaaaactttggccgcgatattgcggataccaccgatgcggtgcgcgatccgcagaccgtgagcgtgattaccccgggcaccaacaccagcaaccaggtgcagattctgccggatccgagcaaaccgagcaaacgcagctttggcattgtgaacaacaccgtgtatgatccgctgcagccggaactggatagctttaaagaagaactggataaatattttaaaaaccataccagcccggatgtggatctgggcgatattagcggcattaacgcgtaa

SEQ ID NO: 208 - protein, 29 aa, synthetic construct
  REGION 1..29: note = Protein secretory signal
KGSPWKGSLLLLLMNIVSNLLCQSVAPLP

SEQ ID NO: 209 - DNA, 87 bp, synthetic construct
  misc_feature 1..87: note = DNA encoding protein secretory signal
  source 1..87: mol_type = other DNA
aaaggcagcccgtggaaaggcagcctgctgctgctgctgatgaacattgtgagcaacctgctgtgccagagcgtggcgccgctgccg

SEQ ID NO: 210 - DNA, 540 bp, synthetic construct
  misc_feature 1..540: note = DNA encoding peptide sequence comprising multiple SARS-Cov-2 epitopes
  source 1..540: mol_type = other DNA
atgatggatgatcagcgcgatctgattagcaaccatgaacagctgccgattctgggcaaccgcccgcgcgaaccggaacgctgcagccgcggcgcgctgtataccggcgtgagcgtgctggtggcgctgctgctggcgggccaggcgaccaccgcgtattttctgtatcagcagcagggccgcctggataaactgaccattaccagccagaacctgcagctggaaagcctgcgcatgaaactgtatgcgtggaaccgcaaacgcattagcaactgcgtggcggattatagcgtgctgtatagcaacaaaaaatttctgccgtttcagcagtttggccgcgatattaacctgctgctgcagtatggcagcttttgcaccgcgggctttattaaacagtatggcgattgcctgggcgatattgcgccgccgctgctgaccgatgaaatgattgcgcagtataccagcgcgctgctggcgggcaccattaccagcaacgcgcaggcgctgaacaccctggtgaaacagctgagcagcaaataa

SEQ ID NO: 211 - DNA, 750 bp, synthetic construct
  misc_feature 1..750: note = DNA encoding peptide sequence comprising multiple SARS-Cov-2 epitopes
  source 1..750: mol_type = other DNA
atgaaaggcagcccgtggaaaggcagcctgctgctgctgctgatgaacattgtgagcaacctgctgctgtgccagagcgtggcgccgctgccggtggtgctgagctttgaactgctgcatgcgccggcgaccgtgtgcggcccgaaaaaaaaatgcgtgaactttaactttaacggcctgaaaggcaccggcgtgctgaccgaaagcctgccgccgctgctgaccgatgaaatgattgcgcagtataccagcgcgctggcgggcctggtgaaacagctgagcagcaactttggcgcgattagcagcgtgctgaacgatattattccgtttgcgatgcagatggcgtatcgctttaacggcattggcgtgacccagaacgcgattccgaccaactttaccattagcgtgaccaccgaaattctgccggtgagcatggtggatttttgcggcaaaggctatcatctgatgagctttccgcagagcgcgccgcatccgaccaaactgaacgatctgtgctttaccaacgtgtatgcggattatcagaccagcaactttcgcgtgcagccgaccgaaagcattgtgcgctttccgaacaccagcaaccaggtggcggtgctgtatcaggatgtgaactgcaccgaagtgccggtgtttacccgcggcgtgtattatccggataaagtgtttcgcagcagcgtgctgcatcgcgaaggcgtgtttgtgagcaacggcacccattggtttgtgtaa

SEQ ID NO: 212 - DNA, 48 bp, synthetic construct
  misc_feature 1..48: note = DNA encoding secretory peptide
  source 1..48: mol_type = other DNA
atgctgctgctgctgctgctgctgctgctgctggcgctggcgctggcg

SEQ ID NO: 213 - protein, 29 aa, synthetic construct
  REGION 1..29: note = Prolactin peptide signal
KGSPWKGSLLLLLMNIVSNLLCQSVAPLP

SEQ ID NO: 214 - DNA, 90 bp, synthetic construct
  misc_feature 1..90: note = DNA encoding prolactin peptide signal
  source 1..90: mol_type = other DNA
aaaggcagcccgtggaaaggcagcctgctgctgctgctgatgaacattgtgagcaacctgctgctgtgccagagcgtggcgccgctgccg

SEQ ID NO: 215 - protein, 20 aa, synthetic construct
  REGION 1..20: note = Viral T2A sequence
GSGEGRGSLLTCGDVEENPG

SEQ ID NO: 216 - DNA, 63 bp, synthetic construct
  misc_feature 1..63: note = DNA encoding viral T2A sequence
  source 1..63: mol_type = other DNA
ggcagcggcgaaggccgcggcagcctgctgacctgcggcgatgtggaagaaaacccgggcccg

SEQ ID NO: 217 - protein, 21 aa, synthetic construct
  REGION 1..21: note = Viral T2A sequence
GSGEGRGSLLTCGDVEENPGP

SEQ ID NO: 218 - DNA, 1346 bp, synthetic construct
  misc_feature 1..1346: note = DNA encoding peptide sequence comprising multiple SARS-Cov-2 epitopes
  source 1..1346: mol_type = other DNA
atgatggatgatcagcgcgatctgattagcaaccatgaacagctgccgattctgggcaaccgcccgcgcgaaccggaacgctgcagccgcggcgcgctgtataccggcgtgagcgtgctggtggcgctgctgctggcgggccaggcgaccaccgcgtattttctgtatcagcagcagggccgcctggataaactgaccattaccagccagaacctgcagctggaaagcctgcgcatgaaactgtatgcgtggaaccgcaaacgcattagcaactgcgtggcggattatagcgtgctgtatagcaacaaaaaatttctgccgtttcagcagtttggccgcgatattaacctgctgctgcagtatggcagcttttgcaccgcgggctttattaaacagtatggcgattgcctgggcgatattgcgccgccgctgctgaccgatgaaatgattgcgcagtataccagcgcgctgctggcgggcaccattaccagcaacgcgcaggcgctgaacaccctggtgaaacagctgagcagcaaaggcagcggcgaaggccgcggcagcctgctgacctgcggcgatgtggaagaaaacccgggcccgaaaggcagcccgtggaaaggcagcctgctgctgctgctgatgaacattgtgagcaacctgctgctgtgccagagcgtggcgccgctgcggtggtgctgagctttgaactgctgcatgcgccggcgaccgtgtgcggcccgaaaaaaaaatgcgtgaactttaactttaacggcctgaaaggcaccggcgtgctgaccgaaagcctgccgccgctgctgaccgatgaaatgattgcgcagtataccagcgcgctggcgggcctggtgaaacagctgagcagcaactttggcgcgattagcagcgtgctgaacgatattattccgtttgcgatgcagatggcgtatcgctttaacggcattggcgtgacccagaacgcgattccgaccaactttaccattagcgtgaccaccgaaattctgccggtgagcatggtggatttttgcggcaaaggctatcatctgatgagctttccgcagagcgcgccgcatccgaccaaactgaacgatctgtgctttaccaacgtgtatgcggattatcagaccagcaactttcgcgtgcagccgaccgaaagcattgtgcgctttccgaacaccagcaaccaggtggcggtgctgtatcaggatgtgaactgcaccgaagtgccggtgtttacccgcggcgtgtattatccggataaagtgtttcgcagcagcgtgctgcatcgcgaaggcgtgtttgtgagcaacggcacccattggtttgtgtaa

SEQ ID NO: 219 - DNA, 1347 bp, synthetic construct
  misc_feature 1..1347: note = DNA encoding peptide sequence comprising multiple SARS-Cov-2 epitopes
  source 1..1347: mol_type = other DNA
atgatggacgaccagagagatctgatcagcaatcacgagcagctgccaatcctgggcaaccggcctagagaacctgagagatgcagcagaggcgctctgtacaccggcgtgagcgtgctggtggctctgttgttggctggccaggccacaaccgcctacttcctgtaccagcagcagggcagactggacaagctgaccatcaccagccagaacctgcagctggaaagcctcagaatgaaactgtacgcctggaacaggaagcggatcagcaattgcgtggctgattacagcgtcctgtactccaacaagaaattcctgccttttcagcaatttggccgggacatcaacctcctgctgcagtacggctctttttgcaccgccggcttcatcaagcaatacggcgactgcctgggcgatatcgcccctccactgctgaccgacgagatgatcgcccagtacacctctgccctgctggccggcaccatcacctccaacgctcaagctctgaacaccctggtcaagcaactgagcagcaagggcagcggcgagggccggggcagcctgctgacctgcggcgacgtggaagagaaccccggccccaaaggtagcccttggaagggaagcctgctgctgctgctcatgaacatcgtgtctaatctgctgctgtgtcagtctgtggcccctctgcctgtggtgctgtctttcgagctgctgcacgccccagccaccgtgtgtggacctaagaagaagtgcgtgaacttcaacttcaacggcctgaagggaaccggcgtgctgaccgaaagcctgcctcctctgctgaccgatgagatgatcgcccagtatacatctgccctggccggcctggtgaaacagctgagcagcaacttcggtgccataagcagcgtgcttaatgatattatccctttcgccatgcagatggcctatagatttaacggcatcggcgtgacacagaacgccatccccacaaacttcaccatctccgtcacaacagaaatcctgcccgtgagcatggtggacttctgcggcaagggctaccacctgatgagcttcccccagagcgcccctcaccccaccaagctgaacgacctgtgcttcacgaacgtgtacgccgactaccagaccagcaacttccgcgtgcagcctaccgagagcatcgtgcggttcccaaacacatctaaccaggtggccgtgctgtaccaggacgttaattgtacagaagtgcccgtgttcacaagaggagtgtactaccctgataaggtgttcagaagctctgtgctgcatagagagggcgtttttgtgtccaatggaacacactggttcgtgtaa

SEQ ID NO: 220 - protein, 8 aa, Betacoronovirus SARS-CoV-2
FLWLLWPV

SEQ ID NO: 221 - protein, 9 aa, Betacoronovirus SARS-CoV-2
RNRFLYIIK

SEQ ID NO: 222 - protein, 9 aa, Betacoronovirus SARS-CoV-2
ATRRIRGGD

SEQ ID NO: 223 - protein, 9 aa, Betacoronovirus SARS-CoV-2
LWLLWPVTL

SEQ ID NO: 224 - protein, 9 aa, Betacoronovirus SARS-CoV-2
ENAFLPFAM

SEQ ID NO: 225 - protein, 9 aa, Betacoronovirus SARS-CoV-2
ATATIPIQA

SEQ ID NO: 226 - protein, 9 aa, Betacoronovirus SARS-CoV-2
LAAVYRINW

SEQ ID NO: 227 - protein, 9 aa, Betacoronovirus SARS-CoV-2
LACFVLAAV

SEQ ID NO: 228 - protein, 9 aa, Betacoronovirus SARS-CoV-2
FAMGIIAMS

SEQ ID NO: 229 - protein, 9 aa, Betacoronovirus SARS-CoV-2
NLLLLFVTV

SEQ ID NO: 230 - protein, 9 aa, Betacoronovirus SARS-CoV-2
TLACFVLAA

SEQ ID NO: 231 - protein, 9 aa, Betacoronovirus SARS-CoV-2
VTLACFVLA

SEQ ID NO: 232 - protein, 9 aa, Betacoronovirus SARS-CoV-2
ELLHAPATV

SEQ ID NO: 233 - protein, 8 aa, Betacoronovirus SARS-CoV-2
RWVLSFEL

SEQ ID NO: 234 - protein, 9 aa, Betacoronovirus SARS-CoV-2
YIIKLIFLW

SEQ ID NO: 235 - protein, 9 aa, Betacoronovirus SARS-CoV-2
GIAIAMACL

SEQ ID NO: 236 - protein, 9 aa, Betacoronovirus SARS-CoV-2
AMACLVGLM

SEQ ID NO: 237 - protein, 9 aa, Betacoronovirus SARS-CoV-2
AFLPFAMGI

SEQ ID NO: 238 - protein, 15 aa, Betacoronovirus SARS-CoV-2
ACFVLAAVYRINWIT

SEQ ID NO: 239 - protein, 15 aa, Betacoronovirus SARS-CoV-2
AFLPFAMGIIAMSAF

SEQ ID NO: 240 - protein, 15 aa, Betacoronovirus SARS-CoV-2
FFLYENAFLPFAMGI

SEQ ID NO: 241 - protein, 15 aa, Betacoronovirus SARS-CoV-2
FLPFAMGIIAMSAFA

SEQ ID NO: 242 - protein, 15 aa, Betacoronovirus SARS-CoV-2
GIAIAMACLVGLMWL

SEQ ID NO: 243 - protein, 15 aa, Betacoronovirus SARS-CoV-2
IGYYRRATRRIRGGD

SEQ ID NO: 244 - protein, 15 aa, Betacoronovirus SARS-CoV-2
IKLIFLWLLWPVTLA

SEQ ID NO: 245 - protein, 15 aa, Betacoronovirus SARS-CoV-2
KLIFLWLLWPVTLAC

SEQ ID NO: 246 - protein, 15 aa, Betacoronovirus SARS-CoV-2
LPFAMGIIAMSAFAM

SEQ ID NO: 247 - protein, 14 aa, Betacoronovirus SARS-CoV-2
NAFLPFAMGHAMSA

SEQ ID NO: 248 - protein, 15 aa, Betacoronovirus SARS-CoV-2
PSDFVRATATIPIQA

SEQ ID NO: 249 - protein, 15 aa, Betacoronovirus SARS-CoV-2
PVTLACFVLAAVYRI

SEQ ID NO: 250 - protein, 15 aa, Betacoronovirus SARS-CoV-2
RNRFLYIIKLIFLWL

SEQ ID NO: 251 - protein, 14 aa, Betacoronovirus SARS-CoV-2
RWVLSFELLHAPAT

SEQ ID NO: 252 - protein, 15 aa, Betacoronovirus SARS-CoV-2
SDFVRATATIPIQAS

SEQ ID NO: 253 - protein, 15 aa, Betacoronovirus SARS-CoV-2
TLACFVLAAVYRINW

SEQ ID NO: 254 - protein, 15 aa, Betacoronovirus SARS-CoV-2
VHFVCNLLLLFVTVY

SEQ ID NO: 255 - protein, 14 aa, Betacoronovirus SARS-CoV-2
WLSFELLHAPATVC

SEQ ID NO: 256 - protein, 14 aa, Betacoronovirus SARS-CoV-2
YRWVLSFELLHAPA

SEQ ID NO: 257 - protein, 16 aa, Betacoronovirus SARS-CoV-2
GIAIAMACLVGLMWLS

SEQ ID NO: 258 - protein, 16 aa, Betacoronovirus SARS-CoV-2
GKNWITGGIAIAMACL

SEQ ID NO: 259 - protein, 16 aa, Betacoronovirus SARS-CoV-2
LWPVTLACFVLAAVYN

SEQ ID NO: 260 - protein, 16 aa, Betacoronovirus SARS-CoV-2
LWPVTLACFVLAAVYR

SEQ ID NO: 261 - protein, 16 aa, Betacoronovirus SARS-CoV-2
RINWITGGIAIAMACL

SEQ ID NO: 262 - protein, 15 aa, Betacoronovirus SARS-CoV-2
WLSFELLHAPATVCG

SEQ ID NO: 263 - protein, 16 aa, Betacoronovirus SARS-CoV-2
YENAFLPFAMGIIAMS

SEQ ID NO: 264 - protein, 16 aa, Betacoronovirus SARS-CoV-2
YFLCWHTNCYDYCIPY

SEQ ID NO: 265 - protein, 20 aa, synthetic construct
  REGION 1..20: note = Viral T2A sequence
GSGEGRGSLLTCGDVEENPG

SEQ ID NO: 266 - DNA, 63 bp, synthetic construct; identical to SEQ ID NO: 216
  misc_feature 1..63: note = DNA encoding viral T2A sequence
  source 1..63: mol_type = other DNA

SEQ ID NO: 267 - DNA, 48 bp, synthetic construct; identical to SEQ ID NO: 212
  misc_feature 1..48: note = DNA encoding secretory peptide
  source 1..48: mol_type = other DNA

SEQ ID NO: 268 - protein, 9 aa, Betacoronovirus SARS-CoV-2
YLATALLTL

SEQ ID NO: 269 - DNA, 27 bp, Betacoronovirus SARS-CoV-2
  source 1..27: mol_type = other DNA
tatctggcgaccgcgctgctgaccctg

SEQ ID NO: 270 - protein, 9 aa, Betacoronovirus SARS-CoV-2
WLMWLIINL

SEQ ID NO: 271 - DNA, 27 bp, Betacoronovirus SARS-CoV-2
  source 1..27: mol_type = other DNA
tggctgatgtggctgattattaacctg

SEQ ID NO: 272 - protein, 9 aa, Betacoronovirus SARS-CoV-2
SLPGVFCGV

SEQ ID NO: 273 - DNA, 27 bp, Betacoronovirus SARS-CoV-2
  source 1..27: mol_type = other DNA
agcctgccgggcgtgttttgcggcgtg

SEQ ID NO: 274 - protein, 9 aa, Betacoronovirus SARS-CoV-2
KLSYGIATV

SEQ ID NO: 275 - DNA, 27 bp, Betacoronovirus SARS-CoV-2
  source 1..27: mol_type = other DNA
aaactgagctatggcattgcgaccgtg

SEQ ID NO: 276 - protein, 9 aa, Betacoronovirus SARS-CoV-2
LLLDDFVEI

SEQ ID NO: 277 - DNA, 27 bp, Betacoronovirus SARS-CoV-2
  source 1..27: mol_type = other DNA
ctgctgctggatgattttgtggaaatt

SEQ ID NO: 278 - protein, 9 aa, Betacoronovirus SARS-CoV-2
FVFLVLLPL

SEQ ID NO: 279 - DNA, 27 bp, Betacoronovirus SARS-CoV-2
  source 1..27: mol_type = other DNA
tttgtgtttctggtgctgctgccgctg

SEQ ID NO: 280 - protein, 9 aa, Betacoronovirus SARS-CoV-2
ALNTLVKQL

SEQ ID NO: 281 - DNA, 27 bp, Betacoronovirus SARS-CoV-2
  source 1..27: mol_type = other DNA
gcgctgaacaccctggtgaaacagctg

SEQ ID NO: 282 - protein, 9 aa, Betacoronovirus SARS-CoV-2
RLQSLQTYV

SEQ ID NO: 283 - DNA, 27 bp, Betacoronovirus SARS-CoV-2
  source 1..27: mol_type = other DNA
cgcctgcagagcctgcagacctatgtg

SEQ ID NO: 284 - protein, 9 aa, Betacoronovirus SARS-CoV-2
FIAGLIAIV

SEQ ID NO: 285 - DNA, 27 bp, Betacoronovirus SARS-CoV-2
  source 1..27: mol_type = other DNA
tttattgcgggcctgattgcgattgtg

SEQ ID NO: 286 - protein, 9 aa, Betacoronovirus SARS-CoV-2
FLAFVVFLL

SEQ ID NO: 287 - DNA, 27 bp, Betacoronovirus SARS-CoV-2
  source 1..27: mol_type = other DNA
tttctggcgtttgtggtgtttctgctg

SEQ ID NO: 288 - protein, 9 aa, Betacoronovirus SARS-CoV-2
FLLNKEMYL

SEQ ID NO: 289 - DNA, 27 bp, Betacoronovirus SARS-CoV-2
  source 1..27: mol_type = other DNA
tttctgctgaacaaagaaatgtatctg

SEQ ID NO: 290 - protein, 9 aa, Betacoronovirus SARS-CoV-2
IIFWFSLEL

SEQ ID NO: 291 - DNA, 27 bp, Betacoronovirus SARS-CoV-2
  source 1..27: mol_type = other DNA
attattttttggtttagcctggaactg

SEQ ID NO: 292 - protein, 9 aa, Betacoronovirus SARS-CoV-2
YIDIGNYTV

SEQ ID NO: 293 - DNA, 27 bp, Betacoronovirus SARS-CoV-2
  source 1..27: mol_type = other DNA
tatattgatattggcaactataccgtg

SEQ ID NO: 294 - protein, 9 aa, Betacoronovirus SARS-CoV-2
YINVFAFPF

SEQ ID NO: 295 - DNA, 27 bp, Betacoronovirus SARS-CoV-2
  source 1..27: mol_type = other DNA
tatattaacgtgtttgcgtttccgttt

SEQ ID NO: 296 - protein, 9 aa, Betacoronovirus SARS-CoV-2
NVFAFPFTI

SEQ ID NO: 297 - DNA, 27 bp, Betacoronovirus SARS-CoV-2
  source 1..27: mol_type = other DNA
aacgtgtttgcgtttccgtttaccatt

SEQ ID NO: 298 - protein, 15 aa, Betacoronovirus SARS-CoV-2
KSAFYILPSIISNEK

SEQ ID NO: 299 - DNA, 45 bp, Betacoronovirus SARS-CoV-2
  source 1..45: mol_type = other DNA
aaaagcgcgttttatattctgccgagcattattagcaacgaaaaa

SEQ ID NO: 300 - protein, 15 aa, Betacoronovirus SARS-CoV-2
PNMLRIMASLVLARK

SEQ ID NO: 301 - DNA, 45 bp, Betacoronovirus SARS-CoV-2
  source 1..45: mol_type = other DNA
ccgaacatgctgcgcattatggcgagcctggtgctggcgcgcaaa

SEQ ID NO: 302 - protein, 15 aa, Betacoronovirus SARS-CoV-2
LSYYKLGASQRVAGD

SEQ ID NO: 303 - DNA, 45 bp, Betacoronovirus SARS-CoV-2
  source 1..45: mol_type = other DNA
ctgagctattataaactgggcgcgagccagcgcgtggcgggcgat

SEQ ID NO: 304 - protein, 15 aa, Betacoronovirus SARS-CoV-2
AEILLIIMRTFKVSI

SEQ ID NO: 305 - DNA, 45 bp, Betacoronovirus SARS-CoV-2
  source 1..45: mol_type = other DNA
gcggaaattctgctgattattatgcgcacctttaaagtgagcatt

SEQ ID NO: 306 - protein, 15 aa, Betacoronovirus SARS-CoV-2
DFYLCFLAFLLFLVL

SEQ ID NO: 307 - DNA, 45 bp, Betacoronovirus SARS-CoV-2
  source 1..45: mol_type = other DNA
gatttttatctgtgctttctggcgtttctgctgtttctggtgctg

SEQ ID NO: 308 - protein, 15 aa, Betacoronovirus SARS-CoV-2
MKFLVFLGIITTVAA

SEQ ID NO: 309 - DNA, 45 bp, Betacoronovirus SARS-CoV-2
  source 1..45: mol_type = other DNA
atgaaatttctggtgtttctgggcattattaccaccgtggcggcg

SEQ ID NO: 310 - protein, 25 aa, Betacoronovirus SARS-CoV-2
SQCVNLTTRTQLPPAYTNSFTRGVY

SEQ ID NO: 311 - DNA, 75 bp, Betacoronovirus SARS-CoV-2
  source 1..75: mol_type = other DNA
agccagtgcgtgaacctgaccacccgcacccagctgccgccggcgtataccaacagctttacccgcggcgtgtat

SEQ ID NO: 312 - protein, 31 aa, Betacoronovirus SARS-CoV-2
DAVDCALDPLSETKCTLKSFTVEKGIYQTSN

SEQ ID NO: 313 - DNA, 93 bp, Betacoronovirus SARS-CoV-2
  source 1..93: mol_type = other DNA
gatgcggtggattgcgcgctggatccgctgagcgaaaccaaatgcaccctgaaaagctttaccgtggaaaaaggcatttatcagaccagcaac

SEQ ID NO: 314 - protein, 40 aa, Betacoronovirus SARS-CoV-2; identical to SEQ ID NO: 165

SEQ ID NO: 315 - DNA, 120 bp, Betacoronovirus SARS-CoV-2
  source 1..120: mol_type = other DNA
ggcaccaacaccagcaaccaggtggcggtgctgtatcaggatgtgaactgcaccgaagtgccggtggcgattcatgcggatcagctgaccccgacctggcgcgtgtatagcaccggcagc

SEQ ID NO: 316 - protein, 75 aa, Betacoronovirus SARS-CoV-2; identical to SEQ ID NO: 163

SEQ ID NO: 317 - DNA, 225 bp, Betacoronovirus SARS-CoV-2
  source 1..225: mol_type = other DNA
gtgtgcggcccgaaaaaaagcaccaacctggtgaaaaacaaatgcgtgaactttaactttaacggcctgaccggcaccggcgtgctgaccgaaagcaacaaaaaatttctgccgtttcagcagtttggccgcgatattgcggataccaccgatgcggtgcgcgatccgcagaccctggaaattctggatattaccccgtgcagctttggcggcgtgagcgtgatt

SEQ ID NO: 318 - DNA, 600 bp, synthetic construct
  misc_feature 1..600: note = DNA encoding peptide sequence comprising multiple SARS-Cov-2 epitopes
  source 1..600: mol_type = other DNA
atggacgaccagagagatctgatcagcaatcacgagcagctgccaatcctgggcaaccggcctagagaacctgagagatgcagcagaggcgctctgtacaccggcgtgagcgtgctggtggctctgttgttggctggccaggccacaaccgcctacttcctgtaccagcagcagggcagactggacaagctgaccatcaccagccagaacctgcagctggaaagcctcagaatgaaactgggcccgggcccgggcaaaagcgcgttttatattctgccgagcattattagcaacgaaaaaggcccgggcccgggcccgaacatgctgcgcattatggcgagcctggtgctggcgcgcaaaggcccgggcccgggcctgagctattataaactgggcgcgagccagcgcgtggcgggcgatggcccgggcccgggcgcggaaattctgctgattattatgcgcacctttaaagtgagcattggcccgggcccgggcgatttttatctgtgctttctggcgtttctgctgtttctggtgctgggcccgggcccgggcatgaaatttctggtgtttctgggcattattaccaccgtggcggcg

SEQ ID NO: 319 - DNA, 582 bp, synthetic construct
  misc_feature 1..582: note = DNA encoding peptide sequence comprising multiple SARS-Cov-2 epitopes
  source 1..582: mol_type = other DNA
gtgctgctgcaggccggcagcctgcacgccaagaaagaagcggcggcgaaatatctggcgaccgcgctgctgaccctggcggcgtattggctgatgtggctgattattaacctggcggcgtatagcctgccgggcgtgttttgcggcgtggcggcgtataaactgagctatggcattgcgaccgtggcggcgtatctgctgctggatgattttgtggaaattgcggcgtattttgtgtttctggtgctgctgccgctggcggcgtatgcgctgaacaccctggtgaaacagctggcggcgtatcgcctgcagagcctgcagacctatgtggcggcgtattttattgcgggcctgattgcgattgtggcggcgtattttctggcgtttgtggtgtttctgctggcggcgtattttctgctgaacaaagaaatgtatctggcggcgtatattattttttggtttagcctggaactggcggcgtattatattgatattggcaactataccgtggcggcgtattatattaacgtgtttgcgtttccgtttgcggcgtataacgtgtttgcgtttccgtttaccatt

SEQ ID NO: 320 - DNA, 582 bp, synthetic construct; identical to SEQ ID NO: 319
  misc_feature 1..582: note = DNA encoding peptide sequence comprising multiple SARS-Cov-2 epitopes
  source 1..582: mol_type = other DNA

SEQ ID NO: 321 - DNA, 1898 bp, synthetic construct
  misc_feature 1..1898: note = DNA encoding peptide sequence comprising multiple SARS-Cov-2 epitopes
  source 1..1898: mol_type = other DNA
atgtggacgaccagagagatctgatcagcaatcacgagcagctgccaatcctgggcaaccggcctagagaacctgagagatgcagcagaggcgctctgtacaccggcgtgagcgtgctggtggctctgttgttggctggccaggccacaaccgcctacttcctgtaccagcagcagggcagactggacaagctgaccatcaccagccagaacctgcagctggaaagcctcagaatgaaactgggcccgggcccgggcaaaagcgcgttttatattctgccgagcattattagcaacgaaaaaggcccgggcccgggcccgaacatgctgcgcattatggcgagcctggtgctggcgcgcaaaggcccgggcccgggcctgagctattataaactgggcgcgagccagcgcgtggcgggcgatggcccgggcccgggcgcggaaattctgctgattattatgcgcacctttaaagtgagcattggcccgggcccgggcgatttttatctgtgctttctggcgtttctgctgtttctggtgctgggcccgggcccgggcatgaaatttctggtgtttctgggcattattaccaccgtggcggcgggcagcggcgaaggccgcggcagcctgctgacctgcggcgatgtggaagaaaacccgggcccggtgctgctgcaggccggcagcctgcacgccaagaaagaagcggcggcgaaatatctggcgaccgcgctgctgaccctggcggcgtattggctgatgtggctgattattaacctggcggcgtatagcctgccgggcgtgttttgcggcgtggcggcgtataaactgagctatggcattgcgaccgtggcggcgtatctgctgctggatgattttgtggaaattgcggcgtattttgtgtttctggtgctgctgccgctggcggcgtatgcgctgaacaccctggtgaaacagctggcggcgtatcgcctgcagagcctgcagacctatgtggcggcgtattttattgcgggcctgattgcgattgtggcggcgtattttctggcgtttgtggtgtttctgctggcggcgtattttctgctgaacaaagaaatgtatctggcggcgtatattattttttggtttagcctggaactggcggcgtattatattgatattggcaactataccgtggcggcgtattatattaacgtgtttgcgtttccgtttgcggcgtataacgtgtttgcgtttccgtttaccattggcagcggcgcgaccaactttagcctgctgaaacaggcgggcgatgtggaagaaaacccgggcccggtgctgctgcaggccggcagcctgcacgccaagaaagaagcggcggcgaaatatctggcgaccgcgctgctgaccctggcggcgtattggctgatgtggctgattattaacctggcggcgtatagcctgccgggcgtgttttgcggcgtggcggcgtataaactgagctatggcattgcgaccgtggcggcgtatctgctgctggatgattttgtggaaattgcggcgtattttgtgtttctggtgctgctgccgctggcggcgtatgcgctgaacaccctggtgaaacagctggcggcgtatcgcctgcagagcctgcagacctatgtggcggcgtattttattgcgggcctgattgcgattgtggcggcgtattttctggcgtttgtggtgtttctgctggcggcgtattttctgctgaacaaagaaatgtatctggcggcgtatattattttttggtttagcctggaactggcggcgtattatattgatattggcaactataccgtggcggcgtattatattaacgtgtttgcgtttccgtttgcggcgtataacgtgtttgcgtttccgtttaccatttaa

SEQ ID NO: 322 - DNA, 602 bp, synthetic construct
  misc_feature 1..602: note = Fragment of DNA encoding peptide sequence comprising multiple SARS-Cov-2 epitopes
  source 1..602: mol_type = other DNA
atgtggacgaccagagagatctgatcagcaatcacgagcagctgccaatcctgggcaaccggcctagagaacctgagagatgcagcagaggcgctctgtacaccggcgtgagcgtgctggtggctctgttgttggctggccaggccacaaccgcctacttcctgtaccagcagcagggcagactggacaagctgaccatcaccagccagaacctgcagctggaaagcctcagaatgaaactgggcccgggcccgggcaaaagcgcgttttatattctgccgagcattattagcaacgaaaaaggcccgggcccgggcccgaacatgctgcgcattatggcgagcctggtgctggcgcgcaaaggcccgggcccgggcctgagctattataaactgggcgcgagccagcgcgtggcgggcgatggcccgggcccgggcgcggaaattctgctgattattatgcgcacctttaaagtgagcattggcccgggcccgggcgatttttatctgtgctttctggcgtttctgctgtttctggtgctgggcccgggcccgggcatgaaatttctggtgtttctgggcattattaccaccgtggcggcg

SEQ ID NO: 323 - DNA, 63 bp, synthetic construct; identical to SEQ ID NO: 216
  misc_feature 1..63: note = Fragment of DNA encoding peptide sequence comprising multiple SARS-Cov-2 epitopes
  source 1..63: mol_type = other DNA

SEQ ID NO: 324 - DNA, 582 bp, synthetic construct; identical to SEQ ID NO: 319
  misc_feature 1..582: note = Fragment of DNA encoding peptide sequence comprising multiple SARS-Cov-2 epitopes
  source 1..582: mol_type = other DNA

SEQ ID NO: 325 - DNA, 66 bp, synthetic construct
  misc_feature 1..66: note = Fragment of DNA encoding peptide sequence comprising multiple SARS-Cov-2 epitopes
  source 1..66: mol_type = other DNA
ggcagcggcgcgaccaactttagcctgctgaaacaggcgggcgatgtggaagaaaacccgggcccg

SEQ ID NO: 326 - DNA, 585 bp, synthetic construct
  misc_feature 1..585: note = Fragment of DNA encoding peptide sequence comprising multiple SARS-Cov-2 epitopes
  source 1..585: mol_type = other DNA
gtgctgctgcaggccggcagcctgcacgccaagaaagaagcggcggcgaaatatctggcgaccgcgctgctgaccctggcggcgtattggctgatgtggctgattattaacctggcggcgtatagcctgccgggcgtgttttgcggcgtggcggcgtataaactgagctatggcattgcgaccgtggcggcgtatctgctgctggatgattttgtggaaattgcggcgtattttgtgtttctggtgctgctgccgctggcggcgtatgcgctgaacaccctggtgaaacagctggcggcgtatcgcctgcagagcctgcagacctatgtggcggcgtattttattgcgggcctgattgcgattgtggcggcgtattttctggcgtttgtggtgtttctgctggcggcgtattttctgctgaacaaagaaatgtatctggcggcgtatattattttttggtttagcctggaactggcggcgtattatattgatattggcaactataccgtggcggcgtattatattaacgtgtttgcgtttccgtttgcggcgtataacgtgtttgcgtttccgtttaccatttaa

SEQ ID NO: 327 - DNA, 1941 bp, synthetic construct
  misc_feature 1..1941: note = DNA encoding peptide sequence comprising multiple SARS-Cov-2 epitopes
  source 1..1941: mol_type = other DNA
atgatggacgaccagcgcgatctgatcagcaaccacgagcagctgcctatcctgggcaatagacctagagagcctgagcggtgcagcaggggcgctctgtacactggagtgtccgtgctggtggctctgctgctggccggccaggccaccaccgcctacttcctgtaccagcagcagggcagactggataagctgaccatcaccagccagaatctgcaactcgagagcctcagaatgaagctgggcccaggccctggaaagtctgccttctacatcctgcctagcatcatcagcaatgagaagggccccggccccggacccaacatgctgagaatcatggccagcctggtactggcccggaagggccccggcccaggcctgagctactacaagctgggcgctagccagagagtggccggcgacggccccggccccggcgccgagattctgctgatcatcatgcggacattcaaggtgtccatcggccccggccctggagacttctacctgtgcttcctggccttcctgctgtttctggtgctgggcccaggcccgggcatgaaattcctggtgtttctgggcatcatcacaacagtggccgctggctctggcgagggcagaggcagcctgctgacatgcggggacgtggaagaaaaccccggacctgtgctgctgcaggccggcagcctgcacgccaagaaagaggccgccgccaagtacctggcaaccgccctgctgaccctggctgcctactggctgatgtggctgatcatcaacctggccgcctatagcctgccaggagtgttctgtggagtggccgcttacaaactgagctacggcatcgccacagtggccgcttacctgctgttagatgatttcgtggaaatcgctgcctacttcgtgttcctggtgctgctgcctctggctgcatacgccctgaacaccctggtcaagcaactggccgcttatagactgcagagcctgcagacctacgtggccgcttacttcatcgccggcctgatcgccattgtggccgcctactttctggcctttgtggtgtttctgctggccgcctacttcctgctcaacaaggaaatgtacctggccgcttacatcatcttctggttcagcctcgagctggccgcctactacatcgacatcggcaactacacggttgctgcctattacatcaacgtgttcgcctttcctttcgccgcttacaacgtgttcgccttccccttcaccatcggcagcggcgccacaaacttcagcctgctcaagcaggccggagatgtggaagagaaccctggacctaagggcagcccttggaagggatctctgctgctgctgctgatgaacatcgtcagcaatctgctgctttgtcagtctgtggcccctctgcctaagaagtctcagtgcgtgaacctgacaaccagaacccagctgccacctgcctataccaacagctttaccagaggggtctacaagaaagacgccgtggactgcgccctggatcctctgtccgagacaaagtgcaccctgaaatccttcaccgtggaaaagggaatctaccagacaagcaacaaaaagggcacaaacacatctaaccaggtggccgtgctctatcaggacgttaactgcaccgaggtgcctgtggcgatccacgccgaccagctgacacctacctggcgggtgtacagcaccggctctaagaaagtgtgcggccctaagaagtccaccaacctggtgaagaataagtgtgtgaatttcaacttcaacggcctgaccggcaccggtgttctgacggaaagcaacaagaagttcctgcctttccagcaatttggcagagacatcgccgatacaaccgacgccgtgcgggacccccagaccctggaaatcctggacattaccccttgttctttcggcggcgtgagcgtgatc

SEQ ID NO: 328 - DNA, 603 bp, synthetic construct
  misc_feature 1..603: note = Fragment of DNA encoding peptide sequence comprising multiple SARS-Cov-2 epitopes
  source 1..603: mol_type = other DNA
atgatggacgaccagcgcgatctgatcagcaaccacgagcagctgcctatcctgggcaatagacctagagagcctgagcggtgcagcaggggcgctctgtacactggagtgtccgtgctggtggctctgctgctggccggccaggccaccaccgcctacttcctgtaccagcagcagggcagactggataagctgaccatcaccagccagaatctgcaactcgagagcctcagaatgaagctgggcccaggccctggaaagtctgccttctacatcctgcctagcatcatcagcaatgagaagggccccggccccggacccaacatgctgagaatcatggccagcctggtactggcccggaagggccccggcccaggcctgagctactacaagctgggcgctagccagagagtggccggcgacggccccggccccggcgccgagattctgctgatcatcatgcggacattcaaggtgtccatcggccccggccctggagacttctacctgtgcttcctggccttcctgctgtttctggtgctgggcccaggcccgggcatgaaattcctggtgtttctgggcatcatcacaacagtggccgct

SEQ ID NO: 329 - DNA, 63 bp, synthetic construct
  misc_feature 1..63: note = Fragment of DNA encoding peptide sequence comprising multiple SARS-Cov-2 epitopes
  source 1..63: mol_type = other DNA
ggctctggcgagggcagaggcagcctgctgacatgcggggacgtggaagaaaaccccggacct

SEQ ID NO: 330 - DNA, 582 bp, synthetic construct
  misc_feature 1..582: note = Fragment of DNA encoding peptide sequence comprising multiple SARS-Cov-2 epitopes
  source 1..582: mol_type = other DNA
gtgctgctgcaggccggcagcctgcacgccaagaaagaggccgccgccaagtacctggcaaccgccctgctgaccctggctgcctactggctgatgtggctgatcatcaacctggccgcctatagcctgccaggagtgttctgtggagtggccgcttacaaactgagctacggcatcgccacagtggccgcttacctgctgttagatgatttcgtggaaatcgctgcctacttcgtgttcctggtgctgctgcctctggctgcatacgccctgaacaccctggtcaagcaactggccgcttatagactgcagagcctgcagacctacgtggccgcttacttcatcgccggcctgatcgccattgtggccgcctactttctggcctttgtggtgtttctgctggccgcctacttcctgctcaacaaggaaatgtacctggccgcttacatcatcttctggttcagcctcgagctggccgcctactacatcgacatcggcaactacacggttgctgcctattacatcaacgtgttcgcctttcctttcgccgcttacaacgtgttcgccttccccttcaccatc

SEQ ID NO: 331 - DNA, 66 bp, synthetic construct
  misc_feature 1..66: note = Fragment of DNA encoding peptide sequence comprising multiple SARS-Cov-2 epitopes
  source 1..66: mol_type = other DNA
ggcagcggcgccacaaacttcagcctgctcaagcaggccggagatgtggaagagaaccctggacct

SEQ ID NO: 332 - DNA, 627 bp, synthetic construct
  misc_feature 1..627: note = Fragment of DNA encoding peptide sequence comprising multiple SARS-Cov-2 epitopes
  source 1..627: mol_type = other DNA
aagggcagcccttggaagggatctctgctgctgctgctgatgaacatcgtcagcaatctgctgctttgtcagtctgtggcccctctgcctaagaagtctcagtgcgtgaacctgacaaccagaacccagctgccacctgcctataccaacagctttaccagaggggtctacaagaaagacgccgtggactgcgccctggatcctctgtccgagacaaagtgcaccctgaaatccttcaccgtggaaaagggaatctaccagacaagcaacaaaaagggcacaaacacatctaaccaggtggccgtgctctatcaggacgttaactgcaccgaggtgcctgtggcgatccacgccgaccagctgacacctacctggcgggtgtacagcaccggctctaagaaagtgtgcggccctaagaagtccaccaacctggtgaagaataagtgtgtgaatttcaacttcaacggcctgaccggcaccggtgttctgacggaaagcaacaagaagttcctgcctttccagcaatttggcagagacatcgccgatacaaccgacgccgtgcgggacccccagaccctggaaatcctggacattaccccttgttctttcggcggcgtgagcgtgatc

SEQ ID NO: 333 - protein, 45 aa, Homo sapiens
GIINTLQKYYCRVRGGRCAVLSCLPKEEQIGKCSTRGRKCCRRKK

SEQ ID NO: 334 - protein, 5 aa, synthetic construct
  REGION 1..5: note = Peptide linker
EAAAK

SEQ ID NO: 335 - protein, 4 aa, Homo sapiens
TKPR

SEQ ID NO: 336 - protein, 6 aa, synthetic construct
  REGION 1..6: note = Targeting peptide
RKRGLK

SEQ ID NO: 337 - protein, 9 aa, synthetic construct
  REGION 1..9: note = Targeting peptide
RLYRKRGLK

SEQ ID NO: 338 - protein, 12 aa, synthetic construct
  REGION 1..12: note = Targeting peptide
CGGKLGRKRYLR